(19) United States
(12) Patent Application Publication
(10) Pub. No.: US 2012/0035214 A1
(43) Pub. Date: Feb. 9, 2012

(54) RENIN INHIBITORS

(75) Inventors: Daniel J. McKay, Lexington, MA (US); Melissa Arbour, Pierrefonds (CA); Renee Aspiotis, Kirkland (CA); Austin Chen, San Diego, CA (US); Pierre-Andre Fournier, Vaudreuil-Dorion (CA); Michel Gallant, Pierrefonds (CA); Yongxin Han, Kirkland (CA); Helene Juteau, St. Laurent (CA); Patrick Lacombe, Montreal (CA); Sebastien Laliberte, St. Lazare (CA); Sophie Lauzon, St. Lazare (CA); Carmela Molinaro, Lansdale, PA (US); Paul O'Shea, Princeton, NJ (US); Yeeman Ramtohul, Pierrefonds (CA); Daniel Simard, Brookline, MA (US); Dwight MacDonald, L'lle Bizard (CA); Bruce Mackay, Dollard-des-Ormeaux (CA); Christophe Mellon, L'lle Bizard (CA); Krista Morley, Brossard (CA); Chit Tsui, Toronto (CA); Sarah Jennifer Dolman, New York, NY (US); Greg Hughes, Scotch Plains, NJ (US); Jacob Janey, New York, NY (US)

(73) Assignees: Merck Canada Inc., Kirkland (CA); Merck Sharp & Dohme Corp., Rahway, NJ (US)

(21) Appl. No.: 13/262,781

(22) PCT Filed: Apr. 1, 2010
(86) PCT No.: PCT/US10/29588

§ 371 Date: Oct. 3, 2011

Related U.S. Application Data
(60) Provisional application No. 61/211,827, filed on Apr. 3, 2009.

(51) Int. Cl. A61K 31/451; C07D 401/04; C07D 401/10; C07D 211/26; C07D 211/28; C07D 401/12; A61K 31/4545; A61K 31/454; A61P 9/12; A61P 9/04; A61P 9/10; A61P 27/06; A61P 25/22; A61P 25/00; A61P 13/12; C07D 211/22

(52) U.S. Cl. 514/318; 546/236; 546/194; 546/199; 546/234; 546/232; 546/211; 546/229; 514/317; 514/322; 514/331; 514/326

(57) ABSTRACT

The present invention relates to biaryl piperidine-based renin inhibitor compounds, and their use in treating cardiovascular events and renal insufficiency.

### CROSS-REFERENCE TO RELATED APPLICATIONS

Not Applicable

### JOINT RESEARCH AGREEMENT

The claimed invention was made as a result of activities undertaken within the scope of a joint research agreement between Merck & Co., Inc. and Actelion Pharmaceuticals Ltd. The agreement was executed on Dec. 4, 2003. The field of the invention is described below.

### FIELD OF THE INVENTION

The invention relates to novel renin inhibitors of the general Formula I, II or The invention also concerns related aspects including processes for the preparation of the compounds, pharmaceutical compositions containing one or more compounds of Formula I, II or III and especially their use as renin inhibitors in cardiovascular events and renal insufficiency.

### BACKGROUND OF THE INVENTION

In the renin-angiotensin system (RAS) the biologically active angiotensin II (Ang II) is generated by a two-step mechanism. The highly specific enzyme renin cleaves of angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J, A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients, inhibition of ACE can lead to bradykinin accumulation, causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis.

The compounds described in this invention represent a novel structural class of renin inhibitors.

### SUMMARY OF THE INVENTION

The present invention is directed to certain compounds and their use in the inhibition of the renin enzyme, including treatment of conditions known to be associated with the renin system.

The invention in particular is directed to compounds of formula (I), (II) and (III) as follows:

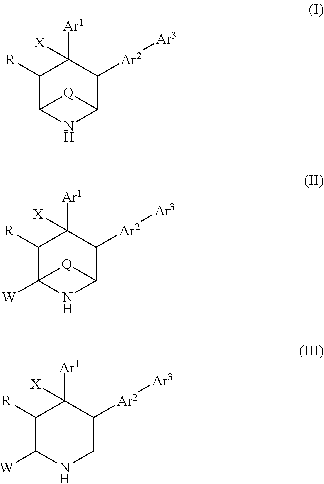

and optically pure enantiomers, mixtures of enantiomers such as racemates, diastereomers, mixtures of diastereomers, diastereomeric racemates, mixtures of diastereomeric racemates, meso-forms, salts, solvates, and morphological forms thereof, wherein constituent members are provided herein.

### DETAILED DESCRIPTION OF THE INVENTION

The present invention provides compounds having formula (I), (II) and (III) as follows:

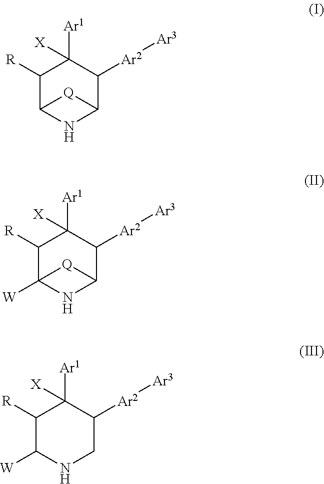

or pharmaceutically acceptable salts thereof, optical isomers thereof, or prodrugs thereof, wherein:

Ar¹ is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of

### 1) oxo (═O)

### 2) OH,

### 3) CN,

4) halogen,

### 5) NH₂,

### 6) COOH,

### 7) OCF₂H,

### 8) OCF₃,

### 9) CF₃,

10) C₁-C₆alkyl,
11) OC₁-C₆alkyl,
12) C₃-C₆cycloalkyl,
13) OC₃-C₆cycloalkyl,
14) C₂-C₆alkenyl,
15) C₁-C₆alkoxy,
16) (CH₂)₁₋₃OC₁-C₃alkyleneOC₁-C₆alkyl,
17) O(CH₂)₁₋₃OC₁-C₃alkyleneOC₁-C₆alkyl,
18) (CH₂)₁₋₃OC₁-C₃alkyleneOC₃-C₆cycloalkyl,
19) C(O)NHC₁-C₆alkyl,
20) NHC(O)C₁-C₆alkyl,
21) S(O)₀₋₂C₁-C₆alkyl,

### 22) O(CH₂)₁₋₄OAr⁴, and

### 23) (CH₂)₁₋₅OAr⁴,

wherein Ar⁴ is as described herein, and wherein substituents (10)-(21) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy, and
i) S(O)₀₋₂C₁-C₆alkyl;

Ar² is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of:

### 1) OH,

### 2) CN,

3) halogen,

### 4) COOH,

### 5) OCF₂H,

### 6) OCF₃,

### 7) CF₃,

8) C₁-C₆alkyl,
9) C₃-C₆cycloalkyl,
10) C₂-C₆alkenyl,
11) C₁-C₆alkoxy,
12) C(O)C₁-C₆alkyl,
13) NHC(O)C₁-C₆alkyl, and
14) S(O)₀₋₂C₁-C₆alkyl,

### 15) R²

### 16) R³

wherein substituents (8)-(14) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy, and
1) S(O)₀₋₂C₁-C₆alkyl,

wherein R² is selected from the group consisting of:

### a) H,

b) C₁-C₆alkyl,
c) C₃-C₆cycloalkyl,
d) C₀-C₃alkyleneC(O)C₁-C₆alkyl,
e) C₀-C₃alkyleneC(O)OR¹, and
f) C₀-C₃alkyleneCONHR¹, and

wherein R¹ is as defined herein,

wherein R³ is selected from the group consisting of:

1) halogen as long as it is not attached to an N atom directly,
2) hydrogen,

### 3) CF₃,

4)
5) C₃-C₆cycloalkyl,

### 6) CH(OH),

7) C(O)C₁-C₆alkyl, and
8) CH(OH)C₁-C₆alkyl;

unsubstituted or substituted with one, two, three or four substituents independently selected from the group consisting of: OH, halogen, CF₃, and C₁-C₃alkyl,

Ar³ is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of:

### 1) OH,

### 2) CN,

3) halogen,

### 4) COOH,

### 5) OCF₂H,

### 6) OCF₃,

### 7) CF₃,

8) C₁-C₆alkyl,
9) C₃-C₆cycloalkyl,
10) C₂-C₆alkenyl,
11) C₁-C₆alkoxy,
12) C₁-C₃alkyleneNH₂,
13) C₁-C₃alkyleneNHC(O)NH₂,
14) C₁-C₃alkyleneOC₁-C₆alkyl,
15) OC₁-C₃alkyleneOC₁-C₆alkyl,
16) C₁-C₃alkyleneNHC(O)C₁-C₆alkyl,
17) C₁-C₃alkyleneNHC(O)OC₁-C₆alkyl,
18) C₁-C₃alkyleneNHC(O)NR¹C₁-C₆alkyl,
19) C₁-C₃alkyleneNHC(O)C₃-C₆cycloalkyl,
20) C₁-C₃alkyleneNHC(O)OC₃-C₆cycloalkyl,
21) C₁-C₃alkyleneNHC(O)NR¹C₃-C₆cycloalkyl,
22) C₁-C₃alkyleneNHS(O)₂C₁-C₆alkyl,
23) C₀-C₃alkyleneC(O)NR¹C₁-C₆alkyl,
24) C₀-C₃alkyleneS(O)₀₋₂C₁-C₆alkyl,
25) C₁-C₃alkyleneNHC(O)Ar⁵,
26) C₀-C₃alkyleneO(CH₂)₀₋₃Ar⁵,
27) C₁-C₃alkyleneNHC(O)O(CH₂)₀₋₃Ar⁵, and
28) C₀-C₃alkyleneHet,

wherein Het, Ar⁵, and R¹ are as described herein, wherein substituents (8)-(22) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy,
i) C₁-C₃alkyleneOC₁-C₆alkyl, and
j) S(O)₀₋₂C₁-C₆alkyl, and

wherein substituents (23)-(26) are unsubstituted or substituted with 1-3 halogens,

Ar⁴ is an unsubstituted or substituted 5- or 6-membered aryl ring, or an unsubstituted or substituted 5 or 6-membered heteroaryl ring containing 1 to 2 heteroatoms independently selected from O, S and N, wherein the substituents on the substituted aryl ring or substituted heteroaryl ring consist of one, two or three substituents independently selected from the group consisting of:

### 1) CN,

2) halogen,

### 3) OCF₂H,

### 4) OCF₃,

### 5) CF₃,

6) C₁-C₆alkyl,
7) C₃-C₆cycloalkyl,
8) C₁-C₆alkoxy, and
9) S(O)₀₋₂C₁-C₆alkyl,

wherein substituents (6)-(9) are unsubstituted or substituted with one, two, three or four substituents independently selected from the group consisting of:

### a) OH,

### b) COOH,

### c) CN,

### d) CF₃,

e) C₁-C₆alkoxy, and
f) S(O)₀₋₂C₁-C₆alkyl, and

Ar⁵ is an unsubstituted or substituted aryl ring, or an unsubstituted or substituted heteroaryl ring containing 1 to 4 heteroatoms independently selected from O, S and N, wherein the substituents on the substituted aryl ring and substituted heteroaryl ring consist of one, two or three substituents independently selected from the group consisting of:

### 1) OH,

### 2) CN,

3) halogen,

### 4) COOH,

### 5) OCF₂H,

### 6) OCF₃,

### 7) CF₃,

8) C₁-C₆alkyl,
9) C₃-C₆cycloalkyl,
10) C₂-C₆alkenyl,
11) C₁-C₆alkoxy,

wherein substituents (8)-(11) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₁-C₆cycloalkyl,
h) C₁-C₆alkoxy, and
i) S(O)₀₋₂C₁-C₆alkyl,

R is selected from the group consisting of:

1) hydrogen,
2) halogen,

### 3) OH,

### 4) COOH,

5) COOC₁-C₆alkyl,

### 6) CF₃,

### 7) CN,

8) C₁-C₆alkyl,
9) C₃-C₆cycloalkyl,
10) (CH₂)₁₋₃OC₁-C₆alkyl,
11) OC₁-C₆alkyl, and
12) CONHC₁-C₆alkyl,

wherein substituents (8)-(12) are unsubstituted or substituted with one, two, three or four substituents independently selected from the group consisting of:

### a) OH,

b) halogen,

### c) COOH,

### d) CN,

### e) CF₃,

f) C₆alkyl,
g) C₃-C₆cycloalkyl,
h) C₁-C₆alkoxy, and
i) S(O)₀₋₂C₁-C₆alkyl;

R¹ is selected from the group consisting of:

1) hydrogen,
2) C₁-C₆alkyl, and
3) C₃-C₆cycloalkyl,

wherein substituents (2)-(3) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₁-C₆alkoxy, and
h) S(O)₀₋₂C₁-C₆alkyl;

W is selected from the group consisting of:

1) hydrogen,

### 2) CN,

### 3) COOR¹,

4) C₁-C₆alkyl, and
5) C₃-C₆cycloalkyl;

wherein alkyl and cycloalkyl are unsubstituted or substituted with 1-3 substituents selected from the group consisting of:

a) halogen,

### b) OR¹

### c) COOR¹,

### d) CN,

### e) CONHR¹,

### f) CON(R¹)R⁴,

### g) OC(O)N(R¹)R⁴,

### h) NHC(O)N(R¹)R⁴

### i) NHC(O)R¹, and

j) NHC(O)Ar⁴;

wherein R⁴ is selected from the group consisting of: hydrogen, C₁-C₆alkyl, C₃-C₆cycloalkyl, and Ar⁴; wherein R¹ and Ar⁴ are as defined herein,

X is selected from the group consisting of

1) hydrogen,

### 2) OH,

### 3) CN,

4) halogen,

### 5) COOH,

### 6) CF₃,

7) COOC₁-C₆alkyl,
8) OC₁-C₆alkyl,

### 9) OC(O)NH₂

### 10) O(CH₂)₁₋₃Ar⁵, and

### 11) O(CH₂)₁₋₃Het,

wherein substituents (7)-(8) and (10)-(11) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) OC₁-C₆alkyl, and
h) S(O)₀₋₂C₁-C₆alkyl,

Het is a 4-7 (and, in specific embodiments, 5-7)-membered substituted or unsubstituted heterocyclic ring containing 1-4 heteroatoms independently selected from O, N and S. Het, in specific embodiments, is selected from, but not limited to, unsubstituted or substituted tetrazole, thiophene, furan, 1,2,3-triazole, 1,2,4-triazole, pyrazole, isoxazole, oxazole, thiazole, isothiazole, 1,2,5-oxadiazole, 1,2,4-oxadiazole, 1,2,5-thiadiazole, pyridine, pyrimidine, pyrazine, pyridazine, imidazole, morpholine, piperidine, piperazine, azetidine, pyrrolidine, oxetane, tetrahydrofuran, tetrahydropyran, 1,3-dioxolan-2-one, 1,3-oxazolidin-2-one, 1,3-thiazolidin-2-one, pyrrolidin-2-one, piperidin-2-one, 1,3-oxazinan-2-one, tetrahydropyrimidin-2(1H)-one, imidazolidin-2-one, imidazolidine-2,4-dione, 1,3-dihydro-2H-imidazol-2-one, and oxo-substituted heterocyclic rings; and

Q is absent or selected from the group consisting of:

1) a bond,

### 2) CH₂,

### 3) CH₂CH₂,

### 4) CH₂OCH₂,

### 5) CH₂S(O)₀₋₂CH₂,

### 6) CH₂NR²CH₂, and

### 7) CONR²CH₂,

wherein R² is as defined above for Art.

‘Absent’ in respect to the above variable Q means that carbons on the piperidine ring of compounds of Formulas (I) and (II) otherwise attached to Q are saturated as exemplified in the following structure where W is as defined elsewhere herein:

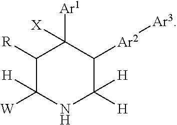

In specific embodiments, Q is absent.

In specific embodiments of compounds of Formula I, II or III, Ar¹ is an unsubstituted or substituted aryl ring, or an unsubstituted or substituted heteroaryl ring containing 1-3 N heteroatoms, wherein the substituents on the substituted aryl ring and substituted heteroaryl ring consist of one, two or three substituents independently selected from the group consisting of: oxo, halogen, C₁-C₆alkyl, OC₁-C₆alkyl, NH₂, (CH₂)₁₋₃OC₁-C₃alkyleneOC₁-C₆alkyl, O(CH₂)₁₋₃OC₁-C₃alkyleneOC₁-C₆alkyl, and (CH₂)₁₋₃OC₁-C₃alkyleneOC₃-C₆cycloalkyl, wherein the alkyl substituents are unsubstituted or substituted with 1-3 halogen or C₁-C₆alkyl substituents and wherein all other variables are as previously defined.

In another embodiment of compounds of Formula I, II or III, Ar¹ is one of the following ring structures:

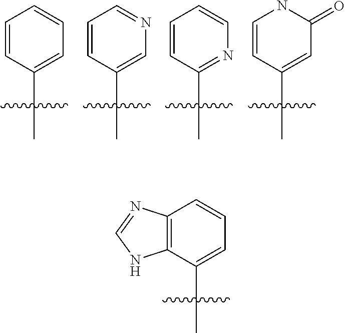

unsubstituted or substituted at any carbon atom with one, two or three substituents independently selected from the group consisting of:

### 1) oxo (═O),

### 2) OH,

### 3) CN,

4) halogen,

### 5) NH₂,

### 6) COOH,

### 7) OCF₂H,

### 8) OCF₃,

### 9) CF₃,

10) C₁-C₆alkyl,
11) OC₁-C₆alkyl,
12) C₃-C₆cycloalkyl,
13) C₃-C₆cycloalkyl,
14) C₂-C₆alkenyl,
15) C₁-C₆alkoxy,
16) (CH₂)₁₋₃OC₁-C₃alkyleneOC₁-C₆alkyl,
17) O(CH₂)₁₋₃OC₁-C₃alkyleneOC₁-C₆alkyl,
18) (CH₂)₁₋₃OC₁-C₃alkyleneOC₃-C₆cycloalkyl,
19) C(O)NHC₁-C₆alkyl,
20) NHC(O)C₁-C₆alkyl,
21) S(O)₀₋₂C₁-C₆alkyl,

### 22) O(CH₂)₁₋₄OAr⁴, and

### 23) (CH₂)₁₋₅OAr⁴,

wherein Ar⁴ is as described herein, and wherein substituents (10)-(21) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy, and
i) S(O)₀₋₂C₁-C₆alkyl;

and all other variables are as defined herein.

In another embodiment of compounds of Formula I, II or III, Ar¹ is one of the following ring structures:

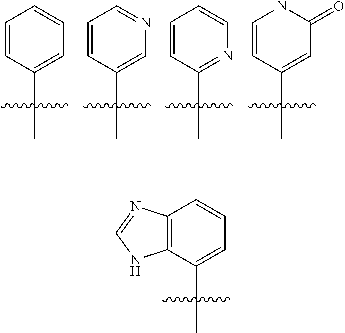

unsubstituted or substituted at any carbon atom with one, two or three substituents independently selected from the group consisting of: OH, NH₂, CN, halogen, C₁-C₆alkyl, OC₁-C₆alkyl, C₃-C₆cycloalkyl, OC₁-C₆alkyl, OC₃-C₆cycloalkyl, (CH₂)₁₋₃OC₁-C₃alkyleneOC₁-C₆alkyl, (CH₂)₁₋₃OC₁-C₃alkyleneOC₃-C₆cycloalkyl, O(CH₂)₁₋₄OAr⁴ and (CH₂)₁₋₅OAr⁴; said C₁-C₆alkyl, C₃-C₆cycloalkyl, OC₁-C₆alkyl, OC₃-C₆cycloalkyl, (CH₂)₁₋₃OC₁-C₃alkyleneOC₁-C₆alkyl, O(CH₂)₁₋₃OC₁-C₃alkyleneOC₁-C₆alkyl, (CH₂)₁₋₃OC₁-C₃alkyleneOC₃-C₆cycloalkyl, O(CH₂)₁₋₄OAr⁴ and (CH₂)₁₋₅OAr⁴ substituents unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy, and
i) S(O)₀₋₂C₁-C₆alkyl,

wherein Ar⁴ is as described herein, and all other variables are as defined herein.

In another embodiment of compounds of Formula I, II or III, Ar¹ is selected from the group consisting of:

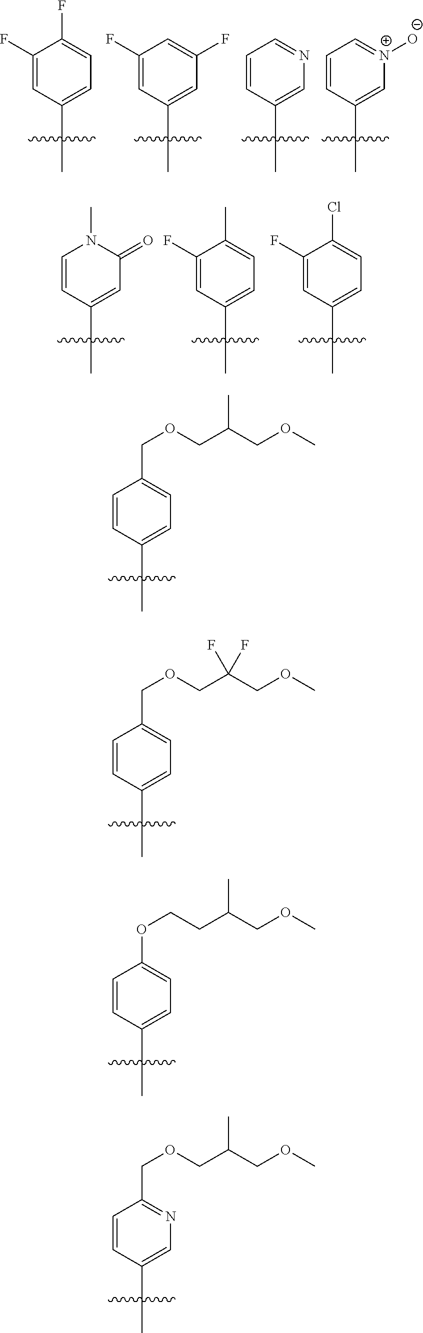

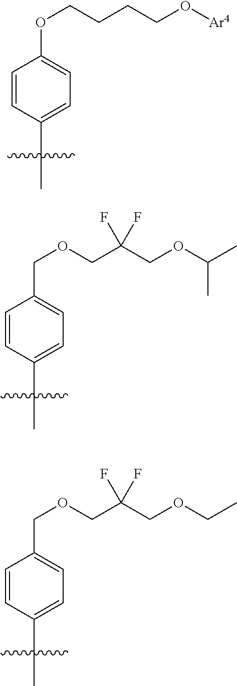

unsubstituted substituted at any carbon atom with one, two, three or four substituents independently selected from the group consisting of: halogen, CF₃ and methyl, where Ar⁴ is as described herein and wherein all other variables are as previously defined.

In another embodiment of compounds of Formula I, II or III, Ar¹ is selected from the group consisting of:

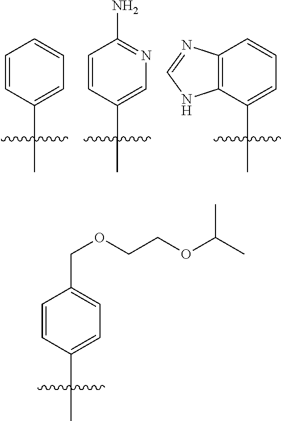

unsubstituted or substituted at any carbon atom with one, two, three or four substituents independently selected from the group consisting of: halogen, CF₃ and methyl, and all other variables are as previously defined.

In specific embodiments, Ar1 is a phenyl or pyridyl, unsubstituted or substituted as described herein.

In another embodiment of compounds of Formula I, II or III, Ar²-Ar³ is selected from one of the following ring structures:

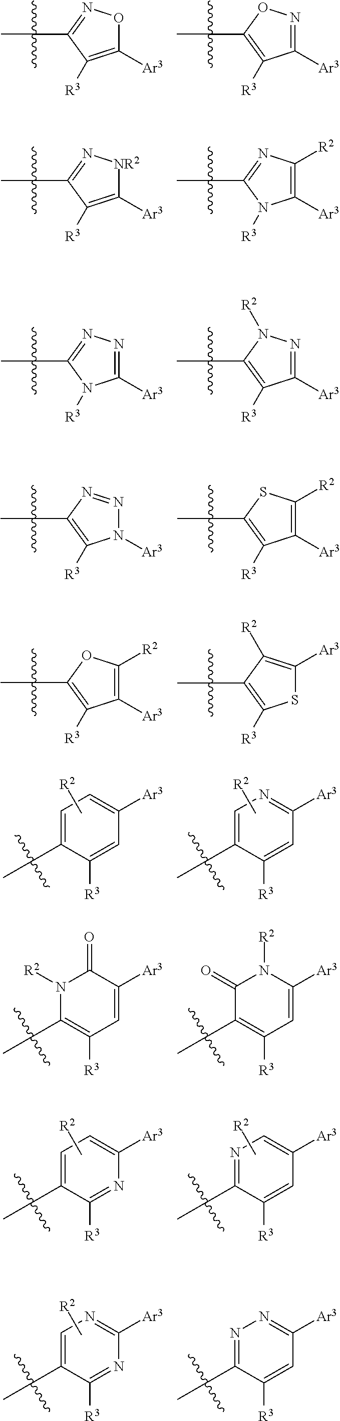

wherein R², R³, and A³ are as defined herein,

and all other variables are as defined herein,

In another embodiment of compounds of Formula I, II or III, Ar²-Ar³ is selected from one of the following ring structures:

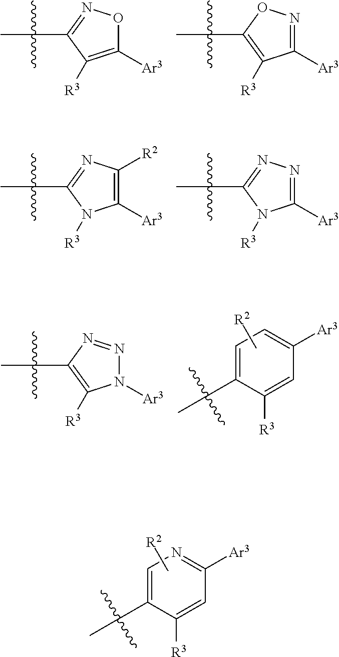

wherein R², R³, and Ar³ are as defined herein, and

wherein all other variables are as defined herein.

In another embodiment of compounds of Formula I, II or III, Ar²-Ar³ is selected from the group consisting of:

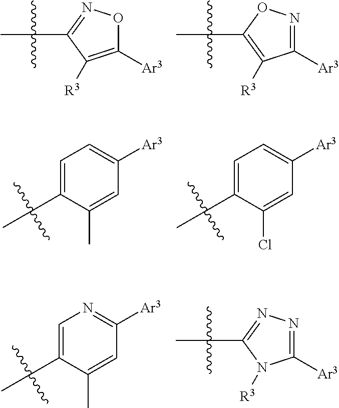

wherein Ar³ is as described herein, wherein R³ is selected from the group consisting of: hydrogen, halogen, Me, Et, isopropyl, cyclopropyl, CF₃, CH₃C(O), CH₃CH₂C(O), CH₃CH(OH) and CH₃CH₂CH(OH); said isopropyl, cyclopropyl, CH₃C(O), CH₃CH₂C(O), CH₃CH(OH) and CH₃CH₂CH(OH) unsubstituted or substituted with 1-3 halogens,

and wherein all other variables are as defined herein.

In specific embodiments of compound of Formula I, II or III, Ar³ is selected from the group consisting of:

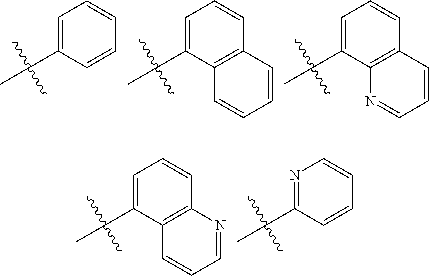

unsubstituted or substituted at any carbon atom with one, two or three substituents independently selected from the group consisting of:

### 1) OH,

### 2) CN,

3) halogen,

### 4) COOH,

### 5) OCF₂H,

### 6) OCF₃,

### 7) CF₃,

8) C₁-C₆alkyl,
9) C₃-C₆cycloalkyl,
10) C₂-C₆alkenyl,
11) C₁-C₆alkoxy,
12) C₁-C₃alkyleneNH₂,
13) C₁-C₃alkyleneNHC(O)NH₂,
14) C₁-C₃alkyleneOC₁-C₆alkyl,
15) C₁-C₃alkyleneNHC(O)C₁-C₆alkyl,
16) C₁-C₃alkyleneNHC(O)OC₁-C₆alkyl,
17) C₁-C₃alkyleneNHC(O)NR¹C₁-C₆alkyl,
18) C₁-C₃alkyleneNHC(O)C₃-C₆cycloalkyl,
19) C₁-C₃alkyleneNHC(O)OC₃-C₆cycloalkyl,
20) C₁-C₃alkyleneNHC(O)NR¹C₃-C₆cycloalkyl,
21) C₁-C₃alkyleneNHS(O)₂C₁-C₆alkyl,
22) C₀-C₃alkyleneC(O)NR¹C₁-C₆alkyl,
23) C₀-C₃alkyleneS(O)₀₋₂C₁-C₆alkyl,
24) C₁-C₃alkyleneNHC(O)Ar⁵,
25) C₀-C₃alkyleneO(CH₂)₀₋₃Ar⁵,
26) C₁-C₃alkyleneNHC(O)O(CH₂)₀₋₃Ar⁵, and
27) C₀-C₃alkyleneHet,
wherein Het, Ar⁵, and R¹ are as described herein, wherein substituents (8)-(21) are unsubstituted or substituted with one, two, three or four substituents independently selected from the group consisting of:

### a) OH,

b) halogen,

### c) COOH,

### d) CN,

### e) CF₃,

f) C₁-C₆alkyl,
g) C₃-C₆cycloalkyl,
h) C₁-C₆alkoxy, and
i) C₁-C₃alkyleneOC₁-C₆alkyl, and
j) S(O)₀₋₂C₁-C₆alkyl,

wherein substituents (22)-(25) are unsubstituted or substituted with 1-3 halogens, and

wherein all other variables are as defined herein.

In specific embodiments of compound of Formula I, II or III, Ar³ is selected from the group consisting of:

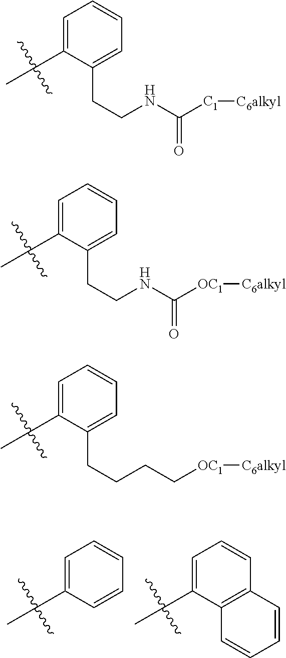

unsubstituted or substituted at any carbon atom with 1-2 groups independently selected from the group consisting of: halogen, CF₃ and Me, wherein R and X are independently selected from the group consisting of: H and OH, Q is absent; and wherein other variables are as defined herein.

In another embodiment of compounds of Formula I, II or III, R is selected from the group consisting of:

### 1) H,

2) halogen,

### 3) OH,

4)
5) COOC₁-C₆alkyl, and
6) CONHC₁-C₆alkyl,
wherein 4)-6) are unsubstituted or substituted with one, two, three or four substituents independently selected from the group consisting of: OH, halogen, CF₃, and C₁-C₃alkyl, and all other variables are as defined herein.

In specific embodiments, R is hydrogen or halogen, and all other variables are as defined herein.

In a specific class, R is hydrogen.

In specific embodiments, W is selected from the group consisting of: hydrogen and C₁-C₆alkyl, unsubstituted or substituted with 1-3 halogen substituents,

In another embodiment of compounds of Formula I, II or III, X is selected from the group consisting of:

### 1) H,

2) halogen,

### 3) OH,

### 4) OC(O)NH₂,

5) OC₁-C₆alkyl, and

### 6) O(CH₂)₁₋₃Het,

wherein Het is as defined herein and wherein 5)-6) are unsubstituted or substituted with one, two, three or four substituents independently selected from the group consisting of: OH, halogen, CF₃, and C₁-C₃alkyl, and all other variables are as defined herein

With respect to any class or subclass of compounds disclosed herein, where a variable is not specifically defined otherwise with respect to that particular class or subclass, it is as defined within the present disclosure for the broader genus. Where specific subclasses are provided for, the various combinations of the different subclasses to arrive at compounds falling with the broadest genus is fully contemplated by the present disclosure. For purposes of exemplification, where narrower subgenuses of different variables are provided, e.g., Ar² and Ar³, one can select a group from a narrow subgenus of Ar² and a group from either the broader genus for Ar³ or a narrower subgenus for Ar³ (and vice versa), in addition to groups falling within the other variables that make up the broader or a more specific genus to select a compound falling with the scope of the present invention. These various combinations are fully contemplated herein.

The compounds of Formula I, II or III above, and pharmaceutically acceptable salts thereof, are renin inhibitors. The compounds are useful for inhibiting renin and treating conditions such as hypertension.

Any reference to a compound of Formula I, II or III is to be understood as referring also to optically pure enantiomers, mixtures of enantiomers such as racemates, diastereomers, mixtures of diastereomers, diastereomeric racemates, mixtures of diastereomeric racemates, meso-forms and tautomers, as well as salts (especially pharmaceutically acceptable salts) and solvates (including hydrates) of such compounds, and morphological forms, as appropriate and expedient. The present invention encompasses all these forms. Mixtures are separated in a manner known per se, e.g. by column chromatography, thin layer chromatography (TLC), high performance liquid chromatography (HPLC), or crystallization. The compounds of the present invention may have chiral centers, e.g. one chiral center (providing for two stereoisomers, (R) and (S)), or two chiral centers (providing for up to four stereoisomers, (R,R), (S,S), (R,S), and (S,R)). This invention includes all of these optical isomers and mixtures thereof. Unless specifically mentioned otherwise, reference to one isomer applies to any of the possible isomers. Whenever the isomeric composition is unspecified, e.g., when bonds to a chiral carbon are depicted as straight lines, it is understood that both (R) and (S) configurations of that chiral carbon and, hence, both enantiomers and mixtures thereof are represented.

Tautomers of compounds defined in Formula I, II or III are also included within the scope of the present invention. For example, compounds including carbonyl —CH₂C(O)— groups (keto forms) may undergo tautomerism to form hydroxyl —CH═C(OH)— groups (enol forms). Both keto and enol forms are included within the scope of the present invention.

In addition, compounds with carbon-carbon double bonds may occur in Z- and E-forms with all isomeric forms of the compounds being included in the present invention.

Compounds of the invention also include nitrosated compounds of Formula I, II or III that have been nitrosated through one or more sites such as oxygen (hydroxyl condensation), sulfur (sulfydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983).

In the compounds of structural Formula I, II or III, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural Formula I, II or III. For example, different isotopic forms of hydrogen (H) include protium (¹H) and deuterium (²H, also denoted as D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples, Isotopically-enriched compounds within structural Formula I, II or III, can be prepared without undue experimentation by conventional techniques well known to those skilled in the art or by processes analogous to those described in the Schemes and Examples herein using appropriate isotopically-enriched reagents and/or intermediates.

Salts are preferably the pharmaceutically acceptable salts of the compounds of Formula I, II or III. The expression “pharmaceutically acceptable salts” encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or, in case the compound of Formula I, II or III is acidic in nature, with an inorganic base like an alkali or earth alkali base, e.g. sodium hydroxide, potassium hydroxide, calcium hydroxide and the like. For other examples of pharmaceutically acceptable salts, reference can be made notably to “Salt selection for basic drugs”, Int. J. Pharm. (1986), 33, 201-217.

The invention also includes derivatives of the compound of Formula I, II or III, acting as prodrugs. These prodrugs, following administration to the patient, are converted in the body by normal metabolic processes to the compound of Formula I. Such prodrugs include those that demonstrate enhanced bioavailability, tissue specificity, and/or cellular delivery, to improve drug absorption of the compound of Formula I, II or III. The effect of such prodrugs may result from modification of physicochemical properties such as lipophilicity, molecular weight, charge, and other physicochemical properties that determine the permeation properties of the drug.

The general terms used hereinbefore in Formula I, II or III and hereinafter preferably have, within this disclosure, the following meanings, unless otherwise indicated. Where the plural form is used for compounds, salts, pharmaceutical compositions, diseases and the like, this is intended to mean also a single compound, salt, or the like.

The term “alkyl”, alone or in combination with other groups, unless indicated otherwise, means saturated, straight and branched chain groups with one to six carbon atoms (which may be represented by “C₁₋₆ alkyl” or “C₁-C₆ alkyl”). When the intended meaning is other than this, for example, when the number of carbon atoms is in the range of one to four carbon atoms, this meaning is represented in like fashion as “C₁₋₄ alkyl” or “C₁-C₄ alkyl”. Examples of alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

Structural depictions of compounds may show a terminal methyl group as: “—CH₃”, “CH₃”, “-Me”, “Me”, or “ [symbol] ” (i.e., these have equivalent meanings). A terminal ethyl group may be depicted as “—CH₂CH₃”, “CH₂CH₃”, “-Et”, “Et” or

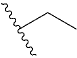

(i.e. these have equivalent meanings).

The term “alkylene” refers to any divalent linear or branched chain aliphatic hydrocarbon radical having a number of carbon atoms in the specified range. Thus, for example, “—C₁-C₆ alkylene-” refers to any of the C₁ to C₆ linear or branched alkylenes, and “—C₁-C₄ alkylene-” refers to any of the C₁ to C₄ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest is an alkylene selected from the group consisting of: —CH₂—, —CH(CH₃)—, and —CH(CH₃)₂—. Expressions such as “C₁-C₄ alkylene-phenyl” and “C₁-C₄ alkylene substituted with phenyl” have the same meaning and are used interchangeably.

The term “alkenyl”, alone or in combination with other groups, unless indicated otherwise, means unsaturated (i.e., having at least one double bond) straight and branched chain groups with two to six carbon atoms (which may be represented by “C₂₋₆ alkenyl” or “C₂-C₆ alkenyl”). When the intended meaning is other than this, for example, when the number of carbon atoms is in the range of two to four carbon atoms, this meaning is represented in like fashion as “C₂₋₄ alkenyl” or “C₂-C₄ alkenyl”.

The term “alkenylene” refers to any divalent linear or branched chain aliphatic mono-unsaturated hydrocarbon radical having a number of carbon atoms in the specified range. The term “alkynyl”, alone or in combination with other groups, unless indicated otherwise, means unsaturated (i.e., having at least one triple bond) straight and branched chain groups with two to six carbon atoms (which may be represented by “C₂₋₆ alkynyl” or “C₂-C₆ alkynyl”). When the intended meaning is other than this, for example, when the number of carbon atoms is in the range of two to four carbon atoms, this meaning is represented in like fashion as “C₂₋₄ alkynyl” or “C₂-C₄ alkynyl”.

The term “alkoxy”, alone or in combination with other groups, refers to an R—O— group, wherein R is an alkyl group. Examples of alkoxy groups are methoxy, ethoxy, propoxy, iso-propoxy, iso-butoxy, sec-butoxy and tert-butoxy.

The term “hydroxy-alkyl”, alone or in combination with other groups, refers to an HO—R— group, wherein R is an alkyl group. Examples of hydroxy-alkyl groups are HO—CH₂—, HO—CH₂CH₂—, HO—CH₂CH₂CH₂— and CH₃CH(OH)—.

The term “halogen” means fluorine, chlorine, bromine or iodine, preferably fluorine, chlorine or bromine, especially fluorine or chlorine.

The term “cycloalkyl”, alone or in combination with other groups, unless indicated otherwise, means a saturated cyclic hydrocarbon ring system with 3 to 8 carbon atoms, e.g., cyclopropyl, cyclobutyl, cyclopentyl, cyclohexyl, cycloheptyl and cyclooctyl. This may be represented by “C₃₋₈ cycloalkyl” or “C₃-C₈ cycloalkyl”). When the intended meaning is other than this, for example, when the number of carbon atoms is in the range of three to six carbon atoms, this meaning is represented in like fashion as “C₃₋₆ cycloalkyl” or “C₃-C₆ cycloalkyl”.

The term “aryl”, alone or in combination, relates to a phenyl, naphthyl or indanyl group, preferably a phenyl group. The abbreviation “Ph” represents phenyl. Unless otherwise specified, when the aryl ring has substituents, it is understood that the substituents may be attached to any atom in the ring, provided that a stable chemical structure results.

The term “heteroaryl”, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing two heteroatoms independently selected from oxygen, nitrogen and sulfur and benzofused derivatives of such rings; five-membered aromatic rings containing three nitrogen atoms and benzofused derivatives thereof; a tetrazolyl ring; a thiazinyl ring; or coumarinyl. Examples of such ring systems are furanyl, thienyl, pyrrolyl, pyridinyl, pyridonyl (e.g., 2-hydroxy-pyridynyl), pyrimidinyl, indolyl, quinolinyl, isoquinolinyl, imidazolyl, triazinyl, thiazolyl, isothiazolyl, pyridazinyl, pyrazolyl, oxazolyl, isoxazolyl, benzothienyl, quinazolinyl and quinoxalinyl. Unless otherwise specified, when the heteroaryl ring has substituents, it is understood that the substituents may be attached to any atom in the ring, whether a heteroatom or a carbon atom, provided that a stable chemical structure results. “Benzofused” as referred to herein means a fused ring system, at least one ring of which is phenyl. Representative benzofused rings include, for purposes of illustration and not limitation, benzimidazole and indole.

Specific examples of compounds of Formula I, II and III, and pharmaceutically acceptable salts thereof, include those listed in the Table 1 below.

TABLE 1

| | | | | | QFRET | |
|---|---|---|---|---|---|---|
| | | | | HRMS | (Buffer) | QFRET |
| STR. | | | STEREO- | (M + 1)⁺ | IC₅₀, | (PLASMA) |
| NO. | EX. | STRUCTURE | CHEMISTRY | M/Z | μM | IC₅₀, μM |
| 1 | 12 | 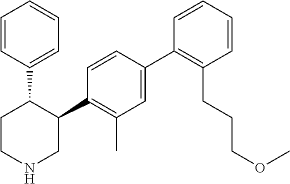 | (+/−) | 400.2629 | 0.62 | >10 |
| 2 | 7 | 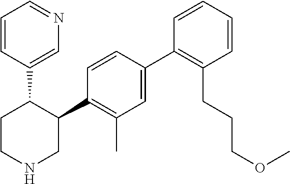 | (+/−) | 401.2592 | 0.018 | 0.31 |
| 3 | 4 | 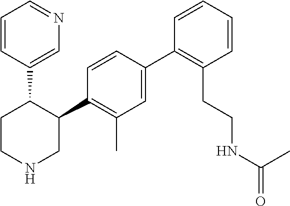 | (+/−) | 414.2542 | 0.0092 | 0.041 |
| 4 | 5 | 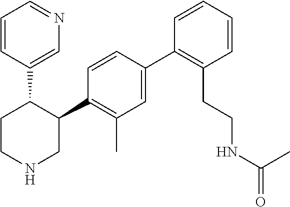 | (3S, 4S) | 414.2549 | 0.0061 | 0.028 |
| 5 | 6 | 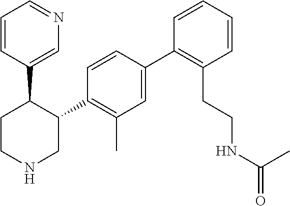 | (3R, 4R) | 414.2557 | 1.41 | 7.28 |
| 6 | 3 | 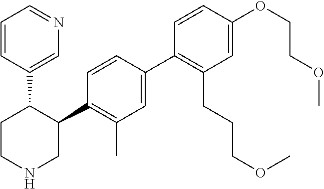 | (+/−) | 475.2962 | 0.38 | 1.80 |
| 7 | 1 | 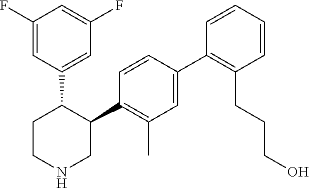 | (+/−) | 422.2288 | 0.085 | 3.15 |
| 8 | 2 | 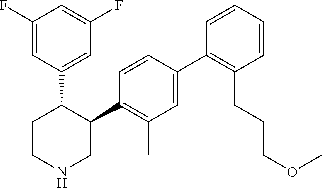 | (+/−) | 436.2451 | 0.0043 | 1.18 |
| 9 | 8 | 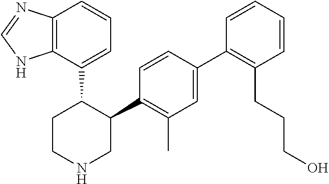 | (+/−) | 426.2549 | 0.998 | >9.5 |
| 10 | 9 | 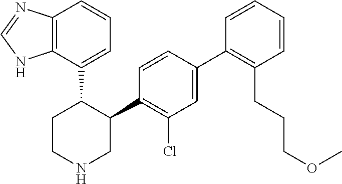 | (+/−) | 460.2159 | 0.0035 | 2.06 |
| 11 | 11 | 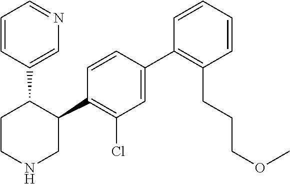 | (+/−) | 421.2056 | 0.0027 | 0.066 |
| 12 | 10 | 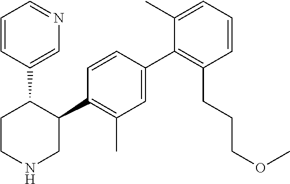 | (+/−) | 415.2764 | 0.010 | 0.15 |
| 13 | 13 | 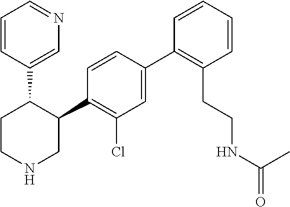 | (3S, 4S) | 434.2019 | 0.0015 | 0.0041 |
| 14 | | 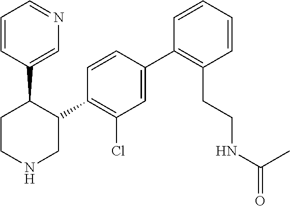 | (3R, 4R) | 434.2003 | 2.63 | >9.5 |
| 15 | | 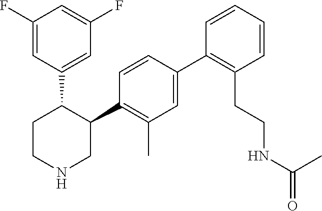 | (+/−) | 449.2428 | 0.0016 | 0.018 |
| 16 | | 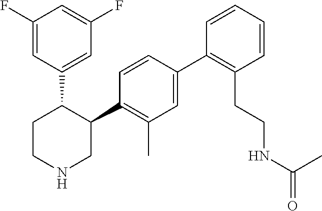 | (3S, 4S) | 449.2400 | 0.00059 | 0.016 |
| 17 | | 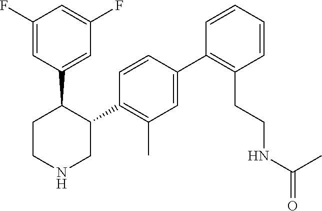 | (3R, 4R) | 449.2411 | 0.53 | 11.65 |
| 18 | | 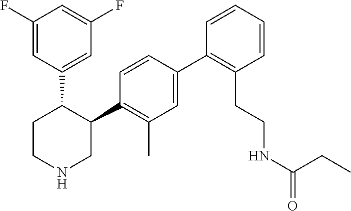 | (+/−) | 463.2585 | 0.00065 | 0.0013 |
| 19 | | 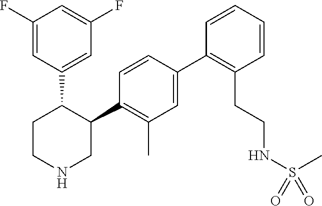 | (+/−) | 485.2078 | 0.071 | 1.81 |
| 20 | | 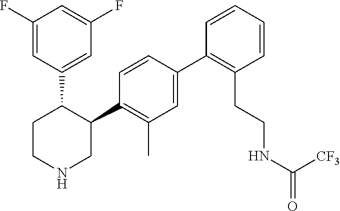 | (+/−) | 503.2120 | 0.00057 | 0.025 |
| 21 | | 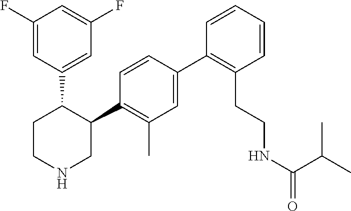 | (+/−) | 477.2707 | 0.014 | 1.15 |
| 22 | | 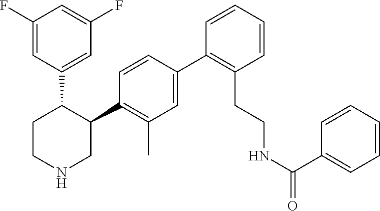 | (+/−) | 511.2575 | 0.020 | 1.17 |
| 23 | | 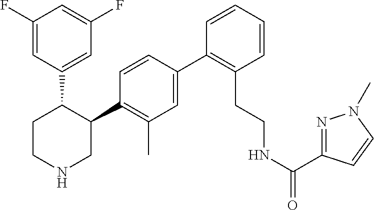 | (+/−) | 515.2618 | 0.026 | 0.069 |
| 24 | | 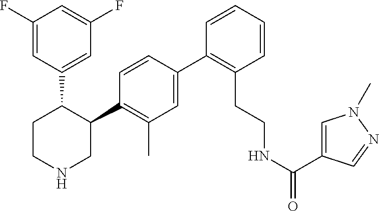 | (+/−) | 515.2623 | 0.073 | 1.01 |
| 25 | | 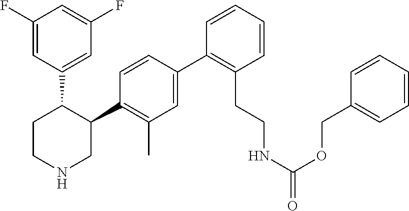 | (+/−) | 541.2678 | 0.018 | 5.47 |
| 26 | | 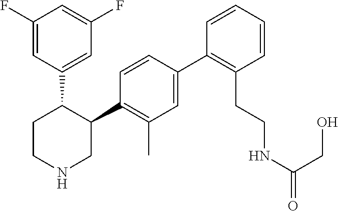 | (+/−) | 465.2348 | 0.0015 | 0.030 |
| 27 | | 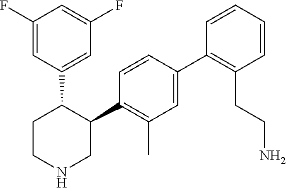 | (+/−) | 407.2318 | 0.081 | 1.04 |
| 28 | | 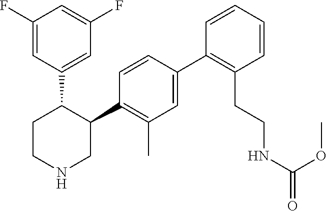 | (+/−) | 465.2341 | 0.00056 | 0.025 |
| 29 | | 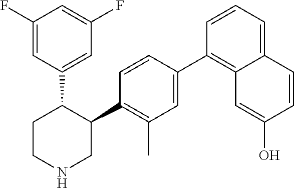 | (+/−) | 430.1977 | 0.093 | >28 |
| 30 | | 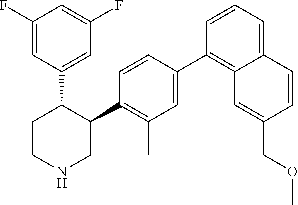 | (+/−) | 458.2283 | 0.10 | 11.3 |
| 31 | | 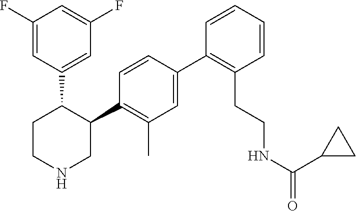 | (+/−) | 475.2555 | 0.0044 | 0.13 |
| 32 | | 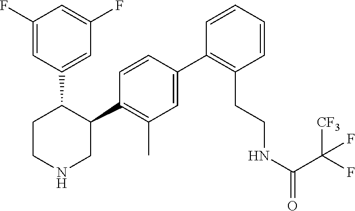 | (+/−) | 553.2081 | 0.023 | 0.075 |
| 33 | | 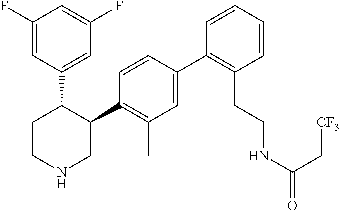 | (+/−) | 517.2296 | 0.0021 | 0.12 |
| 34 | 14 | 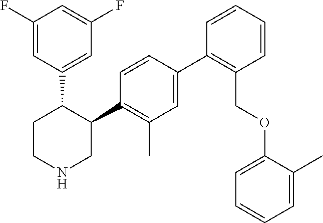 | (+/−) | 484.2453 | 0.0075 | 4.98 |
| 35 | 15 | 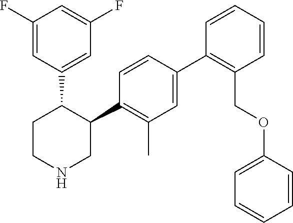 | (+/−) | 470.2285 | 0.0038 | 1.89 |
| 36 | 16 | 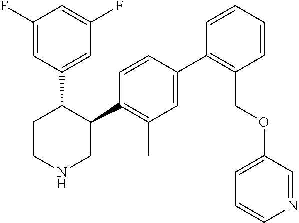 | (+/−) | 471.2247 | 0.00055 | 0.094 |
| 37 | | 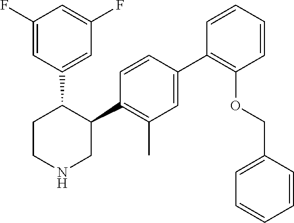 | (+/−) | 470.2313 | 0.087 | 12.41 |
| 38 | | 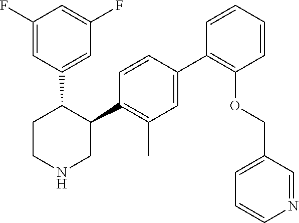 | (+/−) | 471.2198 | 0.15 | >28 |
| 39 | | 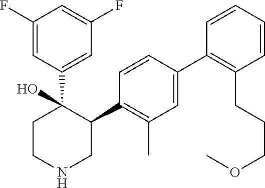 | (+/−) | 452.2394 | 0.0042 | 0.75 |
| 40 | | 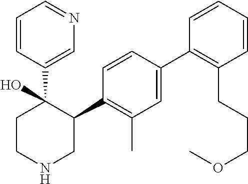 | (+/−) | 417.2553 | 0.047 | 1.14 |
| 41 | | 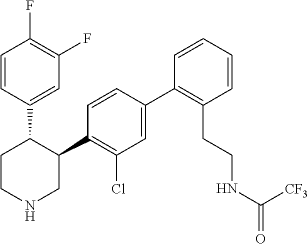 | (+/−) | 523.1597 | 0.0001 | 0.0066 |
| 42 | | 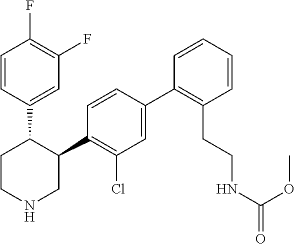 | (+/−) | 485.1796 | 0.00014 | 0.0098 |
| 43 | | 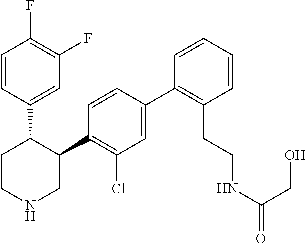 | (+/−) | 485.1785 | 0.00026 | 0.0035 |
| 44 | | 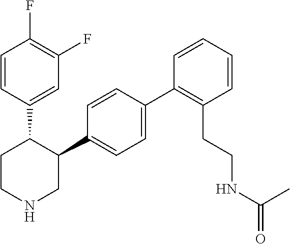 | (+/−) | 435.2250 | 0.0037 | 0.045 |
| 45 | | 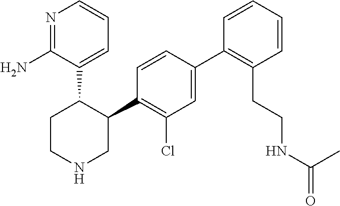 | (+/−) | 449.2115 | 0.71 | 4.58 |
| 46 | | 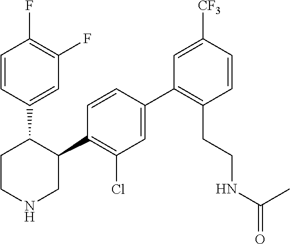 | (+/−) | 537.1756 | 0.00078 | 0.28 |
| 47 | | 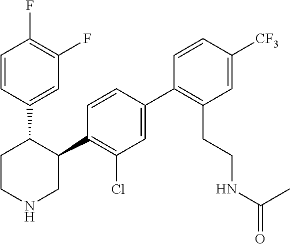 | (+/−) | 537.1735 | 0.00059 | 0.14 |
| 48 | | 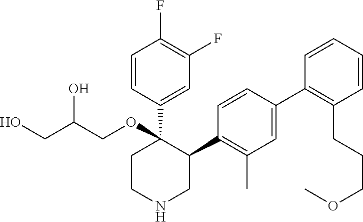 | (+/−) | 526.2769 | 0.016 | 2.02 |
| 49 | | 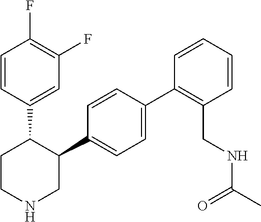 | (+/−) | 421.2087 | 1.71 | 9.04 |
| 50 | | 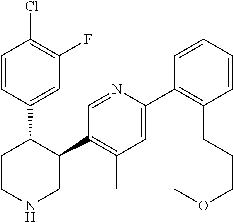 | (+/−) | 453.2103 | 0.0015 | 0.23 |
| 51 | | 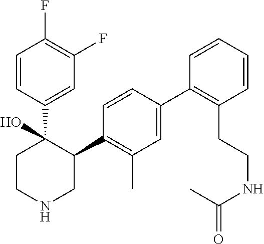 | (+/−) | 465.2360 | 0.0005 | 0.005 |
| 52 | 17 | 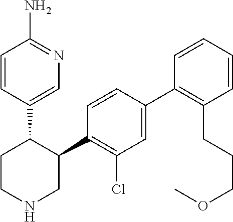 | (+/−) | 436.2175 | 0.15 | 1.63 |
| 53 | 18 | 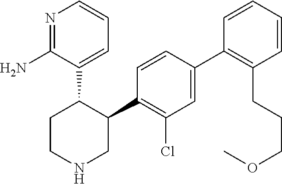 | (+/−) | 436.2209 | 0.012 | 0.25 |
| 54 | 19 | 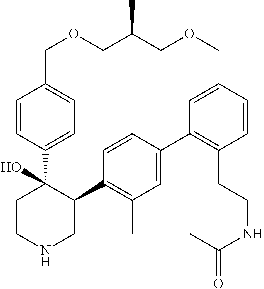 | (+/−) | 545.3380 | 0.00013 | 0.0012 |
| 55 | | 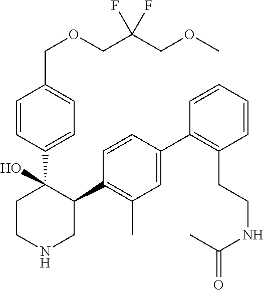 | (+/−) | 567.3027 | 0.00017 | 0.00095 |
| 56 | | 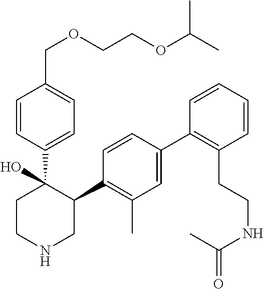 | (+/−) | 545.3399 | 0.00026 | 0.0038 |
| 57 | 23 | 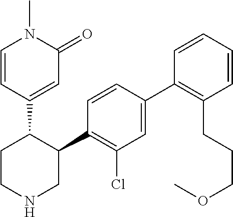 | (+/−) | 451.2176 | 0.0064 | 0.08 |
| 58 | 20 | 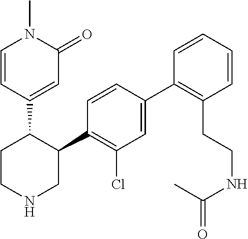 | (+/−) | 464.2131 | 0.0036 | 0.0071 |
| 59 | 21 | 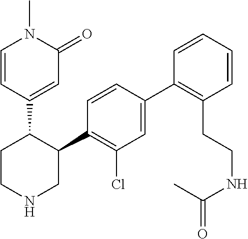 | 3S, 4S | 464.2138 | 0.0021 | 0.0034 |
| 60 | 22 | 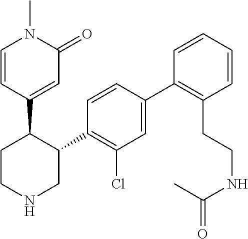 | 3R, 4R | 464.2113 | 0.71 | 1.35 |
| 61 | 68 | 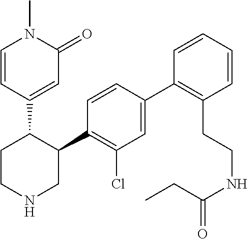 | (+/−) | 478.2281 | 0.0062 | 0.0079 |
| 62 | 69 | 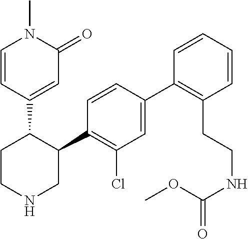 | (+/−) | 480.2088 | 0.0042 | 0.0049 |
| 63 | 70 | 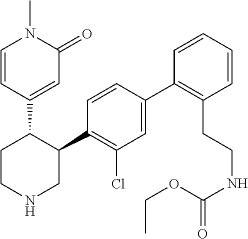 | (+/−) | 494.2215 | 0.016 | 0.024 |
| 64 | 71 | 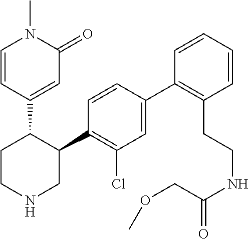 | (+/−) | 494.2215 | 0.11 | 0.077 |
| 65 | 72 | 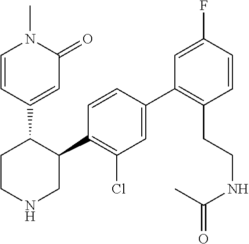 | (+/−) | 482.2015 | 0.0065 | 0.0060 |
| 66 | 73 | 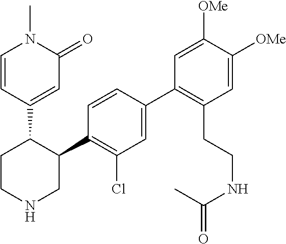 | (+/−) | 524.2323 | 1.32 | 4.90 |
| 67 | | 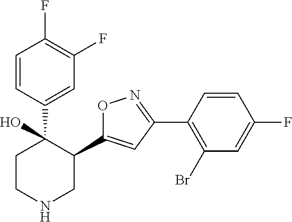 | (3S, 4R) | 453.0424 | 0.40 | 8.65 |
| 68 | 24 | 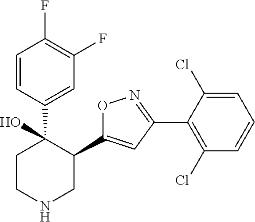 | (3S, 4R) | 425.0641 | 1.21 | >9.5 |
| 69 | 25 | 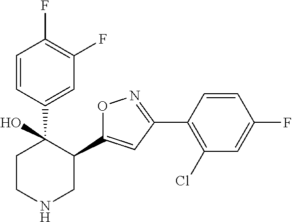 | (3S, 4R) | 409.0926 | 2.50 | >9.5 |
| 70 | 26 | 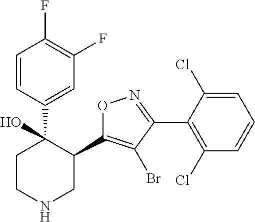 | (3S, 4R) | 504.9743 | 0.0037 | 0.13 |
| 71 | 27 | 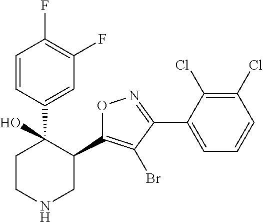 | (3S, 4R) | 504.9712 | 0.00021 | 0.0078 |
| 72 | 28 | 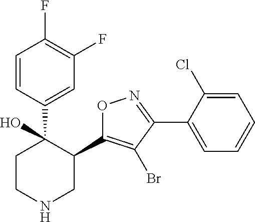 | (3S, 4R) | 469.0134 | 0.0011 | 0.027 |
| 73 | 29 | 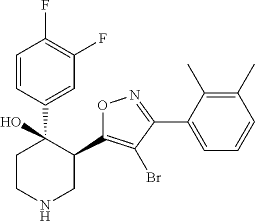 | (3S, 4R) | 463.0838 | 0.00047 | 0.021 |
| 74 | 30 | 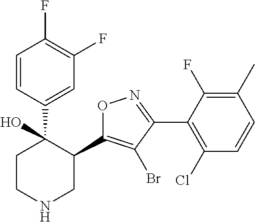 | (3S, 4R) | 501.0196 | 0.0049 | 0.43 |
| 75 | 31 | 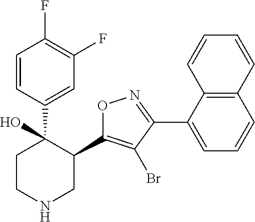 | (3S, 4R) | 485.0721 | 0.000064 | 0.0047 |
| 76 | 32 | 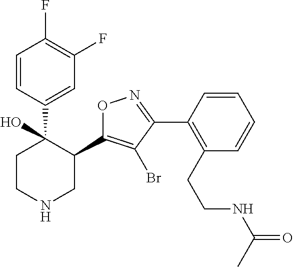 | (3S, 4R) | 520.1084 | 0.00036 | 0.0016 |
| 77 | | 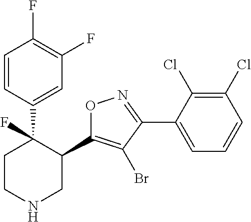 | (3S, 4R) | 506.9666 | 0.00025 | 0.0079 |
| 78 | 37 | 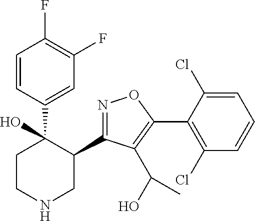 | (3R, 4R) | 469.0865 | 0.0018 | 0.024 |
| 79 | 36 | 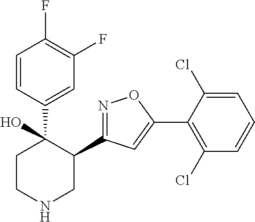 | (3R, 4R) | 425.0621 | 1.33 | >28 |
| 80 | 38 | 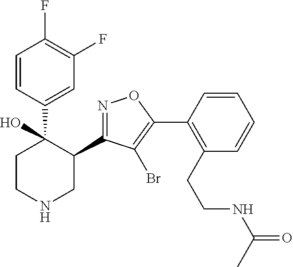 | (3R, 4R) | 520.1053 | 0.00023 | 0.00060 |
| 81 | | 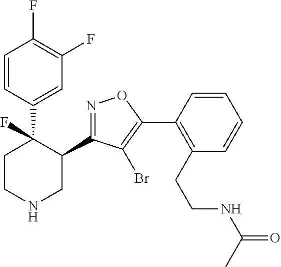 | (3R, 4R) | 522.1002 | 0.000053 | 0.00044 |
| 82 | 39 | 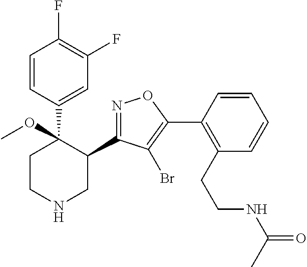 | (3R, 4R) | 534.1199 | 0.00016 | 0.0012 |
| 83 | 40 | 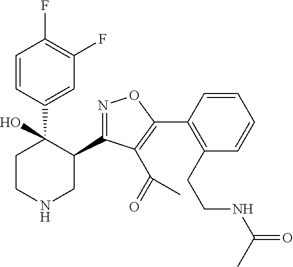 | (3R, 4R) | 484.2009 | 0.0028 | 0.0042 |
| 84 | 43 | 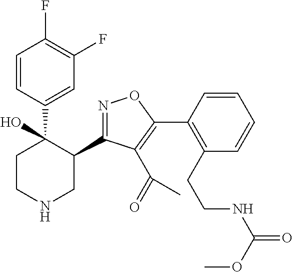 | (3R, 4R) | LRMS (M + H)⁺: 500.5 | 0.010 | 0.093 |
| 85 | 41 | 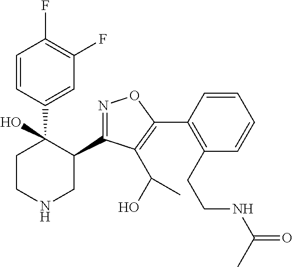 | (3R, 4R) | 486.2227 | 0.0043 | 0.0045 |
| 86 | 33 | 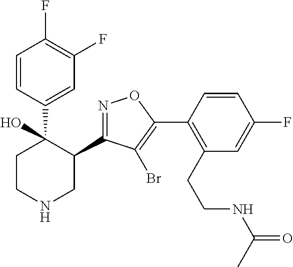 | (3R, 4R) | 538.0922 | 0.00014 | 0.00068 |
| 87 | 34 | 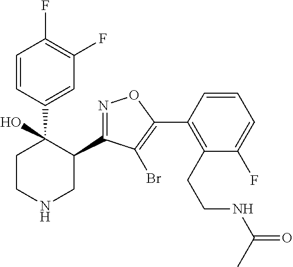 | (3R, 4R) | 538.0952 | 0.00016 | 0.00062 |
| 88 | 42 | 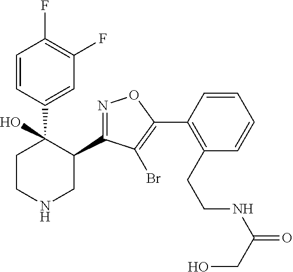 | (3R, 4R) | 536.1014 | 0.00038 | 0.0010 |
| 89 | 35 | 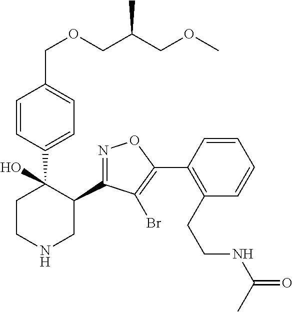 | (3R, 4R) | 600.2087 | 0.00032 | 0.0012 |
| 90 | 44 | 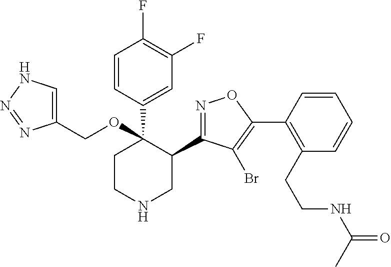 | (3R, 4R) | 601.1403 | 0.00063 | 0.0044 |
| 91 | | 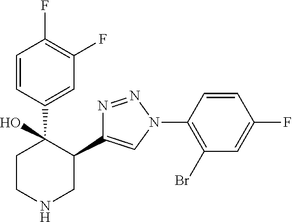 | (3R, 4R) | 453.0527 | 2.90 | 9.47 |
| 92 | | 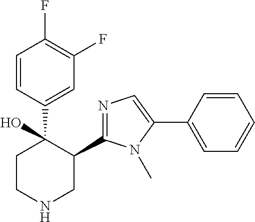 | (3R, 4R) | 370.1755 | 1.99 | >28 |
| 93 | 45 | 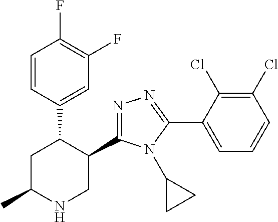 | 3R, 4S | 463.1283 | 0.00014 | 0.00062 |
| 94 | 46 | 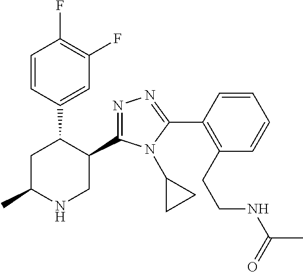 | 3R, 4S | 480.2570 | 0.0015 | 0.003 |
| 95 | 47 | 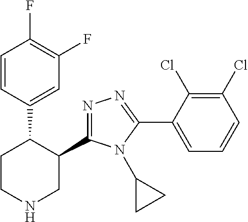 | 3R, 4S | 449.1111 | 0.00015 | 0.00065 |
| 96 | 48 | 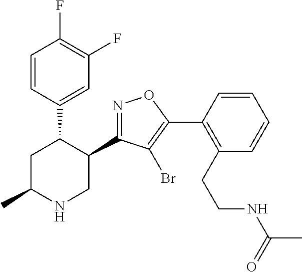 | 3R, 4S | 518.1257 | 0.0007 | 0.0028 |
| 97 | 50 | 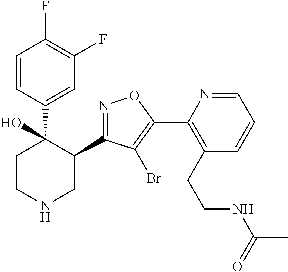 | 3R, 4R | 521.0989 | 0.01 | 0.089 |
| 98 | | 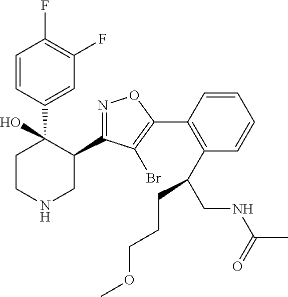 | 3R, 4R | 592.1625 | 0.00058 | 0.0022 |
| 99 | 59 | 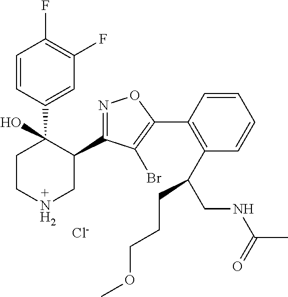 | | | | |
| 100 | | 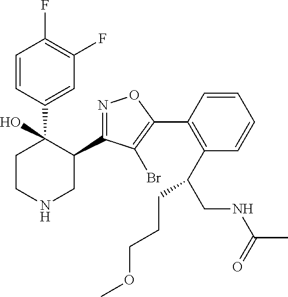 | 3R, 4R | 592.1594 | 0.0012 | 0.0039 |
| 101 | 60 | 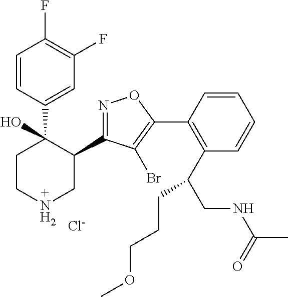 | | | | |
| 102 | | 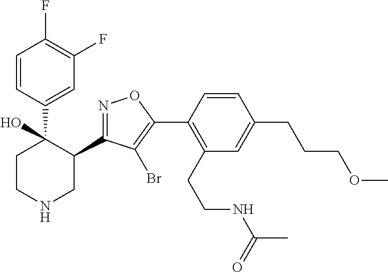 | 3R, 4R | 592.1607 | 0.00027 | 0.0011 |
| 103 | 61 | 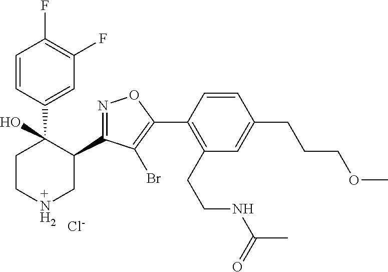 | | | | |
| 104 | 49 | 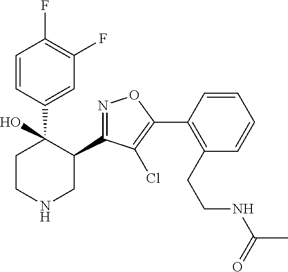 | 3R, 4R | 476.1574 | 0.0007 | 0.0019 |
| 105 | | 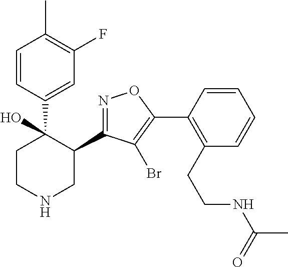 | 3R, 4R | 516.1295 | 0.0049 | 0.029 |
| 106 | 53 | 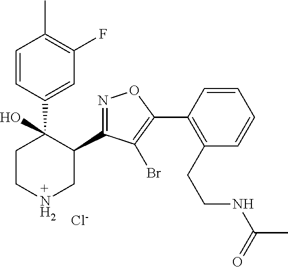 | | | | |
| 107 | 51 | 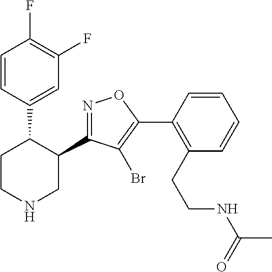 | 3R, 4S | 504.1084 | 0.00026 | 0.0012 |
| 108 | 58 | 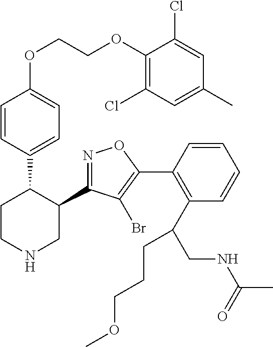 | 3R, 4S | 758.1724 | 0.00007 | 0.002 |
| 109 | 57 | 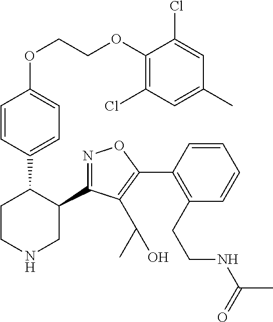 | 3R, 4S | 652.2312 | 0.00007 | 0.0012 |
| 110 | 56 | 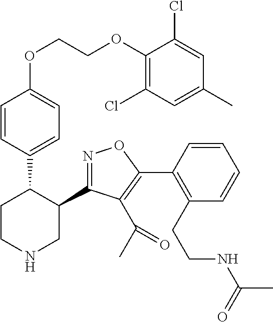 | 3R, 4S | 650.2140 | 0.00009 | 0.0012 |
| 111 | 55 | 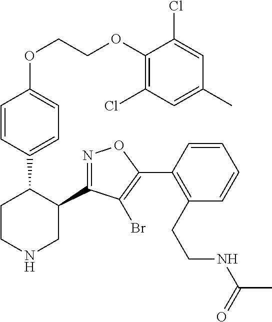 | 3R, 4S | 686.1166 | <0.00005 | 0.0012 |
| 112 | | 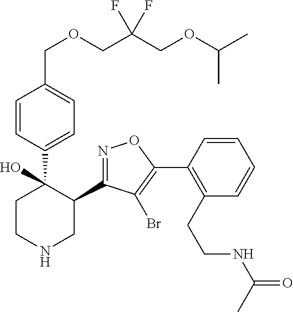 | 3R, 4R | 650.2036 | 0.00044 | 0.0017 |
| 113 | 52 | 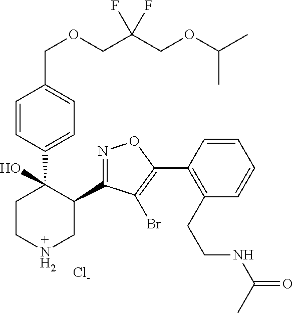 | | | | |
| 114 | | 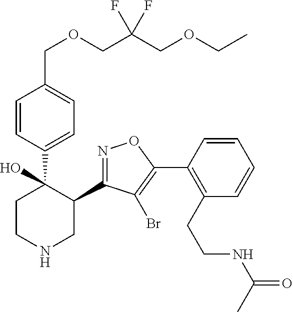 | 3R, 4R | 636.1880 | 0.00019 | 0.00058 |
| 115 | | 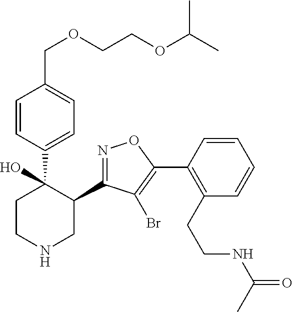 | 3R, 4R | | 0.0065 | 0.016 |
| 116 | 54 | 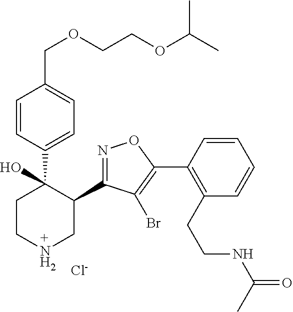 | | | | |
| 117 | | 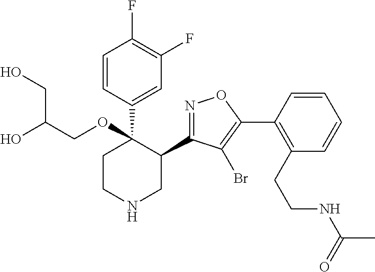 | 3R, 4R | 594.1374 | 0.00037 | 0.0014 |
| 118 | | 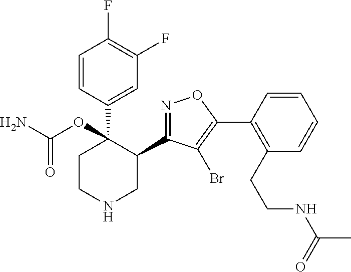 | 3R, 4R | 563.1070 | 0.00022 | 0.00055 |
| 119 | | 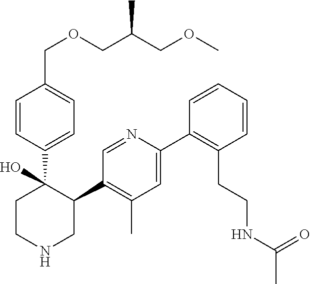 | 3S, 4R | 546.3317 | 0.00015 | 0.0036 |
| 120 | 62 | 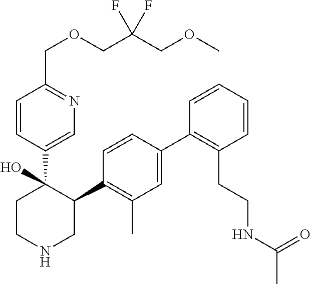 | 3S, 4R | 568.2990 | 0.00066 | 0.0012 |
| 121 | 63 | 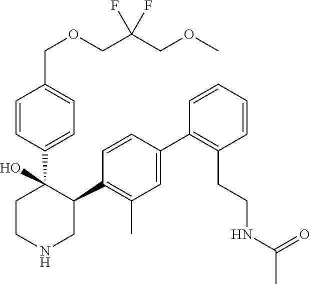 | 3S, 4R | 567.3005 | 0.00008 | 0.00032 |
| 122 | | 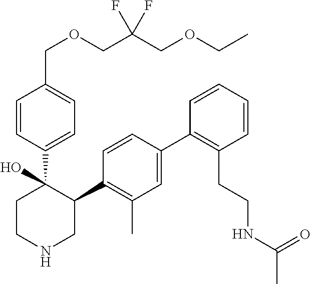 | 3S, 4R | 581.3188 | 0.00009 | 0.00043 |
| 123 | | 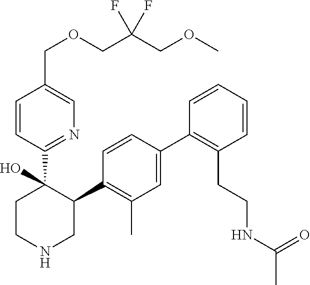 | 3S, 4R | 568.2982 | 0.0012 | 0.0056 |
| 124 | 67 | 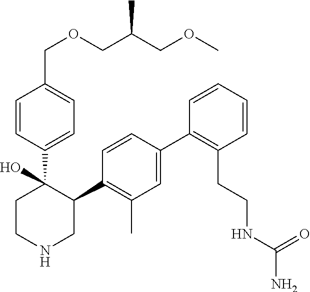 | 3S, 4R | 546.3328 | 0.00063 | 0.0036 |
| 125 | 66 | 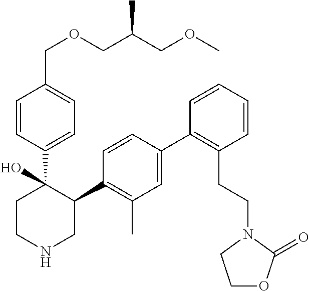 | 3S, 4R | 573.3306 | 0.0046 | 0.076 |
| 126 | | 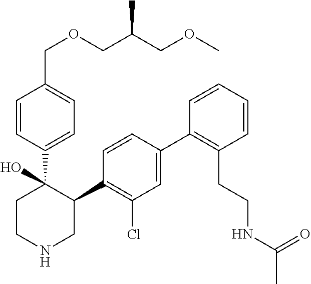 | 3S, 4R | 565.3838 | 0.00008 | 0.00052 |
| 127 | 64 | 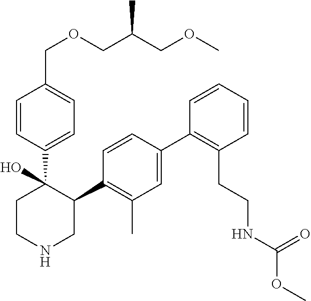 | 3S, 4R | 561.3307 | 0.00003 | 0.0013 |
| 128 | 65 | 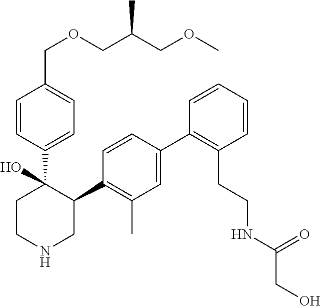 | 3S, 4R | 561.3340 | 0.00007 | 0.00057 |
| 129 | | 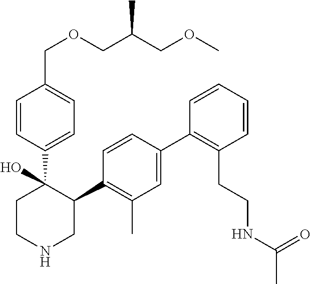 | 3S, 4R | 545.3380 | 0.00005 | 0.00044 |
| 130 | | 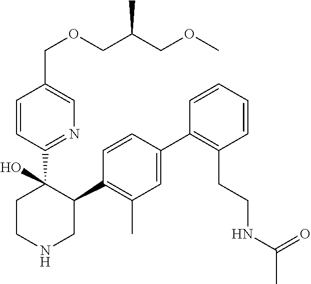 | 3S, 4R | 546.3304 | 0.000075 | 0.0038 |
| 131 | | 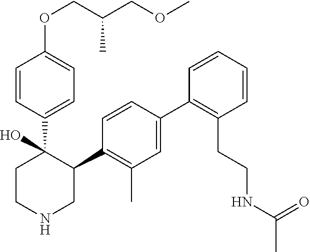 | 3S, 4R | 531.3227 | 0.0012 | 0.018 |
| 132 | | 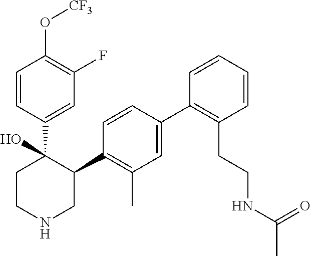 | 3S, 4R | 531.2276 | 0.0004 | 0.018 |

The HRMS data was obtained using standard means available in the art, including the Waters QTOF instrument. The stereochemistry was similarly obtained using standard methods available to those of skill in the art.

The present invention also encompasses a pharmaceutical formulation comprising a pharmaceutically acceptable carrier and the compound of Formula I, II or III or a pharmaceutically acceptable crystal form or hydrate thereof. A preferred embodiment is a pharmaceutical composition of the compound of Formula I, II, or III, comprising, in addition, a second agent.

### LIST OF ABBREVIATIONS

Ac Acetyl group; —C(O)CH₃
ABTS 2,2′-Azino-bis(3-ethylbenzthiazoline-6-sulfonic Acid) 2NH₃

### AIBN Azobisisobutyronitrile

Boc t-butyloxycarbonyl
BSA bovine serum albumin
CBr₄ carbone tetrabromide
CELITE® diatomaceous earth

### DBU 1,8-Diazabicycloundec-7-ene

DCM (CH₂Cl₂) dichloromethane
DIBAL-H diisobutylalumium hydride
DMAP 4-dimethylaminopyridine
DME dimethyl ether

### DMF N,N-dimethylformamide

DMS dimethylsulfide
DMSO dimethylsulfoxide
EDTA ethylenediaminetetraacetic acid
EIA enzyme immunoassay
Eq. equivalent
Et₂O diethylether
EtOAc ethyl acetate
HATU O-(7-azabenzotriazol-1-yl)-N,N,N′,N′-tetramethyluronium hexafluorophosphate
Hex hexane
HMPA hexamethylphosphoramide
LiHMDS lithium hexamethyldisilazide
MeOH methanol
MgSO₄ magnesium sulfate
MTBE methyl tert-butyl ether
NBS N-bromo succinimide
NH₄Cl ammonium chloride
NaB H₄ sodium borohydride
NaHCO₃ sodium bicarbonate
Na₂CO₃ sodium carbonate
Na₂SO₄ sodium sulfate
NCS N-chloro succinimide

### NMO N-methylmorpholine-N-oxide

Pd/C palladium on carbon
PBS phosphate-buffered saline
PPh₃ triphenylphosphine
S-PHOS Dicyclohexylphosphino-2′-6′-dimethoxy-1-1′-biphenyl
TBAF tetrabutylammonium fluoride
TBS tert-butyldimethylsilyl
TBSO tert-butyldimethylsilyloxy
TEA triethylamine
TFA trifluoroacetic acid
THF tetrahydrofuran
Tol toluene
TPAP tetrapropylammonium perruthenate

Unless expressly stated to the contrary, all ranges cited herein are inclusive. For example, an alkyl group described as C₁-C₆ alkyl means the alkyl group can contain 1, 2, 3, 4, 5 or 6 carbon atoms. When a given range includes 0 (e.g., (CH₂)₀₋₃), 0 implies a direct covalent bond.

When any variable occurs more than one time in any constituent or in any formula depicting and describing compounds of the invention, its definition on each occurrence is independent of its definition at every other occurrence.

Also, combinations of substituents and/or variables are permissible only if such combinations result in stable compounds,

The term “substituted” (e.g., as in “aryl which is substituted with 1-3 substituents . . . ”) includes mono- and poly-substitution by a named substituent to the extent such single and multiple substitution (including multiple substitution at the same site) is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

In compounds of the invention having pyridyl N-oxide moieties, the pyridyl-N-oxide portion is structurally depicted using conventional representations such as

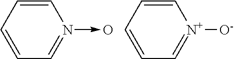

which have equivalent meanings.

The invention relates to a method for the treatment and/or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intraocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of Formula I, II or III for the preparation of a medicament for the treatment and/or prophylaxis of the above-mentioned diseases.

Compounds of Formula I, II or III or the above-mentioned pharmaceutical compositions are also of use in combination with other pharmacologically active compounds comprising ACE-inhibitors, neutral endopeptidase inhibitors, angiotensin H receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases. Specific embodiments employ compounds of Formula I, II or III or the above-mentioned pharmaceutical compositions in combination with cholesterol-lowering drugs, for example, cholesterol absorption inhibitors (e.g., Zetia®) and cholesterol synthesis inhibitors (e.g., Zocor® and Vytorin®), statins (e.g., simvastatin, lovastatin, rosuvastatin, pravastatin, fluvastatin, atorvastatin, rivastatin, and itavastatin) and CETP inhibitors, such as anacetrapib and dalcetrapib.

The term “administration” and variants thereof (e.g., “administering” a compound) in reference to a compound of Formula I, II or III mean providing the compound or a prodrug of the compound to the individual in need of treatment or prophylaxis. When a compound of the invention or a prodrug thereof is provided in combination with one or more other active agents (e.g., an agent such as anangiotensin II receptor antagonist, ACE inhibitor, or other active agent which is known to reduce blood pressure), “administration” and its variants are each understood to include provision of the compound or prodrug and other agents at the same time or at different times. When the agents of a combination are administered at the same time, they can be administered together in a single composition or they can be administered separately. As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.
The term “subject” as used herein refers to an animal, preferably a mammal, most preferably a human, who has been the object of treatment, observation or experiment.
The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit renin and thereby elicit the response being sought (i.e., an “inhibition effective amount”). When the active compound (i.e., active ingredient) is administered as the salt, references to the amount of active ingredient are to the free form (i.e., the non-salt form) of the compound.

In a preferred embodiment, this amount is comprised between 1 mg and 1000 mg per day. In a particularly preferred embodiment, this amount is comprised between 1 mg and 500 mg per day. In a more particularly preferred embodiment, this amount is comprised between 1 mg and 200 mg per day.

In the method of the present invention (i.e., inhibiting renin), the compounds of Formula I, II or III, optionally in the form of a salt or solvate, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, mucosally (including sublingual, buccal, rectal, nasal or vaginal administrations), parenterally (including subcutaneous injection, bolus injection, intraarterial, intravenous, intramuscular, intrasternal injection or infusion administration techniques), by inhalation spray, transdermal, such as passive or iontophoretic delivery, or topical administration, in the form of a unit dosage of a pharmaceutical composition containing an effective amount of the compound and conventional non-toxic pharmaceutically-acceptable carriers, adjuvants and vehicles. Examples of dosage forms include, but are not limited to: tablets, caplets, capsules, such as soft elastic gelatin capsules, cachets, troches, lozenges, dispersions, suppositories, ointments, cataplasms (poultices), pastes, powders, dressings, creams, plasters, solutions, patches, aerosols (e.g., nasal sprays or inhalers), gels, liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs, liquid dosage forms suitable for parenteral administration to a patient, and sterile solids (e.g., crystalline or amorphous solids) that can be reconstituted to provide liquid dosage forms suitable for parenteral administration to a patient. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

The magnitude of prophylactic or therapeutic dose of a compound of Formula I, II or III will, of course, vary with the nature of the severity of the condition to be treated and with the particular compound of Formula I, II or III and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 100 mg in one embodiment from about 0.01 mg to about 50 mg, and in another embodiment from 0.1 mg to 10 mg of a compound of Formula I, II or III per kg of body weight per day.

In the case where an oral composition is employed, a suitable dosage range is, e.g. from about 0.01 mg to about 1000 mg of a compound of Formula I, II or III per day. In one embodiment, the range is from about 0.1 mg to about 10 mg per day. For oral administration, the compositions are preferably provided in the form of tablets containing from 0.01 to 1,000 mg, preferably 0.01, 0.05, 0.1, 0.5, 1, 2, 2.5, 3, 4, 5, 6, 7, 8, 9, 10, 12, 12.5, 15, 20, 25, 30, 40, 50, 100, 250, 500, 750 or 1000 milligrams of the active ingredient for the symptomatic adjustment of the dosage to the patient to be treated.

Another aspect of the present invention provides pharmaceutical compositions which comprises a compound of Formula I, II or III and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, II or III, additional active ingredient(s), and pharmaceutically acceptable excipients.

Any suitable route of administration may be employed for providing a mammal, particularly a human or companion animal such as a dog or cat, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I, II or III as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers, or as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I, II or III in suitable propellants, such as fluorocarbons or hydrocarbons and dry powder inhalation (DPI) aerosol, which may be formulated as a dry powder of a compound of Formula I, II or III with or without additional excipients.

Suitable topical formulations of a compound of Formula I, II or III include transdermal devices, aerosols, creams, solutions, ointments, gels, lotions, dusting powders, and the like. The topical pharmaceutical compositions containing the compounds of the present invention ordinarily include about 0.005% to 5% by weight of the active compound in admixture with a pharmaceutically acceptable vehicle. Transdermal skin patches useful for administering the compounds of the present invention include those well known to those of ordinary skill in that art.

In practical use, the compounds of Formula I, II or III can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I, II or III may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredient, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet cachet or capsule contains from about 0.01 to 1,000 mg, particularly 0.01, 0.05, 0.1, 0.5, 1.0, 2, 2.5, 3, 4, 5, 6, 7, 8, 9, 10, 12, 15, 25, 30, 40, 50, 75, 100, 125, 150, 175, 180, 200, 225, 250, 500, 750 and 1,000 milligrams of the active ingredient for the symptomatic adjustment of the dosage to the patient to be treated.

Additional suitable means of administration of the compounds of the present invention include injection, intravenous bolus or infusion, intraperitoneal, subcutaneous, intramuscular, intranasal, and topical, with or without occlusion.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I, II or III:

| Injectable Suspension (I.M.) | mg/mL | |
|---|---|---|
| Compound of Formula I, II or III | 10 | |
| Methylcellulose | 5.0 | |
| Tween 80 | 0.5 | |
| Benzyl alcohol | 9.0 | |
| Benzalkonium chloride | 1.0 | |
| Water for injection to a total volume of 1 mL | | |
| Tablet | mg/tablet | |
| Compound of Formula I, II or III | 25 | |
| Microcystalline Cellulose | 415 | |
| Povidone | 14.0 | |
| Pregelatinized Starch | 43.5 | |
| Magnesium Stearate | 2.5 | |
| | 500 | |
| Capsule | mg/capsule | |
| Compound of Formula I, II or III | 25 | |
| Lactose Powder | 573.5 | |
| Magnesium Stearate | 1.5 | |
| | 600 | |
| Aerosol | Per canister | |
| Compound of Formula I, II or III | 24 | mg |
| Lecithin, NF Liq. Conc. | 1.2 | mg |
| Trichlorofluoromethane, NF | 4.025 | g |
| Dichlorodifluoromethane, NF | 12.15 | g |

Compounds of Formula I, II or III may be used in combination with other drugs that are used in the treatment/prevention/suppression or amelioration of the diseases or conditions for which compounds of Formula I, II or III are useful. Such other drugs may be administered, by a route and in an amount commonly used therefor, contemporaneously or sequentially with a compound of Formula I, II or M. When a compound of Formula I, II or III is used contemporaneously with one or more other drugs, a pharmaceutical composition containing such other drugs in addition to the compound of Formula I, II or III is preferred. Accordingly, the pharmaceutical compositions of the present invention include those that also contain one or more other active ingredients, in addition to a compound of Formula I, II or III. Examples of other active ingredients that may be combined with a compound of Formula I, II or III include, but are not limited to: antipsychotic agents, cognition enhancing agents, anti-migraine agents, anti-asthmatic agents, antiinflamrnatory agents, anxiolytics, anti-Parkinson's agents, anti-epileptics, anorectic agents, serotonin reuptake inhibitors, other anti-obesity agents, as well as antidiabetic agents, lipid lowering agents, and antihypertensive agents which may be administered separately or in the same pharmaceutical compositions.

### EXAMPLES

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data. Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and Toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and high-pressure liquid chromatography (HPLC). The NMRs provided have been determined at a field strength of 400 MHz or above. Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)), Unless otherwise specified, all variables mentioned below have the meanings as provided above.

Compounds of the present invention, wherein Ar² is a phenyl or a pyridyl, X and R are H and Q is absent (formula Ia), can be prepared according to the following general methods described in Scheme 1. For example, deprotonation of the benzyl nitrile Ia-2 using sodium ethoxide and subsequent addition to an aryl acrylate moiety of type Ia-1, can afford the intermediate Ia-3. The latter can be converted to the imide Ia-4 using acidic conditions (AeOH/H₂SO₄). Reduction of the imide can be performed with borane-DMS to give the intermediate Ia-5. The resulting amine was protected as a t-butyl carbamate to provide intermediate Ia-8. Compound Ia-8 was then homologated via a Suzuki coupling reaction with boronic acids or pinacol boronates Ia-7 to afford compound Ia-9. An alternative route is the palladium catalyzed coupling reaction of intermediate Ia-8 with pinacol diborane to obtain intermediate Ia-10 followed by the subsequent palladium catalyzed coupling reaction with aryl bromide Ia-6 to obtain compound Ia-9. Finally, deprotection of the t-butyl carbamate protective group provided Ia. Alternatively, imide Ia-4 can be reacted with boronic acids or pinacol boronate Ia-7 under the Suzuki coupling conditions to give imide Ia-11, which is consequently reduced to give Ia.

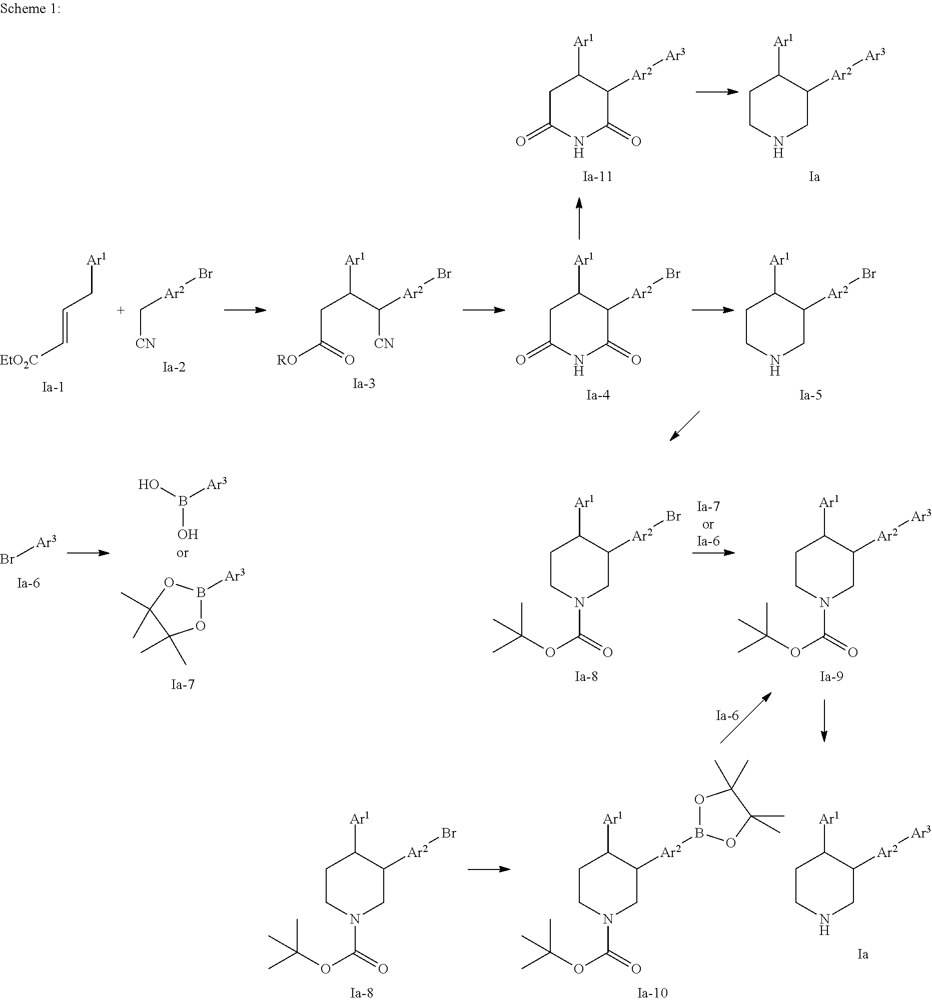

Compounds of the present invention, wherein Ar² is a phenyl or a pyridyl, X is OH, R is H and Q is absent (formula Ib), can be prepared according to the following general methods described in Scheme 2. An appropriately N-protected piperidinone Ib-1 is coupled with of an appropriately substituted 4-hydroxy aryl halide Ib-2 in the presence of a suitable palladium catalyst and a base to afford the arylated product Ib-3. The phenoxy moiety in Ib-3 can be converted into the corresponding triflate Ib-4 via reaction with triflic anhydride and a base. The triflate Ib-4 can be coupled with an appropriately substituted aryl halide Ib-5 in the presence of a suitable palladium catalyst to afford the bis-arylated ketone Ib-6. The ketone Ib-6 can be reacted with a suitable organometallic nucleophile Ib-7 to furnish the desired tertiary alcohol Ib-8. A final deprotection of the amino group would afford the desired product Ib.

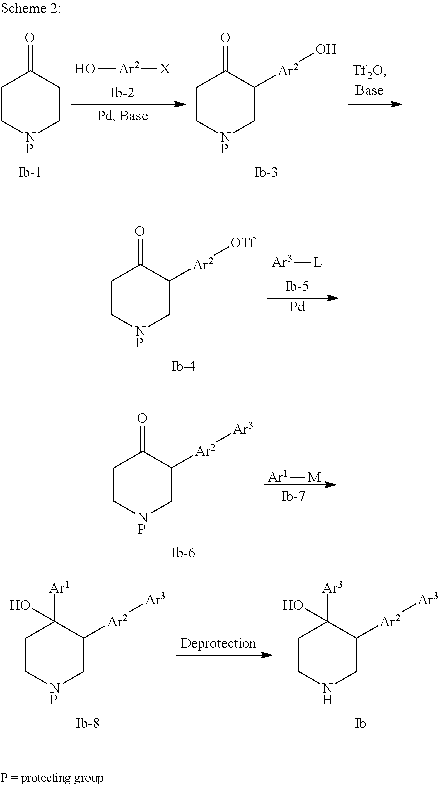

Compounds of the present invention, wherein Ar² is a isoxazolyl, X is H or OH, R is H and Q is absent (formula Ic and Id), can be prepared according to the following general methods described in Scheme 3 and Scheme 4. Reaction of ketone Ic-1 with commercially available 3-(benzylamino)propionitrile gives adduct Ic-2. Treatment of Ic-2 with a suitable base such as potassium tert-butoxide effects the cyclization to give the racemic mixture rac-Ic-3, which is separated by chiral HPLC to give the desired enantiomer Ic-3. Hydrolysis of the nitrile group affords acid Ic-4 which is converted to the corresponding N-Boc-protected product Ic-5 by hydrogenolysis in the presence of di-tert-butyl dicarbonate. The acid functionality in Ic-5 is reduced to the corresponding alcohol Ic-6, which in turn is oxidized with an appropriate oxidizing agent such as the Dess-Martin periodinane to give the corresponding aldehye Ic-7. Ic-7 is reacted with hydroxyamine to give the oxime Ic-8 which is reacted with chloramine-T to form the nitrile N-oxide in situ and subsequent reaction with a suitable alkyne Ic-10 furnishes the [3+2] cycloaddition product Ic-9. Deprotection of the Boc-protecting group gives the desired product Ic. Alternatively, a suitably protected piperidone analog Ie-11 can be treated with an appropriate organometallic reagent Ar¹M to afford intermediate Ic-12. Deprotection of PG₁ followed by chiral separation and protecting group switch as described above, the then oxidation of the corresponding alcohol furnishes aldehyde Ic-7.

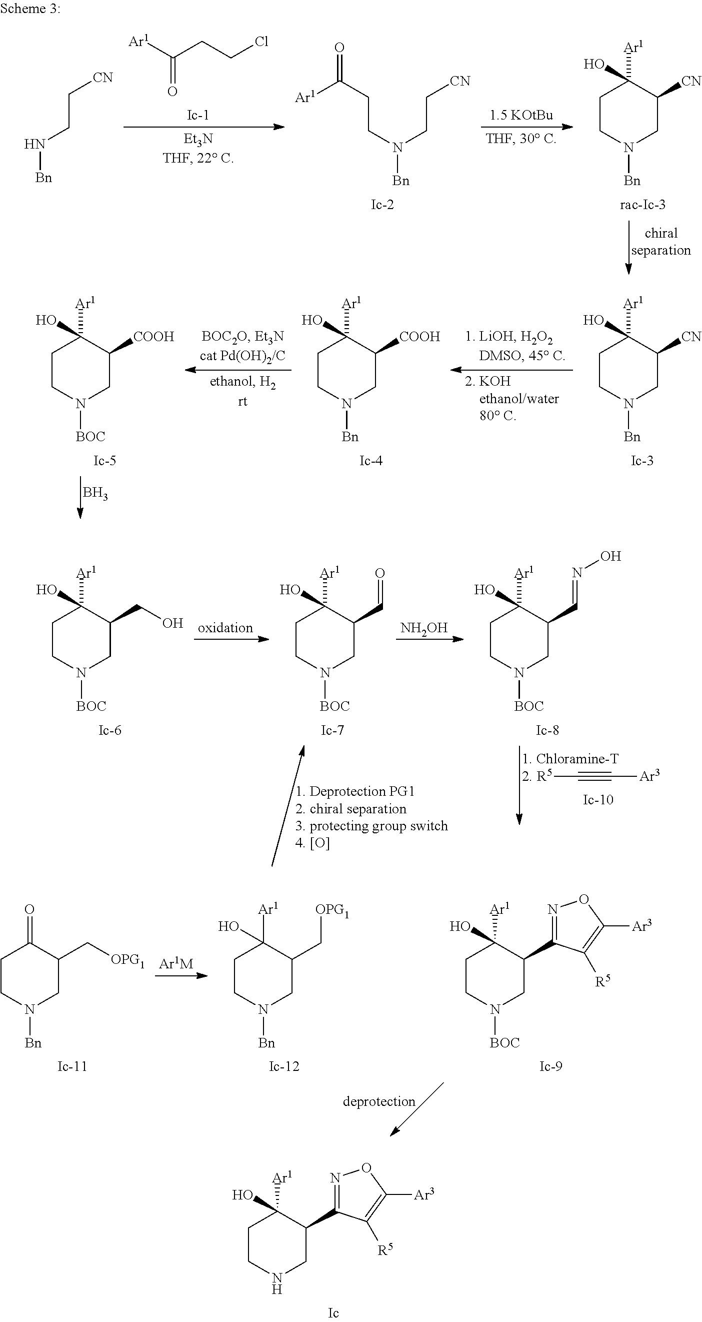

Compounds of the present invention, wherein Ar² is a isoxazolyl, X is OH, R is H and Q is absent (formula Id), can be prepared according to the following general methods described in Scheme 4. Reaction of aldehyde Ic-7 with CBr₄/PPh₃ (the Corey-Fuchs condition) followed by treatment with a base such as DBU, or reaction of Ic-7 with the Bestmann reagent give alkyne Id-1. Reaction oxime Id-3 with chloramine-T gives the corresponding nitrile N-oxide which is reacted with alkyne Id-1 to furnish the cycloaddition product Id-2. Deprotection of Id-2 gives Id.

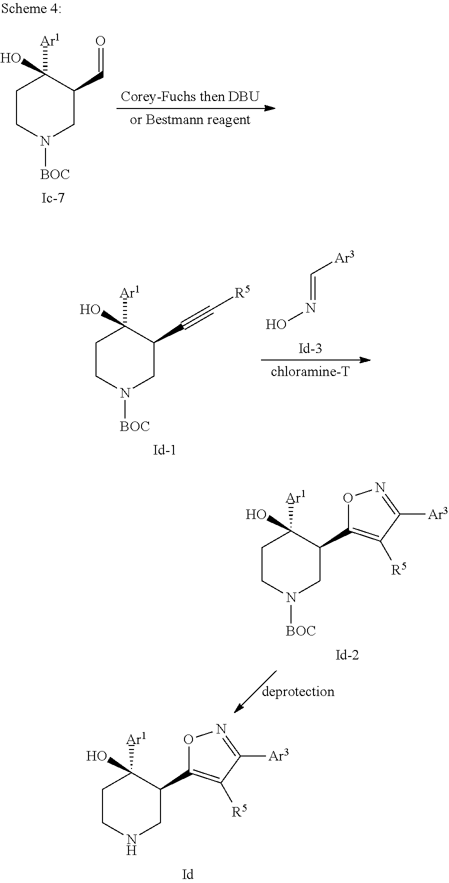

Compounds of the present invention, wherein Ar² is aryl, X is H, R is H and Q is absent (formula Ie and If), can be prepared according to the following general methods described in Scheme 5. Acid intermediate Ie-1 can be prepared according to procedure described in WO 06/125621. Similar transformations can be carried out according to Schemes 4&5 to give desired compounds Ie and If.

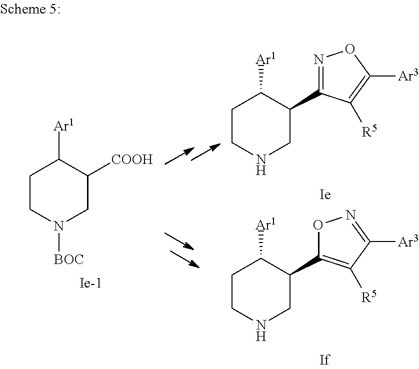

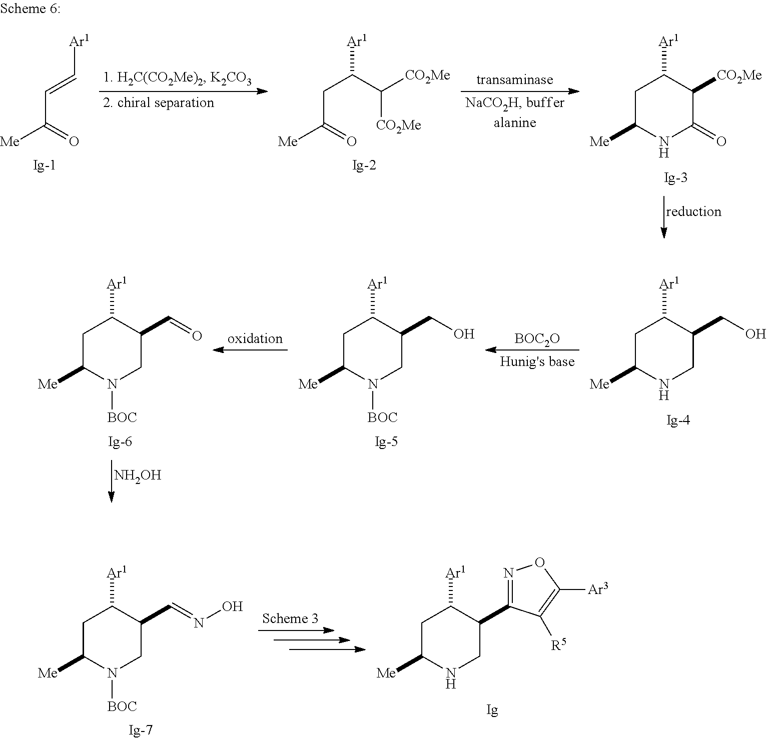

Compounds of the present invention, wherein Ar² is an isoxazole ring, X and R are both hydrogen and W is methyl (formula Ig), can be prepared according to the general methods described in Scheme 6. Heating of α,β-unsaturated ketone Ig-1 with dimethyl malonate in the presence of potassium carbonate, or an appropriate inorganic base, at 50° C. in THF readily furnishes the desired conjugate addition product Ig-2 as a racemic mixture. Chiral separation and subsequent transaminase-mediated asymmetric reductive amination of the ketone carbonyl afford lactam Ig-3. Concomitant reduction of both the lactam and the ester functionalities found in Ig-3 are achieved with a suitable reducing agent, such as borane-dimethyl sulfide complex in refluxing toluene. Selective N—BOC protection of the resulting amino alcohol Ig-4 under typical conditions furnishes carbamate alcohol Ig-5. Oxidation to aldehyde Ig-6 using a suitable oxidant such the Dess-Martin periodinane and subsequent condensation with hydroxylamine would give oxime Ig-7. Using similar chemistry described earlier in Scheme 3 will reveal the desired compound Ig.

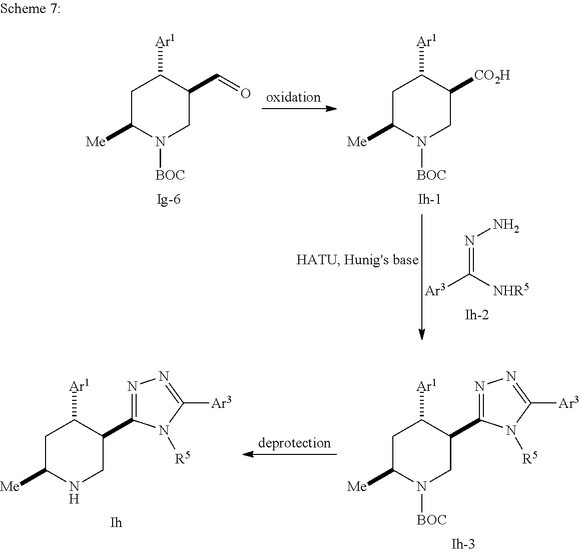

Compounds of the present invention, wherein Ar² is a 1,2,4-triazole ring, X and R are both hydrogen and W is methyl (formula Ih), can be synthesized according to the following general methods described in Scheme 7. Oxidation of aldehyde 1g-6 with appropriately buffered sodium chlorite in the presence of a suitable scavenger furnishes acid 1h-1. Treatment of acid 1h-1 with a suitably functionalized hydrazonamide 1h-2, which is readily prepared from the corresponding amide using well known procedures, in the presence of a coupling agent such as HATU and a base such as Hunig's base, results in 1,2,4-triazole 1h-3. Finally BOC-deprotection with a suitable acid such as HCl affords the desired compound Ih.

Experimental procedures: the following section describes the method of synthesis of representative examples in this invention. It is understood that other examples or analogs not described in the experimental section can be prepared by similar procedures by someone familiar with the art of organic synthesis.

### Benzyl Nitrile Ia-2

| COMPOUND | STRUCTURE |
|---|---|
| IA-2.1 | 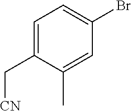 |
| IA-2.2 | 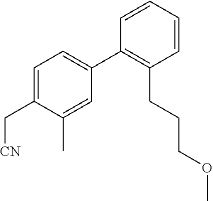 |
| IA-3.3 | 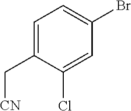 |

### Ia-2.1: (4-bromo-2-methylphenyl)acetonitrile

### Step 1: (4-bromo-2-methylphenyl)methanol

Borane-dimethyl sulfide complex (2.5 eq.; 10 M) was added to a stirred solution of commercially available 4-bromo-2-methylbenzoic acid (1 eq.) in THF (0.3 M). The mixture was refluxed for 3 h without a condensor allowing the SMe₂ to escape. The final reaction concentration was 0.5 M. The mixture was cooled to 0° C. and quenched with a slow addition of 1N HCl. The resulting mixture was extracted with Et₂O. The organic extract was washed with water, brine, dried over MgSO₄, filtered and concentrated to afford the desired material as a yellow solid.

### Step 2: 4-bromo-1-(bromomethyl)-2-methylbenzene

Hydrobromic acid (conc., 2 eq.) was added to a stirred solution of the alcohol from step 1 (1 eq.) in acetic acid (0.22 M). The mixture was stirred at 50° C. for 12 h, cooled down to room temperature, poured in water and extracted with Et₂O. The organic extract was washed with water, a saturated aqueous solution of NaHCO₃ (3×), brine, dried over MgSO₄, filtered and concentrated to afford the desired benzyl bromide as a light yellow solid.

### Step 3: (4-bromo-2-methylphenyl)acetonitrile

Potassium cyanide (1.4 eq.) was added to a stirred solution of the benzyl bromide from step 2 in DMF (0.22 M) and a small amount of water (1%). The suspension was stirred 72 h at 80° C. The reaction was monitored by NMR of small aliquots. The final mixture was cooled down to room temperature, poured in water and extracted with Et₂O. The organic extract was washed with water (2×), brine, dried over MgSO₄, filtered and concentrated. The residue was purified by flash chromatography on silica gel, eluting with Hexanes/EtOAc (5 then 10%) to give the title compound as a yellow solid.

### Ia-2.2: [2′(3-methoxypropyl)-3-methylbiphenyl-4-yl]acetonitrile

To a solution of aryl bromide Ia-2.1 (1 eq.), boronic acid Ia-7.2 (1.2 eq.), prepared as described below in DMF (0.2 M) was added Na₂CO₃ (3 eq.; 2 M) followed by PdCl₂(dppf)-CH₂Cl₂ adduct (0.05 eq.). The mixture was stirred at 90° C. overnight then cooled to room temperature. The reaction was diluted with ether, washed with water, HCl (0.01 N) then brine. The organic phase was dried over MgSO₄, filtered and evaporated. The residue was purified by flash chromatography on silica gel (ISCO COMBI-FLASH®) eluting with 5-30% EtOAc/Hexanes to give the desired product as a colorless oil.

### Ia-2.3; (4-bromo-2-chlorophenyl)acetonitrile

### Step 1: (4-bromo-2-chlorophenyl)methanol

Prepared according to the procedure described in Ia-2.1, step 1 starting from commercially available 4-bromo-2-chlorobenzoic acid The desired material was purified following work-up by trituration in Hexanes to afford a white solid.

### Step 2: 4-bromo-1-(bromomethyl)-2-chlorobenzene

To a solution of the benzyl alcohol from step 1 (1 eq.) in CH₂Cl₂ (0.1 M) at 0° C. was added CBr₄ (1.2 eq.) and PPh₃ (1.2 eq.). The mixture was stirred for 12 h at room temperature and concentrated. Purification of the residue by flash chromatography on silica gel, eluting with Hexanes, afforded the desired compound as an off-white solid.

### Step 3: (4-bromo-2-chlorophenyl)acetonitrile

Prepared according to the procedure described in Ia-2.1, step 3 starting from the benzyl bromide described in step 2.

Aryl bromide Ia-6

| COMPOUND | STRUCTURE |
|---|---|
| Ia-6.1 | 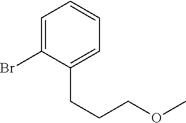 |
| Ia-6.2 | 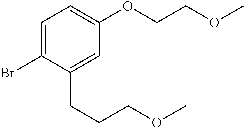 |
| Ia-6.3 | 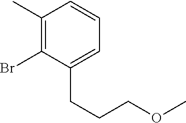 |
| Ia-6.4 | 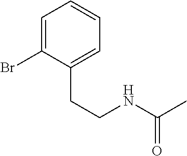 |

### Ia-6.1: 3-(2-bromophenyl)propyl methyl ether

### Step 1: 3-(2-bromophenyl)propan-1-ol

Borane-dimethyl sulfide complex (2.2 eq.) was added to a stirred solution of commercially available 3-(2-bromophenyl)propanoic acid (1 eq.) in THF (0.15 M). The mixture was refluxed for 3 h without a condensor allowing the SMe₂ to escape. The final reaction concentration was 0.3 M. The mixture was cooled to 0° C. and quenched with a slow addition of 1N HCl. The resulting mixture was extracted with Et₂O. The organic extract was washed with water, brine, dried over MgSO₄, filtered and concentrated to afford the desired material as a yellow solid.

### Step 2: 3-(2-bromophenyl)propyl methyl ether

To a solution of 3-(2-bromophenyl)propan-1-ol (1 eq.) in THF/DMF (4/1; 0.12 M) at 0° C. was added NaH (60% dispersion in oil; 1.5 eq). The mixture was stirred for 30 min then CH₃I (2 eq.) was added. The final mixture was stirred for 12 h at room temperature, poored in saturated aqueous solution of NH₄Cl and then extracted with Et₂O. The organic extract was washed with water, brine, dried over MgSO₄, filtered and concentrated. Purification by flash chromatography on silica gel (ISCO COMBI-FLASH8), eluting with Hexanes/EtOAc from 0 to 50%, afforded the desired compound as a colorless oil.

### Ia-6.2: 1-bromo-4-(2-methoxyethoxy)-2-(3-methoxypropyl)benzene

### Step 1: 1-bromo-4-(2-methoxyethoxy)-2-methylbenzene

To a solution of 4-bromo-3-methylphenol (1 eq.) in THF/DMF (4/1; 0.2 M) was added 2-bromoethyl methyl ether (1.3 eq.) and Cs₂CO₃ (1.3 eq,), The mixture was stirred at 80° C. overnight then cooled to room temperature. The reaction was diluted with EtOAc, washed with aqueous solution of NH₄Cl (10%), water and brine. The organic phase was dried over MgSO₄, filtered then evaporated to dryness. The residue was purified by flash chromatography on silica gel using 10% EtOAc/Hexanes.

### Step 2: 1-bromo-2-(bromomethyl)-4-(2-methoxyethoxy)benzene

To a solution of 1-bromo-4-(2-methoxyethoxy)-2-methylbenzene (1 eq.), N-bromosuccinimide (1.1 eq.) in CCl₄ (0.12 M) was added benzoyl peroxide (0.1 eq.). The mixture was stirred at 80° C. with a sun lamp overnight. The reaction was then cooled to room temperature and evaporated. The residue was purified by flash chromatography on silica gel using 10% EtOAc/Hexanes to afford the desired compound.

### Step 3: t-butyl 3-[2-bromo-5-(2-methoxyethoxy)phenyl]propanoate

To a solution of N-isopropylcyclohexylamine (1.3 eq.) in THF at −78 was added n-BuLi (1.2 eq.; 2.5 M). After 30 min, t-butylacetate (1.3 eq.) was added very slowly (int. temp. <−68° C.). The reaction was stirred 30 min at −78° C. then 1-bromo-2-(bromomethyl)-4-(2-methoxyethoxy)benzene (1 eq.) in THF was added. The reaction was stirred at −20° C. for 2 h then brought to room temperature. The reaction was quenched with water, extracted with EtOAc and washed with brine. The organic phase was dried over MgSO₄, filtered then evaporated to dryness. The residue was purified by flash chromatography on silica gel using 5% EtOAc/Toluene.

### Step 4: 3-[2-bromo-5-(2-methoxyethoxy)phenyl]propan-1-ol

To a solution of t-butyl-3-[2-bromo-5-(2-methoxyethoxy)phenyl]propanoate in toluene at −78° C. was added DIBAL-H (5 eq.; 20%/Toluene). The mixture was brought to room temperature for 2 h then quenched with Rochelle's salt 1 M, stirred for 2 h then extracted with EtOAc. The organic phase was dried over MgSO₄, filtered then evaporated to dryness. The residue was purified by flash chromatography on silica gel using 50% EtOAc/Hexanes.

### Step 5: 1-bromo-4-(2-methoxyethoxy)-2-(3-methoxypropyl)benzene

To a solution of 3-[2-bromo-5-(2-methoxyethoxy)phenyl]propan-1-ol (1 eq.) in THF/DMF (4/1; 0.2 M) was added NaH (1.3 eq.; 60% dispersion in oil). The mixture was stirred at room temperature for 30 min then CH₃I was added (1.5 eq.). The reaction was stirred at room temperature overnight then quenched with water, extracted with EtOAc and washed with brine. The organic phase was dried over MgSO₄, filtered then evaporated to dryness to obtain the desired compound.

### Ia-6.3: 3-(2-bromo-3-methylphenyl)propyl methyl ether

### Step 1: ethyl 3-(2-bromo-6-methylphenyl)propanoate

To a solution of commercially available ethyl (2E)-3-(2-bromo-6-methylphenyl)acrylate (1 eq.) in Toluene (0.1 M) at reflux was added benzenesulfonyl hydrazide (3 eq). The reaction mixture was refluxed for 3 h, cooled to room temperature, and diluted with Et₂O. The organic phase was washed with NaOH (1N), brine, dried over MgSO₄, filtered and concentrated. Purification by flash chromatography on silica gel (ISCO COMBI-FLASH®), eluting with Flexanes/EtOAc (0 to 20%, in 30 min) afforded the desired compound.

### Step 2: 3-(2-bromo-6-methylphenyl)propan-1-ol

To a solution of the ester from Step 1 (1 eq.) in THF (0.07 M) at −78° C. was added dropwise DIBAL-H (2.1 eq). The reaction mixture was stirred 1 h at −78° C., warm slowly to 0° C., quenched with HCl (1N) and finally diluted with Et₂O. The organic extract was washed with HCl (6N), water, saturated aqueous solution of NaHCO₃, brine, dried over MgSO₄, filtered and concentrated. Purification by flash chromatography on silica gel (ISCO COMBI-FLASH®), eluting with Hexanes/EtOAc (0 to 50%, in 30 min) afforded the desired compound.

### Step 3: 3-(2-bromo-3-methylphenyl)propyl methyl ether

Prepared according to the procedure described in Ia-6.1, step 2, starting from 3-(2-bromo-6-methylphenyl)propan-1-ol.

### Ia-6.4: N-[2-(2-bromophenyl)ethyl]acetamide

To a solution of commercially available 2-(2-bromophenyl)ethanamine hydrochloride salt (1 eq.) in CH₂Cl₂ (0.3 M) at room temperature was added Et₃N (2.1 eq) then Ac₂O (1.1 eq.). The mixture was stirred at room temperature for 6 h, poured in HCl (0.5 N) and extracted with CH₂Cl₂. The organic extract was dried over MgSO₄, filtered and concentrated to afford the desired compound as a white solid.

Boronic acid Ia-7

| COMPOUND | STRUCTURE |
|---|---|
| Ia-7.1 | 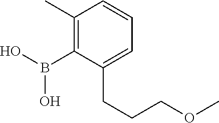 |
| Ia-7.2 | 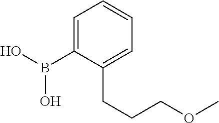 |
| Ia-7.3 | 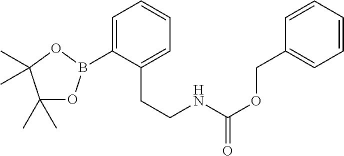 |

### Ia-7.1: [2-(3-methoxypropyl)-6-methylphenyl]boronic acid

To a solution of 3-(2-bromo-3-methylphenyl)propyl methyl ether (1 eq.) in THF (0.16 M) at −78° C. was added n-BuLi (2.5 M; 1.1 eq.), The reaction mixture was stirred for 1 h at −78° C. then tri-isopropylborate (1.2 eq.) was added and the final mixture was allowed to warm slowly to room temperature and stirred for 1 h. The reaction was quenched with the addition of HCl (1N) and extracted with EtOAc. The organic extract was washed with brine, dried over Na₂SO₄, filtered and concentrated. The desired material was purified by crystallization in Hexanes/EtOAc.

### Ia-7.2: [2-(3-methoxypropyl)phenyl]boronic acid

Prepared according to the procedure described for Ia-7.1 using 3-(2-bromophenyl)propyl methyl ether as starting material.

### Ia-7.3: benzyl {2-[2-(4,4,5,5-tetramethyl-1,3,2-dioxaborolan-2-yl)phenyl]ethyl}carbamate

### Step 1: Benzyl [2-(2-bromophenyl)ethyl]carbamate

To a solution of 2-(2-bromophenyl)ethanamine (20 g, 100 mmol) and Hunig's base (20.95 ml, 120 mmol) in Dichloromethane (500 ml) at 0° C. was added benzyl chloroformate (15.70 ml, 110 mmol). The reaction was stirred at room temperature for 3 h. The mixture was quenched with aqueous sodium bicarbonate and diluted with ethyl acetate. The organic layer was washed with water, aqueous sodium bicarbonate, brine, dried (MgSO₄), filtered and the solvent was evaporated under reduced pressure. Trituration in hexanes afforded the desired product (25.4 g, 76 mmol, 76% yield) as a white solid.

### Step 2: Title compound Ia-7.3

To a solution of benzyl [2-(2-bromophenyl)ethyl]carbamate (20.5 g, 61.3 mmol) and bis(pinacolato)diboron (17.13 g, 67.5 mmol) in DMF (350 ml) at room temperature was added potassium acetate (18.06 g, 184 mmol) and PdCl₂(dppf)-CH₂Cl₂ adduct (2.505 g, 3.07 mmol). The reaction was then heated to 80° C. for overnight. The mixture was cooled, quenched with water and diluted with ethyl acetate. The organic layer was washed with water, brine, dried (MgSO₄), filtered and the solvent was evaporated under reduced pressure. The residue was purified by column chromatography on silica gel, eluting with Hexanes/EtOAc 100 to 50% gave the title compound Ia-7.3 as a colorless oil.

### Example 1

### STRUCTURE 7: (+/−)-trans-3-{4′-[4-(3,5-difluorophenyl)piperidin-3-yl]-3′-methylbiphenyl-2-yl}propan-1-ol

### Step 1: Ethyl 4-cyano-3-(3,5-difluorophenyl)-4-[2′-(3-methoxypropyl)-3-methylbiphenyl-4-yl]butanoate

To a mixture of ethyl 3-(3,5-difluorophenyl)acrylate (1 eq.) and [2′-(3-methoxypropyl)-3-methylbiphenyl-4-yl]acetonitrile Ia-2.2 (1 eq.) at 70° C. was added a solution of NaOEt in ethanol (0.4 eq., 0.4 M). The mixture was heated at 70° C. overnight then cooled to room temperature. The reaction was quenched with a saturated aqueous solution of NH₄Cl and diluted with EtOAc. The organic phase was washed with a saturated aqueous solution of NaHCO₃, brine then dried over MgSO₄, filtered and evaporated to dryness to obtain the desired compound.

### Step 2: (+/−)-trans-3-{4′-[4-(3,5-difluorophenyl)-2,6-dioxopiperidin-3-yl]-3′-methylbiphenyl-2-yl}propyl acetate

A solution of ethyl-4-cyano-3-(3,5-difluorophenyl)-4-[2′-(3-methoxypropyl)-3-methylbiphenyl-4-yl]butanoate (1 eq.) in H₂SO₄ (2.4 M) and AcOH (0.24 M) was heated at 150° C. for 4 h. The mixture was then cooled to room temperature and poured into water. The reaction was quenched with KOH (8 N), extracted with CH₂Cl₂, washed with brine, dried over MgSO₄, filtered then evaporated to afford the desired compound.

### Step 3: (+/−)-trans-3-{4′-[4-(3,5-difluorophenyl)piperidin-3-yl]-3′-methylbiphenyl-2-yl}propan-1-ol

To a solution of (+/−)-trans-3-{4′-[4-(3,5-difluorophenyl)-2,6-dioxopiperidin-3-yl]-3′-methylbiphenyl-2-yl}propyl acetate (1 eq.) in THF (0.2 M) was slowly added a solution of BH₃-DMS (10 eq.; 1 M). The mixture was heated under reflux overnight, cooled to room temperature and quenched carefully with MeOH. The mixture was evaporated, 10% HCl was added and the mixture heated under reflux for 1 h, cooled to room temperature and basified with NaOH (10 N) until pH=10. The aqueous phase was extracted with CH₂Cl₂ (3×), dried over MgSO₄, filtered then evaporated to dryness. The residue was purified by flash chromatography on silica gel using 95% CH₂Cl₂/5% 2M NH₃ in MeOH to afford the desired compound. HRMS ESI [M+H]: 422.2288.

### Example 2

### Structure 8: (+/−)-trans-4-(3,5-difluorophenyl)-3-[2′-(3-methoxypropyl)-3-methylbiphenyl-4-yl]piperidine

### Step 1: (+/−)-trans-t-butyl 4-(3,5-difluorophenyl)-3-[2′-(3-hydroxypropyl)-3-methylbiphenyl-4-yl]piperidine-1-carboxylate

To a solution of (+/−)-trans-3-{4′-[4-(3,5-difluorophenyl)piperidin-3-yl]-3′-methylbiphenyl-2-yl}propan-1-ol (1 eq.) in CH₂Cl₂ (0.1 M) was added Hunig's base (1.5 eq.) followed by di-tert-butyl dicarbonate (1.2 eq.). The mixture was stirred at room temperature overnight then diluted with EtOAc, washed with a saturated solution of NaHCO₃, brine, dried over MgSO₄, filtered and evaporated to obtain the desired compound.

### Step 2: (+/−)-trans-t-butyl 4-(3,5-difluorophenyl)-3-[2′-(3-methoxypropyl)-3-methylbiphenyl-4-yl]piperidine-1-carboxylate

To a solution of (+/−)-trans-t-butyl-4-(3,5-difluorophenyl)-3-[2′-(3-hydroxypropyl)-3-methylbiphenyl-4-yl]piperidine-1-carboxylate (1 eq.) in THF (0.13 M) and DMF (0.4 M) was added NaH (1.2 eq.) and the reaction was stirred at room temperature for 30 min. To the mixture was then added CH₃I (1.6 eq.) and it was stirred at 40° C. overnight. After cooling down to room temperature, the mixture was diluted with EtOAc, washed with water, brine, dried over MgSO₄, filtered then evaporated to dryness to obtain the title compound.

### Step 3: (+/−)-trans-4-(3,5-difluorophenyl)-3-[2′-(3-methoxypropyl)-3-methylbiphenyl-4-yl]piperidine

To a mixture of (+/−)-trans-t-butyl-4-(3,5-difluorophenyl)-3-[2′-(3-methoxypropyl)-3-methylbiphenyl-4-yl]piperidine-1-carboxylate (1 eq.) in CH₂Cl₂ (0.2 M) was added HCl (10 eq.; 4 M) and the mixture was stirred at room temperature for 4 h. After evaporation, the crude compound was purified by flash chromatography on silica gel using 95% CH₂Cl₂/5% 2M NH₃ in MeOH to afford the title compound. HRMS ESII [M+H]: 436.2451.

### Example 3

### Structure 6: (+/−)-trans-3-{3-[4′-(2-methoxyethoxy)-2′-(3-methoxypropyl)-3-methylbiphenyl-4-yl]piperidin-4-yl}pyridine

### Step 1: ethyl 4-(4-bromo-2-methylphenyl)-4-cyano-3-pyridin-3-ylbutanoat

Prepared according to the procedure described in EXAMPLE 1, step 1 starting from ethyl 3-pyridin-3-ylacrylate and (4-bromo-2-methylphenyl)acetonitrile Ia-2.1.

### Step 2: (+/−)-trans-3-(4-bromo-2-methylphenyl)-4-pyridin-3-ylpiperidine-2,6-dione

Prepared according to the procedure described in EXAMPLE 1, step 2 starting from ethyl-4-(4-bromo-2-methylphenyl)-4-cyano-3-pyridin-3-ylbutanoate from step 1.

### Step 3: (+/−)-trans-3-[3-(4-bromo-2-methylphenyl)piperidin-4-yl]pyridine

Prepared according to the procedure described in EXAMPLE 1, step 3 starting from (+/−) trans-3-(4-bromo-2-methylphenyl)-4-pyridin-3-ylpiperidine-2,6-dione from step 2. The desired material was obtained after purification by flash chromatography eluting with 0-30% MeOH (5% NH₄OH)/CH₂Cl₂.

### Step 4: (+/−)-trans-t-butyl 3-(4-bromo-2-methylphenyl)-4-pyridin-3-ylpiperidine-1-carboxylate

Prepared according to the procedure described in EXAMPLE 2, step 1 starting from (+/−)-trans-3-[3-(4-bromo-2-methylphenyl)piperidin-4-yl]pyridine from step 3. The desired material was purified by flash chromatography on silica gel eluting with 0-20% MeOH (5% NH₄OH)/CH₂Cl₂.

### Step 5: (+/−)-trans-t-butyl 3-[4′-(2-methoxyethoxy)-2′-(3-methoxypropyl)-3-methylbiphenyl-4-yl]-4-pyridin-3-ylpiperidine-1-carboxylate

To a solution of (+/−)-trans-t-butyl-3-(4-bromo-2-methylphenyl)-4-pyridin-3-ylpiperidine-1-carboxylate from step 4 (1 eq.), bis(pinacolato)diboron (1.1 eq.), and KOAc (3 eq.) in DMF (0.15 M) was added PdCl₂(dppf)-CH₂Cl₂ adduct (0.05 eq,). The mixture was heated at 80° C. overnight then cooled to room temperature. A solution of 1-bromo-4-(2-methoxyethoxy)-2-(3-methoxypropyl)benzene (Ia-2.2) (2 eq.) in DMF was added followed by Na₂CO₃ (5 eq.; 2 M) and PdCl₂(dppf)-CH₂Cl₂ adduct (0.05 eq.). The mixture was heated at 80° C. overnight then cooled to room temperature. The mixture was diluted with EtOAc, washed with water, brine, dried over MgSO₄, filtered and evaporated. The residue was purified by flash chromatography on silica gel using 100% EtOAc.

### Step 6: (+/−)-trans-3-{3-[4′-(2-methoxyethoxy)-2′-(3-methoxypropyl)-3-methylbiphenyl-4-yl]piperidin-4-yl}pyridine

Prepared according to the procedure described in EXAMPLE 2, step 3 starting from (+/−)-trans-t-butyl-3-[4′-(2-methoxyethoxy)-2-(3-methoxypropyl)-3-methylbiphenyl-4-yl]-4-pyridin-3-ylpiperidine-1-carboxylate from step 5. The desired material was purified by flash chromatography on silica gel eluting with 90% CH₂Cl₂/10% 2 M NH₃ in MeOH. HRMS ESI [M+H]: 475.2962.

### Example 4

### Structure 3: (+/−)-trans-N-(2-{3′-methyl-4′-[(3S,4S)-4-pyridin-3-ylpiperidin-3-yl]biphenyl-2-yl}ethyl)acetamide

### Step 1: (+/−)-trans-t-butyl 3-{2′-[2-(acetylamino)ethyl]-3-methylbiphenyl-4-yl}-4-pyridin-3-ylpiperidine-1-carboxy late

Prepared according to the procedure described in EXAMPLE 3, step 5 starting from (+/−)-trans-t-butyl-3-(4-bromo-2-methylphenyl)-4-pyridin-3-ylpiperidine-1-carboxylate and N-[2-(2-bromophenyl)ethyl]acetamide (Ia-2.4). The desired material was obtained after flash chromatography on silica gel eluting with 0-15% MeOH (5% NR₄OH)/CH₂Cl₂.

### Step 2: (+/−)-trans-N-(2-{3′-methyl-4′-[(3S,4S)-4-pyridin-3-ylpiperidin-3-yl]biphenyl-2-yl}ethyl)acetamide

Prepared according to the procedure described in EXAMPLE 2, step 3 starting from (+/−)-trans-t-butyl-3-{2′-[2-(acetylamino)ethyl]-3-methylbiphenyl-4-yl}-4-pyridin-3-ylpiperidine-1-carboxylate. The desired material was obtained after purification by flash chromatography on silica gel eluting with 0-15% MeOH (5% NH₄OH)/CH₂Cl₂. HRMS ESI [M+H]: 414.2542.

### Example 5

### Structure 4: (3S,4S)—N-(2-{3′-methyl-4′-[(3S,4S)-4-pyridin-3-ylpiperidin-3-yl]biphenyl-2-yl}ethyl)acetamide

The two enantiomers of (+/−)-trans-t-butyl-3-{2′-[2-(acetylamino)ethyl]-3-methylbiphenyl-4-yl}-4-pyridin-3-ylpiperidine-1-carboxylate were separated on chiral HPLC using Chiralpak AD column; eluting with 15% 2-propanol/85% Hexanes. The slow eluting enantiomer was isolated and the BOC protecting group was removed using the procedure described for EXAMPLE 2, step 3 to afford the desired compound. HRMS ESI [M+H]: 414.2549.

### Example 6

### Structure 5: (3R,4R)—N-(2-{3′-methyl-4′-[(3S,4S)-4-pyridin-3-ylpiperidin-3-yl]biphenyl-2-yl}ethyl)acetamide

The two enantiomers of (+/−)-trans-t-butyl-3-{2′-[2-(acetylamino)ethyl]-3-methylbiphenyl-4-yl}-4-pyridin-3-ylpiperidine-1-carboxylate were separated on chiral HPLC using Chiralpak AD column; eluting with 15% 2-propanol/85% hexanes. The fast eluting enantiomer was isolated and the BOC protecting group was removed using the procedure described for EXAMPLE 2, step 3 to afford the desired compound. FIRMS ESI [M+H]: 414.2557.

### Example 7

### Structure 2: (+/−)-trans-3-{3-[2′-(3-methoxypropyl)-3-methylbiphenyl-4-yl]piperidin-4-yl}pyridine

### Step 1: (+/−)-trans-t-butyl 3-[2′(3-methoxypropyl)-3-methylphenyl-4-yl]-4-pyridin-3-ylpiperidine-1-carboxylate

Prepared according to the procedure described in EXAMPLE 3, step 5 starting from (+/−)-trans-t-butyl 3-(4-bromo-2-methylphenyl)-4-pyridin-3-ylpiperidine-1-carboxylate and [2-(3-methoxypropyl)phenyl]boronic acid (Ia-7.2) under typical Suzuki coupling conditions. The desired material was obtained after flash chromatography on silica gel (ISCO COMBI-FLASH®) eluting with Hexanes/EtOAc.

### Step 2: (+/−)-trans-3-{3-[2′(3-methoxypropyl)-3-methylbiphenyl-4-yl]piperidin-4-yl}pyridine

Prepared according to the procedure described in EXAMPLE 2, step 3 starting from (+/−)-trans-t butyl-3-[2′-(3-methoxypropyl)-3-methylbiphenyl-4-yl]-4-pyridin-3-ylpiperidine-1-carboxylate. The desired material was obtained after flash chromatography on silica gel eluting with 0-30% MeOH (10% NH₄OH)/CH₂Cl₂. HRMS ESI [M+H]: 401.2592.

### Example 8

### Structure 9: (+/−)-trans-3-{4′-[4-(1H-benzimidazol-4-yl)piperidin-3-yl]-3′-methylbiphenyl-2-yl}propan-1-ol

### Step 1: 2-amino-3-nitrobenzoic acid

To a solution of 2-chloro-3-nitrobenzoic acid (1 eq.) in NH₄OH (7 eq.) was added CuCl (I) (0.02 eq.). The mixture was then stirred at 125° C. for 24 h. The mixture was cooled to room temperature. The residue was dissolved in water and acidified with HCl (3 N). The resulting precipitate was filtered and dried to give the desired product as a yellow powder.

### Step 2: 2-amino-3-(formylamino)benzoic acid

To a solution of 2-amino-3-nitrobenzoic acid (1 eq.) in NaOH (1.12 eq.; 0.1 M) was added Pd/C (10%/weight). The mixture was then stirred for 24 h with 45 psi hydrogen pressure using a Parr apparatus. To the mixture was then added HCl (1 eq.) followed by formic acid (7 eq.). The mixture was heated under reflux for 24 h then cooled to room temperature and concentrated. After filtration the desired compound was obtained.

### Step 3: ethyl 1H-benzimidazole-4-carboxylate

To a suspension of 2-amino-3-(formylamino)benzoic acid 1 eq.) in EtOH (0.03 M) was added a solution of acetyl chloride (1 eq.) in EtOH (0.03 M). The reaction mixture was stirred at 60° C. for 72 h. The mixture was then evaporated to dryness to afford the desired compound.

### Step 4: 1H-benzimidazol-4-ylmethanol

To a solution of ethyl 1H-benzimidazole-4-carboxylate (1 eq.) in THF (0.06 M) at −78° C. was added LiAlH₄ (2.1 eq.; 1 M in THF). The mixture was then warmed to 0° C. for 1 h then to room temperature overnight. The reaction was quenched with HCl (10%) and diluted with EtOAc. The mixture was basified with NaHCO₃. The organic phase was washed with a saturated aqueous solution of NaHCO₃, brine then dried over MgSO₄, filtered and evaporated to dryness. The residue was purified by flash chromatography on silica gel using 90% CH₂Cl₂/10% MeOH to afford the desired product as a yellow solid.

### Step 5: 1H-benzimidazole-4-carbaldehyde

To a solution of 1H-benzimidazol-4-ylmethanol (1 eq.) in a mixture of THF (100 mL)/CH₂Cl₂ (100 mL)/DMSO (10 mL) was added pyridine (1.1 eq.) followed by Dess-Martin periodinane (1.1 eq.). The reaction was stirred for 1 h at room temperature then quenched with a saturated aqueous solution of NaHCO₃ and diluted with EtOAc. The organic phase was washed with NaHCO₃, brine, dried over MgSO₄, filtered then evaporated to dryness to afford the title compound.

### Step 6: ethyl 3-(1H-benzimidazol-4-yl)acrylate

To a solution of 1H-benzimidazole-4-carbaldehyde (1 eq.) in THF (0.04 M) was added triethylphosphonoacetate (1.2 eq.) followed by KOt-13u (1.1 eq.; 1 M). The mixture was stirred 1 h at room temperature. The reaction was quenched with HCl 10% and diluted with EtOAc. The organic phase was washed with a saturated aqueous solution of NaHCO₃, brine, dried over MgSO₄, filtered then evaporated to dryness. The residue was purified by flash chromatography on silica gel using 95% CH₂Cl₂/5% MeOH to obtain the desired compound.

### Step 7: ethyl 3-(1H-benzimidazol-4-yl)-4-cyano-4-[2′-(3-methoxypropyl)-3-methylbiphenyl-4-yl]butanoate

To a solution of ethyl 3-(1H-benzimidazol-4-yl)acrylate (1 eq.) from step 6 and [2′-(3-methoxypropyl)-3-methylbiphenyl-4-yl]acetonitrile Ia-6.2 in THF (4.6 mM) at 60° C. was added NaOEt (0.4 eq.). The reaction was heated at 80° C. for 1 h, cooled to room temperature then quenched with 10% HCl. The mixture was diluted with EtOAc, washed with a saturated solution of NaHCO₃, brine, dried over MgSO₄, filtered then evaporated to dryness. The residue was purified by flash chromatography on silica gel (ISCO COMBI-FLASH®) eluting with 50 to 100% EtOAc/Hexanes to give the desired product as a mixture of diastereoisomers.

### Step 8: (+/−)-trans-4-(1H-benzimidazol-4-yl)-3-[2′-(3-hydroxypropyl)-3-methylbiphenyl-4-yl]piperidine-2,6-dione

Prepared according to the procedure described in EXAMPLE 1, step 2 starting from ethyl 3-(1H-benzimidazol-4-yl)-4-cyano-4-[2′-(3-methoxypropyl)-3-methylbiphenyl-4-yl]butanoate from step 7. The desired material was obtained after flash chromatography on silica gel eluting with 90% CH₂Cl₂/10% MeOH.

### Step 9: (+/−)-trans-3-{4′-[4-(1H-benzimidazol-4-yl)piperidin-3-yl]-3′-methylbiphenyl-2-yl}propan-1-ol

Prepared according to the procedure described in EXAMPLE 1, step 3 starting from (+/−)-trans-4-(1H-benzimidazol-4-yl)-3-[2′-(3-hydroxypropyl)-3-methylbiphenyl-4-yl]piperidine-2,6-dione from step 8. The desired material was obtained after purification by flash chromatography on silica gel eluting with 0-30% MeOH (5% NH₄OH)/CH₂Cl₂. HRMS ESI [M+H]: 426.2549.

### Example 9

### Structure 10: (+/−)-trans-4-{3-[3-chloro-2′-(3-methoxypropyl)biphenyl-4-yl]piperidin-4-yl}-1H-benzimidazole

### Step 1: ethyl 3-(1H-benzimidazol-4-yl)-4-(4-bromo-2-chlorophenyl)-4-cyanobutanoate

Prepared according to the procedure described in EXAMPLE 1, step 1 starting from ethyl 3-(1H-benzimidazol-4-yl)acrylate and (4-bromo-2-chlorophenyl)acetonitrile (Ia-6.3). The desired material was purified by flash chromatography on silica gel (ISCO COMBI-FLASH®) and eluting with 50% to 100% EtOAc/Hexanes.

### Step 2: (+/−)-trans-4-(1H-benzimidazol-4-yl)-3-(4-bromo-2-chlorophenyl)piperidine-2,6-dione

Prepared according to the procedure described in EXAMPLE 1, step 2 starting from ethyl-3-(1H-benzimidazol-4-yl)-4-(4-bromo-2-chlorophenyl)-4-cyanobutanoate from step 1.

### Step 3: (+/−)-trans-4-[3-(4-bromo-2-chlorophenyl)piperidin-4-yl]-1H-benzimidazole

Prepared according to the procedure described in EXAMPLE 1, step 3 starting from (+/−) trans-4-(1H-benzimidazol-4-yl)-3-(4-bromo-2-chlorophenyl)piperidine-2,6-dione from step 2.

### Step 4: (+/−)-trans-t-butyl 4-(1H-benzimidazol-4-yl)-3-(4-bromo-2-chlorophenyl)piperidine-1-carboxylate

Prepared according to the procedure described in EXAMPLE 2, step 1 starting from (+/−) trans-4-[3-(4-bromo-2-chlorophenyl)piperidin-4-yl]-1H-benzimidazole from step 3.

### Step 5: (+/−)-trans-t-butyl 4-(1H-benzimidazol-4-yl)-3-[3-chloro-2′-(3-methoxypropyl)biphenyl-4-yl]piperidine-1-carboxylate

To solution of (+/−)-trans-t-butyl 4-(1H-benzimidazol-4-yl)-3-(4-bromo-2-chlorophenyl)piperidine-1-carboxylate (1 eq.) from step 4, [2-(3-methoxypropyl)phenyl]boronic acid (Ia-7.2) (1.1 eq,), in 2-propanol (0.07 M) was added PPh₃ (0.15 eq.), Pd(OAc)₂ (0.05 eq.) and Na₂CO₃ (3 eq.; 2 M). The mixture was heated at 80° C. for 4 h then cooled to room temperature. The reaction was diluted in EtOAc, washed with water, a saturated aqueous solution of NaHCO₃ and brine. The organic phase was dried over MgSO₄, filtered then evaporated to dryness. The residue was purified by flash chromatography on silica gel eluting with EtOAc/MeOH/NH₄OH.

### Step 6: (+/−)-trans-4-{3-[3-chloro-2′-(3-methoxypropyl)biphenyl-4-yl]piperidin-4-yl}-1H-benzimidazole

Prepared according to the procedure described in EXAMPLE 2, step 3 starting from (+/−) trans-t-butyl 4-(1H-benzimidazol-4-yl)-3-[3-chloro-2′-(3-methoxypropyl)biphenyl-4-yl]piperidine-1-carboxylate from step 5. The desired material was obtained as a beige solid after flash chromatography on silica gel eluting with 90% CH₂Cl₂/10% 2M NH₃ in MeOH. HRMS ESI [M+H]: 460.2159.

### Example 10

### Structure 12: (+/−)-trans-3-{3-[2′-(3-methoxypropyl)-3,6′-dimethylbiphenyl-4-yl]piperidin-4-yl}pyridine

### Step 1: (+/−)-trans-t-butyl 3-[2′-(3-methoxypropyl)-3,6′-dimethylbiphenyl-4-yl]-4-pyridin-3-ylpiperidine-1-carboxylate

To a solution of (+/−)-trans-t-butyl 3-(4-bromo-2-methylphenyl)-4-pyridin-3-ylpiperidine-1-carboxylate (1 eq.), [2-(3-methoxypropyl)-6-methylphenyl]boronic acid (Ia-7.1) (1.1 eq.), Ba(OH)₂-8H₂0 (1.5 eq.) in DME (0.08 M) and water (0.5 M) was added Pd(PPh₃)₄ (0.15 eq.). The reaction was stirred at 80° C. overnight then cooled to room temperature. The mixture was diluted with EtOAc, washed with water, a saturated aqueous solution of NaHCO₃ and brine. The organic phase was dried over MgSO₄, filtered then evaporated to dryness. The residue was purified by flash chromatography on silica gel (ISCO COMBI-FLASH®) eluting with Hexanes/EtOAc.

### Step 2: (+/−)-trans-3-{3-[2′-(3-methoxypropyl)-3,6′-dimethylbiphenyl-4-yl]piperidin-4-yl}pyridine

Prepared according to the procedure described in EXAMPLE 2, step 3 starting from (+/−)-trans-t-butyl 3-[2′-(3-methoxypropyl)-3,6′-dimethylbiphenyl-4-yl]-4-pyridin-3-ylpiperidine-1-carboxylate. The desired material was obtained after flash chromatography on silica gel eluting with 90% CH₂Cl₂/10% 2M NH₃ in MeOH. HRMS ESI [M+H]; 415.2764.

### Example 11

### Structure 11: (+/−)-trans-3-{3-[3-chloro-2′-(3-methoxypropyl)biphenyl-4-yl]piperidin-4-yl}pyridine

### Step 1: ethyl 4-(4-bromo-2-chlorophenyl)-4-cyano-3-pyridin-3-ylbutanoate

Prepared according to the procedure described in EXAMPLE 1, step 1 starting from (4-bromo-2-chlorophenyl)acetonitrile (Ia-2.3) and ethyl-3-pyridin-3-ylacrylate. The desired material was obtained after flash chromatography on silica gel (ISCO COMBI-FLASH®) eluting with 15 to 55% EtOAc/Hexanes.

### Step 2: (+/−)-trans-3-(4-bromo-2-chlorophenyl)-4-pyridin-3-ylpiperidine-2,6-dione

Prepared according to the procedure described in EXAMPLE 1, step 2 starting from ethyl-4-(4-bromo-2-chlorophenyl)-4-cyano-3-pyridin-3-ylbutanoate from step 1.

### Step 3: (+/−)-trans-3-[3-(4-bromo-2-chlorophenyl)piperidin-4-yl]pyridine

Prepared according to the procedure described in EXAMPLE 1, step 3 starting from (+/−)-trans-3-(4-bromo-2-chlorophenyl)-4-pyridin-3-ylpiperidine-2,6-dione from step 2. The desired material was obtained after flash chromatography on silica gel eluting with 90% CH₂Cl₂/10% 2M NH₃ in MeOH.

### Step 4: (+/−)-trans-t-butyl 3-(4-bromo-2-chlorophenyl)-4-pyridin-3-ylpiperidine-1-carboxylate

Prepared according to the procedure described in EXAMPLE 2, step 1 starting from (+/−)-trans-3-[3-(4-bromo-2-chlorophenyl)piperidin-4-yl]pyridine from step 3.

### Step 5: (+/−)-trans-t-butyl 3-[3-chloro-2′-(3-methoxypropyl)biphenyl-4-yl]-4-pyridin-3-ylpiperidine-1-carboxylate

Prepared according to the procedure described in EXAMPLE 9, step 5 starting from (+/−)-trans-t-butyl 3-(4-bromo-2-chlorophenyl)-4-pyridin-3-ylpiperidine-1-carboxylate and [2-(3-methoxypropyl)phenyl]boronic acid (Ia-7.2).

### Step 6: (+/−)-trans-3-{3-[3-chloro-2′-(3-methoxypropyl)biphenyl-4-yl]piperidin-4-yl}pyridine

Prepared according to the procedure described in EXAMPLE 2, step 3 starting from (+/−)-trans-t-butyl-3-[3-chloro-2′-(3-methoxypropyl)biphenyl-4-yl]-4-pyridin-3-ylpiperidine-1-carboxylate. The desired material was obtained after evaporation to dryness of the crude reaction mixture. HRMS ESI [M+H]: 421.2056.

### Example 12

### Structure 1: (+/−)-trans-3-[2′-(3-methoxypropyl)-3-methylbiphenyl-4-yl]-4-phenylpiperidine

### Step 1: ethyl 4-(4-bromo-2-methylphenyl)-4-cyano-3-phenylbutanoate

Prepared according to the procedure described in EXAMPLE 1, step 1 starting from ethyl 3-phenylacrylate and (4-bromo-2-methylphenyl)acetonitrile Ia-2.1. The desired material was obtained after flash chromatography on silica gel (ISCO COMBI-FLASH®) eluting with 0-40% EtOAc/Hexanes.

### Step 2: (+/−)-trans-3-(4-bromo-2-methylphenyl)-4-phenylpiperidine-2,6-dione

Prepared according to the procedure described in EXAMPLE 1, step 2 starting from ethyl-4-(4-bromo-2-methylphenyl)-4-cyano-3-phenylbutanoate from step 1.

### Step 3: (+/−)-trans-3-[2′-(3-methoxypropyl)-3-methylbiphenyl-4-yl]-4-phenylpiperidine-2,6-dione

Prepared according to the procedure described in EXAMPLE 9, step 5 starting from (+/−)-trans-3-(4-bromo-2-methylphenyl)-4-phenylpiperidine-2,6-dione from step 2 and [2-(3-methoxypropyl)phenyl]boronic acid (Ia-7.2). The desired material was obtained after flash chromatography on silica gel (ISCO COMBI-FLASH®) eluting with 0-60% EtOAc/Hexanes.

### Step 4: (+/−)-trans-3-[2′-(3-methoxypropyl)-3-methylbiphenyl-4-yl]-4-phenylpiperidine

Prepared according to the procedure described in EXAMPLE 1, step 3 starting from (+/−)-trans-3-[2′-(3-methoxypropyl)-3-methylbiphenyl-4-yl]-4-phenylpiperidine-2,6-dione from step 3. The desired material was obtained after flash chromatography on silica gel eluting with 95% CH₂Cl₂/5% 2M NH₃ in MeOH. HRMS ESI [M+H]: 400.2629.

### Example 13

### Structure 13: N-(2-{3′-chloro-4′-[(3S,4S)-4-(pyridin-3-yl)piperidin-3-yl]biphenyl-2-yl}ethyl)acetamide

### Step 1: tert-butyl (3S,4S)-3-(4-bromo-2-chlorophenyl)-4-(pyridin-3-yl)piperidine-1-carboxylate

The racemic mixture from Example 11, step 4 was dissolved in 20% EtOH, 20% i-PrOH, 40% CHCl₃, 20% Hexanes at a concentration of 120 mg/ml. It was then injected onto a Chiralcell OD column eluting with 80% Hexanes/20% i-PrOH. The slow eluting enantiomer was the desired isomer.

### Step 2: tert-butyl (3S,4S)-3-{2′-[2-(acetylamino)ethyl]-3-chlorobiphenyl-4-yl}-4-(pyridin-3-yl)piperidine-1-carboxylate

To a degassed room temperature solution of tert-butyl (3S,4S)-3-(4-bromo-2-chlorophenyl)-4-(pyridin-3-yl)piperidine-1-carboxylate from the previous step (2 g, 4.43 mmol) and bis(pinacolato)diboron (1.237 g, 4.87 mmol) in DMF (40 ml) was added potassium acetate (1.303 g, 13.28 mmol) and PdCl₂(dppf)-CH₂Cl₂ adduct (0.181 g, 0.221 mmol). The reaction was then stirred at 80° C. for 3 h. Cooled down then a solution of N-[2-(2-bromophenyl)ethyl]acetamide (Ia-6.4) (1.179 g, 4.87 mmol) in DMF was introduced along with PdCl₂(dppf)-CH₂Cl₂ adduct (0.181 g, 0.221 mmol) and sodium carbonate (8.85 ml, 17.71 mmol). The reaction was stirred at 80° C. overnight. The mixture was cooled, quenched with water and diluted with ethyl acetate. The organic layer was washed with aqueous sodium bicarbonate, brine, dried (MgSO₄), filtered and the solvent was evaporated under reduced pressure. The residue was purified by column chromatography on silica gel eluting with CH₂Cl₂/MeOH/NH₃ (2M.) to give the desired product (2.2 g, 4.12 mmol, 93% yield) as a yellow foam.

### Step 3: N-(2-{3′-chloro-4′-[(3S,4S)-4-(pyridin-3-yl)piperidin-3-yl]biphenyl-2-yl}-ethyl)acetamide

To a room temperature solution of tert-butyl (3S,4S)-3-{2′-[2-(acetylamino)ethyl]-3-chlorobiphenyl-4-yl}-4-(pyridin-3-yl)piperidine-1-carboxylate (9.05 g, 16.94 mmol) in DCM (150 ml) was added hydrochloric acid (42.4 ml, 169 mmol). The reaction was then stirred at room temperature for overnight. The reaction was evaporated to dryness then diluted with MeOH and evaporated to give the title compound (8.6 g, 16.97 mmol) as beige foam.

### Example 14

### Structure 34: (+/−)-trans-4-(3,5-difluorophenyl)-3-{3-methyl-2′-[(2-methylphenoxy)methyl]biphenyl-4-yl}piperidine

### Step 1: (+/−)-trans-tert-butyl 3-[3-chloro-2′-(hydroxymethyl)biphenyl-4-yl]-4-(3,5-difluorophenyl)piperidine-1-carboxylate

To a room temperature solution of racemic mixture (+/−)-trans-tert-butyl-4-(3,5-difluorophenyl)-3-(2-methyl-4-{[trifluoromethyl)sulfonyl]oxy}phenyl)piperidine-1-carboxylate (2 g, 3.60 mmol) and 2,1-benzoxaborol-1(3H)-ol (0.578 g, 4.32 mmol) in DMF (40 ml) was added Pd(PPh₃)₃(OAc) (0.182 g, 0.180 mmol) and sodium carbonate (2M, 7.19 ml, 14.39 mmol). The reaction was stirred at 80° C. for 4 h. The mixture was cooled, quenched with water and diluted with ethyl acetate. The organic layer was washed with aqueous sodium bicarbonate, brine, dried (MgSO₄), filtered and the solvent was evaporated under reduced pressure. The residue was purified by column chromatography on silica gel, eluting with Hexanes/EtOAc to give the desired product (1.81 g, 3.52 mmol) as a white foam.

### Step 2: (+/−)-trans-tert-butyl 4-(3,5-difluorophenyl)-3-{3-methyl-2′-[(2-methylphenoxy)methyl]biphenyl-4-yl}piperidine-1-carboxylate

To a room temperature solution of (+/−)-trans-tert-butyl 3-[3-chloro-2′-(hydroxymethyl)biphenyl-4-yl]-4-(3,5-difluorophenyl)piperidine-1-carboxylate (100 mg, 0.195 mmol) and 2-methylphenol (42.1 mg, 0.389 mmol) in Toluene (2 ml) was added 1,1′-(azodicarbonyl)dipiperidine (108 mg, 0.428 mmol) and tri-n-butylphosphine (87 mg, 0.428 mmol). The reaction was then stirred at 80° C. overnight. The mixture was cooled, quenched with water and diluted with ethyl acetate. The organic layer was washed with aqueous sodium bicarbonate, brine, dried (MgSO₄), filtered and the solvent was evaporated under reduced pressure. The residue was purified by column chromatography on silica gel. Eluting with Hexanes/EtOAc gave the desired product (94 mg, 0.156 mmol, 80% yield) as a colorless oil.

### Step 3: (+/−)-trans-4-(3,5-difluorophenyl)-3-{3-methyl-2′-[(2-methylphenoxy)methyl]biphenyl-4-yl}piperidine

(+/−)-trans-tert-butyl-4-(3,5-dimethylphenyl)-3-[3-methyl-2′-(phenoxymethyl)biphenyl-4-yl]piperidine-1-carboxylate was deprotected as usual to give the title compound.

### Example 15

### Structure 35: (+/−)-trans-4-(3,5-difluorophenyl)-3-[3-methyl-2′-(phenoxymethyl)biphenyl-4-yl]piperidine

This compound was prepared similarly according to Example 14, step 2 and 3.

### Example 16

### Structure 36: (+/−)-trans-3-({4′-[4-(3,5-difluorophenyl)piperidin-3-yl]-3′-methylbiphenyl-2-yl}methoxy)pyridine

To a room temperature solution of (+/−)-trans-tert-butyl-3-[3-chloro-2′-(hydroxymethyl)biphenyl-4-yl]-4-(3,5-difluorophenyl)piperidine-1-carboxylate (100 mg, 0.195 mmol) and 3-hydroxypyridine (37.0 mg, 0.389 mmol) in Toluene (2 ml) was added 1,1′-(azodicarbonyl)dipiperidine (108 mg, 0.428 mmol) and tri-n-butylphosphine (0.107 ml, 0.428 mmol). The reaction was then stirred at 80° C. for overnight. The mixture was cooled, quenched with water and diluted with ethyl acetate. The organic layer was washed with aqueous sodium bicarbonate, brine, dried (MgSO₄), filtered and the solvent was evaporated under reduced pressure. The residue was purified by column chromatography on silica gel. Eluting with Hexanes/EtOAc to give desired product (64 mg, 0.108 mmol, 55.7% yield) as a colorless oil. This product was deprotected according to standard procedure to give the title compound.

### Example 17

### Structure 52: (+/−)-trans-5-{3-[3-chloro-2′-(3-methoxypropyl)biphenyl-4-yl]piperidin-4-yl}pyridin-2-amine

### Step 1: (+/−)-trans-tert-butyl 3-[3-chloro-2′-(3-methoxypropyl)biphenyl-4-yl]-4-(1-oxidopyridin-3-yl)piperidine-1-carboxylate

To a room temperature solution of (+/−)-trans-t-butyl 3-[3-chloro-2′-(3-methoxypropyl)biphenyl-4-yl]-4-pyridin-3-ylpiperidine-1-carboxylate (200 mg, 0.384 mmol) (Example 11, step 5) in CH₂Cl₂ (5 ml) was added m-CPBA (103 mg, 0.461 mmol). The reaction was then stirred at room temperature for overnight. Calcium hydroxide was then added and the residue filtered. Evaporated then purified by column chromatography on silica gel. Eluting with CH₂Cl₂/MeOH 0 to 10% over 20 min to give the desired product (175 mg, 0.326 mmol) as a white foam.

### Step 2: (+/−)-trans-tert-butyl 4-[6-(tert-butylamino)pyridin-3-yl]-3-[3-chloro-2′-(3-methoxypropyl)biphenyl-4-yl]piperidine-1-carboxylate; (+/−)-trans-tert-butyl 4-[2-(tert-butylamino)pyridin-3-yl]-3-[3-chloro-2′-(3-methoxypropyl)biphenyl-4-yl]piperidine-1-carboxylate and (+/−)-trans-tert-butyl 4-[4-(tert-butylamino)pyridin-3-yl]-3-[3-chloro-2′-(3-methoxypropyl)biphenyl-4-yl]piperidine-1-carboxylate

To a 0° C. solution of (+/−)-trans-tert-butyl 3-[3-chloro-2′-(3-methoxypropyl)biphenyl-4-yl]-4-(1-oxidopyridin-3-yl)piperidine-1-carboxylate (125 mg, 0.233 mmol) and t-butylamine (0.122 ml, 1.164 mmol) in PhCF₃ (1 ml) was added p-toluenesulfonic anhydride (152 mg, 0.465 mmol) portion wise. The reaction was then stirred at room temperature for overnight. The mixture was cooled, quenched with aqueous sodium bicarbonate and diluted with ethyl acetate. The organic layer was washed with aqueous sodium bicarbonate, brine, dried (Na₂SO₄), filtered and the solvent was evaporated under reduced pressure. Purification by reverse phase chromatography affords the three isomers.

### Step 3: (+/−)-trans-tert-butyl 4-[6-(tert-butylamino)pyridin-3-yl]-3-[3-chloro-2′-(3-methoxypropyl)biphenyl-4-yl]piperidine-1-carboxylate

To a room temperature solution of (+/−)-trans-tert-butyl 4-[6-(tert-butylamino)pyridin-3-yl]-3-[3-chloro-2′-(3-methoxypropyl)biphenyl-4-yl]piperidine-1-carboxylate (40 mg, 0.068 mmol) in PhCF₃ (1 ml) was added TFA (0.520 ml, 6.75 mmol), The reaction was then stirred at 70° C. overnight and then evaporated. The residue was purified by column chromatography on silica gel eluting with CH₂Cl₂/MeOH/NH₃ (2M.) to give the title compound (5.2 mg, 18% yield).

### Example 18

### Structure 53: (+/−)-trans-3-{3-[3-chloro-2′-(3-methoxypropyl)biphenyl-4-yl]piperidin-4-yl}pyridin-2-amine

(+/−)-trans-tert-butyl 4-[2-(tert-butylamino)pyridin-3-yl]-3-[3-chloro-2′-(3-methoxypropyl)biphenyl-4-yl]piperidine-1-carboxylate from Example 17, step 2, was processed to the title compound similarly.

### Example 19

### Structure 54: (+/−)-cis-N-[2-(4′-{4-hydroxy-4-[4-({[(2S)-3-methoxy-2-methylpropyl]oxy}methyl)phenyl]piperidin-3-yl}-3′-methylbiphenyl-2-yl)ethyl]acetamide

### Step 1: (+/−) tert-Butyl 3-(4-hydroxy-2-methylphenyl)-4-oxopiperidine-1-carboxylate

A mixture of tert-butyl 4-oxopiperidine-1-carboxylate (1.5 g, 7.53 mmol), sodium tert-butoxide (2.53 g, 26.3 mmol), 4-bromo-3-methylphenol (1.83 g, 9.79 mmol) in THF (75 ml) was degassed with N₂. A mixture of degassed Pd₂(dba)₃ (0.345 g, 0.38 mmol) and 1,2,3,4,5-pentaphenyl-1′-(di-tert-butylphosphino)ferrocene (0.268 g, 0.376 mmol) in THF (5 mL) was added and the mixture was heated at 70° C. under reflux for 1 h. The mixture was cooled to RT and the solvent was evaporated. The residue was diluted with 1N HCl (10 mL) and extracted with EtOAc (3×20 mL). The combined organic fractions were dried over Na₂SO₄ and the solvent was evaporated. Purification by ISCO COMBI-FLASH® chromatography (SiO₂-40 g, gradient elution of 10-60% EtOAc/hexanes over 30 min) afforded the title product.

### Step 2: (+/−) tert-Butyl 3-(2-methyl-4-{[(trifluoromethyl)sulfonyl]oxy}phenyl)-4-oxopiperidine-1-carboxylate

To a solution of (+/−) tert-butyl-3-(4-hydroxy-2-methylphenyl)-4-oxopiperidine-1-carboxylate (1.6 g, 5.24 mmol) and pyridine (0.55 ml, 6.81 mmol) in CH₂Cl₂ (26.2 ml) was added triflic anhydride (0.97 ml, 5.76 mmol) at 0° C. and the mixture was stirred at 0° C. for 30 min then at rt for 30 min. The mixture was quenched with sat. NaHCO₃ (10 mL) and extracted with CH₂Cl₂ (3×15 mL). The combined organic fractions were dried over Na₂SO₄ and the solvent was evaporated. Purification by ISCO COMBI-FLASH® chromatography (SiO₂-40 g, gradient elution of 10-50% EtOAc/hexanes over 30 min) afforded the title product. MS (+ESI) m/z 460 (M+Na).

### Step 3: (+/−) tert-Butyl-3-[2′-(2-{[(benzyloxy)carbonyl]amino}ethyl)-3-methylbiphenyl-4-yl]-4-oxopiperidine-1-carboxylate

A solution of (+/−) tert-butyl 3-(2-methyl-4-{[(trifluoromethyl)sulfonyl]oxy}-phenyl)-4-oxopiperidine-1-carboxylate (0.42 g, 0.96 mmol) and benzyl {2-[2-(4,4,5,5-tetramethyl-1,3,2-dioxaborolan-2-yl)phenyl]ethyl}carbamate (Ia-7.3) (0.40 g, 1.06 mmol) in THF (5.82 ml) and water (0.58 ml) was degassed with N₂. Palladium (II) acetate (10.8 mg, 0.048 mmol), 2-dicyclohexylphosphino-2′,6′-dimethoxy-1,1′-biphenyl (0.039 g, 0.096 mmol) and potassium phosphate tribasic (0.611 g, 2.88 mmol) were added and the mixture was degassed again. The mixture was heated at 80° C. for 1 h. The solvent was evaporated, the residue was diluted with water (5 mL) and then extracted with EtOAc (3×5 mL). The combined organic fractions were dried over Na₂SO₄ and the solvent was evaporated. Purification by ISCO COMBI-FLASH® chromatography (SiO₂-24 g, gradient elution of 10-60% EtOAc/hexanes over 20 min) afforded the title product.

MS (+ESI) m/z 565 (M+Na).

### Step 4: (+/−) tert-Butyl 3-{2′-[2-(acetylamino)ethyl]-3-methylbiphenyl-4-yl}-4-oxopiperidine-1-carboxylate

A mixture of (+/−) tert-butyl 3-[2′-(2-{[(benzyloxy)carbonyl]amino}ethyl)-3-methylbiphenyl-4-yl]-4-oxopiperidine-1-carboxylate (0.2 g, 0.369 mmol), acetic anhydride (0.07 ml, 0.737 mmol) and 10% palladium on carbon (0.039 g, 0.037 mmol) in EtOH (1.8 ml) was hydrogenated at RT overnight. The mixture was filtered through a pad of CELITE□ and the solvent was evaporated to afford the title product. MS (+ESI) m/z 473 (M+Na).

### Step 5: (+/−)-cis-tert-butyl-3-{2′-[2-(acetylamino)ethyl]-3-methylbiphenyl-4-yl}-4-hydroxy-4-[4-({[(2S)-3-methoxy-2-methylpropyl]oxy}methyl)phenyl]piperidine-1-carboxylate

A mixture of 1-bromo-4-({[(2S)-3-methoxy-2-methylpropyl]oxy}methyl)benzene (0.485 g, 1.78 mmol) (prepared according to procedure described in WO 08/040,764), magnesium (0.044 g, 1.81 mmol) and dibromoethane (5 uL) in THF (4 mL) was heated at 75° C. until most of the magnesium had dissolved (˜1 h). In a separate flask, lithium chloride (0.226 g, 5.33 mmol) was flamed dried under vacuum and cooled to RT. The Grignard reagent prepared above was added to it and the mixture was stirred at RT until most of the LiCl had dissolved (˜2 h). The mixture was cooled to 0° C. and treated with a solution of (+/−)-tert-butyl-3-{2′-[2-(acetylamino)ethyl]-3-methylbiphenyl-4-yl}-4-oxopiperidine-1-carboxylate (0.16 g, 0.36 mmol) in THF (1.8 ml). The mixture was warmed to RT and stirred for 1 h. The mixture was quenched with water (4 mL) and the THF was evaporated. The aqueous layer was acidified with acetic acid and extracted with EtOAc (3×5 mL). The combined organic fractions were dried over Na₂SO₄ and the solvent was evaporated. Purification by ISCO COMET-FLASH® chromatography (SiO₂-12 g, gradient elution of 60-90% EtOAc/hexanes over 30 min) afforded the title product.

MS (+ESI) m/z 667 (M+Na).

### Step 6: The Title Compound

To a solution of (+/−)-cis-tert-butyl-3-{2′-[2-(acetylamino)ethyl]-3-methylbiphenyl-4-yl}-4-hydroxy-4-[4-({[(2S)-3-methoxy-2-methylpropyl]oxy}methyl)phenyl]piperidine-1-carboxylate (0.09 g, 0.14 mmol) in CH₂Cl₂ (1.4 ml) was added 4N HCl in dioxane (0.7 ml, 2.79 mmol) and the mixture was stirred for 2 h. The solvent was evaporated. The mixture was basified with 2N NaOH and extracted with EtOAc (3×2 mL). The combined organic fractions were dried over Na₂SO₄ and the solvent evaporated. Purification by flash column chromatography (SiO₂, elution with 5% of a solution 7N NH₃/MeOH in CH₂Cl₂) afforded the title product. HRMS (M+H): 545.3380.

### Example 20

### Structure 58: (+/−)-trans-N-(2-{3′-chloro-4′-[4-(1-methyl-2-oxo-1,2-dihydropyridin-4-yl)piperidin-3-yl]biphenyl-2-yl}ethyl)acetamide

### Step 1: (2-methoxypyridin-4-yl)methanol

A mixture of 5-bromo-2-methoxyisonicotinaldehyde (1 eq.), Et₃N (1.05 eq.) and Pd/C (10%/weight) in EtOAc (0.13 M) was subjected to H₂ (1 atm). The mixture was stirred at room temperature overnight, diluted in EtOAc and filtered through a small pad of silica gel. The desired compound was obtained after evaporation to dryness.

### Step 2: 2-methoxyisonicotinaldehyde

To a solution of (2-methoxypyridin-4-yl)methanol (1 eq.) from step 1 in CH₂Cl₂ (0.15 M) was added MnO₂ (10 eq.). The mixture was stirred at room temperature overnight then diluted in EtOAc and filtered through a small pad of silica gel. After evaporation, the residue was purified by flash chromatography on silica gel (ISCO COMBI-FLASH®) eluting with Hexanes/EtOAc.

### Step 3: ethyl-3-(2-methoxypyridin-4-yl)acrylate

Prepared according to the procedure described in EXAMPLE 8, step 6 starting from 2-methoxyisonicotinaldehyde from step 2. The desired material was obtained after flash chromatography on silica gel (ISCO COMBI-FLASH®) and eluting with Hexanes/EtOAc.

### Step 4: Ethyl 4-(4-bromo-2-chlorophenyl)-4-cyano-3-(2-methoxypyridin-4-yl)butanoate

Prepared according to the procedure described in EXAMPLE 1, step 1 starting from ethyl-3-(2-methoxypyridin-4-yl)acrylate from step 3 and (4-bromo-2-chlorophenyl)acetonitrile (Ia-2.3).

### Step 5: (+/−)-trans-3-(4-bromo-2-chlorophenyl)-4-(2-methoxypyridin-4-yl)piperidine-2,6-dione

Prepared according to the procedure described in EXAMPLE 1, step 2 starting from ethyl-4-(4-bromo-2-chlorophenyl)-4-cyano-3-(2-methoxypyridin-4-yl)butanoate. The desired material was obtained after flash chromatography on silica gel (ISCO COMBI-FLASH®) eluting with Hexanes/EtOAc.

### Step 6: (+/−)-trans-4-[3-(4-bromo-2-chlorophenyl)piperidin-4-yl]-2-methoxypyridine

Prepared according to the procedure described in EXAMPLE 3, step 3 starting from (+/−)-trans-3-(4-bromo-2-chlorophenyl)-4-(2-methoxypyridin-4-yl)piperidine-2,6-dione.

### Step 7: (+/−)-trans-tort-butyl 3-(4-bromo-2-chlorophenyl)-4-(2-methoxypyridin-4-yl)piperidine-1-carboxylate

Prepared according to the procedure described in EXAMPLE 2, step 1 starting from (+/−)-trans-4-[3-(4-bromo-2-chlorophenyl)piperidin-4-yl]-2-methoxypyridine. The desired material was obtained after flash chromatography on silica gel (ISCO COMBI-FLASH®) eluting with Hexanes/EtOAc.

### Step 8: (+/−)-trans-t-butyl 3-(4-bromo-2-chlorophenyl)-4-(1-methyl-2-oxo-1,2-dihydropyridin-4-yl)piperidine-1-carboxylate

To a solution of (+/−)-trans-t-butyl-3-(4-bromo-2-chlorophenyl)-4-(2-methoxypyridin-4-yl)-piperidine-1-carboxylate (1 eq.) in CH₃CN (0.1 M) was added NaI (3 eq.) and CH₃I (3 eq.). The mixture was stirred at 45° C. overnight then CH₃I (10 eq.) was added and the mixture stirred at 45° C. overnight. After cooling to room temperature, the mixture was evaporated to dryness, diluted with EtOAc, washed with water, brine, dried over MgSO₄, filtered then evaporated to dryness to obtain the desired product.

### Step 9: (+/−)-trans-t-butyl-3-{2′-[2-(acetylamino)ethyl]-3-chlorobiphenyl-4-yl}-4-(1-methyl-2-oxo-1,2-dihydropyridin-4-yl)piperidine-1-carboxylate

Prepared according to the procedure described in EXAMPLE 3, step 5 starting from (+/−)-trans-t-butyl 3-(4-bromo-2-chlorophenyl)-4-(1-methyl-2-oxo-1,2-dihydropyridin-4-yl)piperidine-1-carboxylate and using N-[2-(2-bromophenyl)ethyl]acetamide Ia-6.4. The desired material was obtained after flash chromatography on silica gel eluting with 95% CH₂Cl₂/5% MeOH.

### Step 10: (+/−)-trans-N-(2-{3′-chloro-4′-[4-(1-methyl-2-oxo-1,2-dihydropyridin-4-yl)piperidin-3-yl]biphenyl-2-yl}ethyl)acetamide

Prepared according to the procedure described in EXAMPLE 2, step 3 starting from (+/−)-trans-t-butyl-3-{2′-[2-(acetylamino)ethyl]-3-chlorobiphenyl-4-yl}-4-(1-methyl-2-oxo-1,2-dihydropyridin-4-yl)piperidine-1-carboxylate. The desired material was obtained after flash chromatography on silica gel eluting with 90% CH₂Cl₂/10% 2M NH₃ in MeOH.

### Example 21

### Structure 59: (3S,4S)—N-(2-{3′-chloro-4′-[(3S,4S)-4-(1-methyl-2-oxo-1,2-dihydropyridin-4-yl)piperidin-3-yl]biphenyl-2-yl}ethyl)acetamide

The two enantiomers of (+/−)-trans-t-butyl-3-{2′-[2-(acetylamino)ethyl]-3-chlorobiphenyl-4-yl}-4-(1-methyl-2-oxo-1,2-dihydropyridin-4-yl)piperidine-1-carboxylate were separated on chiral HPLC using Chiralpak AD column (4.6 mm×250 mm); 30% EtOH/70% Hexanes; flow: 10 mL/min. The slow eluting compound was isolated and the BOC protecting group was removed following procedure described for EXAMPLE 2, step 3 to give the desired product. HRMS (M+H)⁺: 464.2138.

### Example 22

### Structure 60: (3R,4R)—N-(2-{3′-chloro-4′-[(3R,4R)-4-(1-methyl-2-oxo-1,2-dihydropyridin-4-yl)piperidin-3-yl]biphenyl-2-yl}ethyl)acetamide

The two enantiomers of (+/−)-trans-t-butyl-3-{2′-[2-(acetylamino)ethyl]-3-chlorobiphenyl-4-yl}-4-(1-methyl-2-oxo-1,2-dihydropyridin-4-yl)piperidine-1-carboxylate were separated on chiral HPLC using Chiralpak AD column (4.6 mm×250 mm); 30% EtOH/70% Hexanes; flow: 10 mL/min. The fast eluting compound was isolated and the BOC protecting group was removed following procedure described for EXAMPLE 2, step 3 to give the desired product. HRMS (M+H)⁺: 464.2113.

### Example 23

### Structure 57: (+/−)-trans-4-{3-[3-chloro-2′-(3-methoxypropyl)biphenyl-4-yl]piperidin-4-yl}-1-methylpyridin-2(1H)-one

### Step 1: (+/−)-trans-t-butyl-3-[3-chloro-2′-(3-methoxypropyl)biphenyl-4-yl]-4-(1-methyl-2-oxo-1,2-dihydropyridin-4-yl)piperidine-1-carboxylate

To a solution of (+/−)-trans-t-butyl-3-(4-bromo-2-chlorophenyl)-4-(1-methyl-2-oxo-1,2-dihydropyridin-4-yl)piperidine-1-carboxylate (1 eq.), [2-(3-methoxypropyl)phenyl]boronic acid (Ia-7.2) (2.3 eq.), Na₂CO₃ (5 eq.; 2 M) in t-BuOH (0.05 M) was added Pd(PPh₃)₄ (0.05 eq.). The mixture was stirred at 80° C. for 3 h then cooled to room temperature. After diluting with ethyl acetate, the organic phase was washed with water, a saturated aqueous solution of NaHCO₃ and brine. The solution was dried over MgSO₄, filtered then evaporated to dryness. The desired product was obtained as a white foam after flash chromatography on silica gel using 100% EtOAc.

### Step 2: (+/−)-trans-4-{3-[3-chloro-2′-(3-methoxypropyl)biphenyl-4-yl]piperidin-4-yl}-1-methylpyridin-2(1H)-one

Prepared according to the procedure described in EXAMPLE 2, step 3 starting from (+/−)-trans-t-butyl-3-[3-chloro-2′-(3-methoxypropyl)biphenyl-4-yl]-4-(1-methyl-2-oxo-1,2-dihydropyridin-4-yl)piperidine-1-carboxylate. The desired material was obtained as a white foam after purification by reverse phase chromatography. HRMS ESI [M+H]; 451.2176.

### Example 24

### Structure 68: (3S,4R)-3-[5-(2,6-dichlorophenyl)isoxazol-3-yl]-4-(3,4-difluorophenyl)piperidin-4-ol

### Step 1: (3S,4R)-1-(tert-butoxycarbonyl)-4-(3,4-difluorophenyl)-4-hydroxypiperidine-3-carboxylic acid acid (5) according to Scheme 6

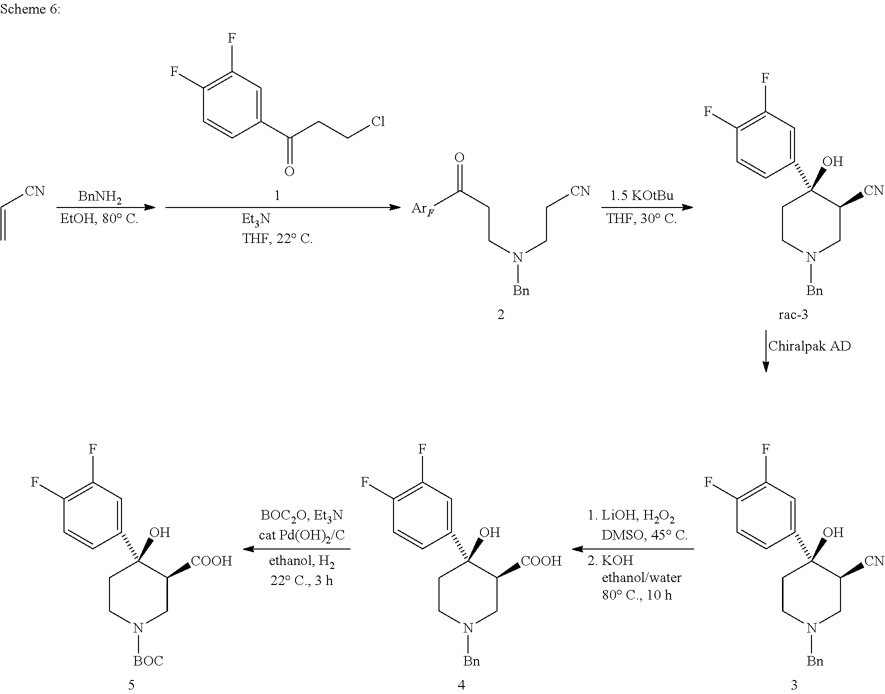

A) To a stirred solution of aluminum chloride (40.0 g, 0.300 mol) in CS₂ (125 ml) was added 1,2-difluorobenzene (24.63 ml, 0.250 mol). 3-Cloropropionylchloride (23.87 ml, 0.250 mol) was then added drop-wise and the resulting solution was stirred at reflux (45° C.) for a period of 20 h. The reaction mixture was cooled to 0° C. and quenched by slow addition of 1N HCl. The biphasic mixture was then extracted with 3×MTBE. The combined organics were washed once with brine, dried (MgSO₄), filtered and concentration to afford a brown oil. The residue was purified over a pad of silica using 25% EtOAc/Hex to afford 48.5 g of ketone 1 as a clear orange oil.

B) A stirred solution of acrylonitrile (81 ml, 1225 mmol) and benzylamine (127 ml, 1164 mmol) in Ethanol (325 ml) was heated to reflux for 2.5 h. The reaction mixture was cooled to rt and concentrated in vacuo to afford 185.45 g of N-3-(benzylamino)propionitrile as clear oil which was used without further purification.

C) To a stirred solution of 3-N-(benzylamino)proprionitrile (150 g, 936 mmol) and triethylamine (326 ml, 2341 mmol) in THF (750 ml) was added 3-chloroproprio-3′,4′-difluorophenone (1) (201 g, 983 mmol) via an addition funnel while keeping the temperature below 25° C. over a period of 30 min. This reaction mixture (slurry) was stirred at rt for 30 min and was then quenched with sat. NaHCO₃, and extracted with 3×MTBE. The combined organics were washed once with brine, dried (MgSO₄), filtered and concentrated to afford 307.6 g of compound 2 as brown oil which was used without purification.

D) Ketonitrile 2 (30 g, 91 mmol) was diluted in 250 mL THF, charged into a 3-neck RBF which was immersed into an oil bath set at 30° C. KOtBu (15.4 g, 137 mmol) was dissolved in 150 mL THF with vigorous stirring and this solution was added to the above solution over 15 min via an addition funnel, and the reaction mixture was stirred at the temperature for 1 h. The mixture was poured into a mixture of 1N HCl/MTBE. The phases were separated and the aqueous layer was extracted 2× with MTBE (100 mL each). The organic layers were combined, washed with saturated NaHCO₃ and brine, dried (Na₂SO₄) and concentrated in vacuo to give product 3 as a brown oil. The crude product was separated using a ChiralPak AD column to give the pair of enantiomers. The desired (3S,4R)-isomer was hydrolyzed as described below.

E) (3S,4R)-Hydroxy nitrile 3 (73 g, crude), LiOH (11.6 g) and DMSO (350 mL) were charged to a 3-neck RBF fitted with reflux a condenser, an addition funnel and an internal temperature probe. The flask was heated in an oil bath until the internal temperature reached 58° C. Hydrogen peroxide (30%, 99 mL) was then added via the addition funnel slowly over a 75 min period. During the first 20 min, the internal temperature rapidly climbed to 116° C., then stayed above 90° C. for the remainder of the peroxide addition. After addition was complete, the mixture was cooled to rt, and to it was added conc. HCl until pH ˜1. MTBE was added to the flask, and the biphasic mixture was transferred to a separatory funnel. The phases were separated and the aqueous layer was extracted 3× with 2-methyltetrahydrofuran (MeTHF). The acidic aqueous layer was neutralized to pH 8 with 10N NaOH, and then extracted 3× with MeTHF. All organics were combined, washed with brine, dried (Na₂SO₄) and concentrated in vacuo to give a dark viscous oil, which was used directly for the next step.

F) Hydroxy-amide from above (80 g, crude), potassium hydroxide (8M, 75 mL) and ethanol (140 mL) were charged into a RBF flask fitted with a reflux condenser and a nitrogen inlet. The mixture was heated in an 80° C. oil bath for 10 h, cooled to rt and diluted with MeTHF and water. Cone. HCl was added until pH ˜1 and the mixture was transferred to a separatory funnel. The aqueous layer was separated and extracted with 3×MeTHF (250 mL each). All organics were combined, wash with brine, dried (Na₂SO₄) and concentrated in vacuo. The residue was treated with heptane to afford hydroxy acid 4 as a viscous brown glassy-oil which was used without further purification.

G) Hydroxy-acid 4 (68 g, crude), Pd(OH)₂ on carbon (27.4 g, 39 mmol), Di-tert-butyl-dicarbonate (51.1 g, 234 mmol), triethylamine (27.2 mL, 195 mmol) in ethanol (340 mL) were charged to a steel-bomb which was pressurized to 200 psi with H₂. The bomb was vented, then re-pressurized to 200 psi twice with H₂ and stirred at rt for 2 h. The crude slurry was filtered over a solka-floc pad which was washed with EtOH and MTBE. The filtrate was concentrated in vacuo to give a brown oil, which was diluted with MeTHF and MTBE, and then washed with 1N HCl. The aqueous layer was separated and extracted with MeTHF/MTBE (1/1). The organic extracts were combined, washed with brine, dried (Na₂SO₄) and concentrated in vacuo to give a viscous brown oil (49 g). The oil was diluted with 250 mL MTBE and treated with 100 mL 1N NaOH. The solid was collected by filtration. The biphasic filtrate was poured into a separatory funnel, and the phases were separated. The organic phase was washed twice with 1N NaOH. The aqueous phases were acidified to pH ˜1 with conc. HCl. The solid from above was suspended in MeTHF/water and then treated with conc. HCl until all material was dissolved, and combined with acidified aqueous from above. The phases were separated and the aqueous layer was extracted twice with MeTHF/MTBE (1/1). The organic layers were combined, washed with brine, dried (Na₂SO₄) and concentrated in vacuo to give acid 5 as an amber colored glass (25 g).

### Step 2: Tert-butyl (3R,4R)-4-(3,4-difluorophenyl)-4-hydroxy-3-(hydroxymethyl)piperidine-1-carboxylate

To a solution of (3S,4R)-1-tert-butoxycarbonyl-4-(3,4-difluorophenyl)-4-hydroxy-piperidine-3-carboxylic acid (5) (1.0 eq.) in THF (0.14 M) was added borane-dimethyl sulfide complex (3 eq.) and the solution was refluxed for 4 h. The solution was cooled to room temperature and methanol is carefully added to consume the remaining borane. The solvents were removed under reduced pressure. The residue was purified by a silica-gel plug eluting with EtOAc/Hexanes (50%) to give the desired product as a white foam.

### Step 3: Tert-butyl (3S,4R)-4-(3,4-difluorophenyl)-3-formyl-4-hydroxy-piperidine-1-carboxylate

To a solution of tert-butyl (3R,4R)-4-(3,4-difluorophenyl)-4-hydroxy-3-(hydroxymethyl)piperidine-1-carboxylate (1.0 eq.) in dichloromethane (0.1 M) was added Dess-Martin Periodinane (1.5 eq.) and sodium bicarbonate (3 eq.), and the solution was stirred at room temperature for 16 h. Water was added and the phases were separated. The aqueous phase was extracted with dichloromethane and the organic layers were combined and concentrated under reduced pressure. The residue was purified by a silica-gel plug eluting with EtOAc/Hexanes (50%) to give the desired product as a white foam.

### Step 4: Tert-butyl (3S,4R)-4-(3,4-difluorophenyl)-3-ethynyl-4-hydroxy-piperidine-1-carboxylate

To a solution of tert-butyl (3S,4R)-4-(3,4-difluorophenyl)-3-formyl-4-hydroxy-piperidine-1-carboxylate (1.0 eq.) in methanol (0.146 M) was added potassium carbonate (2 eq.) and Bestmann's reagent (1.2 eq.). The solution was stirred for 811 at room temperature under a nitrogen atmosphere. The solvent was removed under reduced pressure and the residue was partitioned between ethyl acetate and saturated sodium bicarbonate. The organic extract was washed with brine, dried over MgSO₄, filtered and concentrated. The residue was purified by flash chromatography on silica gel, eluting with EtOAc/Hexanes (10 to 60%) to give the desired product as a white solid.

### Step 5: tert-butyl (3S,4R)-3-[5-(2,6-dichlorophenyl)isoxazol-3-yl]-4-(3,4-difluorophenyl)-4-hydroxy-piperidine-1-carboxylate

To a solution of 2,6-dichlorobenzaldehyde oxime (1.75 eq.) in methanol (0.11 M) was added chloramine-T (1.75 eq.) and the mixture was stirred for 5 minutes. Tert-butyl (3S,4R)-4-(3,4-difluorophenyl)-3-ethynyl-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 100%) to give the desired product as a white foam.

### Step 6: (3S,4R)-3-[5-(2,6-dichlorophenyl)isoxazol-3-yl]-4-(3,4-difluorophenyl)piperidin-4-ol

To a solution of tert-butyl (3R,4R)-3-[5-(2,6-dichlorophenyl)isoxazol-3-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and the solution was stirred for 2 h at rt. The solvent was removed under reduced pressure. The residue was purified by flash chromatography on silica gel, eluting with (5% NH₄OH in MeOH)/DCM (0 to 20%) to give the title compound as a white foam.

### Example 25

### Structure 69: (3S,4R)-3-[5-(2-chloro-4-fluoro-phenyl)isoxazol-3-yl]-4-(3,4-difluorophenyl)piperidin-4-ol

### Step 1: Tert-butyl (3S,4R)-3-[5-(2-chloro-4-fluoro-phenyl)isoxazol-3-yl]-4-(3,4-difluorophenyl)-4-hydroxy-piperidine-1-carboxylate

To a solution of 2-chloro-4-fluoro-benzaldehyde oxime (2 eq.) in methanol (0.11 M) was added chloramine-T (2 eq.) and to solution was stirred for 5 minutes. Tert-butyl (3S,4R)-4-(3,4-difluorophenyl)-3-ethynyl-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (10 to 75%) to give the desired product as a white foam.

### Step 2: (3S,4R)-3-[5-(2-chloro-4-fluoro-phenyl)isoxazol-3-yl]-4-(3,4-difluorophenyl)piperidin-4-ol

To a solution of tert-butyl (3S,4R)-3-[5-(2-chloro-4-fluoro-phenyl)isoxazol-3-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white foam.

### Example 26

### Structure 70: (3S,4R)-3-[4-bromo-3-(2,6-dichlorophenyl)isoxazole-5-yl]-4-(3,4-difluorophenyl)piperidin-4-ol

### Step 1: tert-butyl (3S,4R)-3-(2,2-dibromovinyl)-3-(3,4-difluorophenyl)-4-hydroxypiperidine-1-carboxylate

Carbon tetrabromide (2 eq.) was dissolved in dichloromethane and cooled to 0° C. Triphenylphosphine (4 eq.) in dichloromethane (2 M) was added and the mixture stirred for 15 min. Triethylamine (1 eq.) was added and the reaction mixture cooled to −78° C. A solution of tert-butyl (3S,4R)-4-(3,4-difluorophenyl)-3-formyl-4-hydroxy-piperidine-1-carboxylate (1 eq.) in dichloromethane was added and the mixture stirred at −78° C. for 1.5 h, then at room temperature for 3 h. Aq. NaHCO₃ was added, the phases were separated and the aq. phase was extracted with CH₂Cl₂. The combined organic phases were dried over Na₂SO₄, filtered and concentrated in vacuo. The residue was purified by flash chromatography, eluting with 10-100% EtOAc in hexanes, to afford the product as a white solid.

### Step 2: tert-butyl (3S,4R)-3-(bromoethynyl)-3-(3,4-difluorophenyl)-4-hydroxypiperidine-1-carboxylate

To a solution of olefin from step 1 (1 eq.) in THF (0.2 M) at −78° C. was added potassium tert-butoxide (1.2 eq.). The reaction mixture was stirred at −78° C. for 30 min, warmed to rt and stirred for a further 30 min. Another portion of potassium tert-butoxide (0.55 eq.) was added, the reaction mixture was stirred for 30 min more, and quenched with water. The reaction mixture was extracted with EtOAc, washed with brine, dried over Na₂SO₄, filtered and concentrated in vacuo. The residue was purified by column chromatography. Eluting with 10-70% EtOAc in hexanes afforded the title compound as a white foam.

### Step 3: tert-butyl (3S,4R)-3-[4-bromo-3-(2,6-dichlorophenyl)isoxazole-5-yl]-4-(3,4-difluorophenyl)-4-hydroxylpiperidine-1-carboxylate

Prepared according to the general procedure for cycloadditions described in Example 24, step 5 using tert-butyl (3S,4R)-3-(bromoethynyl)-3-(3,4-difluorophenyl)-4-hydroxypiperidine-1-carboxylate and 2,6-dichlorobenzaldehye oxime.

### Step 4: (3S,4R)-3-[4-bromo-3-(2,6-dichlorophenyl)isoxazole-5-yl]-4-(3,4-difluorophenyl)piperidin-4-ol

Prepared according to the general procedure for deprotection with HCl in dioxane as described in Example 24, step 6.

### Example 27

### Structure 71: (3S,4R)-3-[4-bromo-3-(2,3-dichlorophenyl)isoxazol-5-yl]-4-(3,4-difluorophenyl)piperidin-4-ol

### Step 1: 2,3-dichlorobenzaldehyde oxime

To a solution of 2,3-dichlorobenzaldehyde (1 eq.) and pyridine (3.6 eq.) in ethanol (0.1 M) was added hydroxilamine hyrochloride (1.5 eq.) and the solution was stirred at room temperature for 18 h. The solvents were removed under reduced pressure and the crude mixture was purified by flash chromatography on silica gel, eluting with EtOAc/Hexanes to give the desired oxime as a white solid.

### Step 2: Tert-butyl (3S,4R)-3-[4-bromo-3-(2,3-dichlorophenyl)isoxazol-5-yl]-4-(3,4-difluorophenyl)-4-hydroxy-piperidine-1-carboxylate

To a solution of 2,3-dichlorobenzaldehyde oxime (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 70%) to give the desired product as a white solid.

### Step 3: (3S,4R)-3-[4-bromo-3-(2,3-dichlorophenyl)isoxazol-5-yl]-4-(3,4-difluorophenyl)piperidin-4-ol

To a solution of tert-butyl (3S,4R)-3-[4-bromo-3-(2,3-dichlorophenyl)isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white foam.

### Example 28

### Structure 72: (3S,4R)-3-[4-bromo-3-(2-chlorophenyl)isoxazol-5-yl]-4-(3,4-difluorophenyl)piperidin-4-ol

### Step 1: Tert-butyl (3S,4R)-3-[4-bromo-3-(2-chlorophenyl)isoxazol-5-yl]-4-(3,4-difluorophenyl)-4-hydroxy-piperidine-1-carboxylate

To a solution of 2-chlorobenzaldehyde oxime (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 50%) to give the desired product as a white solid.

### Step 2: (3S,4R)-3-[4-bromo-3-(2-chlorophenyl)isoxazol-5-yl]-4-(3,4-difluorophenyl)piperidin-4-ol

To a solution of tert-butyl (3S,4R)-3-[4-bromo-3-(2-chlorophenyl)isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white solid.

### Example 29

### Structure 73: (3S,4R)-3-[4-bromo-3-(2,3-dimethylphenyl)isoxazol-5-yl]-4-(3,4-difluorophenyl)piperidin-4-ol

### Step 1: 2,3-dimethylbenzaldehyde oxime

Using general procedure described in Example 27, step 1,2,3-dimethylbenzaldehyde was used to give the desired oxime as a white solid.

### Step 2: Tert-butyl (3S,4R)-3-[4-bromo-3-(2,3-dimethylphenyl)isoxazol-5-yl]-4-(3,4-difluorophenyl)-4-hydroxy-piperidine-1-carboxylate

To a solution of 2,3-dimethylbenzaldehyde oxime (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 50%) to give the desired product as a white solid.

### Step 3: (3S,4R)-3-[4-bromo-3-(2,3-dimethylphenyl)isoxazol-5-yl]-4-(3,4-difluorophenyl)piperidin-4-ol

To a solution of tert-butyl (3S,4R)-3-[4-bromo-3-(2,3-dimethylphenyl)isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white solid.

### Example 30

### Structure 74: (3S,4R)-3-[4-bromo-3-(6-chloro-2-fluoro-3-methyl-phenyl)isoxazol-5-yl]-4-(3,4-difluorophenyl)piperidin-4-ol

### Step 1: 6-chloro-2-fluoro-3-methyl-benzaldehyde oxime

Using general procedure described in Example 27, step 1, 6-chloro-2-fluoro-3-methyl-benzaldehyde was used to give the desired oxime as a white solid.

### Step 2: Tert-butyl (3S,4R)-3-[4-bromo-3-(6-chloro-2-fluoro-3-methyl-phenyl)isoxazol-5-yl]-4-(3,4-difluorophenyl)-4-hydroxy-piperidine-1-carboxylate

To a solution of 6-chloro-2-fluoro-3-methyl-benzaldehyde oxime (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 50%) to give the desired product as a white solid.

### Step 3: (3S,4R)-3-[4-bromo-3-(6-chloro-2-fluoro-3-methyl-phenyl)isoxazol-5-yl]-4-(3,4-difluorophenyl)piperidin-4-ol

To a solution of tert-butyl (3S,4R)-3-[4-bromo-3-(6-chloro-2-fluoro-3-methyl-phenyl)isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white foam.

### Example 31

### Structure 75: (3S,4R)-3-[4-bromo-3-(1-naphthyl)isoxazol-5-yl]-4-(3,4-difluorophenyl)piperidin-4-ol

### Step 1: naphthalene-1-carbaldehyde oxime

Using general procedure described in Example 27, step 1, naphthalene-1-carbaldehyde was used to give the desired oxime as a white solid.

### Step 2: Tert-butyl (3S,4R)-3-[4-bromo-3-(1-naphthyl)isoxazol-5-yl]-4-(3,4-difluorophenyl)-4-hydroxy-piperidine-1-carboxylate

To a solution of naphthalene-1-carbaldehyde oxime (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 50%) to give the desired product as a white solid.

### Step 3: (3S,4R)-3-[4-bromo-3-(1-naphthyl)isoxazol-5-yl]-4-(3,4-difluorophenyl)piperidin-4-ol

To a solution of tert-butyl (3S,4R)-3-[4-bromo-3-(1-naphthyl)isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (5% NH₄OH in MeOH)/DCM (0 to 20%) to give the title compound as a white solid.

### Example 32

### Structure 76: N-[2-[2-[4-bromo-5-[(3S,4R)-4-(3,4-difluorophenyl)-4-hydroxy-3-piperidyl]isoxazol-3-yl]phenyl]ethyl]acetamide

### Step 1: Methyl 2-[hydroxyiminomethyl]benzoate

Methyl 2-formylbenzoate (1 eq.) and hydroxylamine hydrochloride (1.5 eq.) were dissolved in methanol and water (7:3, 0.1 M) and the solution was stirred at room temperature for 2 hours. Ethyl acetate was added and the phases were separated. The aqueous phase was washed with ethyl acetate and the combined organic phase was dried over MgSO₄, filtered and the filtrate evaporated under reduced pressure. The residue was purified by silica-gel plug eluting with EtOAc/Hexanes (5% to 60%) to give the desired product as a white solid.

### Step 2: Tert-butyl (3S,4R)-3-[4-bromo-3-(2-methoxycarbonylphenyl)isoxazol-5-yl]-4-(3,4-difluorophenyl)-4-hydroxy-piperidine-1-carboxylate

To a solution of methyl 2-[hydroxyiminomethyl]benzoate (2 eq.) in methanol (0.1 M) was added chloramine-T (2 eq.) and to solution was stirred for 5 minutes. Tert-butyl (3S,4R)-3-(2-bromoethynyl)-4-(3,4-difluorophenyl)-4-hydroxy-piperidine-1-carboxylate (1 eq.) was added and the solution was refluxed overnight. The solvent was removed under reduced pressure. The residue was purified by flash chromatography on silica gel, eluting with EtOAc/Hexanes (0 to 50%) to give the desired product as a white solid.

### Step 3: Tert-butyl (3S,4R)-3-[4-bromo-3-[2-(hydroxymethyl)phenyl]isoxazol-5-yl]-4-(3,4-difluorophenyl)-4-hydroxy-piperidine-1-carboxylate

To a solution of tert-butyl (3S,4R)-3-[4-bromo-3-(2-methoxycarbonylphenyl)isoxazol-5-yl]-4-(3,4-difluorophenyl)-4-hydroxy-piperidine-1-carboxylate (1 eq.) in dichloromethane (0.1 M) at −78° C. was added DIBAL-H (1.5 M in toluene, 3 eq.) dropwise. The solution was stirred at −78° C. for 10 minutes before the cold bath was removed. The solution is stirred for another hour at room temperature and an aqueous solution of 1M HCl was added. The aqueous phase was washed with dichloromethane and the combined organic phase was dried over MgSO₄, filtered and the filtrate evaporated under reduced pressure. The residue was purified by silica-gel plug eluting with EtOAc/Hexanes (0% to 50%) to give the desired product as a white solid.

### Step 4: Tert-butyl (3S,4R)-3-[4-bromo-3-[2-(methylsulfonyloxymethyl)phenyl]-isoxazol-5-yl]-4-(3,4-difluorophenyl)-4-hydroxy-piperidine-1-carboxylate

To a solution of tert-butyl (3S,4R)-3-[4-bromo-3-[2-(hydroxymethyl)phenyl]isoxazol-5-yl]-4-(3,4-difluorophenyl)-4-hydroxy-piperidine-1-carboxylate (1 eq.) in dichloromethane (0.1 M) at 0° C. was added Hunig's base (1.5 eq.) and methanesulfonyl chloride (1.2 eq.). The solution was stirred at 0° C. for 90 minutes. The solvent was removed under reduced pressure before ethyl acetate and water were added. The aqueous phase was washed with ethyl acetate and the combined organic phase was dried over MgSO₄, filtered and the filtrate evaporated under reduced pressure, yielding the desired mesylate as a crude mixture.

### Step 5: Tert-butyl (3S,4R)-3-[4-bromo-3-[2-(cyanomethyl)phenyl] isoxazol-5-yl]-4-(3,4-difluorophenyl)-4-hydroxy-piperidine-1-carboxylate

To a solution of tert-butyl (3S,4R)-3-[4-bromo-3-[2-(methylsulfonyloxymethyl)phenyl]-isoxazol-5-yl]-4-(3,4-difluorophenyl)-4-hydroxy-piperidine-1-carboxylate (1 eq.) in acetonitrile (0.25 M) was added 18-crown-6 (1.5 eq.) followed by potassium cyanide (3 eq.). The solution was stirred at room temperature for 18 hours. Brine was added, and the aqueous phase was separated and extracted with diethyl ether. The combined organic phases were dried over MgSO₄, filtered and the filtrate evaporated under reduced pressure. The residue was purified by flash chromatography on silica gel, eluting with EtOAc/Hexanes (20 to 80%) to give the desired product as a colorless oil.

### Step 6: Tert-butyl (3S,4R)-3-[3-[2-(2-aminoethyl)phenyl]-4-bromo-isoxazol-5-yl]-4-(3,4-difluorophenyl)-4-hydroxy-piperidine-1-carboxylate

Tert-butyl (3S,4R)-3-[4-bromo-3-[2-(cyanomethyl)phenyl]isoxazol-5-yl]-4-(3,4-difluorophenyl)-4-hydroxy-piperidine-1-carboxylate (1 eq.) was dissolved in tetrahydrofuran (0.1 M) and borane-dimethyl sulfide complex (3 eq.) was added. The solution was refluxed for 2 hours. The solution was then cooled to 0° C. and water (100 eq.) was added followed by aq. KOH (3.5 M, 6.5 eq.). The mixture was refluxed for another 2 hours, cooled to room temperature and diluted with dichloromethane and water. The aqueous phase was washed with diethyl ether, the combined organic phase was dried over MgSO₄, filtered and the filtrate evaporated under reduced pressure, yielding the desire compound as a yellow solid which was used without further purification.

### Step 7: Tert-butyl (3S,4R)-3-[3-[2-(2-acetamidoethyl)phenyl]-4-bromo-isoxazol-5-yl]-4-(3,4-difluorophenyl)-4-hydroxy-piperidine-1-carboxylate

To a solution of tert-butyl (3S,4R)-3-[3-[2-(2-aminoethyl)phenyl]-4-bromo-isoxazol-5-yl]-4-(3,4-difluorophenyl)-4-hydroxy-piperidine-1-carboxylate (1 eq.) in dichloromethane (0.1 M) was added triethylamine (2 eq.) followed by acetyl chloride (1.05 eq.). The solution was stirred at room temperature for 30 minutes. An aqueous solution of sodium hydroxide (1M) was added and the aqueous phase was washed with dichloromethane, the combined organic phase was dried over MgSO₄, filtered and the filtrate evaporated under reduced pressure. The residue was purified by flash chromatography on silica gel, eluting with EtOAc/Hexanes (50 to 100%) to give the desired product as a pale yellow solid.

### Step 8: N-[2-[2-[4-bromo-5-[(3S,4R)-4-(3,4-difluorophenyl)-4-hydroxy-3-piperidyl]isoxazol-3-yl]phenyl]ethyl]acetamide

To a solution of tert-butyl (3S,4R)-3-[3-[2-(2-acetamidoethyl)phenyl]-4-bromo-isoxazol-5-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure to give the title compound as a white solid.

### Example 33

### Structure 86: N-[2-[2-[4-bromo-5-(3R,4R)-4-(3,4-difluorophenyl)-4-hydroxy-3-piperidyl]isoxazol-3-yl-5-fluoro-phenyl]ethyl]acetamide

### Step 1: 2-(2-bromo-5-fluoro-phenyl)ethanamine

2-(2-bromo-5-fluoro-phenyl)acetonitrile (1 eq.) was dissolved in THF (0.1 M) and borane-dimethyl sulfide complex (3 eq.) was added. The solution was refluxed for 2 hours. The solution was then cooled to 0° C. and water (100 eq.) was added followed by aq. KOH (3.5 M, 6.5 eq.). The mixture was refluxed for another 2 hours, cooled to room temperature and diluted with ether and water. The aqueous phase was washed with diethyl ether, the combined organic phase was dried over MgSO₄, filtered and the filtrate evaporated under reduced pressure, yielding the desire compound a yellow solid which was used without further purification.

### Step 2: N-[2-(2-bromo-5-fluoro-phenyl)ethyl]acetamide

To a solution of 2-(2-bromo-5-fluoro-phenyl)ethanamine (1 eq.) in dichloromethane (0.1 M) was added triethylamine (2 eq.) followed by acetyl chloride (1.05 eq.). The solution was stirred at room temperature for 30 minutes. An aqueous solution of sodium hydroxide (1M) was added and the aqueous phase was washed with dichloromethane, the combined organic phase was dried over MgSO₄, filtered and the filtrate evaporated under reduced pressure. The residue was purified by flash chromatography on silica gel, eluting with EtOAc/Hexanes (50 to 100%) to give the desired product as a pale yellow solid.

### Step 3: N-[2-[5-fluoro-2-(2-trimethylsilylethynyl)phenyl]ethyl]acetamide

N-[2-(2-bromo-5-fluoro-phenyl)ethyl]acetamide (1 eq.), copper (I) iodide (0.05 eq.) and PdCl₂(PPh₃)₂ (0.05 eq.) were dissolved in DMF (0.35 M) in a sealed tube before triethylamine (5 eq.) and ethynyl(trimethyl)silane (1.5 eq.) were added. The tube was sealed and heated to 100° C. for 20 h. The tube was cooled to rt and more copper (I) iodide (0.05 eq.) and PdCl₂(PPh₃)₂ (0.05 eq.) and ethynyl(trimethyl)silane (1.5 eq.) were added. The tube was sealed and heated for further 20 h. An aqueous solution of sodium hydroxide (1M) was added and the aqueous phase was washed with dichloromethane, the combined organic phase was dried over MgSO₄, filtered and the filtrate evaporated under reduced pressure. The residue was purified by flash chromatography on silica gel, eluting with EtOAc/Hexanes (0 to 100%) to give the desired product as a brown oil.

### Step 4: N-[2-[5-fluoro-2-(bromoethynyl)phenyl]ethyl]acetamide

To a solution of N-[2-[5-fluoro-2-(2-trimethylsilylethynyl)phenyl]ethyl]acetamide (1 eq.) and silver (I) nitrate (0.3 eq.) in acetone (0.185 M) was added NBS. The solution was stirred at room temperature, in the dark, for 2 h. Solvent was removed under reduced pressure. The residue was purified by flash chromatography on silica gel, eluting with EtOAc/Hexanes (0 to 100%) to give the desired product as a brown solid.

### Step 5: Tert-butyl (3R,4R)-3-[5-[2-(2-acetamidoethyl)-4-fluoro-phenyl]-4-bromo-isoxazol-3-yl]-4-(3,4-difluorophenyl)-4-hydroxy-piperidine-1-carboxylate

To a solution of tert-butyl (3R,4R)-4-(3,4-difluorophenyl)-4-hydroxy-3-[hydroxyiminomethyl]-piperidine-1-carboxylate (see EXAMPLE 37, step 2) (1 eq.) in methanol (0.1 M) was added chloramine-T (1 eq.) and to solution was stirred for 5 minutes. N-[2-[5-fluoro-2-(bromoethynyl)phenyl]ethyl]acetamide (1.5 eq.) was added and the solution was refluxed overnight. The solvent was removed under reduced pressure. The residue was purified by flash chromatography on silica gel, eluting with EtOAc/Hexanes (50 to 100%) to give the desired product as a white solid.

### Step 6: N-[2-[2-[4-bromo-3-[(3R,4R)-4-(3,4-difluorophenyl)-4-hydroxy-3-piperidyl]isoxazol-5-yl]-5-fluoro-phenyl]ethyl]acetamide

To a solution of tert-butyl (3R,4R)-3-[5-[2-(2-acetamidoethyl)-4-fluoro-phenyl]-4-bromo-isoxazol-3-yl]-4-(3,4-difluorophenyl)-4-hydroxy-piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (2M ammonia in MeOH)/DCM (0 to 20%) to give the title compound as a white solid.

### Example 34

### Structure 87: N-[2-[2-[4-bromo-5-[(3R,4R)-4-(3,4-difluorophenyl)-4-hydroxy-3-piperidyl]isoxazol-3-yl]-6-fluoro-phenyl]ethyl]acetamide

Following the procedure described for compound Example 33, the title compound was obtained as a white solid starting from 2-(2-bromo-6-fluoro-phenyl)acetonitrile.

### Example 35

### Structure 89: N-[2-[2-[4-bromo-3-[(3R,4R)-4-hydroxy-4-[4-[[(2S)-3-methoxy-2-methyl-propoxy]methyl]phenyl]-3-piperidyl]isoxazol-5-yl]phenyl]ethyl]acetamide

### Step 1: 1-benzyl-3-(R,S)-(hydroxymethyl)-4-[4-({[(2S)-3-methoxy-2-methylpropyl]oxy}methyl)phenyl]piperidin-4-(R,S)-ol

1-benzyl-4-[4-({[(2S)-3-methoxy-2-methylpropyl]oxy}methyl)phenyl]-3-(R,S)-[(trityloxy)methyl]piperidin-4-(R,S)-ol (prepared according to procedures described in WO 08/040,764) (1 eq.) was dissolved in tetrahydrofuran and methanol (1:4, 0.085 M) and pTsOH hydrate (1.5 eq.) was added. The solution was stirred at room temperature for 18 h. The solution was then cooled to 0° C. and water (100 eq.) was added followed by aq. KOH (3.5 M, 6.5 eq.). The mixture was diluted with dichloromethane and water. The aqueous phase was washed with dichloromethane, the combined organic phase was dried over MgSO₄, filtered and the filtrate evaporated under reduced pressure, yielding the desire compound as a white solid which was used without further purification.

### Step 2: Tert-butyl (3R,4R)-4-hydroxy-3-(hydroxymethyl)-4-[4-({[(2S)-3-methoxy-2-methylpropyl]oxy}methyl)phenyl]piperidine-1-carboxylate

In a round-bottom flask was added 1-benzyl-3-(R,S)-(hydroxymethyl)-4-[4-({[(2S)-3-methoxy-2-methylpropyl]oxy}methyl)phenyl]piperidin-4-(R,S)-ol (1 eq.) followed by palladium on carbon (0.1 eq.) and di-tert-butyl dicarbonate (1.2 eq.). The flask was evacuated and back-filled with nitrogen before methanol (0.67 M) was added. The nitrogen was evacuated and back-filled with hydrogen. The suspension was stirred at rt under an atmosphere of hydrogen for 18 h. The hydrogen was evacuated by blowing nitrogen through the flask. The suspension was then filtered on CELITE□, washed with DCM and the solvents were removed under reduced pressure. The residue was purified by flash chromatography on silica gel, eluting with EtOAc/Hexanes (30 to 100%) to give the desired product as a pale yellow solid. The diastereoisomers were then separated by preparative HPLC (Chiralpak AD, 50×500 mm, 50 mL/min). The slower eluting isomer was the desired enantiomer.

### Step 3: Tert-butyl (3S,4R)-3-formyl-4-hydroxy-4-[4[[(2S)-3-methoxy-2-methyl-propoxy]methyl]phenyl]piperidine-1-carboxylate

To a solution of Tert-butyl (3R,4R)-4-hydroxy-3-(hydroxymethyl)-4-[4-[[(2S)-3-methoxy-2-methyl-propoxy]methyl]phenyl]piperidine-1-carboxylate (1.0 eq.) in dichloromethane (0.05 M) was added Dess-Martin Periodinane (3 eq.), pyridine (30 eq.) and tert-butanol (50 eq.) and the solution was stirred at room temperature for 16 h. Water was added and the phases were separated. The aqueous phase was washed with dichloromethane and the combined organic phase was dried over MgSO₄, filtered and the filtrate evaporated under reduced pressure. The residue was purified by silica-gel chromatographie eluting with EtOAc/Hexanes (30% to 85%) to give the desired product as a colorless gum.

### Step 4: Tert-butyl (3R,4R)-4-hydroxy-3-(hydroxyiminomethyl)-4-[4-[[(2S)-3-methoxy-2-methyl-propoxy]methyl]phenyl]piperidine-1-carboxylate

To a solution of tert-butyl (3S,4R)-3-formyl-4-hydroxy-4-[4-[[(2S)-3-methoxy-2-methyl-propoxy]methyl]phenyl]piperidine-1-carboxylate in ethanol/water (9:1) (0.1 M) was added hydroxylamine hydrochloride (1.5 eq.) and sodium carbonate (1.6 eq.). The solution was stirred at room temperature for 2 h. The mixture was diluted with ethyl acetate and water. The aqueous phase was washed with ethyl acetate, the combined organic phase was dried over MgSO₄, filtered and the filtrate evaporated under reduced pressure, yielding the desire compound a colorless gum which was used without further purification.

### Step 5: Tert-butyl (3R,4R)-3-[5-[2-(2-acetamidoethyl)phenyl]-4-bromo-isoxazol-3-yl]-4-hydroxy-4-[4-[[(2S)-3-methoxy-2-methyl-propoxy]methyl]phenyl]piperidine-1-carboxylate

To a solution of Tert-butyl (3R,4R)-4-hydroxy-3-(hydroxyiminomethyl)-4-[4-[[(2S)-3-methoxy-2-methyl-propoxy]methyl]phenyl]piperidine-1-carboxylate (1 eq.) in methanol (0.1 M) was added chloramine-T (1 eq.) and to solution was stirred for 30 min. N-[2-[2-(2-bromoethynyl)phenyl]ethyl]acetamide (2.5 eq.) was added and the solution was refluxed overnight. The solvent was removed under reduced pressure. The residue was purified by flash chromatography on silica gel, eluting with EtOAc/Hexanes (50 to 100%) to give the desired product as a yellow solid.

### Step 6: N-[2-[2-[4-bromo-3-[(3R,4R)-4-hydroxy-4-[4-[[(2S)-3-methoxy-2-methyl-propoxy]methyl]phenyl]-3-piperidyl]isoxazol-5-yl]phenyl]ethyl]acetamide

To a solution of tert-butyl (3R,4R)-3-[5-[2-(2-acetamidoethyl)phenyl]-4-bromo-isoxazol-3-yl]-4-hydroxy-4-[4-[[(2S)-3-methoxy-2-methyl-propoxy]methyl]phenyl]piperidine-1-carboxylate (1 eq.) in dichloromethane (0.05 M) was added a 4N solution of HCl in 1,4-dioxane (40 eq.) and to solution was stirred for 2 hours at room temperature. The solvent was removed under reduced pressure. The residue was purified by flash chromatography on silica gel, eluting with (2M ammonia in MeOH)/DCM (0 to 20%) to give the title compound as a white solid.

### Example 36

### Structure 79: (3R,4R)-3-[5-(2,6-dichlorophenyl)isoxazol-3-yl]-4-(3,4-difluorophenyl)piperidin-4-ol

### Step 1: [(2,6-dichlorophenyl)ethynyl](trimethyl)silane (general procedure for Sonogashira coupling)

A mixture of 2-bromo-1,3-dichlorobenzene (1 eq.), Pd(PPh₃)₂Cl₂ (0.05 eq.) and triethylamine (4.2 eq.) in DMF (0.4 M) was degassed by three vacuum/N₂ cycles. Trimethylsilylacetylene (1.4 eq.) was added, and the mixture was heated to 70° C. for 20 h. The mixture was taken in Et₂O, washed with water three times and once with brine, dried over Na₂SO₄, filtered and concentrated in vacuo. The residue was purified by flash chromatography on silica gel using hexanes to afford the title compound as a light yellow oil.

### Step 2: 1,3-dichloro-2-ethynylbenzene

A mixture of [(2,6-dichlorophenyl)ethynyl](trimethyl)silane from step 1 (1 eq.), K₂CO₃ (0.2 eq.) and MeOH (0.8 M) was stirred at rt for 18 h, and concentrated in vacuo. The residue was taken in CH₂Cl₂ and aq. NaHCO₃, the phases were separated and the aq. phase extracted with CH₂Cl₂. The combined organic phases were dried over Na₂SO₄, filtered and concentrated in vacuo, to afford the title compound as a tan solid,

### Step 3: tert-butyl (3R,4R)-3-[5-(2,6-dichlorophenyl)isoxazole-3-yl]-4-(3,4-difluorophenyl)-4-hydroxypiperidine-1-carboxylate

To a solution of oxime from example 37 step 2 (1 eq.) in MeOH (0.1 M) was added chloramine-T trihydrate (1 eq.) After 5 min, a solution of 1,3-dichloro-2-ethylylbenzene from step 2 (1.3 eq.) in MeOH (1 M) was added and the reaction heated to 80° C. for 3 hours, The reaction mixture was cooled to rt, extracted with Et₂O from water, dried over Na₂SO₄, filtered and concentrated in vacuo. The residue was purified by flash chromatography on silica gel using 0-100% EtOAc in hexanes to afford the desired compound as a clear colorless oil.

### Step 4: (3R,4R)-3-[5-(2,6-dichlorophenyl)isoxazol-3-yl]-4-(3,4-difluorophenyl)piperidin-4-ol

To a solution of tert-butyl (3R,4R)-3-[5-(2,6-dichlorophenyl)isoxazole-3-yl]-4-(3,4-difluorophenyl)-4-hydroxypiperidine-1-carboxylate from step 3 (1 eq.) in CH₂Cl₂ (0.05 M) was added HCl (4 M in dioxane, 39 eq.) and the solution stirred at rt for 1 h and concentrated in vacuo. The residue was purified by flash chromatography on silica gel using 5-10% (2M NH₃ in MeOH) in CH₂Cl₂ to afford the desired compound.

### Example 37

### Structure 78: (3R,4R)-3-[5-(2,6-dichlorophenyl)-4-(R,S)-(1-hydroxyethyl)isoxazol-3-yl]-443A-difluorophenyl)piperidin-4-ol

### Step 1: 4-(2,6-dichlorophenyl)but-3-yn-2-one (general procedure for alkynyl ketone formation)

To a suspension of AlCl₃ (1.3 eq.) in CH₂Cl₂ (0.51 M) at −10° C. was added a mixture of [(2,6-dichlorophenyl)ethynyl](trimethyl)silane from example 36, step 1 (1.0 eq) and acetyl chloride (1.0 eq.) in CH₂Cl₂ (0.4 M) dropwise. The reaction mixture was allowed to warm to rt and stirred at rt for 1.3 h, cooled to −78° C. and quenched with 1M HCl. The reaction mixture was warmed to rt, the phases were separated, and the aqueous phase was extracted with CH₂Cl₂. The combined organic phases were dried over Na₂SO₄, filtered and concentrated in vacuo. The residue was purified by flash chromatography on silica gel using 0-100% EtOAc in hexanes to afford the title compound.

### Step 2: tert-butyl (3R,4R)-4-(3,4-difluorophenyl)-4-hydroxy-3-[(hydroxyimino)-methyl]piperidine-1-carboxylate

To a solution of tert-butyl (3S,4R)-4-(3,4-difluorophenyl)-3-formyl-4-hydroxy-piperidine-1-carboxylate (1 eq.) in EtOH (0.4 M) at rt was added sodium carbonate (2 eq.) and hydroxylamine hydrochloride (2 eq.). The reaction mixture was stirred at rt for 3 h, and extracted with Et₂O from water, dried over Na₂SO₄, filtered and concentrated in vacuo. The crude product was used without further purification.

### Step 3: tert-butyl (3R,4R)-3-[4-acetyl-5-(2,6-dichlorophenyl)isoxazole-3-yl]-4-(3,4-difluorophenyl)-4-hydroxypiperidine-1-carboxylate

To a solution of oxime from above (1 eq.) in MeOH (0.1 M) was added chloramine-T trihydrate (1 eq.) After 5 min, a solution of 4-(2,6-dichlorophenyl)but-3-yn-2-one from step 1 (4 eq.) in MeOH (0.4 M) was added. The reaction mixture was stirred at rt for 2 h, diluted with EtOH (0.02 M) and the reaction heated to 55° C. for 2 h, cooled to rt, and concentrated in vacuo. The residue was extracted with ether from water, the organic phase was washed with brine, dried over Na₂SO₄, filtered and concentrated in vacuo. The residue was purified by flash chromatography on silica gel using 0-100% EtOAc in hexanes to afford the title compound as a clear colorless oil.

### Step 4: tert-butyl (3R,4R)-3-[5-(2,6-dichlorophenyl)-4-(1-hydroxyethyl)isoxazole-3-yl]-4-(3,4-difluorophenyl)-4-hydroxypiperidine-1-carboxylate

To a solution of tert-butyl (3R,4R)-3-[4-acetyl-5-(2,6-dichlorophenyl)isoxazole-3-yl]-4-(3,4-difluorophenyl)-4-hydroxypiperidine-1-carboxylate from step 2 (1 eq.) in MeOH (0.03 M) was added NaBH₄ (1 eq), and the reaction mixture stirred at rt 30 min. A second portion of NaBH₄ (1.8 eq) was added, and the reaction mixture stirred for a further 20 min, and quenched with HCl (aq., 2 M), stirred for 10 min, and extracted with ether from water. The organic phase was washed with brine, dried over Na₂SO₄, filtered and concentrated in vacuo to afford the title compound as a clear colorless oil.

### Step 5: (3R,4R)-3-[5-(2,6-dichlorophenyl)-4-(1-hydroxyethyl)isoxazol-3-yl]-4-(3,4-difluorophenyl)piperidin-4-ol

To a solution of tert-butyl (3R,4R)-3-[5-(2,6-dichlorophenyl)-4-(1-hydroxyethyl)isoxazole-3-yl]-4-(3,4-difluorophenyl)-4-hydroxypiperidine-1-carboxylate from step 3 (1 eq.) in CH₂Cl₂ (0.04 M) was added HCl (4 M in dioxane, 53 eq.) and the solution stirred at rt for 50 min and concentrated in vacuo. The residue was purified by flash chromatography on silica gel using 5-10% (2M NH₃ in MeOH) in CH₂Cl₂ to afford the desired compound.

### Example 38

### Structure 80: N-[2-(2-{4-bromo-3-[(3R,4R)-4-(3,4-difluorophenyl)-4-hydroxypiperidin-3-yl]isoxazole-5-yl}phenyl)ethyl]acetamide

### Step 1: N-(2-{2-[(trimethylsilyl)ethynyl]phenyl}ethyl)acetamide

Prepared according to the general procedure for Sonogashira couplings (see, e.g., step 1, example 36), starting from N-[2-[2-(bromoethynyl)phenyl]ethyl]acetamide.

### Step 2: N-(2-{2-[bromoethynyl]phenyl}ethyl)acetamide (general procedure for alkynyl bromide formation)

To a solution of N-(2-{2-[(trimethylsilyl)ethynyl]phenyl}ethyl)acetamide from step 1 (1 eq.) and AgNO₃ (0.3 eq.) in acetone (0.16 M) was added dropwise a solution of NBS (1.2 eq.) in acetone (0.16 M). The reaction mixture was stirred in the dark for 1 h, filtered through CELITE□, washing with acetone, and concentrated in vacuo. The crude product was purified by column chromatography on silica gel, eluting with 40-100% EtOAc in hexanes to afford the title compound.

### Step 3: tert-butyl (3R,4R)-3-(2-{2-[2-(acetylamino)ethyl]phenyl}-4-bromoisoxazol-3-yl)-4-(3,4-difluorophenyl)-4-hydroxypiperidine-1-carboxylate

Prepared according to the general procedure for cycloadditions (see, e.g., step 5, example 24).

### Step 4: N-[2-(2-{4-bromo-3-[(3R,4R)-4-(3,4-difluorophenyl)-4-hydroxypiperidin-3-yl]isoxazole-5-yl}phenyl)ethyl]acetamide

Prepared according to the general procedure for deprotection (see, e.g., step 6, example 24).

### Example 39

### Structure 82: N-[2-(2-{4-bromo-3-[(3R,4R)-4-(3,4-difluorophenyl)-4-methoxypiperidin-3-yl]isoxazole-5-yl}phenyl)ethyl]acetamide

Step 1 tert-butyl (3R,4R)-3-(5-{2-[2-(acetylamino)ethyl]phenyl}-4-bromoisoxazol-3-yl)-4-(3,4-difluorophenyl)-4-methoxypiperidine-1-carboxylate

To a solution of tert-butyl (3R,4R)-3-(2-{2-[2-(acetylamino)ethyl]phenyl}-4-bromoisoxazol-3-yl)-4-(3,4-difluorophenyl)-4-hydroxypiperidine-1-carboxylate from step 3 of example 38 (1 eq.) in DMF (0.07 M) at rt was added MeI (3 eq.) followed by NaH (60% disp. in oil, 1 eq.) and the reaction mixture was stirred at rt for 25 min. The reaction mixture was separated by preparative HPLC (Gemini phenyl column, 50% MeCN: 50% 50 mM NH₄OAc) to afford the title compound as a clear, colorless oil.

### Step 2: N-[2-(2-{4-bromo-3-[(3R,4R)-4-(3,4-difluorophenyl)-4-methoxypiperidin-3-yl]isoxazole-5-yl}phenyl)ethyl]acetamide

Prepared according to the general procedure for deprotection (see, e.g., step 6, example 24).

### Example 40

### Structure 83: N-[2-(2-{4-acetyl-3-[(3R,4R)-4-(3,4-difluorophenyl)-4-hydroxypiperidin-3-yl]isoxazole-5-yl}phenyl)ethyl]acetamide

### Step 1: N-{2-[2-(3-oxobut-1-yn-1-yl)phenyl]ethyl}acetamide

Prepared according to the general procedure for alkynyl ketone formation (EXAMPLE 37, step 1), starting from N-[2-[2-(bromoethynyl)phenyl]ethyl]acetamide.

### Step 2: tert-butyl (3R,4R)-3-(4-acetyl-5-{2-[2-(acetylamino)ethyl]phenyl}-isoxazol-3-yl)-4-(3,4-difluorophenyl)-4-hydroxypiperidine-1-carboxylate

Prepared according to the general procedure for cycloadditions (see, e.g., step 5, example 24).

### Step 3: N-[2-(2-{4-acetyl-3-[(3R,4R)-4-(3,4-difluorophenyl)-4-hydroxypiperidin-3-yl]isoxazole-5-yl}phenyl)ethyl]acetamide

Prepared according to the general procedure for deprotection (see, e.g., step 6, example 24).

### Example 41

### Structure 85: N-(2-{2-[3-[(3R,4R)-4-(3,4-difluorophenyl)-4-hydroxypiperidin-3-yl]-4-(hydroxyethyl)isoxazole-5-yl]phenyl}ethyl)acetamide

### Step 1: tert-butyl (3R,4R)-3-[5-{2-[2-(acetylamino)ethyl]phenyl}-4-(1-hydroxyethyl)isoxazol-3-yl)-4-(3,4-difluorophenyl)-4-hydroxypiperidine-1-carboxylate

Prepared according to the general procedure for NaBH₄ reductions (e.g., solvent can be either MeOH as used in Example 37 or Ethanol).

### Step 2: N-(2-{2-[3-[(3R,4R)-4-(3,4-difluorophenyl)-4-hydroxypiperidin-3-yl]-4-(1-hydroxyethyl)isoxazole-5-yl]phenyl}ethyl)acetamide

Prepared according to the general procedure for deprotection (see, e.g., step 6, example 24).

### Example 42

### Structure 88: N-[2-(2-{4-bromo-3-[(3R,4R)-4-(3,4-difluorophenyl)-4-hydroxypiperidin-3-yl]isoxazole-5-yl}phenyl)ethyl]-2-hydroxyacetamide

### Step 1: 2-{[2-(2-bromophenyl)ethyl]amino}-2-oxoethyl acetate

To a solution of 2-bromophenethylamine (1 eq.), acetoxyacetic acid (1.1 eq.), and HATU (1.1 eq.) in DMF (0.2 M) was added Hunig's base (3 eq.). The reaction mixture was stirred at rt 2.5 days, extracted with Et₂O from water, dried over Na₂SO₄, filtered and concentrated in vacuo. The residue was purified by column chromatography on silica gel (20-100% EtOAc in hexanes) to afford the title compound as a clear colorless oil.

### Step 2: 2-oxo-2-[(2-{2-[(trimethylsilyl)ethynyl]phenyl}ethyl)amino}ethyl acetate

Prepared by the general procedure for Sonogashira couplings (see, e.g., step 1, example 36).

### Step 3: 2-({2-[2-(bromoethynyl)phenyl]ethyl}amino)-2-oxoethyl acetate

Prepared by the general procedure for alkynyl bromide formation (see, e.g., step 2, example 38).

### Step 4: tert-butyl (3R,4R)-3-{5-[2-(2-{[(acetyloxy)acetyl]amino}ethyl)phenyl]-4-bromoisoxazol-3-yl)-4-(3,4-difluorophenyl)-4-hydroxypiperidine-1-carboxylate

Prepared according to the general procedure for cycloadditions (see, e.g., step 5, example 24).

### Step 5: tert-butyl (3R,4R)-3-(4-bromo-5-{2-[2-(glycoloylamino)ethyl}-phenyl}isoxazol-3-yl)-4-(3,4-difluorophenyl)-4-hydroxypiperidine-1-carboxylate

A solution of tert-butyl (3R,4R)-3-{5-[2-(2-{[(acetyloxy)acetyl]amino}ethyl)phenyl]-4-bromoisoxazol-3-yl)-4-(3,4-difluorophenyl)-4-hydroxypiperidine-1-carboxylate from step 4 (1 eq.) in 2 N aq. LiOH (24 eq.) and THF (0.03 M) was stirred at rt for 30 min. The reaction mixture was extracted with 2×CH₂Cl₂ from NaHCO₃, dried over Na₂SO₄, filtered and concentrated in vacuo, to afford the title compound which was used without further purification.

### Step 6: N-[2-(2-{4-bromo-3-[(3R,4R)-4-(3,4-difluorophenyl)-4-hydroxypiperidin-3-yl]isoxazole-5-yl}-phenyl)ethyl]-2-hydroxyacetamide

Prepared according to the general procedure for deprotection (see, e.g., step 6, example 24).

### Example 43

### Structure 84: methyl [2-(2-{4-acetyl-3-[(3R,4R)-4-(3,4-difluorophenyl)-4-hydroxypiperidin-3-yl]isoxazole-5-yl}phenyl)ethyl]carbamate

### Step 1: methyl [2-(2bromophenyl)ethyl]carbamate

To a solution of 2-bromophenethylamine (1 eq.) and triethylamine (1.3 eq.) in dichloromethane (0.17 M) at rt was added slowly methyl chloroformate (1.15 eq.). The reaction mixture was stirred at rt for 1 h, 1 M aq. HCl was added and the phases were separated. The organic phase was dried over Na₂SO₄, filtered and concentrated in vacuo, to afford the title compound as a pale yellow oil, which was used without further purification.

### Step 2: methyl (2-{2-[(trimethylsilyl)ethynyl]phenyl}ethyl)carbamate

Prepared by the general procedure for Sonogashira couplings (see, e.g., step 1, example 36).

### Step 3: methyl {2-[2-(3-oxobut-1-yn-1-yl)phenyl]ethyl}carbamate

Prepared by the general procedure for acetylation (see, e.g., preparation of Ia-6.4).

### Step 4: tert-butyl (3R,4R)-3-[4-acetyl-5-(2-{2-[(methoxycarbonyl)amino]-ethyl}phenyl)isoxazol-3-yl)-4-(3,4-difluorophenyl)-4-hydroxypiperidine-1-carboxylate

Prepared according to the general procedure for cycloadditions (see, e.g., step 5, example 24).

### Step 5: methyl [2-(2-{4-acetyl-3-[(3R,4R)-4-(3,4-difluorophenyl)-4-hydroxypiperidin-3-yl]isoxazole-5-yl}phenyl)ethyl]carbamate

Prepared according to the general procedure for deprotection (see, e.g., step 6, example 24).

### Example 44

### Structure 90: N-[2-(2-{4-bromo-3-[(3R,4R)-4-(3,4-difluorophenyl)-4-(1H-1,2,3-triazoyl-ylmethoxy)piperidin-3-yl]isoxazole-5-yl}phenyl)ethyl]acetamide

Step 1 tert-butyl (3R,4R)-3-(5-{2-[2-(acetylamino)ethyl]phenyl}-4-bromoisoxazol-3-yl)-4-(3,4-difluorophenyl)-4-(prop-2-yn-1-yloxy)piperidine-1-carboxylate

To a solution of tert-butyl (3R,4R)-3-(2-{2-[2-(acetylamino)ethyl]phenyl}-4-bromoisoxazol-3-yl)-4-(3,4-difluorophenyl)-4-hydroxypiperidine-1-carboxylate from step 3 of EXAMPLE 38 (1 eq.) in DMF (0.1 M) at rt was added propargyl bromide (2 eq.) followed by NaH (60% disp. in oil, 1 eq.) and the reaction mixture was stirred at rt overnight. The reaction mixture was taken in Et₂O and water, the phases were separated and the organic phase was dried over Na₂SO₄, filtered and concentrated in vacuo, to afford the title compound in crude form, which was used without further purification in the next step.

Step 2 tert-butyl (3R,4R)-3-(5-{2-[2-(acetylamino)ethyl]phenyl}-4-bromoisoxazol-3-yl)-4-(3,4-difluorophenyl)-4-(1H-1,2,3-triazol-ylmethoxy)piperidine-1-carboxylate

A mixture of crude tert-butyl (3R,4R)-3-(5-{2-[2-(acetylamino)ethyl]phenyl}-4-bromoisoxazol-3-yl)-4-(3,4-difluorophenyl)-4-(prop-2-yn-1-yloxy)piperidine-1-carboxylate from step 1 (1 eq.), trimethylsilyl azide (1 eq.), and copper iodide (1 eq.) in DMF/MeOH (9:1, 0.1 M) was heated to 100° C. for 4 h. The reaction mixture was cooled to rt, extracted with Et₂O from NH₄OH, washed twice with brine, dried over Na₂SO₄, filtered and concentrated in vacuo. The crude product was purified by flash chromatography on silica gel (70-100% EtOAc in hexanes, followed by 10% (2N NH₃ in MeOH) in CH₂Cl₂ to afford the title compound.

### Step 3: N-[2-(2-{4-bromo-3-[(3R,4R)-4-(3,4-difluorophenyl)-4-(1H-1,2,3-triazol-ylmethoxy)piperidin-3-yl]isoxazole-5-yl}phenyl)ethyl]acetamide

Prepared according to the general procedure for deprotection (see, e.g., step 6, example 24).

### Example 45

### Structure 93: (3S,4S,5R)-5-[4-cyclopropyl-5-(2,3-dichlorophenyl)-4H-1,2,4-triazol-3-yl]-4-(3,4-difluorophenyl)-2-methylpiperidine

Step 1 2,3-dichloro-N-cyclopropylbenzamide

To a solution of 2,3-dichlorobenzoic acid (1 eq.) in dichloromethane (0.25M) was added oxalyl chloride (1.5 eq.) and a few drops of DMF. The resulting bubbling solution was allowed to stir at room temperature for 2.5 hours before the volatiles were removed in vacuo. The resulting residue was suspended in hexanes and filtered through a bed of CELITE®. Concentration of the filtrate in vacuo furnished the crude acid chloride as a yellow oil. This was then immediately taken up in dichloromethane (0.5M) and to it was added slowly neat cyclopropylamine (2.3 eq.) over a period of 20 minutes. After another 20 minutes of stirring at room temperature following the completion of addition, the volatiles were removed in vacuo and the resulting residue was partitioned between ether and water. The aqueous layer was separated and back-extracted with ether. The combined organic extracts were washed further with 1 N aq. HCl, 2 N aq. Na₂CO₃ and brine, dried over MgSO₄, and filtered. Concentration of the filtrate in vacuo furnished the crude title compound as a white solid.

### Step 2 2,3-dichloro-N″-cyclopropylbenzenecarboximidohydrazide

Crude 2,3-dichloro-N-cyclopropylbenzamide from the previous step (1 eq.) was heated with thioniyl chloride (11 eq.) at 80° C. for 16 hours. The volatiles were then removed in vacuo to furnish the crude imidoyl chloride as a viscous, yellow oil. At 0° C., this was added dropwise to neat, vigorously stirred hydrazine (22 eq.). Following the completion of addition, the resulting reaction mixture was then quenched with water and extracted with EtOAc. The combined organic extracts were washed further with water and brine, dried over MgSO₄ and filtered. Concentration of the filtrate in vacuo furnished a white semi-solid which was then triturated with methanol. The resulting suspension was then filtered through a bed of CELITE®. Concentration of the filtrate in vacuo afforded the crude title compound as a yellow oil that is contaminated with a small amount of its corresponding dimer.

### Step 3: [(3R,4S,6S)-1-(tert-butoxycarbonyl)-4-(3,4-difluorophenyl)-6-methylpiperidine-3-carboxylic acid

tert-Butyl (2S,4S,5R)-4-(3,4-difluorophenyl)-5-formyl-2-methylpiperidin-1-carboxylate from (Example 48, Step 5, 1 eq.) was added sodium dihydrogen phosphate (3 eq.) and 2-methyl-2-butene (6 eq.) were combined in tert-butanol (0.1 M). To this was then added sodium chlorite (0.9 M aq. solution, 3 eq.) and the resulting solution was allowed to stir at room temperature for 45 minutes. The reaction mixture was then diluted with water and extracted with EtOAc. The combined organic extracts were washed with brine, dried over MgSO₄, filtered and the filtrate concentrated in vacuo to afford the title compound as a white solid.

### Step 4: tert-butyl (2S,4S,5R)-5-[4-cyclopropyl-5-(2,3-dichlorophenyl)-4H-1,2,4-triazol-3-yl]-4-(3,4-difluorophenyl)-2-methylpiperidin-1-carboxylate

[(3R,4S,6S)-1-(tert-butoxycarbonyl)-4-(3,4-difluorophenyl)-6-methylpiperidine-3-carboxylic acid from the previous step (1 eq.), Hunig's base (4 eq.), HATU (1.2 eq.) and 2,3-dichloro-N″-cyclopropylbenzenecarboximidohydrazide (Step 2, 5 eq.) were combined in DMF (0.13 M). The resulting solution was allowed to stir at room temperature for 16 hours before the reaction was quenched with water and extracted with EtOAc. The combined organic extracts were washed with brine, dried over MgSO₄, filtered and the filtrate concentrated in vacuo. The resulting residue was then taken up in toluene (0.1 M) and heated at 100° C. for 16 hours. The volatiles were removed in vacuo and the crude product thus obtained was directly subjected to purification by way of column chromatography (SiO₂, CH₂Cl₂→4:1 (v/v) CH₂Cl₂: 2.0 M NH₃ in MeOH). The title compound was isolated as a yellow solid.

### Step 5: (3S,4S,5R)-5-[4-cyclopropyl-5-(2,3-dichlorophenyl)-4H-1,2,4-triazol-3-yl]-4-(3,4-difluorophenyl)-2-methylpiperidine

To a dichloromethane solution (0.05 M) of tert-butyl (2S,4S,5R)-5-[4-cyclopropyl-5-(2,3-dichlorophenyl)-4H-1,2,4-triazol-3-yl]-4-(3,4-difluorophenyl)-2-methylpiperidin-1-carboxylate from the previous step (1 eq.) was added HCl (4 M dioxane solution, 50 eq.) at room temperature. The resulting mixture was allowed to stir at room temperature for 1.5 hours. The volatiles were removed in vacuo and the crude product thus obtained was directly subjected to purification by way of column chromatography (SiO₂, CH₂Cl₂→4:1 (v/v) CH₂Cl₂: 2.0 M NH₃ in MeOH). The title compound was isolated as a white solid. ¹H NMR δ (ppm) (CH₃OH-d₄): 7.75 (1H, dd, J=8.09, 1.56 Hz), 7.46 (1H, t, J=7.89 Hz), 7.38 (1H, d, J=7.68 Hz), 7.20-7.13 (2H, m), 7.08 (1H, s), 3.58-3.41 (4H, m), 3.10 (1H, dd, J=12.75, 3.62 Hz), 2.63 (1H, s), 2.23 (1H, dt, J=18.42, 6.60 Hz), 1.86 (1H, d, J=13.54 Hz), 1.49 (3H, d, J=7.03 Hz), 1.09 (1H, s), 0.70 (2H, d, J=33.97 Hz), 0.32-0.25 (1H, m). MS (ESI+, M+H): 463.3.

### Example 46

### Structure 94: N-[2-(2-{4-cyclopropyl-5-[(3R,4S,6S)-4-(3,4-difluorophenyl)-6-methylpiperidin-3-yl]-4H-1,2A-triazol-3-yl}phenyl)-ethyl]acetamide

### Step 1 N-cyclopropyl-2-methylbenzamide

Prepared according to the procedure described earlier (Example 45, Step 1) except using 2-methylbenzoic acid (1 eq.) instead of 2,3-dichlorobenzoic acid as the starting material.

### Step 2 N″-cyclopropyl-2-methylbenzenecarboximidohydrazide

Prepared according to the procedure described earlier (Example 45, Step 2) except using N-cyclopropyl-2-methylbenzamide from the previous step (1 eq.) instead of 2,3-dichloro-N-cyclopropylbenzamide as the starting material.

### Step 3: tert-butyl (2S,4S,5R)-5-[4-cyclopropyl-5-(2-methylphenyl)-4H-1,2,4-triazol-3-yl]-4-(3,4-difluorophenyl)-2-methylpiperidin-1-carboxylate

Prepared according to the procedure described earlier (Example 45, Step 4) except using N″-cyclopropyl-2-methylbenzenecarboximidohydrazide from the previous step (1.4 eq.) instead of 2,3-dichloro-N″-cyclopropylbenzenecarboximidohydrazide as the starting material.

### Step 4: tert-butyl (2S,4S,5R)-5-{5-[2-(bromomethyl)lphenyl]-4-cyclopropyl-4H-1,2,4-triazol-3-yl]-4-(3,4-difluorophenyl)-2-methylpiperidin-1-carboxylate

To a carbon tetrachloride solution (0.1 M) of tert-butyl (2S,4S,5R)-5-[4-cyclopropyl-5-(2-methylphenyl)-4H-1,2,4-triazol-3-yl]-4-(3,4-difluorophenyl)-2-methylpiperidin-1-carboxylate from the previous step (1 eq.) was added NBS (2.2 eq.) and AIBN (0.2 eq.). The resulting solution was heated at reflux for 2 hours. The reaction mixture was then quenched with saturated aqueous NaHCO₃ and extracted with EtOAc. The combined organic extracts were dried over Na₂SO₄ and filtered. Concentration of the filtrate concentrated in vacuo afforded the crude title compound.

### Step 5: tert-butyl (2S,4S,5R)-5-{5-[2-(cyanomethyl)lphenyl]-4-cyclopropyl-4H-1,2,4-triazol-3-yl}-4-(3,4-difluorophenyl)-2-methylpiperidin-1-carboxylate

To a DMF solution (0.1 M) of tort-butyl (2S,4S,5R)-5-{5-[2-(bromomethyl)lphenyl}-4-cyclopropyl-4H-1,2,4-triazol-3-yl]-4-(3,4-difluorophenyl)-2-methylpiperidin-1-carboxylate from the previous step (1 eq.) was added sodium cyanide (2 eq.). The resulting solution was allowed to stir at room temperature for 16 hours. The reaction mixture was then quenched with saturated aqueous NaHCO₃ and extracted with EtOAc. The combined organic extracts were washed further with brine, dried over Na₂SO₄ and filtered. Concentration of the filtrate in vacuo and purification of the crude product thus obtained by way of column chromatography (SiO₂, CH₂Cl₂→10:1 (v/v) CH₂Cl₂: MeOH) afforded the title compound.

### Step 6: tert-butyl (2S,4S,5R)-5-(5-{2-[2-(acetylamino)ethyl]phenyl}-4-cyclopropyl-4H-1,2,4-triazol-3-yl)-4-(3,4-difluorophenyl)-2-methylpiperidin-1-carboxylate

To an ethanol solution (0.1 M) of tert-butyl (2S,4S,5R)-5-{5-[2-(cyanomethyl)lphenyl]-4-cyclopropyl-4H-1,2,4-triazol-3-yl]-4-(3,4-difluorophenyl)-2-methylpiperidin-1-carboxylate from the previous step (1 eq.) was added at 0° C. cobalt(II) chloride hexahydrate (2 eq.) and sodium borohydride (8 eq.). The resulting solution was allowed to warm to room temperature and stirred at room temperature for another 2 hours. The reaction mixture was then quenched with saturated aqueous NaHCO₃ and extracted with EtOAc. The combined organic extracts were washed further with brine, dried over Na₂SO₄ and filtered, Concentration of the filtrate in vacuo afforded the crude amine which was immediately taken up in dichloromethane (0.1 M) and added Hunig's base (2 eq.) and acetyl chloride (1.3 eq.). The reaction mixture was stirred at room temperature for 1 hour before it was quenched with saturated aqueous NaHCO₃ and extracted with EtOAc. The combined organic extracts were washed further with brine, dried over Na₂SO₄ and filtered. Concentration of the filtrate in vacuo and purification of the crude product thus obtained by way of column chromatography (SiO₂, CH₂Cl₂→10:1 (v/v) CH₂Cl₂:MeOH) afforded the title compound.

Step 7 N-[2-(2-{4-cyclopropyl-5-[(3R,4S,6S)-4-(3,4-difluorophenyl)-6-methylpiperidin-3-yl]-4H-1,2,4-triazol-3-yl}phenyl)-ethyl]acetamide

Prepared according to the procedure described earlier (Example 45, Step 5) except using tert-butyl (2S,4S,5R)-5-(5-{2-[2-(acetylamino)ethyl]phenyl}-4-cyclopropyl-4H-1,2,4-triazol-3-yl)-4-(3,4-difluorophenyl)-2-methylpiperidin-1-carboxylate (1 eq.) instead of tert-butyl (2S,4S,5R)-5-[4-cyclopropyl-5-(2,3-dichlorophenyl)-4H-1,2,4-triazol-3-yl]-4-(3,4-difluorophenyl)-2-methylpiperidin-1-carboxylate as the starting material. ¹H NMR δ (ppm) (CHCl₃-d): 7.89 (1H, s), 7.48-7.37 (2H, m), 7.32-7.25 (1H, m), 7.12-7.05 (2H, m), 7.00 (1H, d, J=7.8 Hz), 6.92 (1 H, s), 3.78-3.65 (2H, m), 3.59-3.42 (2H, m), 3.22 (1H, d, J=12.7 Hz), 2.68-2.32 (3H, m), 2.20-2.15 (1H, m), 1.98-1.88 (4H, m), 1.53 (3H, d, J=6.93 Hz), 1.00-0.88 (1H, m), 0.67-0.50 (2H, m), 0.10-0.02 (1H, m). MS (ESI+, M+H): 480.4.

### Example 47

### Structure 95: (3R,4S)-3-[4-cyclopropyl-5-(2,3-dichlorophenyl)-4H-1,2,4-triazol-3-yl]-4-(3,4-difluorophenyl)piperidine

### Step 1: (+/−)-trans-1-tert-butoxycarbonyl-4-(3,4-difluorophenyl)piperidine-3-carboxylic acid

To a solution of (+/−)-Trans-1-tort-butyl 3-ethyl-4-(3,4-difluorophenyl)piperidine-1,3-dicarboxylate (3.6 g, 9.75 mmol) (see EXAMPLE 51, step 3) in THF (32.5 mL) and MeOH (16.2 mL) was added a solution of LiOH (2M in water, 10.2 mL) and the solution was stirred at room temperature for 24 hours. The solvents were removed under reduced pressure and the residue was taken up in DCM. The aqueous phase was washed with 1N NaOH and the organic phase was discarded. The aqueous phase was acidified with 6N HCl and washed 3 times with DCM. The combined organic phase was dried over MgSO₄, filtered and the filtrate concentrated under reduced pressure to yield the title compound as a white solid.

### Step 2: Teri-butyl (3R,4S)-3-[4-cyclopropyl-5-(2,3-dichlorophenyl)-1,2,4-triazol-3-yl]-4-(3,4-difluorophenyl)piperidine-1-carboxylate

To a solution of trans-1-tert-butoxycarbonyl-4-(3,4-difluorophenyl)piperidine-3-carboxylic acid in DMF (0.075 M) was added HATU (1.2 eq.), 2,3-dichloro-N″-cyclopropylbenzenecarboximidohydrazide (Example 45, Step 2, 2 eq.) and Hunig's base (3 eq.), and the solution was stirred at room temperature overnight. Water was added and the organic phase was washed with ethyl acetate. The combined organic phase is dried over MgSO₄, filtered and the filtrate evaporated under reduced pressure. The crude product was dissolved in toluene (0.075 M) and refluxed for 12 hours. Solvents were removed and the residue was separated by chiral HPLC (Chiralcel OD, 50×400 mm, 5% MeOH, 5% i-PrOH, 0.25% Et₃N, 50 mL min) to yield the desired product as a single stereoisomer.

### Step 3: (3R,4S)-3-[4-cyclopropyl-5-(2,3-dichlorophenyl)-1,2,4-triazol-1-ium-3-yl]-4-(3,4-difluorophenyl)piperidin-1-ium dichloride

To a solution of tert-butyl (3R,4S)-3-[4-cyclopropyl-5-(2,3-dichlorophenyl)-1,2,4-triazol-3-yl]-4-(3,4-difluorophenyl)piperidine-1-carboxylate (of previous step, 1 eq.) in dichloromethane (0.05 M) was added a 4N solution of HCl in 1,4-dioxane (40 eq.) and the solution was stirred for 2 hours at room temperature. The solvent was removed under reduced pressure to give the corresponding HCl salt of the title compound as a white solid.

### Example 48

### Structure 96: N-[2-(2-{4-bromo-3-[(3R,4S,6S)-4-(3,4-difluorophenyl)-6-methylpiperidin-3-yl]isoxazol-5-yl}phenyl)ethyl]acetamide

Step 1 dimethyl [(1S)-1-(3,4-difluorophenyl)-3-oxobutyl]malonate

To a solution of (3E)-4-(3,4-difluorophenyl)but-3-en-2-one (J. Chem. Res., Synopses 2007, 6, 336.) (1 eq.) in acetonitrile (0.26M) was added dimethyl malonate (2 eq.) and potassium carbonate (2.5 eq.). The resulting suspension was heated at 50° C. for 5 hours and then slowly cooled to room temperature overnight. The reaction was then quenched with water and extracted with ether. The combined organic extracts were washed further with brine, dried over MgSO₄, filtered and the filtrate concentrated in vacuo. Purification of the crude reaction product by way of column chromatography (SiO₂, 95:5 (v/v) Hex:EtOAc→2:3 (v/v) Hex:EtOAc) afforded a viscous gum. The material thus obtained can be purified further by swishing in ether and hexanes to furnish a free-flowing, white powder. Finally, preparatory chiral separation using supercritical fluid chromatography (CO₂, 35° C., 100 barr) equipped with a chiral AD column using 90:5:2.5:2.5 CO₂ (35° C., 100 barr): heptane: methanol: ethanol as eluent (50 mL/min) afforded the title compound in >98% optical purity.

Step 2 methyl (3R,4S,6S)-4-(3,4-difluorophenyl)-6-methyl-2-oxopiperidine-3-carboxylate

To an aqueous solution (0.06 M) of Na₂HPO₄ (1.3 eq.), sodium formate (6.3 eq.) and L-alanine (12.6 eq.) was added P1G5 transaminase enzyme (using the Codex Transaminase Panel P1G5, available from Codexis, Inc. Redwood City, Calif., USA, 0.3 g per mmole of substrate [dimethyl [(1S)-1-(3,4-difluorophenyl)-3-oxobutyl]malonate from step 1]; alternative S-selective transaminase enzymes including those derived from vibrios may be suitable), pyridoxal-5′-phosphate (16 mg per mmol of substrate, nicotinamide adenine dinucleotide (8 mg per mmol of substrate), lactate dehydrogenase (1.6 mg per mmol of substrate), and formate dehydrogenase (16 mg per mmol of substrate). The resulting mixture was heated at 45° C. before dimethyl [(1S)-1-(3,4-difluorophenyl)-3-oxobutyl]malonate from the previous step (1 eq.) was added as a DMSO solution (0.3 M). After 17 hours, the reaction mixture was rendered basic (pH=11) with 5 N aq. NaOH and then extracted with isopropyl acetate. The organic extracts were combined and centrifuged. The supernatant layer was separated and the solid residue was extracted further with isopropyl acetate. The combined organic extracts were washed further with brine, dried over Na₂SO₄, filtered and the filtrate concentrated in vacuo to afford the crude title compound as a pale yellow solid.

### Step 3: [(3R,4S,6S)-4-(3,4-difluorophenyl)-6-methylpiperidin-3-yl]methanol

To a toluene solution (0.11 M) of methyl (3R,4S,6S)-4-(3,4-difluorophenyl)-6-methyl-2-oxopiperidine-3-carboxylate from the previous step (1 eq.) was added dropwise neat borane-dimethyl sulfide complex (4 eq.) over a period of 30 minutes. The resulting mixture was heated to 70° C. for 16 hours. The reaction mixture thus obtained was first carefully quenched with 4 N aqueous HCl at room temperature. The resulting biphasic mixture was heated at reflux for 5 hours to completely break the boron-nitrogen complex. The excess HCl was then quenched with the addition of sodium carbonate and the resulting suspension was extracted with EtOAc. The combined organic extracts were washed further with brine, dried over Na₂SO₄ and filtered. Concentration of the filtrate in vacuo afforded the crude title compound as a viscous, yellow oil.

### Step 4: tert-butyl (2S,4S,5R)-4-(3,4-difluorophenyl)-5-(hydroxymethyl)-2-methylpiperidin-1-carboxylate

To a dichloromethane solution (0.11 M) of [(3R,4S,6S)-4-(3,4-difluorophenyl)-6-methylpiperidin-3-yl]methanol from the previous step (1 eq.) and BOC anhydride (0.9 eq.) was added triethylamine (3 eq.). The resulting mixture was allowed to stir at room temperature for 16 hours. The volatiles were then removed in vacuo and the resulting residue was partitioned between ether and 10% aq. HCl. The aqueous layer was separated and back-extracted further with ether. The combined organic extracts were washed sequentially with 1 N aqueous NaOH, water and brine, dried over Na₂SO₄ and filtered. Concentration of the filtrate in vacuo afforded a viscous oil. Further purification by way of column chromatography (SiO₂, 95:5 (v/v) Hex:EtOAc→3:7 (v/v) Hex:EtOAc) afforded the title compound as a colorless oil.

### Step 5: tert-butyl (2S,4S,5R)-4-(3,4-difluorophenyl)-5-formyl-2-methylpiperidin-1-carboxylate

To a dichloromethane solution (0.1 M) of tort-butyl (2S,4S,5R)-4-(3,4-difluorophenyl)-5-(hydroxymethyl)-2-methylpiperidin-1-carboxylate from the previous step (1 eq.) and pyridine (20 eq.) was added Dess-Martin periodinane (2 eq.) portionwise at 0° C. The resulting mixture was allowed to warm slowly to room temperature over 4 hours. The volatiles were then removed in vacuo and the resulting residue was partitioned between ether and water. The aqueous layer was separated and back-extracted further with ether. The combined organic extracts were washed sequentially with 10% aqueous HCl, 1 N aqueous NaOH, water and brine, dried over Na₂SO₄ and filtered. Concentration of the filtrate in vacuo afforded a viscous oil. Further purification by way of column chromatography (SiO₂, 95:5 (v/v) Hex:EtOAc→1:1 (v/v) Hex:EtOAc) afforded the title compound as a pale yellow oil.

### Step 6: tert-butyl (2S,4S,5R)-4-(3,4-difluorophenyl)-5-[(E)-(hydroxyimino)methyl]-2-methylpiperidin-1-carboxylate

To an ethanol solution (0.1 M) of tert-butyl (2S,4S,5R)-4-(3,4-difluorophenyl)-5-formyl-2-methylpiperidin-1-carboxylate from the previous step (1 eq.) and hydroxylamine hydrochloride (4 eq.) was added sodium carbonate (1.5 M aq. solution, 4 eq.). The resulting mixture was stirred at room temperature for 4 hours. The volatiles were then removed in vacuo and the resulting residue was partitioned between ethyl acetate and water. The aqueous layer was separated and back-extracted further with ethyl acetate. The combined organic extracts were washed with brine, dried over Na₂SO₄ and filtered. Concentration of the filtrate in vacuo afforded the crude title compound as a white solid.

### Step 7: tert-butyl (2S,4S,5R)-5-(5-{2-[2-(acetylamino)ethyl]phenyl}-4-bromoisoxazol-3-yl)-4-(3,4-difluorophenyl)-2-methylpiperidin-1-carboxylate

To a methanol solution (0.11 M) of tert-butyl (2S,4S,5R)-4-(3,4-difluorophenyl)-5-[(E)-(hydroxyimino)methyl]-2-methylpiperidin-1-carboxylate from the previous step (1 eq.) was added Chloramine-T (1 eq.) and the resulting mixture was stirred at room temperature for 30 minutes. Then, N-[2-[2-(2-bromoethynyl)phenyl]ethyl]acetamide (1.5 eq.) (EXAMPLE 38, step 2) was added and the mixture was heated at reflux for 14 hours. The volatiles were then removed in vacuo and the crude product thus obtained was directly subjected to purification by way of column chromatography (SiO₂, 95:5 (v/v) Hex:EtOAc→1:1 (v/v) Hex:EtOAc) to afford the title compound as a white solid.

### Step 8: N-[2-(2-{4-bromo-3-[(3R,4S,6S)-4-(3,4-difluorophenyl)-6-methylpiperidin-3-yl]isoxazol-5-yl}phenyl)ethyl]acetamide

To a dichloromethane solution (0.4 M) of tert-butyl (2S,4S,5R)-5-(5-{2-[2-(acetylamino)ethyl]phenyl}-4-bromoisoxazol-3-yl)-4-(3,4-difluorophenyl)-2-methylpiperidin-1-carboxylate from the previous step (1 eq.) was added HCl (4 M dioxane solution, 30 eq.) and the resulting mixture was stirred at room temperature for 3 hours. The reaction was then quenched with 1 N aqueous NaOH and extracted with EtOAc. The combined organic extracts were washed further with brine, dried over Na₂SO₄ and filtered. Concentration of the filtrate in vacuo afforded the crude product as a pale yellow solid. Further purification by way of column chromatography (SiO₂, 95:5 (v/v) CH₂Cl₂: 2.0 M NH₃ in MeOH) afforded the title compound as a white solid. ¹H NMR δ (ppm) (CHCl₃-d): 7.47 (1H, t, J=7.5 Hz), 7.40-7.32 (3H, m), 7.10-7.01 (2H, m), 6.94-6.99 (1H, m), 5.40 (1H, s), 3.56-3.26 (5H, m), 3.20-3.12 (2H, m), 2.69 (2 H, t, J=7.18 Hz), 2.10-2.02 (1H, m), 1.94 (3H, s), 1.90-1.82 (1H, m), 1.79 (1H, s), 1.42 (3H, d, J=6.91 Hz). MS (ESI+, M+H): 520.0, 521.0.

### Example 49

### Structure 104: N-[2-(2-{4-chloro-3-[(3R,4R)-4-(3,4-difluorophenyl)-4-hydroxypiperidin-3-yl]isoxazol-5-yl}phenyl)ethyl]acetamide

### Step 1: Benzyl N-[2-[2-(2-trimethylsilylethynyl)phenyl]ethyl]carbamate

Benzyl N-[2-(2-bromophenyl)ethyl]carbamate (1 eq.), Copper (I) iodide (0.05 eq.) and PdCl₂(PPh₃)₂ (0.05 eq.) were dissolved in DMF (0.35 M) in a sealed tube before triethylamine (5 eq.) and ethynyl(trimethyl)silane (1.5 eq.) were added. The tube was sealed and heated to 100° C. for 20 hours. The tube was cooled to room temperature and more Copper (I) iodide (0.05 eq.), PdCl₂(PPh₃)₂ (0.05 eq.) and ethynyl(trimethyl)silane (1.5 eq.) were added. The tube was re-sealed and heated for further 20 hours and cooled to room temperature. An aqueous solution of sodium hydroxide (1M) was added and the aqueous phase was washed with dichloromethane, the combined organic phase was dried over MgSO₄, filtered and the filtrate evaporated under reduced pressure. The residue was purified by flash chromatography on silica gel, eluting with EtOAc/Hexanes (0 to 100%) to give the desired product as a brown oil.

### Step 2: Benzyl N-[2-[2-(2-chloroethynyl)phenyl]ethyl]carbamate

To a solution of benzyl N-[2-[2-(2-trimethylsilylethynyl)phenyl]ethyl]carbamate (1 eq.) and silver (1) nitrate (0.3 eq.) in acetone (0.185 M) was added NCS. The solution was stirred at room temperature, in the dark, for 2 hours. Solvent was removed under reduced pressure. The residue was purified by flash chromatography on silica gel, eluting with EtOAc/Hexanes (0 to 100%) to give the desired product as a brown solid.

### Step 3: Tert-butyl (3R,4R)-3-[5-[2-(2-benzyloxycarbonylaminoethyl)phenyl]-4-chloro-isoxazol-3-yl]-4-(3,4-difluorophenyl)-4-hydroxy-piperidine-1-carboxylate

To a solution of tert-butyl (3R,4R)-4-(3,4-difluorophenyl)-4-hydroxy-3-[(E)-(hydroxyimino)methyl]piperidine-1-carboxylate (1 eq.) in methanol (0.1 M) was added chloramine-T (1 eq.) and the solution was stirred for 5 minutes. Benzyl N-[2-[2-(2-chloroethynyl)phenyl]ethyl]carbamate (2.5 eq.) was added and the solution was refluxed overnight. The solvent was removed under reduced pressure and the residue was purified by flash chromatography on silica gel, eluting with EtOAc/Hexanes (50 to 100%) to give the desired product as a yellow solid.

### Step 4: Tert-butyl (3R,4R)-3-[5-[2-(2-aminoethyl)phenyl]-4-chloro-isoxazol-3-yl]-4-(3,4-difluorophenyl)-4-hydroxy-piperidine-1-carboxylate

To a solution of tert-butyl (3R,4R)-3-[5-[2-(2-benzyloxycarbonylaminoethyl)phenyl]-4-chloro-isoxazol-3-yl]-4-(3,4-difluorophenyl)-4-hydroxy-piperidine-1-carboxylate from previous step (1 eq.) in DME (0.06 M) was added barium hydroxide (4 eq.) and to solution was refluxed for 72 hours. The heterogeneous solution was filtered on CELITE® and washed with chloroform. The solvents were removed under reduced pressure. The residue was purified by flash chromatography on silica gel, eluting with 2M ammonia in MeOH/DCM (1 to 20%) to give the desired product as a yellow solid.

### Step 5: Tert-butyl (3R,4R)-3-[5-[2-(2-acetamidoethyl)phenyl]-4-chloro-isoxazol-3-yl]-4-(3,4-difluorophenyl)-4-hydroxy-piperidine-1-carboxylate

To a solution of tert-butyl (3R,4R)-3-[5-[2-(2-aminoethyl)phenyl]-4-chloro-isoxazol-3-yl]-4-(3,4-difluorophenyl)-4-hydroxy-piperidine-1-carboxylate from previous step (1 eq.) and triethylamine (2 eq.) in DCM (0.1 M) was added acetic anhydride (1.5 eq.) and the solution was stirred at room temperature for 2 hours. Water was added and the aqueous layer was washed with dichloromethane. The combined organic phase was dried over MgSO₄, filtered and the filtrate evaporated under reduced pressure. The residue was purified by flash chromatography on silica gel, eluting with EtOAc/Hexanes (70 to 100%) to give the desired product as a yellow solid.

### Step 6: Title Compound

To a solution of tert-butyl (3R,4R)-3-(5-{2-[2-(acetylamino)ethyl]phenyl}-4-chloroisoxazol-3-yl)-4-(3,4-difluorophenyl)-4-hydroxypiperidine-1-carboxylate (1 eq.) in DCM (0.05 M) was added a 4N solution of HCl in 1,4-dioxane (40 eq.) and the solution was stirred for 2 hours at room temperature. The solvent was removed under reduced pressure. The residue was purified by flash chromatography on silica gel, eluting with 2M ammonia in MeOH/DCM (1 to 20%) to give the title compound as a white solid.

### Example 50

### Structure 97: N-[2-(2-{4-Bromo-3-[(3R,4R)-4-(3,4-difluorophenyl)-4-hydroxypiperidin-3-yl]isoxazol-5-yl}pyridin-3-yl)ethyl]acetamide

### Step 1: (2-Bromopyridin-3-yl)acetonitrile

To a suspension of potassium t-butoxide (1.24 g, 11.0 mmol) in DME (4.5 mL), at −60° C., was slowly added a solution of 1-[(isocyanomethyl)sulfonyl]-4-methylbenzene (1.10 g, 5.64 mmol) in DME (4.5 mL). A solution of 2-bromopyridine-3-carbaldehyde (1.00 g, 5.38 mmol) in DME (4.5 mL) was then added and the mixture was stirred at −60° C. for 1 hour. MeOH (15 mL) was added and the reaction mixture was heated at reflux for 2 hours. The reaction mixture was then concentrated in vacuo, and dissolved in EtOAc and water. The organic fraction was washed with brine, dried over MgSO₄ and concentrated under in vacuo. The desired material was obtained after flash chromatography on silica gel (ISCO COMBI-FLASH®) eluting with EtOAc/Hexanes (0-30%).

### Step 2: N-[2-(2-Bromopyridin-3-yl)ethyl]acetamide

To a solution of (2-bromopyridin-3-yl)acetonitrile (467 mg, 2.37 mmol) from previous step in THF (11.9 mL) was added BH₃.DMS (0.68 mL, 7.11 mmol) and the reaction mixture was heated at 50° C. for 2 hours. The reaction mixture was cooled to room temperature, quenched with 2N NaOH and heated again for 1 hour. The volatiles were evaporated and the mixture was extracted with EtOAc (3×). The combined organic fractions were washed with brine, dried over Na₂SO₄ and the solvent was concentrated in vacuo. To the crude product dissolved in THF (11.9 mL) was added triethylamine (0.66 mL, 4.74 mmol) and acetic anhydride (0.34 mL, 3.56 mmol), and the mixture was stirred at room temperature for 1 hour, 10 minutes. The solvent was evaporated, and the residue was diluted with water and extracted with EtOAc (3×). The combined organic fractions were washed with brine, dried over Na₂SO₄ and the solvent was concentrated in vacuo. The desired material was obtained after flash chromatography on silica gel (ISCO COMBI-FLASH®) eluting with MeOH/CH₂Cl₂ (0-10%).

### Step 3: N-(2-{2-[(Trimethylsilyl)ethynyl]pyridin-3-yl}ethyl)acetamide

A mixture of N-[2-(2-bromopyridin-3-yl)ethyl]acetamide from previous step (633 mg, 2.60 mmol), trimethylsilylacetylene (1.1 mL, 7.81 mmol) and triethylamine (1.8 mL, 13.02 mmol) in DMF (8.68 mL) was degassed with N₂ for 3 minutes. Copper(I) iodide (50 mg, 0.260 mmol) and bis(triphenylphosphine)palladium(II) chloride (91 mg, 0.130 mmol) were added and the reaction mixture was heated at 70° C. for 1 hour, 10 minutes. The mixture was diluted with water and extracted with EtOAc (3×). The combined organic fractions were washed with water, dried over MgSO₄ and the solvent was concentrated in vacuo. The desired material was obtained after flash chromatography on silica gel (ISCO COMBI-FLASH®) eluting with MeOH/CH₂Cl₂ (0-10%).

### Step 4: N-{2-[2-(Bromoethynyl)pyridin-3-yl]ethyl}acetamide

To a solution of N-(2-{2-[(trimethylsilyl)ethynyl]pyridin-3-yl}ethyl)acetamide (235 mg, 0.902 mmol) and silver nitrate (46 mg, 0.271 mmol) in acetone (4.5 mL) in dark was added dropwise, at 0° C., a solution of NBS (225 mg, 1.263 mmol) in acetone (4.5 mL). The mixture was stirred at room temperature for 4 hours. The solvent was concentrated under reduced pressure. The desired compound was obtained after flash chromatography on silica gel (ISCO COMBI-FLASH®) eluting with EtOAc/hexanes (80-100%).

### Step 5: t-Butyl (3R,4R)-3-(5-{3-[2-(acetylamino)ethyl]pyridin-2-yl}-4-bromoisoxazol-3-yl)-4-(3,4-difluorophenyl)-4-hydroxypiperidine-1-carboxylate

To a solution of t-butyl (3R,4R)-4-(3,4-difluorophenyl)-4-hydroxy-3-[(hydroxyimino)methyl]piperidine-1-carboxylate (80 mg, 0.224 mmol) from step 2, example 37 in MeOH (2.2 mL) was added chloramine-T (72 mg, 0.292 mmol) and the mixture was stirred at room temperature for 0.5 hours. 1.1 mL of this solution was added to N-{2-[2-(bromoethynyl)pyridin-3-yl]ethyl}acetamide from the previous step (120 mg, 0.449 mmol) and the mixture was heated at 70° C. for 3 hours. This was repeated one more time. The solvent was evaporated, and the residue was diluted with water and extracted with EtOAc (3×). The combined organic fractions were washed with brine, dried over MgSO₄ and the solvent was concentrated in vacuo. The desired product was obtained from HPLC purification (Max-RP column, 70×30 mm, 25 mL/min).

### Step 6: N-[2-(2-{4-Bromo-3-[(3R,4R)-4-(3,4-difluorophenyl)-4-hydroxypiperidin-3-yl]isoxazol-5-yl}pyridin-3-yl)ethyl]acetamide

To a solution of t-butyl (3R,4R)-3-(5-{3-[2-(acetylamino)ethyl]pyridin-2-yl}-4-bromoisoxazol-3-yl)-4-(3,4-difluorophenyl)-4-hydroxypiperidine-1-carboxylate from previous step (17.9 mg, 0.029 mmol) in CH₂Cl₂ (0.14 mL) was added 4M HCl in dioxane (0.22 mL, 0.864 mmol) and the resulting mixture was stirred at room temperature for 2 hours. The solvent was concentrated under reduced pressure and the residue was purified by HPLC (Max-RP column, 50×21 mm, 25 mL/min) to give the title compound.

### Example 51

### Structure 107: N-[2-(2-{4-bromo-3-[(3R,4S)-4-(3,4-difluorophenyl)piperidin-3-yl]isoxazol-5-yl}phenyl)ethyl]acetamide

### Step 1: 1-tert-butyl 3-ethyl 4-(3,4-difluorophenyl)-5,6-dihydropyridine-1,3(2H)-dicarboxylate

A mixture of 1-tert-butyl 3-ethyl 4-{[(trifluoromethyl)sulfonyl]oxy}-5,6-dihydropyridine-1,3(2H)-dicarboxylate (10 g, 24.79 mmol) (WO 061129237), (3,4-difluorophenyl)boronic acid (4.70 g, 29.7 mmol), PdCl₂(dppf)₂ (1.0 g, 1.24 mmol) and 2M sodium carbonate (18.59 ml, 37.2 mmol) in dioxane (124 ml) was degassed with N₂ then heated at 90° C. for 2 hours. The dioxane was evaporated, and the residue was diluted with water (200 mL) and extracted with EtOAc (3×100 mL). The combined organic fractions were dried over Na₂SO₄ and the solvent was evaporated. Purification by ISCO COMBI-FLASH® chromatography (SiO₂-120 g, gradient elution of 0-40% EtOAc/hexanes over 30 min) afforded the title product. MS (ESI, Q⁺) m/z 390 (M+Na⁺)

### Step 2: (+/−)-Cis-1-tart-butyl 3-ethyl (3S,4S)-4-(3,4-difluorophenyl)piperidine-1,3-dicarboxylate

A mixture of 1-tert-butyl 3-ethyl 4-(3,4-difluorophenyl)-5,6-dihydropyridine-1,3(2H)-dicarboxylate from the previous step (9 g, 24.50 mmol) in anhydrous MeOH (122 ml) and magnesium metal (1.786 g, 73.5 mmol) was sonicated for 1 hour. The solvent was evaporated, the residue was diluted with water (100 mL), acidified with 1N HCl then extracted with EtOAc (3×100 mL). The combined organic fractions were dried over Na₂SO₄ and the solvent evaporated to afford the title product. MS (ESI, Q⁺) m/z 392 (M+Na⁺)

### Step 3: (+/−)-Trans-1-tert-butyl 3-ethyl-4-(3,4-difluorophenyl)piperidine-1,3-dicarboxylate

To a solution of (+/−)-cis-1-tert-butyl 3-ethyl (3S,4S)-4-(3,4-difluorophenyl)piperidine-1,3-dicarboxylate from previous step (8.5 g, 23.01 mmol) in EtOH (15 ml) was added sodium metal (0.793 g, 34.5 mmol). After 5 minutes, the mixture was heated at 80° C. under reflux overnight. The solvent was evaporated, the residue was acidified with 1N HCl and extracted with EtOAc (3×100 mL). The combined organic fractions were dried over Na₂SO₄ and the solvent evaporated to afford the title product. MS (ESI, Q⁺) m/z 392 (M+Na⁺)

### Step 4: (+/−)-Trans-tert-butyl-4-(3,4-difluorophenyl)-3-(hydroxymethyl)piperidine-1-carboxylate

To a solution of (+/−)-trans-1-tert-butyl 3-ethyl-4-(3,4-difluorophenyl)piperidine-1,3-dicarboxylate from previous step (8 g, 21.66 mmol) in THF (72.2 ml) was added DIBAL-H in Toluene (31.8 ml, 47.6 mmol) dropwise at −78° C. The mixture was stirred for 2 hours, quenched with a saturated sodium/potassium tartrate solution (100 ml) then warmed to room temperature and stirred for 1 hour. The volatiles were evaporated and the residue was extracted with EtOAc (3×100 mL). The combined organic fractions were dried over Na₂SO₄ and the solvent was evaporated. Purification by ISCO COMBI-FLASH® chromatography (SiO₂-80 g, gradient elution of 10-60% EtOAc/hexanes over 30 min) afforded the title product. MS (ESI, Q⁺) m/z 350 (M+Na⁺)

### Step 5: (+/−)-Trans-tert-butyl-4-(3,4-difluorophenyl)-3-formylpiperidine-1-carboxylate

To a solution of (+/−)-trans-tert-butyl-4-(3,4-difluorophenyl)-3-(hydroxymethyl)piperidine-1-carboxylate from previous step (6.2 g, 18.94 mmol) and pyridine (30.6 ml, 379 mmol) in CH₂Cl₂ (189 ml) was added Dess-Martin Periodinane (16.07 g, 37.9 mmol). The mixture was stirred at room temperature for 6 hours. The solvent was evaporated, the residue was diluted with saturated NaHCO₃ (100 mL) and extracted with EtOAc (3×100 mL). The combined organic fractions were washed with 1N HCl (100 ml), dried over Na₂SO₄ and the solvent was evaporated. Purification by ISCO COMBI-FLASH® chromatography (SiO₂-80 g, gradient elution of 0-40% EtOAc/hexanes over 30 min) afforded the title product. MS (ESI, m/z 348 (M+Na⁺)

### Step 6: (+/−)-Trans-tert-butyl-4-(3,4-difluorophenyl)-3-[(E)-(hydroxyimino)methyl]piperidine-1-carboxylate

To a solution of (+/−)-trans-tert-butyl-4-(3,4-difluorophenyl)-3-formylpiperidine-1-carboxylate from previous step (0.5 g, 1.537 mmol) in ethanol (15.37 ml) was added hydroxylamine hydrochloride (0.21 g, 3.07 mmol) and Na₂CO₃ (0.326 g, 3.07 mmol) and the mixture was stirred at room temperature for 2 hours. The solvent was evaporated and the residue diluted with water (20 mL) and extracted with EtOAc (3×10 mL). The combined organic fractions were dried over Na₂SO₄ and the solvent evaporated to afford the title product. MS (ESI, Q⁺) m/z 363 (M+Na⁺

### Step 7: tert-butyl (3R,4S)-3-(5-{2-[2-(acetylamino)ethyl]phenyl}-4-bromoisoxazol-3-yl)-4-(3,4-difluorophenyl)piperidine-1-carboxylate

Prepared according to the general procedure for cycloadditions described in Example 36, step 3 using (+/−)-trans-tert-butyl-4-(3,4-difluorophenyl)-3-[(E)-(hydroxyimino)methyl]-piperidine-1-carboxylate from previous step and N-{2-[2-(bromoethynyl)phenyl]ethyl}acetamide. The crude product was purified by reverse phase semi-prep HPLC on Max-RP column 100×30 mm using 55-80% CH₃CN/30 mM NH₄HCO₃ over 8.3 minutes. The enantiomers were separated by chiral HPLC on Chiralpak AD column 50×500 mm using 10% EtOH/5% Et3N/90% hexanes with a flow rate of 50 ml/min to afford tert-butyl (3S,4R)-3-(5-{2-[2-(acetylamino)ethyl]phenyl}-4-bromoisoxazol-3-yl)-4-(3,4-difluorophenyl)piperidine-1-carboxylate tᵣ=38 min and tert-butyl (3R,4S)-3-(5-{2-[2-(acetylamino)ethyl]phenyl}-4-bromoisoxazol-3-yl)-4-(3,4-difluorophenyl)piperidine-1-carboxylate, tᵣ=55 min. MS (ESI, Q⁺) m/z 626, 628 (M+Na⁺).

### Step 8: N-[2-(2-{4-bromo-3-[(3R,4S)-4-(3,4-difluorophenyl)piperidin-3-yl]isoxazol-5-yl}phenyl)ethyl]acetamide

To a solution of tert-butyl (3R,4S)-3-(5-{2-[2-(acetylamino)ethyl]phenyl}-4-bromoisoxazol-3-yl)-4-(3,4-difluorophenyl)piperidine-1-carboxylate from previous step (0.029 g, 0.048 mmol) in CH₂Cl₂ (0.480 ml) was added 4N HCl in dioxane (0.120 ml, 0.480 mmol) and the mixture was stirred for 2 hours. The solvent was evaporated and the crude product was purified by reverse phase semi-prep HPLC on X-Bridge column 100×21 mm using 55-80% MeOH/30 mM NH₄HCO₃ over 8.3 min with a flow rate of 25 ml/min to afford title product, tᵣ=7.5 min. MS (ESI, Q⁺) m/z 504, 506 (M+H⁺).

### Example 52

### Structure 113: (3R,4R)-3-(5-{2-[2-(Acetylamino)ethyl]phenyl}-4-bromoisoxazol-3-yl)-4-(4-{[2,2-difluoro-3-(propan-2-yloxy)propoxy]methyl}phenyl)-4-hydroxypiperidinium chloride

### Step 1: {3-[(4-Bromobenzyl)oxy]-2,2-difluoropropoxy}(t-butyl)dimethylsilane

To a solution of 3-{[t-butyl(dimethyl)silyl]oxy}-2,2-difluoropropan-1-ol (5.0 g, 22.1 mmol) in THF (74 mL), at 0° C., was added NaH (1.15 g, 60% dispersion in mineral oil, 28.7 mmol) and the resulting mixture was stirred at 0° C. for 15 minutes. Then, 1-bromo-4-(bromomethyl)benzene (6.07 g, 24.30 mmol) was added and the reaction mixture was stirred at room temperature overnight. The reaction mixture was quenched slowly with water and extracted with EtOAc (3×). The combined organic fractions were washed with brine, dried over MgSO₄ and concentrated under reduced pressure.

### Step 2: 3-[(4-Bromobenzyl)oxy]-2,2-difluoropropan-1-ol

To a solution of {3-[(4-bromobenzyl)oxy]-2,2-difluoropropoxy}(t-butyl)dimethylsilane from previous step (9.61 g, 24.3 mmol) in THF (122 mL) was added TBAF (31.6 mL, 31.6 mmol) and the resulting mixture was stirred at room temperature for 4 hours. The reaction mixture was concentrated, diluted in EtOAc and 10% HCl and extracted with EtOAc (3×). The combined organic fractions were washed with brine, dried over MgSO₄ and concentrated under reduced pressure. The desired material was obtained after flash chromatography on silica gel (ISCO COMBI-FLASH®) eluting with EtOAc/hexanes (0-30%).

### Step 3: 1-Bromo-4-{[2,2-difluoro-3-(propan-2-yloxy)propoxy]methyl}benzene

A light suspension of KOH (17.9 g, 319 mmol) in DMSO (159 mL) was stirred at room temperature for 15 minutes. Then, 3-[(4-bromobenzyl)oxy]-2,2-difluoropropan-1-ol from the previous step (4.48 g, 15.9 mmol) followed by 2-iodopropane (15.9 mL, 159 mmol) were added and the resulting mixture was stirred at room temperature for 1 hour. The reaction mixture was heated at 70° C. overnight. The reaction mixture was cooled down to room temperature, diluted in water and extracted with Et₂O (3×). The combined organic fractions were washed with water, followed by brine, dried over MgSO₄ and concentrated under reduced pressure. The desired material was obtained after flash chromatography on silica gel (ISCO COMBI-FLASH®) eluting with EtOAc/hexanes (0-40%).

### Step 4: t-Butyl 4-hydroxy-3-(hydroxymethyl)piperidine-1-carboxylate

Sodium borohydride (33.5 g, 885 mmol) was added in small portions to a stirred solution of 1-t-butyl 3-ethyl 4-oxopiperidine-1,3-dicarboxylate (24 g, 88 mmol) in anhydrous MeOH (450 mL) over 1 hour at 0° C. The solution was then stirred at room temperature overnight. The mixture was quenched by slow addition of water (200 mL) and stirred for 0.5 hours. The MeOH was evaporated and the mixture was diluted with brine (200 mL). The mixture was extracted with EtOAc (3×200 mL) and combined organic fractions were dried over Na₂SO₄ and the solvent was evaporated to afford the title product.

### Step 5: t-Butyl 4-hydroxy-3-{[(tripropan-2-ylsilyl)oxy]methyl}piperidine-1-carboxylate

To a solution of t-butyl 4-hydroxy-3-(hydroxymethyl)piperidine-1-carboxylate from the previous step (11.6 g, 50.2 mmol), triethylamine (10.5 mL, 75 mmol) and DMAP (613 mg, 5.02 mmol), at 0° C., was added TIPS-Cl (11.2 mL, 52.7 mmol). The reaction mixture was warmed and stirred at room temperature for 3 days. The reaction mixture was quenched with saturated aqueous NH₄Cl and water and was extracted with CH₂Cl₂ (3×). The combined organic fractions were washed with 10% HCl, dried over MgSO₄ and concentrated under reduced pressure. The title compound was obtained after flash chromatography on silica gel (ISCO COMM-FLASH®)) eluting with EtOAc/hexanes (0-100%).

### Step 6: t-Butyl 4-oxo-3-{[(tripropan-2-ylsilyl)oxy]methyl}piperidine-1-carboxylate

To a stirred solution of t-butyl 4-hydroxy-3-{[(tripropan-2-ylsilyl)oxy]methyl}piperidine-1-carboxylate from the previous step (13.9 g, 35.9 mmol) and NMO (16.8 g, 143 mmol) in CH₂Cl₂ (450 mL) and acetonitrile (50 mL) was added 4 Å molecular sieves (13.9 g) under N₂ and was stirred at room temperature for 10 minutes. Then, TPAP (1.26 g, 3.59 mmol) was added and the reaction mixture was stirred at room temperature for 2 hours. The reaction mixture was concentrated under reduced pressure and filtered on cotton. The title compound was obtained after flash chromatography on silica gel (ISCO COMBI-FLASH®) eluting with EtOAc/hexanes (0-20%).

### Step 7: (+/−)-trans-t-Butyl 4-(4-{[2,2-difluoro-3-(propan-2-yloxy)propoxy]methyl}phenyl)-4-hydorxy-3-{[(tripropan-2-ylsilyl)oxy]methyl}piperidine-1-carboxylate

To a solution of 1-bromo-4-{[2,2-difluoro-3-(propan-2-yloxy)propoxy]methyl}benzene from step 3, example 52 (2.51 g, 7.78 mmol) in THF (19.5 mL), at −78° C., was slowly added BuLi (3.3 mL, 8.17 mmol) and the resulting mixture was stirred at −78° C. for 15 minutes. Then, t-butyl 4-oxo-3-{[(tripropan-2-ylsilyl)oxy]methyl}piperidine-1-carboxylate from the previous step (1.5 g, 3.89 mmol) in THF (2 mL) was slowly added and the reaction mixture was stirred at the same temperature for 1 hour. The reaction mixture was quenched at −78° C. with saturated aqueous NH₄Cl and was extracted with EtOAc (3×). The combined organic fractions were dried over MgSO₄ and concentrated under reduced pressure.

### Step 8: (+/−)-trans-1-Butyl 4-(4-{[2,2-difluoro-3-(propan-2-yloxy)propoxy]methyl}phenyl)-4-hydroxy-3-(hydroxymethyl)piperidine-1-carboxylate

To a solution of (+/−)-trans-t-butyl 4-(4-{[2,2-difluoro-3-(propan-2-yloxy)propoxy]methyl}phenyl)-4-hydroxy-3-{[(tripropan-2-ylsilyl)oxy]methyl)piperidine-1-carboxylate from the previous step (2.45 g, 3.89 mmol) in THF (19.45 mL) was added TBAF (5.84 mL, 5.84 mmol) and the reaction mixture was stirred at room temperature for 5 hours. The reaction mixture was concentrated, diluted in EtOAc and 10% HCl and was extracted with EtOAc (3×). The combined organic fractions were washed with brine, dried over MgSO₄ and concentrated under reduced pressure. The desired material was obtained after flash chromatography on silica gel (ISCO COMBI-FLASH®) eluting with EtOAc/hexanes (0-40%).

### Step 9: (+/−)-trans-t-Butyl 4-(4-{[2,2-difluoro-3-(propan-2-yloxy)propoxy]methyl}phenyl)-3-formyl-4-hydroxypiperidine-1-carboxylate

To a solution of (+/−)-trans-t-butyl 4-(4-{[2,2-difluoro-3-(propan-2-yloxy)propoxy]methyl}phenyl)-4-hydroxy-3-(hydroxymethyl)piperidine-1-carboxylate from the previous step (1.45 g, 3.06 mmol) and Dess-Martin Periodinane (3.90 g, 9.19 mmol) in CH₂Cl₂ (61 mL) was added pyridine (7.4 mL, 92 mmol) and t-BuOH (14.6 mL, 153 mmol). The resulting mixture was stirred at room temperature overnight. The reaction mixture was quenched with saturated aqueous NaHCO₃ and was extracted with CH₂Cl₂ (3×). The combined organic fractions were washed with water, dried over MgSO₄ and concentrated under reduced pressure. The desired material was obtained after flash chromatography on silica gel (ISCO COMBI-FLASH®) eluting with EtOAc/hexanes (0-60%).

### Step 10: (+/−)-trans-t-Butyl 4-(4-{[2,2-difluoro-3-(propan-2-yloxy)propoxy]methyl}phenyl)-4-hydroxy-3-[(hydroxyimino)methyl]piperidine-1-carboxylate

Hydroxylamine hydrochloride (241 mg, 3.47 mmol) and sodium carbonate (380 mg, 3.58 mmol) were dissolved in water (3.3 mL) before a solution of (+/−)-trans-t-butyl 4-(4-{[2,2-difluoro-3-(propan-2-yloxy)propoxy]methyl}phenyl)-3-formyl-4-hydroxypiperidine-1-carboxylate from the previous step (1.09 g, 2.31 mmol) in EtOH (30 mL) was added. The reaction mixture was stirred at room temperature for 4 hours. Water and Et₂O were added to the reaction mixture and the organic layer was decanted, dried over MgSO₄ and concentrated under reduced pressure to give the desired product.

### Step 11: t-Butyl (3R,4R)-3-(5-{2-[2-(acetylamino)ethyl]phenyl}-4-bromoisoxazol-3-yl)-4-(4-{[2,2-difluoro-3-(propan-2-yloxy)propoxy]methyl}phenyl)-4-hydroxypiperidine-1-carboxylate

To a solution of (+/−)-trans-t-butyl 4-(4-{[2,2-difluoro-3-(propan-2-yloxy)propoxy]methyl}phenyl)-4-hydroxy-3-[(hydroxyimino)methyl]piperidine-1-carboxylate from the previous step (606 mg, 1.246 mmol) in MeOH (12.5 mL) was added chloramine-T (337 mg, 1.370 mmol) and was stirred at room temperature for 30 minutes. Then, N-{2-[2-(bromoethynyl)phenyl]ethyl}acetamide (497 mg, 1.868 mmol) was added and the reaction mixture was heated at 80° C. for 7 hours. The reaction mixture was concentrated, diluted in water and EtOAc and was extracted with EtOAc (3×). The combined organic fractions were washed with brine, dried over MgSO₄ and concentrated under reduced pressure. The crude was purified by HPLC separation (Max-RP column, 100×30 mm, 25 mL/min) to give the title compound. The enantiomers were separated by HPLC (Chiralpak AD column, 50×500 mm, 60 mL/min) and the slower enantiomer was the desired one.

### Step 12: (3R,4R)-3-(5-{2-[2-(Acetylamino)ethyl]phenyl}-4-bromoisoxazol-3-yl)-4-(4-{[2,2-difluoro-3-(propan-2-yloxy)propoxy]methyl}phenyl)-4-hydroxypiperidinium chloride

To a solution of t-butyl (3R,4R)-3-(5-{2-[2-(acetylamino)ethyl]phenyl}-4-bromoisoxazol-3-yl)-4-(4-{[2,2-difluoro-3-(propan-2-yloxy)propoxy]methyl}phenyl)-4-hydroxypiperidine-1-carboxylate from the previous step (79.9 mg, 0.106 mmol) in CH₂Cl₂ (0.54 mL) was added 4M HCl in dioxane (0.80 mL, 3.19 mmol) and the reaction mixture was stirred at room temperature for 1 hour. The solvent was evaporated, and the residue was triturated with Et₂O (2×) and dried under vacuum to give the title compound as the corresponding hydrochloride salt.

### Example 53

### Structure 106: (3R,4R)-3-(5-{2-[2-(Acetylamino)ethyl]phenyl}-4-bromoisoxazol-3-yl)-4-(3-fluoro-4-methylphenyl)-4-hydroxypiperidinium chloride

### Step 1: (+/−)-trans-t-Butyl 4-(3-fluoro-4-methylphenyl)-4-hydroxy-3-{[(tripropan-2-ylsilyl)oxy]methyl}piperidine-1-carboxylate

Prepared following the same procedure as Step 7, example 52, starting from t-butyl 4-oxo-3-{[(tripropan-2-ylsilyl)oxy]methyl}piperidine-1-carboxylate from step 6, example 52 and 4-bromo-2-fluoro-1-methylbenzene.

### Step 2: (+/−)-trans-t-Butyl 4-(3-fluoro-4-methylphenyl)-4-hydroxy-3-(hydroxymethyl)piperidine-1-carboxylate

Prepared following the same procedure as Step 8, example 52, starting from (+/−)-trans-t-butyl 4-(3-fluoro-4-methylphenyl)-4-hydroxy-3-{[(tripropan-2-ylsilyl)oxy]methyl}piperidine-1-carboxylate from the previous step.

### Step 3: (+/−)-trans-t-Butyl 4-(3-fluoro-4-methylphenyl)-3-formyl-4-hydroxypiperidine-1-carboxylate

Prepared following the same procedure as Step 9, example 52, starting from (+/−)-trans-t-butyl 4-(3-fluoro-4-methylphenyl)-4-hydroxy-3-(hydroxymethyl)piperidine-1-carboxylate from the previous step.

### Step 4: (+/−)-trans-t-Butyl 4-(3-fluoro-4-methylphenyl)-4-hydroxy-3-[(hydroxyimino)methyl]piperidine-1-carboxylate

Prepared following the same procedure as Step 10, example 52, starting from (+/−)-trans-t-butyl 4-(3-fluoro-4-methylphenyl)-3-formyl-4-hydroxypiperidine-1-carboxylate from the previous step.

### Step 5: t-Butyl (3R,4R)-3-(5-{2-[2-(acetylamino)ethyl]phenyl}-4-bromoisoxazol-3-yl)-4-(3-fluoro-4-methylphenyl)-4-hydroxypiperidine-1-carboxylate

Prepared following the same procedure as Step 11, example 52, starting from (+/−)-trans-t-butyl 4-(3-fluoro-4-methylphenyl)-4-hydroxy-3-[(hydroxyimino)methyl]piperidine-1-carboxylate from the previous step. The crude was purified by HPLC separation (Gemini Phenyl Hexyl column, 100×21 mm, 25 mL/min) to give the title compound. The enantiomers were separated by HPLC separation (Chiralpak AD column, 50×500 mm, 60 mL/min) and the slower enantiomer was the desired one.

### Step 6: (3R,4R)-3-(5-{2-[2-(Acetylamino)ethyl]phenyl}-4-bromoisoxazol-3-yl)-4-(3-fluoro-4-methylphenyl)-4-hydroxypiperidinium chloride

Prepared following the same procedure as Step 12, example 52, starting from t-butyl (3R,4R)-3-(5-{2-[2-(acetylamino)ethyl]phenyl}-4-bromoisoxazol-3-yl)-4-(3-fluoro-4-methylphenyl)-4-hydroxypiperidine-1-carboxylate from the previous step.

### Example 54

### Structure 116: (3R,4R)-3-(5-{2-[2-(Acetylamino)ethyl]phenyl}-4-bromoisoxazol-3-yl)-4-hydroxy-4-(4-{[2-(propan-2-yloxy)ethoxy]methyl}piperidinium chloride

### Step 1: 1-Bromo-4-{[2-(propan-2-yloxy)ethoxy]methyl}benzene

To a solution of 2-(propan-2-yloxy)ethanol (2.0 g, 19.2 mmol) in THF, at 0° C., was added NaH (998 mg, 60% dispersion in mineral oil, 24.96 mmol) and the resulting suspension was stirred at 0° C. for 15 min. Then, 1-bromo-4-(bromomethyl)benzene (5.28 g, 21.1 mmol) was added and the reaction mixture was warmed to room temperature and stirred for 2 days. The reaction mixture was quenched with water and extracted with EtOAc (3×). The combined organic fractions were dried over MgSO₄ and concentrated under reduced pressure. The title compound was obtained after flash chromatography on silica gel (ISCO COMBI-FLASH®) eluting with EtOAc/hexanes (0-10%).

### Step 2: (+/−)-trans-1-Benzyl-4-(4-{[2-(propan-2-yloxy)ethoxy]methyl}phenyl)-3-[(trityloxy)methyl]piperidin-4-ol

Prepared following the same procedure as Step 7, example 52, starting from 1-bromo-4-{[2-(propan-2-yloxy)ethoxy]methyl}benzene from the previous step and 1-benzyl-3-[(trityloxy)methyl]piperidin-4-one (WO 08/040,764). The title compound was obtained after flash chromatography on silica gel (ISCO COMBI-FLASH®) eluting with EtOAc/hexanes (10-50%).

### Step 3: (+/−)-trans-1-Benzyl-3-(hydroxymethyl)-4-(4-{[2-(propan-2-yloxy)ethoxy]methyl}phenyl)piperidin-4-ol

To a solution of (+/−)-trans-1-benzyl-4-(4-{[2-(propan-2-yloxy)ethoxy]methyl}phenyl)-3-[(trityloxy)methyl]piperidin-4-ol from the previous step (1.34 g, 2.05 mmol) in THF (4.8 mL) and MeOH (19.3 mL) was added p-toluenesulfonic acid monohydrate (585 mg, 3.07 mmol) and the reaction mixture was stirred at room temperature overnight. The reaction mixture was quenched with saturated aqueous NaHCO₃ and concentrated under reduced pressure. The residue was extracted with CH₂Cl₂ (3×). The combined organic fractions were washed with water, dried over MgSO₄ and concentrated under reduced pressure.

### Step 4: (+/−)-trans-t-Butyl 4-hydroxy-3-(hydroxymethyl)-4-(4-{[2-(propan-2-yloxy)ethoxy]methyl}phenyl)piperidine-1-carboxylate

A solution of (+/−)-trans-1-benzyl-3-(hydroxymethyl)-4-(4-{[2-(propan-2-yloxy)ethoxy]methyl}phenyl)piperidin-4-ol from the previous step (847 mg, 2.05 mmol), Pd/C (218 mg, 0.205 mmol) and BOC₂O (0.57 mL, 2.46 mmol) in MeOH (30 mL) was stirred at room temperature under an atmosphere of H₂ for 3 hours. The reaction mixture was filtered on CELITE® and the filtrate was concentrated under reduced pressure. The title compound was obtained after flash chromatography on silica gel (ISCO COMBI-FLASH®) eluting with EtOAc/hexanes (10-100%).

### Step 5: (+/−)-trans-t-Butyl 3-formyl-4-hydroxy-4-(4-{[2-(propan-2-yloxy)ethoxy]methyl}phenyl)piperidine-1-carboxylate

Prepared following the same procedure as Step 9, example 52, starting from (+/−)-trans-t-butyl 4-hydroxy-3-(hydroxymethyl)-4-(4-{[2-(propan-2-yloxy)ethoxy]methyl}phenyl)piperidine-1-carboxylate from the previous step.

### Step 6: (+/−)-trans-t-Butyl 4-hydroxy-3-[(hydroxyimino)methyl]-4-(4-{[2-(propan-2-yloxy)ethoxy]methyl}phenyl)piperidine-1-carboxylate

Prepared following the same procedure as Step 10, example 52, starting from (+/−)-trans-t-butyl 3-formyl-4-hydroxy-4-(4-{[2-(propan-2-yloxy)ethoxy]methyl}phenyl)piperidine-1-carboxylate from the previous step.

### Step 7: t-Butyl (3R,4R)-3-(5-{2-[2-(acetylamino)ethyl]phenyl}-4-bromoisoxazol-3-yl)-4-hydroxy-4-(4-{[2-(propan-2-yloxy)ethoxy]methyl}phenyl)piperidine-1-carboxylate

Prepared following the same procedure as Step 11, example 52, starting from (+/−)-trans-t-butyl 4-hydroxy-3-[(hydroxyimino)methyl]-4-(4-{[2-(propan-2-yloxy)ethoxy]methyl}phenyl)piperidine-1-carboxylate from the previous step. The crude was purified by HPLC separation (Gemini Phenyl Hexyl column, 100×21 mm, 25 mL/min) to give the title compound. The enantiomers were separated by HPLC separation (Chiralpak AD column, 50×500 mm, 60 mL/min) and the slower enantiomer was the desired one.

### Step 8: (3R,4R)-3-(5-{2-[2-(Acetylamino)ethyl]phenyl}-4-bromoisoxazol-3-yl)-4-hydroxy-4-(4-{[2-(propan-2-yloxy)ethoxy]methyl}phenyl)piperidinium chloride

Prepared following the same procedure as Step 12, example 52, starting from t-butyl (3R,4R)-3-(5-{2-[2-(acetylamino)ethyl]phenyl}-4-bromoisoxazol-3-yl)-4-hydroxy-4-(4-{[2-(propan-2-yloxy)ethoxy]methyl}phenyl)piperidine-1-carboxylate from the previous step.

### Example 55

### Structure 111: N-[2-(2-{4-bromo-3-[(3R,4S)-4-{4-[2-(2,6-dichloro-4-methylphenoxy)ethoxy]phenyl}piperidin-3-yl]isoxazol-5-yl}phenyl)ethyl]acetamide

### Step 1: tert-butyl (3R,4S)-4-{4-[2-(2,6-dichloro-4-methylphenoxy)ethoxy]phenyl}-3-(hydroxymethyl)piperidine-1-carboxylate

To a solution of (3R,4S)-1-(tert-butoxycarbonyl)-4-{4-[2-(2,6-dichloro-4-methylphenoxy)ethoxy]phenyl}piperidine-3-carboxylic acid (see: WO 08/058,787 (10 g) in 60 mL THF was added borane-methyl sulfide complex (4.35 g) portionwise and the mixture was heated to 70° C. for 1 h and cooled to room temperature, quenched carefully with MeOH until gas evolution ceased. The mixture was evaporated in vacuo and co-evaporated with MeOH (3×) to give the desired product as a white foamy solid which was used directly.

### Step 2: tert-butyl (3R,4S)-4-{4-[2-(2,6-dichloro-4-methylphenoxy)ethoxy]phenyl}-3-formylpiperidine-1-carboxylate

To a solution of the alcohol from the previous step (7.7 g) in DCM at room temperature was added the Dess-Martin Periodinane (7 g) in one portion as a solid. The suspension was stirred at room temperature for 1 hour and stored at −15° C. for 2 days and concentrated in vacuo. The residue was diluted with ether and filtered. The filtrate was concentrated and purified by flash chromatography to give the desired product.

### Step 3: tert-butyl (3R,4S)-4-{4-[2-(2,6-dichloro-4-methylphenoxy)ethoxy]phenyl}-3-[(E,Z)-(hydroxyimino)methyl]piperidine-1-carboxylate

To a solution of the aldehyde from the previous step (6.5 g) in ethanol (90 mL) and water (10 mL) was added hydroxylamine hydrochloride (1.33 g) and sodium carbonate (2.2 g), and the mixture was stirred at room temperature for 2 hours. The mixture was diluted with water/EtOAc and extracted with EtOAc. The organic layers were combined, washed with brine, dried over Na₂SO₄, filtered and concentrated in vacuo to give a white foam as a mixture of cis/trans isomers in a ration of 1.9:1 from NMR analysis.

### Step 4: tert-butyl (3R,4S)-3-(5-{2-[2-(acetylamino)ethyl]phenyl}-4-bromoisoxazol-3-yl)-4-{4-[2-(2,6-dichloro-4-methylphenoxy)ethoxy]phenyl}-piperidine-1-carboxylate

To a solution of the oxime from the previous step (767 mg) in MeOH was added Chloramine-T (360 mg), and the mixture was stirred at room temperature for 30 minutes. To it was added N-{2-[2-(bromoethynyl)phenyl]ethyl}acetamide (300 mg) and the solution was heated to 80° C. for 4 hours, cooled to room temperature, concentrated, and the residue was purified by flash chromatography eluting with 10-30% acetone in toluene to give the desired product.

To a solution of product from above (90 mg) in DCM (2 mL) was added TFA (0.88 mL) and the mixture was stirred at room temperature for 2 hours and concentrated in vacuo. The residue was triturated with ether/hexanes and dried under high vacuum to give a white foamy solid. LCMS (50-100% ACN/0.1% aqueous formic acid in 3 min): retention time: 1.53 min; MS (+ESI): m/z 688.0.

### Example 56

### Structure 110: N-[2-(2-{4-acetyl-3-[(3R,4S)-4-{4-[2-(2,6-dichloro-4-methylphenoxy)ethoxy]phenyl}piperidin-3-yl]isoxazol-5-yl}phenyl)ethyl]acetamide

### Step 1: tert-butyl (3R,4S)-3-(4-acetyl-5-{2-[2-(acetylamino)ethyl]phenyl}isoxazol-3-yl)-4-{4-[2-(2,6-dichloro-4-methylphenoxy)ethoxy]phenyl}piperidine-1-carboxylate

To a solution of oxime described above (890 mg) in MeOH was added Chloramine-T (890 mg) and the mixture was stirred at room temperature for 30 minutes. To it was added N-{2-[2-(3-oxobut-1-yn-1-yl)phenyl]ethyl}acetamide (260 mg) and the solution was heated to 80° C. overnight. The mixture was cooled to room temperature, concentrated in vacuo, and the residue was purified by flash chromatography (10-35% acetone in toluene) to give the desired product.

### Step 2: Title Compound

To a solution of above product (90 mg) in DCM was added TFA (1.38 mL) and the mixture was stirred at room temperature for 2 hours and concentrated in vacuo. The residue was co-evaporated with EtOAc, and the residue was triturated with ether and dried under high vacuum to give the desired product as an off-white foamy solid. LCMS: (50-100% ACN/0.1% aqueous formic acid in 3 min): retention time: 1.60 min; MS (+ESI): m/z 650.1.

### Example 57

### Structure 109: N-[2-(2-{3-[(3R,4S)-4-{4-[2-(2,6-dichloro-4-methylphenoxy)ethoxy]phenyl}piperidin-3-yl]-4-(1-hydroxyethyl)isoxazol-5-yl}phenyl)ethyl]acetamide

### Step 1: tert-butyl (3R,4S)-3-[5-{2-[2-(acetylamino)ethyl]phenyl}-4-(1-hydroxyethyl)isoxazol-3-yl]-4-{4-[2-(2,6-dichloro-4-methylphenoxy)ethoxy]phenyl}piperidine-1-carboxylate

To a solution of tert-butyl (3R,4S)-3-(4-acetyl-5-{2-[2-(acetylamino)ethyl]phenyl}-isoxazol-3-yl)-4-{4-[2-(2,6-dichloro-4-methylphenoxy)ethoxy]phenyl}piperidine-1-carboxylate (59 mg) in ethanol (1 mL) was added sodium borohydride (9 mg) and the mixture was stirred at room temperature for 30 minutes. Another 9 mg sodium borohydride was added. The mixture was stirred for an additional 10 minutes, and quenched with saturated NH₄Cl/water. The mixture was extracted with DCM (3×) and the organic phases were washed with water, dried over Na₂SO₄ and filtered. The filtrate was concentrated in vacuo to give the crude desired product which was used directly.

### Step 2: Title Compound

To a solution of above product (54 mg) in DCM was added a 4M solution of HCl in dioxane (0.54 mL) and the mixture was stirred at room temperature for 1 hour and concentrated. The residue was triturated with ether and dried under high vacuum to give the title compound. LCMS: (30-95% ACN/0.1% aqueous formic acid in 3 min): retention time: 2.65 min; MS (+ESI): m/z 652.2.

### Example 58

### Structure 108: N-[2-(2-{4-bromo-3-[(3R,4S)-4-{4-[2-(2,6-dichloro-4-methylphenoxy)ethoxy]phenyl}piperidin-3-yl]isoxazol-5-yl}phenyl)-5-methoxypentyl]acetamide

### Step 1: 2-(2-iodophenyl)-5-methoxypentanenitrile

In a round bottom (“RB”) flask was charged with solid NaHMDS (2.02 g) and to it was added THF (30 mL), and the mixture was stirred until all solid dissolved. To it was then added 2-iodophenylacetonitrile (2.43 g) as a solid and the resultant mixture was stirred at room temperature for 30 minutes. To it was added 3-bromo-methoxypropane (1.7 g) in one portion (precipitate formation quickly) and the suspension was stirred at room temperature for 2 hours and quenched with NR₄Cl. Workup as usual gave the crude product which was purified by the ISCO combi-flash to give 1.77 g of the desired product as a light yellowish oil.

### Step 2: 2-(2-iodophenyl)-5-methoxypentan-1-amine

To a solution of the nitrile from the previous step (1.4 g) in DCM was added 1.1 mL borane-methyl sulfide complex and the mixture was heated to 41° C. overnight. After cooling to room temperature, the mixture was quenched carefully with MeOH and concentrated. The residue was co-evaporated with MeOH (3×) in vacuo and the crude amine was used directly without purification.

### Step 3: N-[2-(2-iodophenyl)-5-methoxypentyl]acetamide

The crude amine from above (1.7 g) was dissolved in DCM (15 mL). To it was added TEA (1.1 mL) followed by Ac₂O (0.6 mL) dropwise at room temperature. The mixture was stirred at room temperature for 3 hours and then washed with saturated NaHCO₃. The organic phase was dried over Ma₂SO₄, filtered and concentrated. The crude was purified by the ISCO combi-flash (0-100% EA/hex) to give the desired product as a colorless oil.

### Step 4: N-(5-methoxy-2-{2-[(trimethylsilyl)ethynyl]phenyl}pentyl)acetamide

A solution of the iodide from above (1 g) in DMF (5 mL) and TEA (5 mL) was degassed by bubbling a stream of nitrogen for 5 min. To it was then added bis-(triphenylphosphine)-palladium (II) chloride (97 mg), CuI (53 mg) and trimethylsilylacetylene (1.2 mL). The mixture was sealed and heated to 60° C. for 2.5 hours and cooled to room temperature. The mixture was quenched with water, extracted with ether and the ether layer was washed with water (2×) and brine, dried over MgSO₄ and filtered. The filtrate was concentrated in vacuo and the residue was purified by the ISCO combi-flash (20-100% EA/hex) to give the desired product.

### Step 5: N-{2-[2-(bromoethynyl)phenyl]-5-methoxypentyl}acetamide

To a solution of trimethylsilylacetylene product from above (0.82 g) in acetone was added silver nitrate (0.13 g) and NBS (0.53 g) and the mixture was stirred at room temperature in dark for 1.5 hours. The acetone was evaporated in vacuo in dark and the residue was re-suspended in EtOAc and filtered. The filtrate was washed with 10% Na₂S₂O₃, water and brine. The crude was purified by ISCO combi-flash (0-100% EA/hex) to give the desired product as a light yellow glass.

### Step 6: tert-butyl (3R,4S)-3-(5-{2-[1-(acetylamino)-5-methoxypentan-2-yl]phenyl}-4-bromoisoxazol-3-yl)-4-{4-[2-(2,6-dichloro-4-methylphenoxy)ethoxy]phenyl}piperidine-1-carboxylate

To a solution of tert-butyl (3R,4S)-4-{4-[2-(2,6-dichloro-4-methylphenoxy)ethoxy]phenyl}-3-[(E,Z)-(hydroxyimino)methyl]piperidine-1-carboxylate (150 mg) in MeOH was added Chloramine-T (251 mg) and the mixture was stirred at room temperature for 30 minutes. To it was added N-{2-[2-(bromoethynyl)phenyl]-5-methoxypentyl}acetamide and the and the solution was heated to 80° C. for 5.5 hours and then cooled to room temperature, concentrated in vacuo. The residue was diluted with EtOAc/toluene and water, and extracted with EtOAc. The organic layer was dried, filtered and concentrated. The residue was purified by flash chromatography (10-30% acetone/toluene) to give the desired product as a mixture of diastereomers.

### Step 7: the Title Compound

The product from above was treated with 1.6 mL 4N HCl in dioxane for 2 hours and diluted with EtOAc. The solution was concentrated in vacuo and then co-evaporated with toluene (2×). The residue was triturated with ether/hexanes and the solid dried under high vacuum to give the desired product. LCMS: (50-100% ACN/0.1% aqueous formic acid in 3 min): retention time: 1.70 min; MS (+ESI): m/z 760.0.

### Example 59

### Structure 99: (3R,4R)-3-[5-(2-{(1S)-1-[(acetylamino)methyl]-4-methoxybutyl}phenyl)-4-bromoisoxazol-3-yl]-4-(3,4-difluorophenyl)-4-hydroxypiperidinium chloride

### Step 1: tert-butyl (3R,4R)-3-[5-(2-{(1S)-1-[(acetylamino)methyl]-4-methoxybutyl}phenyl)-4-bromoisoxazol-3-yl]-4-(3,4-difluorophenyl)-4-hydroxypiperidine-1-carboxylate and tert-Butyl (3R,4R)-3-[5-(2-{(1R)-1-[(acetylamino)methyl]-4-methoxybutyl}phenyl)-4-bromoisoxazol-3-yl]-4-(3,4-difluorophenyl)-4-hydroxypiperidine-1-carboxylate

Prepared according to the general procedure for cycloaddition described in Example 36, step 3 using tert-butyl (3R,4R)-4-(3,4-difluorophenyl)-4-hydroxy-3-[(E)-(hydroxyimino)methyl]piperidine-1-carboxylate and N-{2-[2-(bromoethynyl)phenyl]-5-methoxypentyl}acetamide and N-{2-[2-(bromoethynyl)phenyl]-5-methoxypentyl}acetamide (EXAMPLE 58, step 5).

The crude product was purified by reverse phase semi-prep HPLC on Max-RP column 50×21 mm using 50-90% CH₃CN/30 mM NH₄HCO₃ over 4.8 min, tᵣ=3 min to afford a mixture of the above diasteromers.

The diastereomers were then separated by chiral HPLC on Chiralpak AD column 50×500 mm using 10-15% MeOH/10-15% iPrOH/90-85% Hexanes and 0.25% Et₃N with a flow rate of 60 ml/min to afford tert-butyl (3R,4R)-3-[5-(2-{(1S)-1-[(acetylamino)methyl]-4-methoxybutyl}phenyl)-4-bromoisoxazol-3-yl]-4-(3,4-difluorophenyl)-4-hydroxypiperidine-1-carboxylate, tᵣ=20.8 min and tert-Butyl (3R,4R)-3-[5-(2-{(1R)-1-[(acetylamino)methyl]-4-methoxybutyl}phenyl)-4-bromoisoxazol-3-yl]-4-(3,4-difluorophenyl)-4-hydroxypiperidine-1-carboxylate, tᵣ=41.5 min. MS (ESI, Q⁺) m/z 691, 692 (M+14⁺). The stereochemistry of the sides were arbitrarily assigned.

### Step 2: (3R,4R)-3-[5-(2-{(1S)-1-[(acetylamino)methyl]-4-methoxybutyl}phenyl)-4-bromoisoxazol-3-yl]-4-(3,4-difluorophenyl)-4-hydroxypiperidinium chloride

To a solution of tert-butyl (3R,4R)-3-[5-(2-{(1S)-1-[(acetylamino)methyl]-4-methoxybutyl}phenyl)-4-bromoisoxazol-3-yl]-4-(3,4-difluorophenyl)-4-hydroxypiperidine-1-carboxylate from the previous step (42 mg, 0.061 mmol) in CH₂Cl₂ (607 W) was added 4N HCl in dioxane (455 μl, 1.822 mmol) and the mixture was stirred at room temperature for 2 hours. The solvent was evaporated to afford the title product. MS (ESI, Q⁺) m/z 592, 594 (M+H⁺)

### Example 60

### Structure 101: (3R,4R)-3-[5-(2-{(1R)-1-[(acetylamino)methyl]-4-methoxybutyl}phenyl)-4-bromoisoxazol-3-yl]-4-(3,4-difluorophenyl)-4-hydroxypiperidinium chloride

To a solution of tert-butyl (3R,4R)-3-[5-(2-{(1R)-1-[(acetylamino)methyl]-4-methoxybutyl}phenyl)-4-bromoisoxazol-3-yl]-4-(3,4-difluorophenyl)-4-hydroxypiperidine-1-carboxylate (45 mg, 0.065 mmol) in CH₂Cl₂ (651 μl,) was added 4N HCl in dioxane (488 μl, 1.952 mmol) and the mixture was stirred at room temperature for 2 hours. The solvent was evaporated to afford title product. MS (ESI, Q⁺) m/z 592, 594 (M+H⁺)

### Example 61

### Structure 103: (3R,4R)-3-{5-[2-[2-(acetylamino)ethyl]-4-(3-methoxypropyl)phenyl]-4-bromoisoxazol-3-yl}-4-(3,4-difluorophenyl)-4-hydroxypiperidinium chloride

### Step 1: Methyl 2-amino-5-[(1E)-3-methoxyprop-1-en-1-yl]benzoate

A mixture of methyl 2-amino-5-bromobenzoate (11 g, 47.8 mmol), (E)-2-(3-methoxypropenyl)-4,4,5,5-tetramethyl-(1,3,2)-dioxaboroane (10.42 g, 52.6 mmol), potassium phosphate tribasic (20.30 g, 96 mmol), palladium(II) acetate (0.215 g, 0.956 mmol), 2-dicyclohexylphosphino-2′,6′-dimethoxy-1,1′-biphenyl (0.785 g, 1.913 mmol) in THF (217 ml) and Water (22 ml) was degassed with N₂ and the mixture was heated at 80° C. for 4 hours. The solvent was evaporated, the residue was diluted with water (100 mL) and extracted with EtOAc (3×100 mL). The combined organic fractions were dried over Na₂SO₄ and the solvent was evaporated. Purification by ISCO COMBI-FLASH® chromatography (SiO₂-120 g, gradient elution of 0-30% EtOAc/hexanes over 20 min) afforded the title product.

### Step 2: Methyl 2-amino-5-(3-methoxypropyl)benzoate

A mixture of methyl 2-amino-5-[(1E)-3-methoxyprop-1-en-1-yl]benzoate from the previous step (10.2 g, 46.1 mmol) and 10% palladium on carbon (4.9 g, 4.61 mmol) in methanol (231 ml) was hydrogenated for 3 hours. The mixture was filtered through CELITE® and the solvent was evaporated to afford the title product which was used without purification.

### Step 3: Methyl 2-bromo-5-(3-methoxypropyl)benzoate

To a mixture of methyl 2-amino-5-(3-methoxypropyl)benzoate from the previous step (10 g, 44.8 mmol) in acetonitrile (149 ml) was added copper(II) bromide (13.00 g, 58.2 mmol) followed by tert-butyl nitrite (11.82 ml, 90 mmol). After 15 minutes, the mixture was heated at 70° C. for 1 hour. The solvent was evaporated and the residue was diluted with 1N HCl (200 mL) and extracted with EtOAc (3×100 mL). The combined organic fractions were washed with water (100 mL) then dried over Na₂SO₄. The solvent was evaporated and the crude product was purified by ISCO COMBI-FLASH® chromatography (SiO₂-120 g, gradient elution of 0-10% EtOAc/hexanes over 20 min) to afford the title product. MS (ESI, Q⁺) m/z 309, 311 (M+Na⁺)

### Step 4: [2-bromo-5-(3-methoxypropyl)phenyl]methanol

To a solution of methyl 2-bromo-5-(3-methoxypropyl)benzoate from the previous step (10.5 g, 36.6 mmol) in THF (183 ml) was added 1.5 M Dibal-H in toluene (60.9 ml, 91 mmol) dropwise at −78° C. and the mixture was stirred for 1 hour. The reaction mixture was carefully quenched with 1N HCl (50 mL) at −78° C., stirred for 15 minutes then allowed to warm to room temperature. The volatiles were evaporated and extracted with EtOAc (3×100 mL). The combined organic fractions were dried over Na₂SO₄ and the solvent was evaporated. Purification by ISCO COMBI-FLASH® chromatography (SiO₂-120 g, gradient elution of 10-40% EtOAc/hexanes over 30 min) afforded the title product. MS (ESI, Q⁺) m/z 281, 283 (M+Na⁺).

### Step 5: [2-bromo-5-(3-methoxypropyl)phenyl]acetonitrile

To a solution of [2-bromo-5-(3-methoxypropyl)phenyl]methanol from the previous step (9 g, 34.7 mmol) and methanesulfonyl chloride (3.25 ml, 41.7 mmol) in CH₂Cl₂ (174 ml) was added Et₃N (7.26 ml, 52.1 mmol) at 0° C. The reaction mixture was then warmed to room temperature and stirred for 1 hour. The mixture was washed with water (200 mL) and dried over Na₂SO₄. The solvent was evaporated and the crude mesylate was dissolved in DMF (100 mL). KCN (5.65 g, 87 mmol) in water (10 mL) was then added and the mixture was heated at 80° C. for 3 hours. The mixture was diluted with water (200 mL) and extracted with Et₂O (3×100 mL). The combined organic fractions were washed with water (100 mL) then dried over Na₂SO₄. The solvent was evaporated and purification by ISCO COMBI-FLASH® chromatography (SiO₂-120 g, gradient elution of 0-20% EtOAc/hexanes over 40 mins) afforded the title product. MS (ESI, Q⁺) m/z 290, 292 (M+H⁴)

### Step 6: [N-{2-[2-bromo-5-(3-methoxypropyl)phenyl]ethyl}acetamide

To a solution of 2-bromo-5-(3-methoxypropyl)phenyl]acetonitrile from the previous step (3.3 g, 12.31 mmol) in THF (61.5 ml) was added BH₃.DMS (3.51 ml, 36.9 mmol) and the mixture was heated at 50° C. for 2 hours. The reaction mixture was cooled to room temperature, quenched with 2N NaOH (20 mL) and the mixture was heated again for 1 hour. The volatiles were evaporated and the mixture was extracted with EtOAc (3×50 mL). The combined organic fractions were dried over Na₂SO₄ and the solvent was evaporated.

The crude product was dissolved in THF (61.5 ml), triethylamine (3.43 ml, 24.61 mmol) and acetic anhydride (1.74 ml, 18.46 mmol) were added and the reaction mixture was stirred at room temperature overnight. The solvent was evaporated, the residue was diluted with water (50 mL) and extracted with EtOAc (3×50 mL). The combined organic fractions were dried over Na₂SO₄ and the solvent was evaporated. Purification by ISCO COMBI-FLASH® chromatography (SiO₂-40 g, gradient elution of 100% EtOAc over 20 min) afforded the title product. MS (ESI, Q⁺) m/z 336, 338 (M+H⁺)

### Step 7: N-(2-{5-(3-methoxypropyl)-2-[(trimethylsilyl)-ethynyl]phenyl}ethyl)acetamide

A mixture of [N-{2-[2-bromo-5-(3-methoxypropyl)phenyl]ethyl}acetamide from the previous step (2.2 g, 7.00 mmol), trimethylsilylacetylene (2.95 ml, 21.00 mmol) and N-methyldicyclohexylamine (7.43 ml, 35.0 mmol) in DMF (23.34 ml) was degassed with N₂ for 3 minutes. Copper (I) iodide (0.133 g, 0.700 mmol) and bis(triphenylphosphine)palladium(II) chloride (0.246 g, 0.350 mmol) were added and the reaction mixture was heated at 70° C. After 3 hours, liquid chromatography mass spectrometry (LCMS) still showed starting material, another portion of bis(triphenylphosphine)palladium(II) chloride (0.246 g, 0.350 mmol) was added and reaction was heated for an additional 5 hours. The mixture was diluted with water (100 mL) and extracted with EtOAc (3×25 mL). The combined organic fractions were washed with water (50 mL) and dried over Na₂SO₄. The solvent was evaporated and purification by ISCO COMBI-FLASH® chromatography (SiO₂-40 g, gradient elution of 30-80% EtOAc/hexanes over 20 min) afforded the title product. MS (ESI, Q⁺) m/z 332 (M+H⁺),

### Step 8: N-{2-[2-(bromoethynyl)-5-(3-methoxypropyl)phenyl]ethyl}acetamide

To a solution of N-(2-{5-(3-methoxypropyl)-2-[(trimethylsilyl)ethynyl]phenyl}ethyl)acetamide from the previous step (1.5 g, 4.52 mmol) and silver nitrate (0.231 g, 1.357 mmol) in acetone (22.62 ml) was added NBS (0.966 g, 5.43 mmol) in acetone (22.62 ml). The flask was wrapped in aluminum foil and the mixture was stirred at room temperature for 2 hours. The solvent was evaporated, the residue was diluted with water (20 mL) and extracted with EtOAc (3×20 mL). The combined organic fractions were dried over Na₂SO₄ and the solvent was evaporated. The crude product was purified by reverse phase semi-prep HPLC on Max-RP column 50×21 mm using 25-65% MeCN/H₂O in 0.6% HCO₂H over 4.8 min with a flow rate of 25 ml/min to afford title product tᵣ=3.52 min. MS (ESI, Q⁺) m/z 338, 340 (M+H⁺)

### Step 9: tert-butyl (3R,4R)-3-{5-[2-[2-(acetylamino)ethyl]-4-(3-methoxypropyl)phenyl]-4-bromoisoxazol-3-yl}-4-(3,4-difluorophenyl)-4-hydroxypiperidine-1-carboxylate

Prepared according to the general procedure for cycloadditions described in Example 36, step 3 using tert-butyl (3R,4R)-4-(3,4-difluorophenyl)-4-hydroxy-3-[(E)-(hydroxyimino)methyl]piperidine-1-carboxylate and N-{2-[2-(bromoethynyl)-5-(3-methoxypropyl)phenyl]ethyl}acetamide from the previous step. MS (ESI, Q⁺) m/z 714, 716 (M+Na⁺).

### Step 10: (3R,4R)-3-{5-[2-[2-(acetylamino)ethyl]-4-(3-methoxypropyl)phenyl]-4-bromoisoxazol-3-yl}-4-(3,4-difluorophenyl)-4-hydroxypiperidinium chloride

To a solution of tert-butyl (3R,4R)-3-{5-[2-[2-(acetylamino)ethyl]-4-(3-methoxypropyl)phenyl]-4-bromoisoxazol-3-yl}-4-(3,4-difluorophenyl)-4-hydroxypiperidine-1-carboxylate from the previous step (23 mg, 0.033 mmol) in CH₂Cl₂ (664 μl) was added 4N HCl in doxane (166 μl, 0.664 mmol) and the mixture was stirred at room temperature for 2 hours. The solvent was evaporated to afford the title product. MS (ESI, Q⁺) m/z 592, 594 (M+H⁺).

### Example 62

### Structure 120: N-(2-{4′-[(3S,4R)-4-{6-[(2,2-Difluoro-3-methoxypropoxy)methyl]pyridin-3-yl}-4-hydroxypiperidin-3-yl]-3′-methylbiphenyl-2-yl}ethyl)acetamide

### Step 1: 5-Bromo-2-(bromomethyl)pyridine

To a solution of 5-bromo-2-methylpyridine (780 mg, 4.53 mmol) in CCl₄ (15.1 mL) was added NBS (807 mg, 4.53 mmol) and AIBN (15 mg, 0.091 mmol). The reaction mixture was heated at reflux for 4 hours, 30 minutes. After 2 hours of heating, another portion of AIBN (15 mg, 0.091 mmol) was added. The reaction mixture was cooled to room temperature, filtered and the filtrate was concentrated under reduced pressure. The title compound was obtained after flash chromatography on silica gel (ISCO COMBI-FLASH®) eluting with EtOAc/hexanes (0-15%).

### Step 2: t-Butyl(2,2-difluoro-3-methoxypropoxy)dimethylsilane

To a solution of 3-{[t-butyl(dimethyl)silyl]oxy}-2,2-difluoropropan-1-ol (5 g, 22.09 mmol) in THF (74 mL) was added sodium hydride (1.33 g, 33.1 mmol) at 0° C. After 15 minutes, methyl iodide (2.76 mL, 44.2 mmol) was added and the mixture was stirred at room temperature for 1 hour. The mixture was carefully quenched with water (1 mL), the volatiles were evaporated. The residue was diluted with water (50 mL) and extracted with Et2O (3×25 mL). The combined organic fractions were dried over Na2SO4 and the solvent was evaporated to afford the title product.

### Step 3: 2,2-Difluoro-3-methoxypropan-1-ol

To a solution of t-butyl(2,2-difluoro-3-methoxypropoxy)dimethylsilane from the previous step (5.1 g, 21.22 mmol) in THF (106 mL) was added TBAF (25.5 mL, 25.5 mmol). The mixture was stirred at room temperature for 1 hour. The solvent was evaporated, the residue was diluted with 1N HCl (100 mL) and extracted with EtOAc (3×50 mL). The combined organic fractions were dried over Na₂SO₄ and the solvent was evaporated. The desired material was obtained after flash chromatography on silica gel (Combi-Flash ISCO) eluting with EtOAc/hexanes (0-15%).

### Step 4: 5-Bromo-2-[(2,2-difluoro-3-methoxypropoxy)methyl]pyridine

To a solution of 2,2-difluoro-3-methoxypropan-1-ol (250 mg, 1.983 mmol) in THF (6.6 mL) was added, at 0° C., NaH (103 mg, 60% dispersion in mineral oil, 2.58 mmol). After 15 minutes, 5-bromo-2-(bromomethyl)pyridine (547 mg, 2.181 mmol) was added at 0° C. and the reaction mixture was warmed to room temperature and stirred for 4 hours. The reaction mixture was carefully quenched with water, the volatiles were evaporated, the residue was diluted with water and extracted with Et₂O (3×). The combined organic fractions were dried over MgSO₄ and the solvent was concentrated under reduced pressure. The title compound was obtained after flash chromatography on silica gel (ISCO COMBI-FLASH®) eluting with EtOAc/hexanes (0-10%).

### Step 5: t-Butyl (3S,4R)-3-{2′-[2-(acetylamino)ethyl]-3-methylbiphenyl-4-yl}-4-{6-[(2,2-difluoro-3-methoxypropoxy)methyl]pyridin-3-yl}-4-hydroxypiperidine-1-carboxylate

To a solution of 5-bromo-2-[(2,2-difluoro-3-methoxypropoxy)methyl]pyridine from the previous step (476 mg, 1.609 mmol) in THF (3.2 mL) was added BuLi (0.67 mL, 1.673 mmol) at −78° C. and the reaction mixture was stirred for 15 minutes. A solution of t-butyl 3-{2′-[2-(acetylamino)ethyl]-3-methylbiphenyl-4-yl}-4-oxopiperidine-1-carboxylate from step 4, example 19 (290 mg, 0.644 mmol) in THF (0.5 mL) was then added and the mixture was stirred for a further 45 minutes at the same temperature. The reaction mixture was quenched with saturated aqueous NH₄Cl and warmed to room temperature. The solvent was evaporated, the residue was diluted with water and extracted with EtOAc (3×). The combined organic fractions were dried over Na₂SO₄ and the solvent was concentrated under reduced pressure. The title compound was obtained after flash chromatography on silica gel (ISCO COMBI-FLASH®) eluting with EtOAc/hexanes (60-70%). The enantiomers were separated by HPLC separation (Chiralpak AD column, 50×500 mm, 65 mL/min) and the slower enantiomer was the desired one.

### Step 6: N-(2-{4′-[(3S,4R)-4-{6-[(2,2-Difluoro-3-methoxypropoxy)methyl]pyridin-3-yl}-4-hydroxypiperidin-3-yl]-3′-methylbiphenyl-2-yl}ethyl)acetamide

To a solution of t-butyl (3S,4R)-3-{2′-[2-(acetylamino)ethyl]-3-methylbiphenyl-4-yl}-4-{6-[(2,2-difluoro-3-methoxypropoxy)methyl]pyridin-3-yl}-4-hydroxypiperidine-1-carboxylate from the previous step (35.9 mg, 0.054 mmol) in CH₂Cl₂ (0.27 mL) was added 4M HCl in dioxane (0.40 mL, 1.613 mmol) and the reaction mixture was stirred at room temperature for 2 hours. The reaction mixture was concentrated, diluted in EtOAc and basified with 2N NaOH and extracted with EtOAc (3×). The combined organic fractions were dried over Na₂SO₄ and concentrated under reduced pressure. The title compound was obtained after flash chromatography on silica gel eluting with MeOH(NH₃)/CH₂Cl₂ (0-10%).

### Example 63

### Structure 121: N-(2-{4′-[(3S,4R)-4-{4-[(2,2-Difluoro-3-methoxypropoxy)methyl]phenyl}-4-hydroxypiperidin-3-yl]-3′-methylbiphenyl-2-yl}ethyl)acetamide

### Step 1: 1-Bromo-4-[(2,2-difluoro-3-methoxypropoxy)methyl]benzene

To a solution of 2,2-difluoro-3-methoxypropan-1-ol (600 mg, 4.76 mmol) in THF (15.9 mL) was added NaH (247 mg, 60% dispersion in mineral oil, 6.19 mmol) at room temperature. After 15 minutes, 4-bromobenzyl bromide (1.31 g, 5.23 mmol) was added at 0° C. and the mixture was stirred 15 minutes and then, warmed at room temperature for 1 hour. The mixture was then heated at 50° C. for 1 hour. The mixture was carefully quenched with water, the volatiles were evaporated, the residue was diluted with water and extracted with Et₂O (3×). The combined organic fractions were dried over MgSO₄ and the solvent was concentrated under reduced pressure. The title compound was obtained after flash chromatography on silica gel (ISCO COMBI-FLASH®) eluting with EtOAc/hexanes (0-10%).

### Step 2: t-Butyl (3S,4R)-3-{2′-[2-(acetylamino)ethyl]-3-methylbiphenyl-4-yl}-4-{4-[(2,2-difluoro-3-methoxypropoxy)methyl]phenyl}-4-hydroxypiperidine-1-carboxylate

Prepared following the same procedure as Step 5, example 62, starting from 1-bromo-4-[(2,2-difluoro-3-methoxypropoxy)methyl]benzene from the previous step and t-butyl 3-{2′-[2-(acetylamino)ethyl]-3-methylbiphenyl-4-yl}-4-oxopiperidine-1-carboxylate from step 4, example 19. The enantiomers were separated by HPLC separation (Chiralpak AD column, 50×500 mm, 50 mL/min) and the slower enantiomer was the desired one.

### Step 3: N-(2-{4′-[(3S,4R)-4-{4-[(2,2-Difluoro-3-methoxypropoxy)methyl]phenyl}-4-hydroxypiperidin-3-yl]-3′-methylbiphenyl-2-yl}ethyl)acetamide

Prepared following the same procedure as Step 6, example 62, starting from t-butyl (3S,4R)-3-{2′-[2-(acetylamino)ethyl]-3-methylbiphenyl-4-yl}-4-{4-[(2,2-difluoro-3-methoxypropoxy)methyl]phenyl}-4-hydroxypiperidine-1-carboxylate from the previous step.

### Example 64

### Structure 127: Methyl [2-(4′-{(3S,4R)-4-hydroxy-4-[4-({[(2S)-3-methoxy-2-methylpropyl]oxy}methyl)phenyl]piperidin-3-yl}-3′-methylbiphenyl-2-yl)ethyl]carbamate

### Step 1: t-Butyl (3S,4R)-3-[2′-(2-{[(benzyloxy)carbonyl]amino}ethyl)-3-methylbiphenyl-4-yl]-4-hydroxy-4-[4-({[(2S)-3-methoxy-2-methylpropyl]oxy}methyl)phenyl]piperidine-1-carboxylate

Prepared following the same procedure as Step 5, example 62, starting from 1-bromo-4-({[(2S)-3-methoxy-2-methylpropyl]oxy}methyl)benzene prepared according to precodure described in WO 08/040,764 and t-butyl 3-[2′-(2-{[(benzyloxy)carbonyl]amino}ethyl)-3-methylbiphenyl-4-yl]-4-oxopiperidine-1-carboxylate from stp 3, example 19. The crude was purified by flash chromatography on silica gel (ISCO COMBI-FLASH®) eluting with EtOAc/hexanes (5-30%). The enantiomers were separated by HPLC separation (Chiralcel OD column, 50×400 mm, 50 mL/min) and the slower enantiomer was the desired one.

### Step 2: t-Butyl (3S,4R)-3-[2′-(2-aminoethyl)-3-methylbiphenyl-4-yl]-4-hydroxy-4-[4-({[(2S)-3-methoxy-2-methylpropyl]oxy}methyl)phenyl]piperidine-1-carboxylate

A suspension of t-butyl (3S,4R)-3-[2′-(2-{[(benzyloxy)carbonyl]amino}ethyl)-3-methylbiphenyl-4-yl]-4-hydroxy-4-[4-({[(2S)-3-methoxy-2-methylpropyl]oxy}methyl)phenyl]piperidine-1-carboxylate from the previous step (770 mg, 1.045 mmol) and Pd/C (111 mg, 0.104 mmol) in MeOH (5.2 mL) was stirred at room temperature under an atmosphere of H₂ for 6 hours, 30 minutes. The reaction mixture was filtered on CELITE® and the filtrate was concentrated under reduced pressure to give the title compound.

### Step 3: t-Butyl (3S,4R)-4-hydroxy-3-(2′-{2-[(methoxycarbonyl)amino]ethyl}-3-methylbiphenyl-4-yl)-4-[4-({[(2S)-3-methoxy-2-methylpropyl]oxy}methyl)phenyl]piperidine-1-carboxylate

To a solution of t-butyl (3S,4R)-3-[2′-(2-aminoethyl)-3-methylbiphenyl-4-yl]-4-hydroxy-4-[4-({[(2S)-3-methoxy-2-methylpropyl]oxy}methyl)phenyl]piperidine-1-carboxylate from the previous step (40 mg, 0.066 mmol) in THF (0.3 mL) was added Et₃N (18 μl, 0.133 mmol) followed by methyl carbonochloridate (8 μl, 0.100 mmol) and the reaction mixture was stirred at room temperature overnight. The reaction mixture was concentrated under reduced pressure, the residue was diluted in water and extracted with EtOAc (3×). The combined organic fractions were dried over Na₂SO₄ and concentrated under reduced pressure to give the title compound.

### Step 4: Methyl [2-(4′-{(3S,4R)-4-hydroxy-4-[4-({[(2S)-3-methoxy-2-methylpropyl]oxy}methyl)phenyl]piperidin-3-yl}-3′-methylbiphenyl-2-yl)ethyl]carbamate

Prepared following the same procedure as described above for general deprotection of the BOC protection group (see also, step 6, example 62).

### Example 65

### Structure 128: 2-Hydroxy-N-[2-(4′-{(3S,4R)-4-hydroxy-4-hydroxy-4-[4-({[(2S)-3-methoxy-2-methylpropyl]oxy}methyl)phenyl]piperidin-3-yl}-3′-methylbiphenyl-2-yl)ethyl]acetamide

### Step 1: t-Butyl (3S,4R)-4-hydroxy-3-(2′-{2-[(hydroxyacetyl)amino]ethyl}-3-methylbiphenyl-4-yl)-4-[4-({[(2S)-3-methoxy-2-methylpropyl]oxy}methyl)phenyl]piperidine-1-carboxylate

To a solution of t-butyl (3S,4R)-3-[2′-(2-aminoethyl)-3-methylbiphenyl-4-yl]-4-hydroxy-4-[4-({[(2S)-3-methoxy-2-methylpropyl]oxy}methyl)phenyl]piperidine-1-carboxylate from step 2, example 64 (40 mg, 0.066 mmol) in THF (0.3 mL) was added Et₃N (18 μL, 0.133 mmol) followed by 2-chloro-2-oxoethyl acetate (11 μL, 100 mmol) and the reaction mixture was stirred at room temperature overnight. MeOH (0.3 mL) and 2N NaOH (0.2 mL) were added to the reaction mixture and was stirred at room temperature for 4 hours. After 2 hours, another amount of 2N NaOH (0.2 mL) was added. The reaction mixture was concentrated under reduced pressure, diluted in water and extracted with EtOAc (3×). The combined organic fractions were dried over Na₂SO₄ and concentrated under reduced pressure to give the title compound.

### Step 2: 2-Hydroxy-N-[2-(4′-{(3S,4R)-4-hydroxy-4-[4-({[(2S)-3-methoxy-2-methylpropyl]oxy}methyl)phenyl]piperidin-3-yl}-3′-methylbiphenyl-2-yl)ethyl]acetamide

Prepared following the same procedure as described above, starting from t-butyl (3S,4R)-4-hydroxy-3-(2′-{2-[(hydroxyacetyl)amino]ethyl}-3-methylbiphenyl-4-yl)-4-[4-({[(2S)-3-methoxy-2-methylpropyl]oxy}methyl)phenyl]piperidine-1-carboxylate from the previous step.

### Example 66

### Structure 125: 3-[2-(4′-{(3S,4R)-4-Hydroxy-4-[4-({[2S)-3-methoxy-2-methylpropyl]oxy}methyl)phenyl]piperidin-3-yl}-3′-methylbiphenyl-2-yl)ethyl]-1,3-oxazolidin-2-one

### Step 1: t-Butyl (3S,4R)-3-[2′-(2-{[(2-bromoethoxy)carbonyl]amino}ethyl)-3-methylbiphenyl-4-yl]-4-hydroxy-4-[4-({[(2S)-3-methoxy-2-methylpropyl]oxy}methyl)phenyl]piperidine-1-carboxylate

To a solution of t-butyl (3S,4R)-3-[2′-(2-aminoethyl)-3-methylbiphenyl-4-yl]-4-hydroxy-4-[4-({[(2S)-3-methoxy-2-methylpropyl]oxy}methyl)phenyl]piperidine-1-carboxylate from step 2, example 64 (40 mg, 0.066 mmol) in THF (0.3 mL) was added Et₃N (18 μL, 0.133 mmol) followed by 2-bromoethyl carbonochloridate (11 μL, 0.100 mmol) and the reaction mixture was stirred at room temperature overnight. The reaction mixture was concentrated under reduced pressure. The residue was diluted in water and extracted with EtOAc (3×). The combined organic fractions were dried over Na₂SO₄ and concentrated under reduced pressure to give the title compound.

### Step 2: t-Butyl (3S,4R)-4-hydroxy-4-[4-({[(2S)-3-methoxy-2-methylpropyl]oxy}methyl)phenyl]-3-{3-methyl-2′-[2-(2-oxo-1,3-oxazolidin-3-yl)ethyl]biphenyl-4-yl}piperidine-1-carboxylate

To a solution of t-butyl (3S,4R)-3-[2′-(2-{[(2-bromoethoxy)carbonyl]amino}ethyl)-3-methylbiphenyl-4-yl]-4-hydroxy-4-[4-({[(2S)-3-methoxy-2-methylpropyl]oxy}methyl)phenyl]piperidine-1-carboxylate from the previous step (49.7 mg, 0.066 mmol) in DMF (660 μL), at 0° C., was added NaH (5.3 mg, 60% dispersion in mineral oil, 0.132 mmol). The resulting mixture was then stirred at room temperature for 3 hours. The reaction mixture was quenched with water and extracted with EtOAc (3×). The combined organic fractions were washed with water, dried over Na₂SO₄ and concentrated under reduced pressure to give the title compound.

### Step 3: 3-[2-(4′-{(3S,4R)-4-Hydroxy-4-[4-({[(2S)-3-methoxy-2-methylpropyl]oxy}methyl)phenyl]piperidin-3-yl}-3′-methylbiphenyl-2-yl)ethyl]-1,3-oxazolidin-2-one

The Boc group was deprotected according to procedure described above (step 6, example 62) to give the title compound.

### Example 67

### Structure 124: 1-[2-(4′4-{(3S,4R)-4-hydroxy-4-[4-({[(2S)-3-methoxy-2-methylpropyl]oxy}methyl)phenyl]piperidin-3-yl}-3′-methylbiphenyl-2-yl)ethyl]urea

### Step 1: t-Butyl (3S,4R)-3-{2′-[2-(carbamoylamino)ethyl]-3-methylbiphenyl-4-yl}-4-hydroxy-4-[4-({[(2S)-3-methoxy-2-methylpropyl]oxy}methyl)phenyl]piperidine-1-carboxylate

To a solution of t-butyl (3S,4R)-3-[2′-(2-aminoethyl)-3-methylbiphenyl-4-yl]-4-hydroxy-4-[4-({[(2S)-3-methoxy-2-methylpropyl]oxy}methyl)phenyl]piperidine-1-carboxylate from step 2, example 64 (40 mg, 0.066 mmol) in THF (0.3 mL) was added Et₃N (18 μL, 0.133 mmol) followed by isocyanato(trimethyl)silane (13 pt, 0.100 mmol) and the reaction mixture was stirred at room temperature for 5 hours, 30 minutes. The reaction mixture was then heated at 40° C. for 19 hours. Another portion of Et₃N (18 μL, 0.133 mmol) and isocyanato(trimethyl)silane (13 μL, 0.100 mmol) were added and the reaction mixture was stirred at room temperature for 2 days. The reaction mixture was concentrated under reduced pressure, the residue was dissolved with water and extracted with EtOAc (3×). The combined organic fractions were dried over Na₂SO₄ and concentrated under reduced pressure. The title compound was obtained after flash chromatography on silica gel eluting with MeOH/CH₂Cl₂ (0-5%).

### Step 2: 1-[2-(4′-{(3S,4R)-4-Hydroxy-4-[4-({[(2S)-3-methoxy-2-methylpropyl]oxy}methyl)phenyl]piperidin-3-yl}-3′-methylbiphenyl-2-yl)ethyl]urea

Prepared following the same procedure, starting from t-butyl (3S,4R)-3-{2′-[2-(carbamoylamino)ethyl]-3-methylbiphenyl-4-yl}-4-hydroxy-4-[4-({[(2S)-3-methoxy-2-methylpropyl]oxy}methyl)phenyl]piperidine-1-carboxylate from the previous step. The title compound was obtained after flash chromatography on silica gel eluting with 2M NH₃ in MeOH/CH₂Cl₂ (0-5%).

### Example 68

### Structure 61: (+/−)-trans-N-(2-{3′-chloro-4′-[4-(1-methyl-2-oxo-1,2-dihydropyridin-4-yl)piperidin-3-yl]biphenyl-2-yl}ethyl)propanamide

### Step 1: (+/−)-trans-t-butyl 3-[2′-(2-{[(benzyloxy)carbonyl]amino}ethyl)-3-chlorobiphenyl-4-yl]-4-(1-methyl-2-oxo-1,2-dihydropyridin-4-yl)piperidine-1-carboxylate

A solution of (+/−)-trans-t-butyl 3-(4-bromo-2-chlorophenyl)-4-(1-methyl-2-oxo-1,2-dihydropyridin-4-yl)piperidine-1-carboxylate (from EXAMPLE 20, step 8) (500 mg, 1.04 mmol) and benzyl {2-[2-(4,4,5,5-tetramethyl-1,3,2-dioxaborolan-2-yl)phenyl]ethyl}carbamate (Ia-7.3) (435 mg, 1.14 mmol) in THF (9 mL) and water (0.9 mL) was degassed with N₂. Palladium acetate (12 mg, 0.052 mmol), 2-dicyclohexylphosphino-2′,6′-dimethoxy-1,1′-biphenyl (43 mg, 0.104 mmol) and potassium phosphate tribasic (661 mg, 3.11 mmol) were added and the mixture was degassed again with N₂. The mixture was heated at 80° C. for 3 hours, 30 minutes. The solvent was evaporated, the residue was diluted with water and extracted with EtOAc (3×), dried over MgSO₄ and the solvent was concentrated under reduced pressure. The desired material was obtained after flash chromatography on silica gel (ISCO COMBI-FLASH®) eluting with EtOAc/Hexanes (0-100%).

### Step 2: (+/−)-trans-t-butyl 3-[2′-(2-aminoethyl)-3-chlorobiphenyl-4-yl]-4-(1-methyl-2-oxo-1,2-dihydropyridin-4-yl)piperidine-1-carboxylate

A mixture of (+/−)-trans-t-butyl 3-[2′-(2-{[(benzyloxy)carbonyl]amino}ethyl)-3-chlorobiphenyl-4-yl]-4-(1-methyl-2-oxo-1,2-dihydropyridin-4-yl)piperidine-1-carboxylate from the previous step (490 mg, 0,747 mmol) and 10% palladium on carbon (79 mg, 0.075 mmol) in MeOH (3.7 mL) was hydrogenated at room temperature overnight. The mixture was filtered through a pad of CELITE® and the solvent was concentrated under reduced pressure to afford the desired product.

### Step 3: (+/−)-trans-t-butyl 3-{3-chloro-2′-[2-(propanoylamino)ethyl]biphenyl-4-yl}-4-(1-methyl-2-oxo-1,2-dihydropyridin-4-yl)piperidine-1-carboxylate

To a solution of (+/−)-trans-t-butyl 3-[2′-(2-aminoethyl)-3-chlorobiphenyl-4-yl]-4-(1-methyl-2-oxo-1,2-dihydropyridin-4-yl)piperidine-1-carboxylate from the previous step (75 mg, 0.144 mmol) and Et₃N (40 mL, 0.287 mmol) in THF (0.72 mL) was added propanoyl chloride (15 mL, 0.172 mmol). The resulting mixture was stirred at room temperature for 1 hour. The solvent was evaporated and the residue was diluted in water and was extracted with EtOAc (3×), dried over MgSO₄ and concentrated under reduced pressure. The desired material was obtained after flash chromatography on silica gel (ISCO COMBI-FLASH®) eluting with MeOH/CH₂Cl₂ (0-5%).

### Step 4: (+/−)-trans-N-(2-{3′-chloro-4′-[4-(1-methyl-2-oxo-1,2-dihydropyridin-4-yl)piperidin-3-yl]biphenyl-2-yl}ethyl)propanamide

To a solution of (+/−)-trans-t-butyl 3-{3-chloro-2′-[2-(propanoylamino)ethyl]biphenyl-4-yl}-4-(1-methyl-2-oxo-1,2-dihydropyridin-4-yl)piperidine-1-carboxylate from the previous step (57 mg, 0.099 mmol) in CH₂Cl₂ (0.49 mL) was added 4 M HCl in dioxane (0.24 mL, 0.98 mmol) and the reaction mixture was stirred at room temperature for 24 hours. The solvent was evaporated, the residue was taken in 2N NaOH and extracted with EtOAc (4×), dried over MgSO₄ and concentrated under reduced pressure. The title compound was obtained after flash chromatography on silica gel (ISCO COMBI-FLASH®) eluting with MeOH(NH₃)/CH₂Cl₂ (0-5%).

### Example 69

### Structure 62: (+/−)-trans-methyl (2-{3′-chloro-4′-[4-(1-methyl-2-oxo-1,2-dihydropyridin-4-yl)piperidin-3-yl]biphenyl-2-yl}ethyl)carbamate

### Step 1: (+/−)-trans-t-butyl-3-(3-chloro-2′-{2-[(methoxycarbonyl)amino]ethyl}biphenyl-4-yl)-4-(1-methyl-2-oxo-1,2-dihydropyridin-4-yl)piperidine-1-carboxylate

To a solution of (+/−)-trans-t-butyl 3-[2′-(2-aminoethyl)-3-chlorobiphenyl-4-yl]-4-(1-methyl-2-oxo-1,2-dihydropyridin-4-yl)piperidine-1-carboxylate from step 2, example 68 (75 mg, 0.144 mmol) and Et₃N (40 μL, 0.287 mmol) in THF (0.72 mL) was added methyl carbonochloridate (13 μL, 0.172 mmol). The resulting mixture was stirred at room temperature for 1 hour. The solvent was evaporated, the residue was diluted in water and was extracted with EtOAc (3×), dried over MgSO₄ and concentrated under reduced pressure. The desired material was obtained after flash chromatography on silica gel (ISCO COMBI-FLASH®) eluting with MeOH/CH₂Cl₂ (0-5%).

### Step 2: (+/−)-trans-methyl (2-{3′-chloro-4′-[4-(1-methyl-2-oxo-1,2-dihydropyridin-4-yl)piperidin-3-yl]biphenyl-2-yl}ethyl)carbamate

To a solution of (+/−)-trans-t-butyl-3-(3-chloro-2′-{2-[(methoxycarbonyl)amino]ethyl}biphenyl-4-yl)-4-(1-methyl-2-oxo-1,2-dihydropyridin-4-yl)piperidine-1-carboxylate from the previous step (50 mg, 0.086 mmol) in CH₂Cl₂ (0.43 mL) was added 4M HCl in dioxane (0.11 mL, 0.43 mmol) and the reaction mixture was stirred at room temperature for 2 hours. The solvent was evaporated, the residue was taken in 2N NaOH and extracted with EtOAc (4×), dried over MgSO₄ and concentrated under reduced pressure. The title compound was obtained after flash chromatography on silica gel eluting with MeOH(NH₃)/CH₂Cl₂ (0-7%).

### Example 70

### Structure 63: (+/−)-trans-ethyl (2-{3′-chloro-4′-[4-(1-methyl-2-oxo-1,2-dihydropyridin-4-yl)piperidin-3-yl]biphenyl-2-yl}ethyl)carbamate

### Step 1: (+/−)-trans-t-butyl 3-(3-chloro-2′-{2-[(ethoxycarbonyl)amino]ethyl}biphenyl-4-yl)-4-(1-methyl-2-oxo-1,2-dihydropyridin-4-yl)piperidine-1-carboxylate

To a solution of (+/−)-trans-t-butyl 3-[2′-(2-aminoethyl)-3-chlorobiphenyl-4-yl]-4-(1-methyl-2-oxo-1,2-dihydropyridin-4-yl)piperidine-1-carboxylate from step 2, example 68 (75 mg, 0.144 mmol) and Et₃N (40 μL, 0.287 mmol) in THF (0.72 mL) was added ethyl carbonochloridate (16 μL, 0.172 mmol). The resulting mixture was stirred at room temperature for 1 hour. The solvent was evaporated, the residue was extracted with EtOAc (3×), dried over MgSO₄ and concentrated under reduced pressure. The desired material was obtained after flash chromatography on silica gel (ISCO COMBI-FLASH®) eluting with MeOH/CH₂Cl₂ (0-5%).

### Step 2: (+/−)-trans-ethyl (2-{3′-chloro-4′-[4-(1-methyl-2-oxo-1,2-dihydropyridin-4-yl)piperidin-3-yl]biphenyl-2-yl}ethyl)carbamate

To a solution of (+/−)-trans-t-butyl-3-(3-chloro-2′-{2-[(ethoxycarbonyl)amino]ethyl}biphenyl-4-yl)-4-(1-methyl-2-oxo-1,2-dihydropyridin-4-yl)piperidine-1-carboxylate from the previous step (62 mg, 0.104 mmol) in CH₂Cl₂ (0.52 mL) was added 4M HCl in dioxane (0.13 mL, 0.52 mmol) and the reaction mixture was stirred at room temperature for 4 hours. The solvent was evaporated, the residue was taken in 2N NaOH and extracted with EtOAc (4×), dried over MgSO₄ and concentrated under reduced pressure. The title compound was obtained after flash chromatography on silica gel eluting with MeOH(NH₃)/CH₂Cl₂ (0-5%).

### Example 71

### Structure 64: (+/−)-trans-N-(2-{3′-chloro-4′-[4-(1-methyl-2-oxo-1,2-dihydropyridin-4-yl)piperidin-3-yl]biphenyl-2-yl}ethyl)-2-methoxyacetamide

### Step 1: (+/−)-trans-t-butyl 3-(3-chloro-2′-{2-[(methoxyacetyl)amino]ethyl}biphenyl-4-yl)-4-(1-methyl-2-oxo-1,2-dihydropyridin-4-yl)piperidine-1-carboxylate

To a solution of (+/−)-trans-t-butyl 3-[2′-(2-aminoethyl)-3-chlorobiphenyl-4-yl]-4-(1-methyl-2-oxo-1,2-dihydropyridin-4-yl)piperidine-1-carboxylate from step 2, example 68 (75 mg, 0.144 mmol) and Et₃N (40 μL, 0.287 mmol) in THF (0.72 mL) was added methoxyacetyl chloride (16 μL, 0.172 mmol). The resulting mixture was stirred at room temperature for 1 hour. The solvent was evaporated, the residue was diluted in water, was extracted with EtOAc (3×), dried over MgSO₄ and concentrated under reduced pressure. The desired material was obtained after flash chromatography on silica gel (ISCO COMBI-FLASH®) eluting with MeOH/CH₂Cl₂ (0-5%).

### Step 2: (+/−)-trans-N-(2-{3′-chloro-4′-[4-(1-methyl-2-oxo-1,2-dihydropyridin-4-yl)piperidin-3-yl]biphenyl-2-yl}ethyl)-2-methoxyacetamide

To a solution of (+/−)-trans-t-butyl 3-(3-chloro-2′-{2-[(methoxyacetyl)amino]ethyl}biphenyl-4-yl)-4-(1-methyl-2-oxo-1,2-dihydropyridin-4-yl)piperidine-1-carboxylate from the previous step (51 mg, 0.086 mmol) in CH₂Cl₂ (0.43 mL) was added 4M HCl in dioxane (0.11 mL, 0.43 mmol) and the reaction mixture was stirred at room temperature for 22 hours. The solvent was evaporated, the residue was taken in 2N NaOH, extracted with EtOAc (4×), dried over MgSO₄ and concentrated under reduced pressure. The title compound was obtained after flash chromatography on silica gel eluting with MeOH(NH₃)/CH₂Cl₂ (0-5%).

### Example 72

### Structure 65: (+/−)-trans-N-(2-{3′-chloro-5-fluoro-4′-[4-(1-methyl-2-oxo-1,2-dihydropyridin-4-yl)piperidin-3-yl]biphenyl-2-yl}ethyl)acetamide

### Step 1: N-[2-(2-bromo-4-fluorophenyl)ethyl]acetamide

To a solution of commercially available 2-bromo-4-fluorophenylacetonitrile (300 mg, 1.40 mmol) in THF (11.2 mL) was added BH₃.DMS (0.40 mL, 4.20 mmol) and the mixture was heated at reflux for 3 hours. At 0° C., the reaction mixture was quenched with water (4 mL) and 2N NaOH (4 mL). The resulting mixture was heated at reflux for 1 hours, 30 minutes. After cooling to room temperature, the aqueous layer was extracted with CH₂Cl₂ (3×), dried over MgSO₄ and concentrated under reduced pressure to afford the desired material (2-(2-bromo-4-fluorophenyl)ethanamine. To a solution of (2-(2-bromo-4-fluorophenyl)ethanamine (305 mg, 1.40 mmol) and Et₃N (0.39 mL, 2.80 mmol) was slowly added AcCl (104 μL, 1.47 mmol) and the resulting mixture was stirred at room temperature for 3 hours. The reaction mixture was quenched with water, extracted with CH₂Cl₂ (3×), washed with 10% HCl, dried over MgSO₄ and concentrated under reduced pressure. The desired material was obtained after flash chromatography on silica gel (ISCO COMBI-FLASH®) eluting with MeOH/CH₂Cl₂ (0-10%).

### Step 2: (+/−)-trans-t-butyl 3-[2-chloro-4-(4,4,5,5-tetramethyl-1,3,2-dioxaborolan-2-yl)phenyl]-4-(1-methyl-2-oxo-1,2-dihydropyridin-4-yl)piperidine-1-carboxylate

A mixture of 4,4,4′,4′,5,5,5′,5′-octamethyl-2,2′-bi-1,3,2-dioxaborolane (443 mg, 1.74 mmol), (+/−)-trans-t-butyl 3-(4-bromo-2-chlorophenyl)-4-(1-methyl-2-oxo-1,2-dihydropyridin-4-yl)piperidine-1-carboxylate from step 8, example 20 (600 mg, 1.25 mmol), potassium acetate (367 mg, 3.74 mmol) and 1,1′-bis(diphenylphosphino)ferrocene-palladium(II) dichloride dichoromethane complex (31 mg, 0.037 mmol) in dioxane (6.2 mL) was degassed with nitrogen and heated under reflux for 24 hours. The solvent was evaporated, the residue was diluted with saturated aqueous solution of NH₄Cl and extracted with EtOAc (3×). The combined organic fractions were dried over MgSO₄ and the solvent was concentrated under reduced pressure. The desired material was obtained after flash chromatography on silica gel (ISCO COMBI-FLASH®) eluting with MeOH/CH₂Cl₂ (0-5%).

### Step 3: (+/−)-trans-t-butyl 3-{2′-[2-(acetylamino)ethyl]-3-chloro-5′-fluorobiphenyl-4-yl}-4-(1-methyl-2-oxo-1,2-dihydropyridin-4-yl)piperidine-1-carboxylate

A solution of (+/−)-trans-t-butyl 3-[2-chloro-4-(4,4,5,5-tetramethyl-1,3,2-dioxaborolan-2-yl)phenyl]-4-(1-methyl-2-oxo-1,2-dihydropyridin-4-yl)piperidine-1-carboxylate from the previous step (179 mg, 0.338 mmol) and N-[2-(2-bromo-4-fluorophenyl)ethyl]acetamide from step 1, example 72 (80 mg, 0.308 mmol) in THF (2.8 mL) and water (0.28 mL) was degassed with N₂. Palladium(II) acetate (3 mg, 0.015 mmol), 2-dicyclohexylphosphino-2′,6′-dimethoxy-1,1′-biphenyl (13 mg, 0.031 mmol) and potassium phosphate tribasic (196 mg, 0.923 mmol) were added and the mixture was degassed again with N₂. The mixture was heated at 80° C. for 1 hour, 40 minutes. The solvent was evaporated, the residue was diluted with water and extracted with EtOAc (3×), dried over MgSO₄ and the solvent was concentrated under reduced pressure. The desired material was obtained after flash chromatography on silica gel (ISCO COMBI-FLASH®) eluting with MeOH/CH₂Cl₂ (0-5%).

### Step 4: (+/−)-trans-N-(2-{3′-chloro-5-fluoro-4′-[4-(1-methyl-2-oxo-1,2-dihydropyridin-4-yl)piperidin-3-yl]biphenyl-2-yl}ethyl)acetamide

To a solution of (+/−)-trans-t-butyl 3-{2′-[2-(acetylamino)ethyl]-3-chloro-5′-fluorobiphenyl-4-yl}-4-(1-methyl-2-oxo-1,2-dihydropyridin-4-yl)piperidine-1-carboxylate from the previous step (50 mg, 0.086 mmol) in CH₂Cl₂ (0.43 mL) was added 4M HCl in dioxane (0.15 mL, 0.60 mmol) and the reaction mixture was stirred at room temperature for 16 hours. The solvent was evaporated, the residue was taken in 2N NaOH, was extracted with EtOAc (4×), dried over Na₂SO₄ and concentrated under reduced pressure. The title compound was obtained after flash chromatography on silica gel eluting with MeOH(NH₃)/CH₂Cl₂ (3-5%).

### Example 73

### Structure 66: (+/−)-trans-N-(2-{3′-chloro-4,5-dimethoxy-4′-[4-(1-methyl-2-oxo-1,2-dihydropyridin-4-yl)piperidin-3-yl]biphenyl-2-yl}ethyl)acetamide

### Step 1: N-[2-(2-bromo-4,5-dimethoxyphenyl)ethyl]acetamide

To a solution of commercially available 2-(2-bromo-4,5-dimethoxyphenyl)ethanamine (200 mg, 0.769 mmol) and Et₃N (0.21 mL, 1.54 mmol) in CH₂Cl₂ (7.7 mL) was slowly added AcCl (57 μL, 0.807 mmol) and the resulting mixture was stirred at room temperature for 2 hours. The reaction mixture was quenched with water, extracted with CH₂Cl₂ (3×), washed with 10% HCl, dried over MgSO₄ and concentrated under reduced pressure. The desired material was obtained after flash chromatography on silica gel (ISCO COMBI-FLASH®) eluting with EtOAc/Hexanes (0-100%).

### Step 2: (+/−)-trans-t-butyl 3-{2′-[2-(acetylamino)ethyl]-3-chloro-4′,5′-dimethoxybiphenyl-4-yl}-4-(1-methyl-2-oxo-1,2-dihydropyridin-4-yl)piperidine-1-carboxylate

A solution of (+/−)-trans-t-butyl 3-[2-chloro-4-(4,4,5,5-tetramethyl-1,3,2-dioxaborolan-2-yl)phenyl]-4-(1-methyl-2-oxo-1,2-dihydropyridin-4-yl)piperidine-1-carboxylate from step 2, example 72 (135 mg, 0.255 mmol) and N-[2-(2-bromo-4,5-dimethoxyphenyl)ethyl]acetamide from the previous step (70 mg, 0.232 mmol) in THF (2.1 mL) and water (0.21 mL) was degassed with N₂. Palladium(II) acetate (2.6 mg, 0.012 mmol), 2-dicyclohexylphosphino-2′,6′-dimethoxy-1,1′-biphenyl (9.5 mg, 0.023 mmol) and potassium phosphate tribasic (0.148 g, 0.695 mmol) were added and the mixture was degassed again with N₂. The mixture was heated at 80° C. for 2 hours. The solvent was evaporated, the residue was diluted with water and extracted with EtOAc (3×), dried over MgSO₄ and concentrated under reduced pressure. The desired material was obtained after flash chromatography on silica gel (ISCO COMBI-FLASH®) eluting with MeOH/CH₂Cl₂ (0-5%).

### Step 3: (+/−)-trans-N-(2-{3′-chloro-4,5-dimethoxy-4′-[4-(1-methyl-2-oxo-1,2-dihydropyridin-4-yl)piperidin-3-yl]biphenyl-2-yl}ethyl)acetamide

To a solution of (+/−)-trans-t-butyl 3-{2′-[2-(acetylamino)ethyl]-3-chloro-4′,5′-dimethoxybiphenyl-4-yl}-4-(1-methyl-2-oxo-1,2-dihydropyridin-4-yl)piperidine-1-carboxylate from the previous step (41 mg, 0.066 mmol) in CH₂Cl₂ (0.33 mL) was added 4M HCl in dioxane (0.20 mL, 0.80 mmol) and the reaction mixture was stirred at room temperature for 20 hours. The solvent was evaporated, the residue was taken in 2N NaOH, was extracted with EtOAc (4×), dried over Na₂SO₄ and concentrated under reduced pressure. The title compound was obtained after flash chromatography on silica gel eluting with MeOH(NH₃)/CH₂Cl₂ (0-5%).

### Example 74

### Assays Demonstrating Biological Activity

### Inhibition of Human Recombinant Renin

Human recombinant renin (Proteos) in 50 mM MOPS pH 7.4, 100 mM NaCl, 0.002% Tween 20 at a final concentration of 100 μM is incubated with inhibitors from a 50 fold concentrated DMSO solution and 6 μM of an internally-quenched fluorescent peptide: DNP-Lys-His-Pro-Phe-His-Leu-Val-Ile-His-D,L-Amp (SEQ ID NO: 1); Paschalidou K. et al., Biochem J., 2004, 382, 1031). The reactions take place in a Costar 384 well black plate (#3573) at 37° C. for 3 hours. Fluorescence is measured at times 0 and 3 hours with a SpectraMax Gemini EM reader set at an excitation wavelength of 328 nm and at an emission wavelength of 388 nm. Background fluorescence at t=0 is subtracted from the measurement at t=3 hours. Inhibitory activity of the compounds is expressed as IC₅₀.

### Inhibition of Renin in Human Plasma

Human EDTA-collected plasma is rapidly thawed in warm water and centrifuged at 2900 g for 15 minutes at 4° C. The supernatant is collected and recombinant renin (Proteos) is added at a final concentration of 1 nM. The plasma is transferred to a Costar black 384 well plate (#3573). Renin inhibitors are added from a 17.5 fold concentrated DMSO solution and pre-incubated at 37° C. for 10 minutes. The internally-quench fluorescent peptide QXL520™-Lys-His-Pro-Phe-His-Leu-Val-Ile-His-Lys (5-FAM) (Anaspec), SEQ ID NO: 2, is diluted in 3M Tris pH 7.2, 200 mM EDTA and added to the plasma. The final concentrations are: 6 μM substrate, 342 mM Tris, 23 mM EDTA. The plate is incubated at 37° C. for 1 hour. The plate is read in a SpectraMax Gemini EM reader set at an excitation wavelength of 490 nm and an emission wavelength of 520 nM at times 0 and 1 hour. Background fluorescence at t=0 is subtracted from the measurement at t=1 hour. Inhibitory activity of the compounds is expressed as IC₅₀.

1-20. (canceled)

21. A compound of Formula III, or a pharmaceutically acceptable salt thereof, having the formula 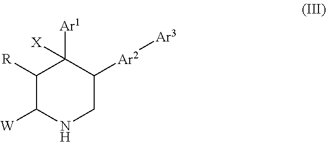
wherein:
Ar¹ is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of:
1) oxo,
2) OH,
3) CN,
4) halogen,
5) NH₂,
6) COOH,
7) OCF₂H,
8) OCF₃,
9) CF₃,
10) C₁-C₆alkyl,
11) OC₁-C₆alkyl,
12) C₃-C₆cycloalkyl,
13) C₃-C₆cycloalkyl,
14) C₂-C₆alkenyl,
15) C₁-C₆alkoxy,
16) (CH₂)₁₋₃OC₁-C₃alkyleneOC₁-C₆alkyl,
17) O(CH₂)₁₋₃OC₁-C₃alkyleneOC₁-C₆alkyl,
18) (CH₂)₁₋₃OC₁-C₃alkyleneOC₃-C₆cycloalkyl,
19) C(O)NHC₁-C₆alkyl,
20) NHC(O)C₁-C₆alkyl,
21) S(O)₀₋₂C₁-C₆alkyl,
22) O(CH₂)₁₋₄OAr⁴, and
23) (CH₂)₁₋₅OAr⁴,
wherein Ar⁴ is as described herein and wherein substituents (10)-(21) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) S(O)₀₋₂C₁-C₆alkyl,
Ar² is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of:
1) OH,
2) CN,
3) halogen,
4) COOH,
5) OCF₂H,
6) OCF₃,
7) CF₃,
8) C₁-C₆alkyl,
9) C₃-C₆cycloalkyl,
10) C₂-C₆alkenyl,
11) C₁-C₆alkoxy,
12) C(O)C₁-C₆alkyl,
13) NHC(O)C₁-C₆alkyl, and
14) S(O)₀₋₂C₁-C₆alkyl,
15) R²
16) R³
wherein substituents (8)-(14) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) S(O)₀₋₂C₁-C₆alkyl,
wherein R² is selected from the group consisting of
a) H,
b) C₁-C₆alkyl,
c) C₃-C₆cycloalkyl,
d) C₀-C₃alkyleneC(O)C₁-C₆alkyl,
e) C₀-C₃alkyleneC(O)OR¹, and
f) C₀-C₃alkyleneCONHR¹,
wherein R³ is selected from the group consisting of:
1) halogen as long as it is not attached to a N atom directly,
2) hydrogen,
3) CF₃,
4) C₁-C₆alkyl,
5) C₃-C₆cycloalkyl,
6) CH(OH),
7) C(O)C₁-C₆alkyl, and
8) CH(OH)C₁-C₆alkyl;
unsubstituted or substituted with one, two, three or four substituents independently selected from the group consisting of: OH, halogen, CF₃, and C₁-C₃alkyl,
Ar³ is an unsubstituted or substituted aryl ring, or an unsubstituted or substituted heteroaryl ring containing 1 to 3 heteroatoms independently selected from O, S and N, wherein the substituents on the substituted aryl ring and substituted heteroaryl ring consist of one, two or three substituents independently selected from the group consisting of:
1) OH,
2) CN,
3) halogen,
4) COOH,
5) OCF₂H,
6) OCF₃,
7) CF₃,
8) C₁-C₆alkyl,
9) C₃-C₆cycloalkyl,
10) C₂-C₆alkenyl,
11) C₁-C₆alkoxy,
12) C₁-C₃alkyleneNH₂,
13) C₁-C₃alkyleneNHC(O)NH₂,
14) C₁-C₃alkyleneOC₁-C₆alkyl,
15) OC₁-C₃alkyleneOC₁-C₆alkyl,
16) C₁-C₃alkyleneNHC(O)C₁-C₆alkyl,
17) C₁-C₃alkyleneNHC(O)OC₁-C₆alkyl,
18) C₁-C₃alkyleneNHC(O)NR¹C₁-C₆alkyl,
19) C₁-C₃alkyleneNHC(O)C₃-C₆cycloalkyl,
20) C₁-C₃alkyleneNHC(O)OC₃-C₆cycloalkyl,
21) C₁-C₃alkyleneNHC(O)NR¹C₃-C₆cycloalkyl,
22) C₁-C₃alkyleneNHS(O)₂C₁-C₆alkyl,
23) C₀-C₃alkyleneC(O)NR¹C₁-C₆alkyl,
24) C₀-C₃alkyleneS(O)₀₋₂C₁-C₆alkyl,
25) C₁-C₃alkyleneNHC(O)Ar⁵,
26) C₀-C₃alkyleneO(CH₂)₀₋₃Ar⁵,
27) C₁-C₃alkyleneNHC(O)O(CH₂)₀₋₃Ar⁵, and
28) C₀-C₃alkyleneHet,
wherein Het, Ar⁵, and R¹ are as described herein, wherein substituents (8)-(22) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy,
i) C₁-C₃alkyleneOC₁-C₆alkyl, and
j) S(O)₀₋₂C₁-C₆alkyl, and
wherein substituents (23)-(26) are unsubstituted or substituted with 1-3 halogens,
Ar⁴ is an unsubstituted or substituted 5- or 6-membered aryl ring, or an unsubstituted or substituted 5 or 6-membered heteroaryl ring containing 1 to 2 heteroatoms independently selected from O, S and N, wherein the substituents on the substituted aryl ring or substituted heteroaryl ring consist of one, two or three substituents independently selected from the group consisting of:
1) CN,
2) halogen,
3) OCF₂H,
4) OCF₃,
5) CF₃,
6) C₁-C₆alkyl,
7) C₃-C₆cycloalkyl,
8) C₁-C₆alkoxy, and
9) S(O)₀₋₂C₁-C₆alkyl,
wherein substituents (6)-(9) are unsubstituted or substituted with one, two, three or four substituents independently selected from the group consisting of:
a) OH,
b) COOH,
c) CN,
d) CF₃,
e) C₁-C₆alkoxy, and
f) S(O)₀₋₂C₁-C₆alkyl,
Ar⁵ is an unsubstituted or substituted aryl ring, or an unsubstituted or substituted heteroaryl ring containing 1 to 4 heteroatoms independently selected from O, S and N, wherein the substituents on the substituted aryl ring and substituted heteroaryl ring consist of one, two or three substituents independently selected from the group consisting of:
1) OH,
2) CN,
3) halogen,
4) COOH,
5) OCF₂H,
6) OCF₃,
7) CF₃,
8) C₁-C₆alkyl,
9) C₃-C₆cycloalkyl,
10) C₂-C₆alkenyl, and
11) C₁-C₆alkoxy,
wherein substituents (8)-(11) are unsubstituted or substituted with one, two, three or four substituents independently selected from the group consisting of:
a) OH,
b) halogen,
c) COOH,
d) CN,
e) CF₃,
f) C₁-C₆alkyl,
g) C₁-C₆cycloalkyl,
h) C₁-C₆alkoxy, and
i) S(O)₀₋₂C₁-C₆alkyl,
R is selected from the group consisting of:
1) hydrogen,
2) halogen,
3) OH,
4) COOH,
5) COOC₁-C₆alkyl,
6) CF₃,
7) CN,
8) C₁-C₆alkyl,
9) C₃-C₆cycloalkyl,
10) (CH₂)₁₋₃OC₁-C₆alkyl,
11) OC₁-C₆alkyl, and
12) CONHC₁-C₆alkyl,
wherein substituents (8)-(12) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) S(O)₀₋₂C₁-C₆alkyl,
R¹ is selected from the group consisting of:
1) hydrogen,
2) C₁-C₆alkyl, and
3) C₃-C₆cycloalkyl,
wherein substituents (2)-(3) are unsubstituted or substituted with one, two, three or four substituents independently selected from the group consisting of:
a) OH,
b) halogen,
c) COOH,
d) CN,
e) CF₃,
f) C₁-C₆alkyl,
g) C₁-C₆alkoxy, and
h) S(O)₀₋₂C₁-C₆alkyl,
W is selected from the group consisting of:
1) hydrogen,
2) CN,
3) COOR¹,
4) C₁-C₆alkyl, and
5) C₃-C₆cycloalkyl;
wherein alkyl and cycloalkyl are unsubstituted or substituted with 1-3 substitutents selected from the group consisting of:
a) halogen,
b) OR¹
c) COOR¹,
d) CN,
e) CONHR¹,
f) CON(R¹)R⁴,
g) OC(O)N(R¹)R⁴,
h) NHC(O)N(R¹)R⁴
i) NHC(O)R¹, and
j) NHC(O)Ar⁴;
wherein R⁴ is selected from the group consisting of: hydrogen, C₁-C₆alkyl, C₃-C₆cycloalkyl, and Ar⁴;
X is selected from the group consisting of:
1) hydrogen,
2) OH,
3) CN,
4) halogen,
5) COOH,
6) CF₃,
7) COOC₁-C₆alkyl,
8) OC₁-C₆alkyl,
9) OC(O)NH₂
10) O(CH₂)₁₋₃Ar⁵, and
11) O(CH₂)₁₋₃Het,
wherein substituents (7)-(8) and (10)-(11) are unsubstituted or substituted with one, two, three or four substituents independently selected from the group consisting of:
a) OH,
b) halogen,
c) COOH,
d) CN,
e) CF₃,
f) C₁-C₆alkyl,
g) OC₁-C₆alkyl, and
h) S(O)₀₋₂C₁-C₆alkyl,
Het is a 5-7-membered substituted or unsubstituted heterocyclic ring containing 1-4 heteroatoms independently selected from O, N and S; and
Q is absent or selected from the group consisting of:
1) a bond,
2) CH₂,
3) CH₂CH₂,
4) CH₂OCH₂,
5) CH₂S(O)₀₋₂CH₂,
6) CH₂NR²CH₂, and
7) CONR²CH₂,
wherein R² is as defined above for Ar².

22. The compound of claim 21 wherein Ar¹ is an unsubstituted or substituted aryl ring, or an unsubstituted or substituted heteroaryl ring containing 1-3 N heteroatoms, wherein the substituents on the substituted aryl ring and substituted heteroaryl ring consist of one, two or three substituents independently selected from the group consisting of: oxo, halogen, C₁-C₆alkyl, OC₁-C₆alkyl, NH₂, (CH₂)₁₋₃OC₁-C₃alkyleneOC₁-C₆alkyl, O(CH₂)₁₋₃OC₁-C₃alkyleneOC₁-C₆alkyl, and (CH₂)₁₋₃OC₁-C₃alkyleneOC₃-C₆cycloalkyl, wherein the alkyl substituents are unsubstituted or substituted with 1-3 halogen or C₁-C₆alkyl substituents, or a pharmaceutically acceptable salt thereof.

23. The compound of claim 21 wherein Ar¹ is selected from the group consisting of: 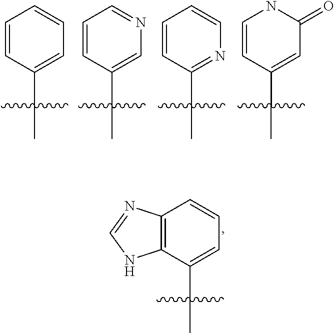
unsubstituted or substituted at any carbon atom with one, two or three substituents independently from the group consisting of:
1) oxo,
2) OH,
3) CN,
4) halogen,
5) NH₂,
6) COOH,
7) OCF₂H,
8) OCF₃,
9) CF₃,
10) C₁-C₆alkyl,
11) OC₁-C₆alkyl,
12) C₃-C₆cycloalkyl,
13) C₃-C₆cycloalkyl,
14) C₂-C₆alkenyl,
15) C₁-C₆alkoxy,
16) (CH₂)₁₋₃OC₁-C₃alkyleneOC₁-C₆alkyl,
17) O(CH₂)₁₋₃OC₁-C₃alkyleneOC₁-C₆alkyl,
18) (CH₂)₁₋₃OC₁-C₃alkyleneOC₃-C₆cycloalkyl,
19) C(O)NHC₁-C₆alkyl,
20) NHC(O)C₁-C₆alkyl,
21) S(O)₀₋₂C₁-C₆alkyl,
22) O(CH₂)₁₋₄OAr⁴, and
23) (CH₂)₁₋₅OAr⁴,
wherein Ar⁴ is described in claim 21 and wherein substituents (10)-(21) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) S(O)₀₋₂C₁-C₆alkyl,
or a pharmaceutically acceptable salt thereof.

24. The compound of claim 21 wherein Ar¹ is selected from the group consisting of: 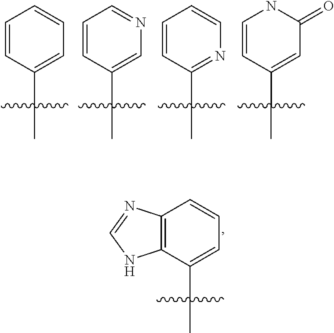
unsubstituted or substituted at any carbon atom with one, two or three substituents independently selected from the group consisting of: OH, NH₂, CN, halogen, C₁-C₆alkyl, C₃-C6cycloalkyl, OC₁-C₆alkyl, OC₃-C₆cycloalkyl, (CH₂)₁₋₃OC₁-C₃alkyleneOC₁-C₆alkyl, (CH₂)₁₋₃OC₁-C₃alkyleneOC₃-C₆cycloalkyl, O(CH₂)₁₋₄OAr⁴ and (CH₂)₁₋₅OAr⁴; said C₁-C₆alkyl, C₃-C₆cycloalkyl, OC₁-C₆alkyl, OC₃-C₆cycloalkyl, (CH₂)₁₋₃OC₁-C₃alkyleneOC₁-C₆alkyl, O(CH₂)₁₋₃OC₁-C₃alkyleneOC₁-C₆alkyl, (CH₂)₁₋₃OC₁-C₃alkyleneOC₃-C₆cycloalkyl, O(CH₂)₁₋₄OAr⁴ and (CH₂)₁₋₅OAr⁴ substituents unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) S(O)₀₋₂C₁-C₆alkyl,
wherein Ar⁴ is described in claim 21, or a pharmaceutically acceptable salt thereof.

25. The compound of claim 21 wherein Ar¹ is selected from the group consisting of: 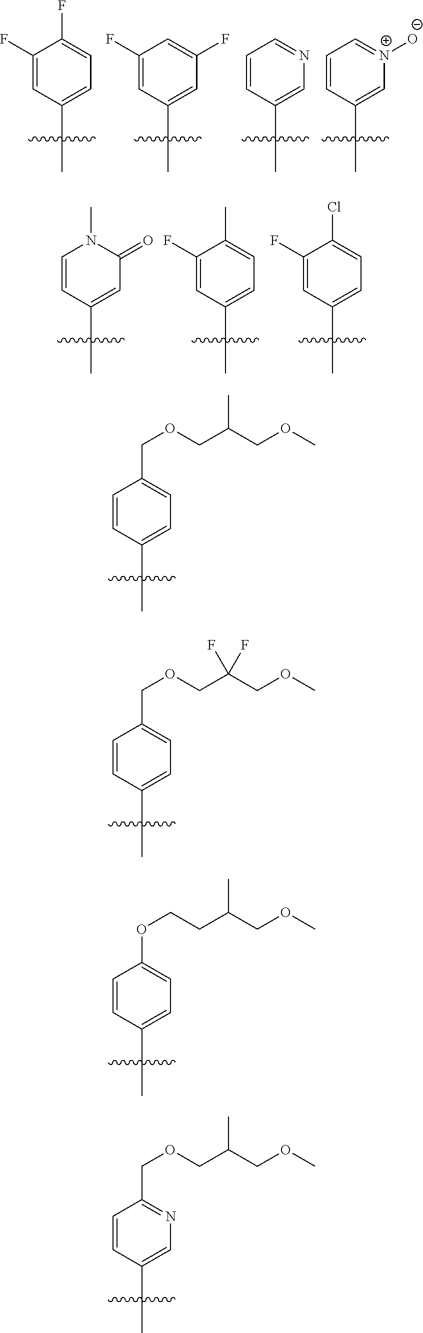 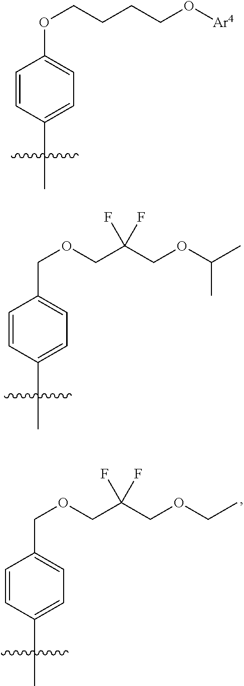
unsubstituted or substituted at any carbon atom with one, two, three or four substituents independently selected from the group consisting of: halogen, CF₃ and methyl, where Ar⁴ is described in claim 21, or a pharmaceutically acceptable salt thereof.

26. The compound of claim 21 wherein Ar¹ is selected from the group consisting of: 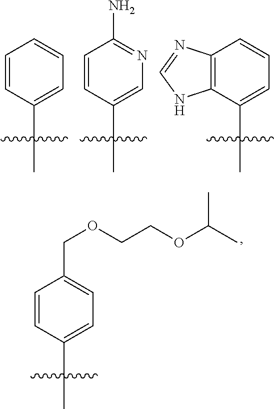
unsubstituted or substituted at any carbon atom with one, two, three or four substituents independently selected from the group consisting of: halogen, CF₃ and methyl, or a pharmaceutically acceptable salt thereof.

27. The compound of claim 21 wherein Ar²-Ar³ is selected from the group consisting of: 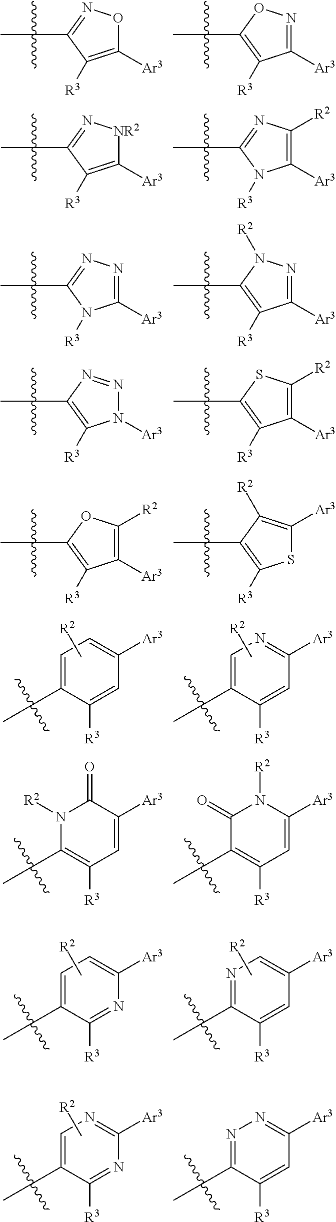
wherein R², R³, and Ar³ are described in claim 21, or a pharmaceutically acceptable salt thereof.

28. The compound of claim 21 wherein Ar²-Ar³ is selected from the group consisting of: 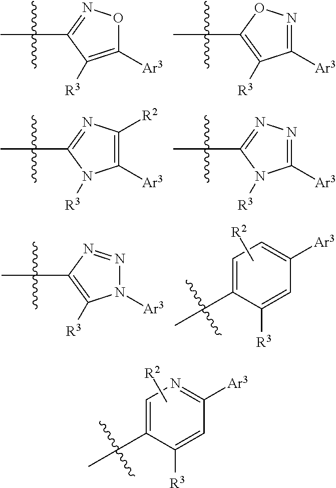
wherein R², R³, and Ar³ are described in claim 21, or a pharmaceutically acceptable salt thereof.

29. The compound of claim 21 wherein Ar²-Ar³ is selected from the group consisting of: 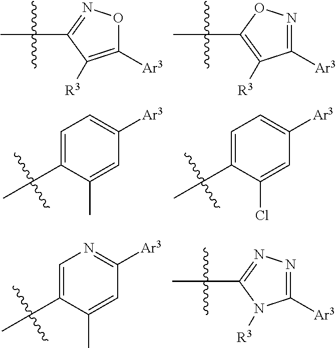
wherein Ar³ is described in claim 21, wherein R³ is selected from the group consisting of: hydrogen, halogen, Me, Et, isopropyl, cyclopropyl, CF₃, CH₃C(O), CH₃CH₂C(O), CH₃CH(OH) and CH₃CH₂CH(OH); said isopropyl, cyclopropyl, CH₃C(O), CH₃CH₂C(O), CH₃CH(OH) and CH₃CH₂CH(OH) unsubstituted or substituted with 1-3 halogens, or a pharmaceutically acceptable salt thereof.

30. The compound of claim 21 wherein Ar³ is selected from the group consisting of: 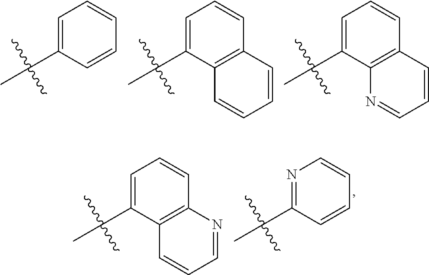
unsubstituted or substituted at any carbon atom with one, two or three substituents independently selected from the group consisting of:
1) OH,
2) CN,
3) halogen,
4) COOH,
5) OCF₂H,
6) OCF₃,
7) CF₃,
8) C₁-C₆alkyl,
9) C₃-C₆cycloalkyl,
10) C₂-C₆alkenyl,
11) C₁-C₆alkoxy,
12) C₁-C₃alkyleneNH₂,
13) C₁-C₃alkyleneNHC(O)NH₂,
14) C₁-C₃alkyleneOC₁-C₆alkyl,
15) C₁-C₃alkyleneNHC(O)C₁-C₆alkyl,
16) C₁-C₃alkyleneNHC(O)OC₁-C₆alkyl,
17) C₁-C₃alkyleneNHC(O)NR¹C₁-C₆alkyl,
18) C₁-C₃alkyleneNHC(O)C₃-C₆cycloalkyl,
19) C₁-C₃alkyleneNHC(O)OC₃-C₆cycloalkyl,
20) C₁-C₃alkyleneNHC(O)NR¹C₃-C₆cycloalkyl,
21) C₁-C₃alkyleneNHS(O)₂C₁-C₆alkyl,
22) C₀-C₃alkyleneC(O)NR¹C₁-C₆alkyl,
23) C₀-C₃alkyleneS(O)₀₋₂C₁-C₆alkyl,
24) C₁-C₃alkyleneNHC(O)Ar⁵,
25) C₀-C₃alkyleneO(CH₂)₀₋₃Ar⁵,
26) C₁-C₃alkyleneNHC(O)O(CH₂)₀₋₃Ar⁵, and
27) C₀-C₃alkyleneHet,
wherein Het, Ar⁵, and R¹ are described in claim 21, wherein substituents (8)-(21) are unsubstituted or substituted with one, two, three or four substituents independently selected from the group consisting of:
a) OH,
b) halogen,
c) COOH,
d) CN,
e) CF₃,
f) C₁-C₆alkyl,
g) C₃-C₆cycloalkyl,
h) C₁-C₆alkoxy, and
i) C₁-C₃alkyleneOC₁-C₆alkyl, and
j) S(O)₀₋₂C₁-C₆alkyl,
and substituents (20)-(23) are unsubstituted or substituted with 1-3 halogens, or a pharmaceutically acceptable salt thereof.

31. The compound of claim 21 wherein Ar³ is selected from the group consisting of: 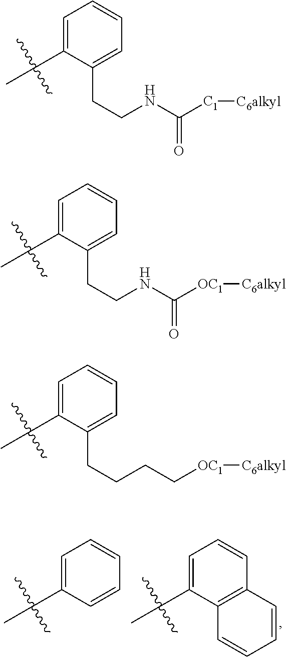
unsubstituted or substituted at any carbon atom with 1-2 groups independently selected from the group consisting of: halogen, CF₃ and Me, wherein R and X are independently selected from the group consisting of: H and OH, or a pharmaceutically acceptable salt thereof.

32. The compound of claim 21 wherein R is selected from the group consisting of:
1) H,
2) halogen,
3) OH,
4) OC₁-C₆alkyl,
5) COOC₁-C₆alkyl, and
6) CONHC₁-C₆alkyl,
wherein 4)-6) are unsubstituted or substituted with one, two, three or four substituents independently selected from the group consisting of: OH, halogen, CF₃, and C₁-C₃alkyl, or a pharmaceutically acceptable salt thereof.

33. The compound of claim 21 wherein R is hydrogen or halogen, or a pharmaceutically acceptable salt thereof.

34. The compound of claim 21 wherein W is selected from the group consisting of: hydrogen and C₁-C₆alkyl, unsubstituted or substituted with 1-3 halogen substitutents, or a pharmaceutically acceptable salt thereof.

35. The compound of claim 21 wherein X is selected from the group consisting of:
1) H,
2) halogen,
3) OH,
4) OC(O)NH₂,
5) OC₁-C₆alkyl, and
6) O(CH₂)₁₋₃Het,
wherein Het is defined in claim 21 and wherein 5)-6) are unsubstituted or substituted with one, two, three or four substituents independently selected from the group consisting of: OH, halogen, CF₃, and C₁-C₃alkyl, or a pharmaceutically acceptable salt thereof.

36. A compound from the following table:
Structure 1 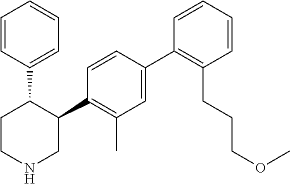
Structure 2 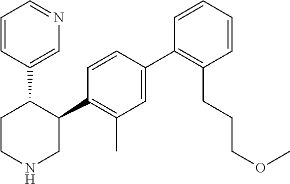
Structure 3 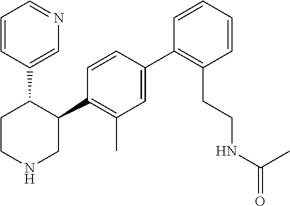
Structure 4 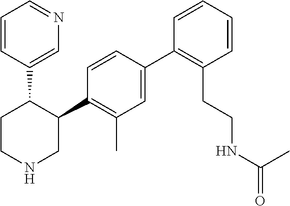
Structure 5 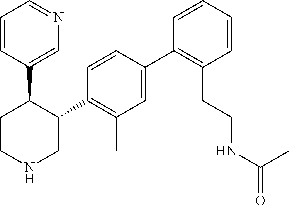
Structure 6 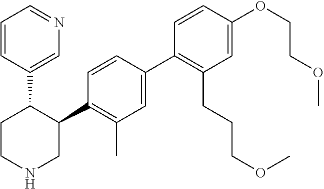
Structure 7 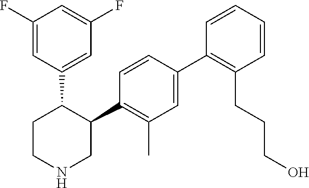
Structure 8 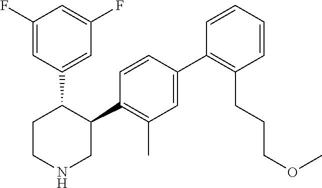
Structure 9 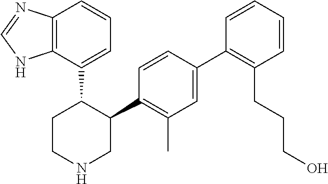
Structure 10 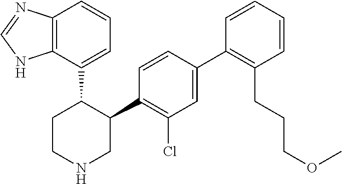
Structure 11 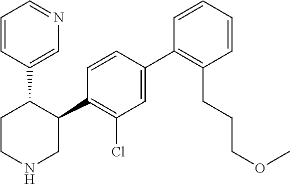
Structure 12 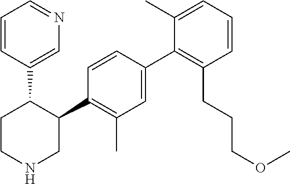
Structure 13 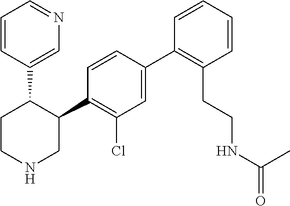
Structure 14 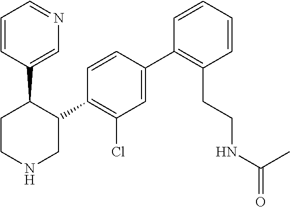
Structure 15 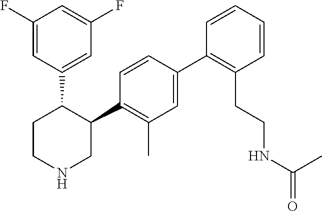
Structure 16 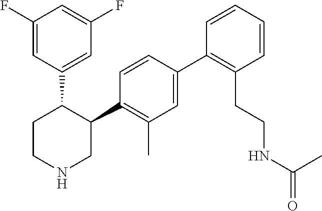
Structure 17 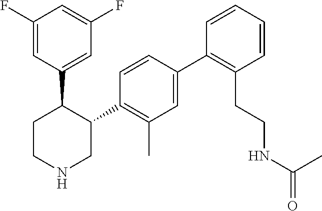
Structure 18 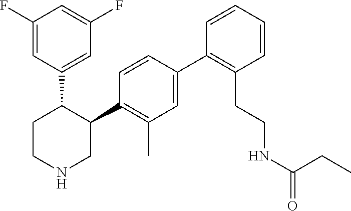
Structure 19 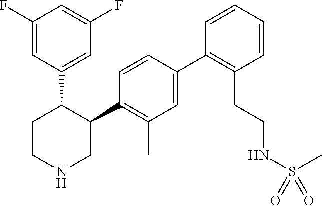
Structure 20 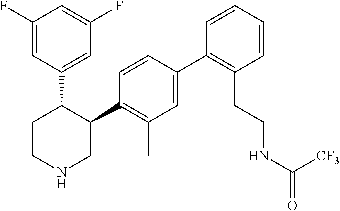
Structure 21 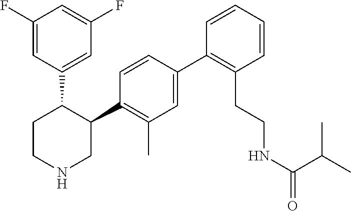
Structure 22 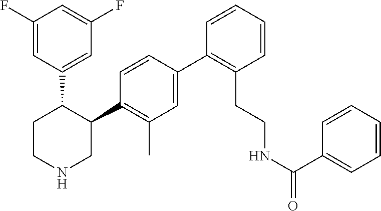
Structure 23 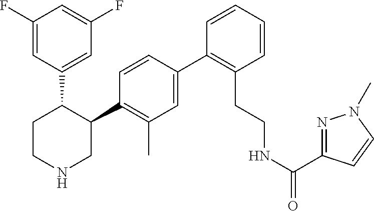
Structure 24 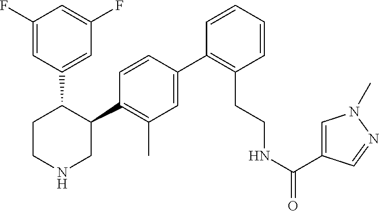
Structure 25 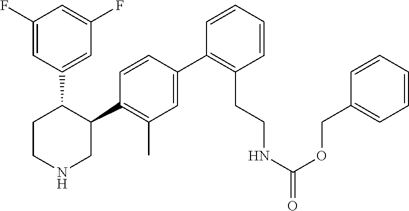
Structure 26 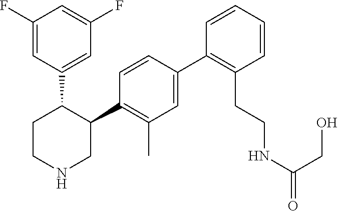
Structure 27 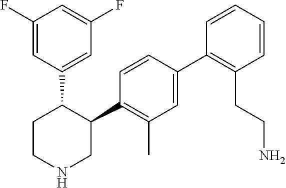
Structure 28 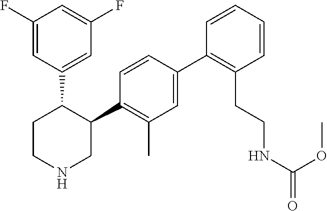
Structure 29 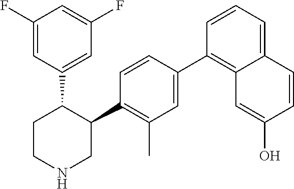
Structure 30 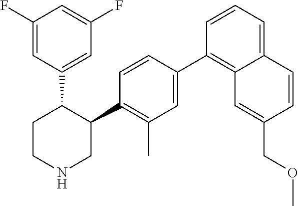
Structure 31 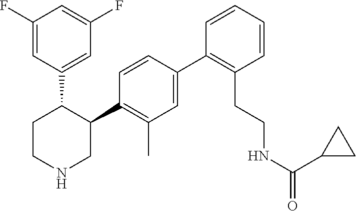
Structure 32 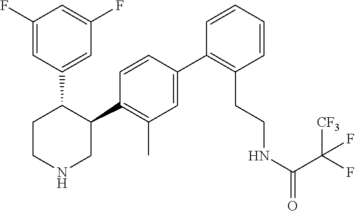
Structure 33 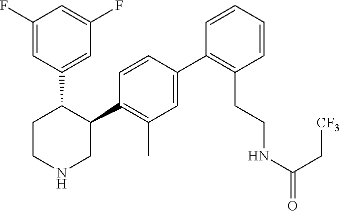
Structure 34 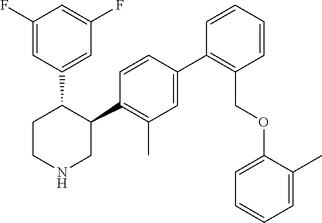
Structure 35 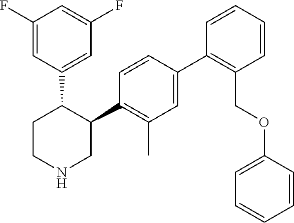
Structure 36 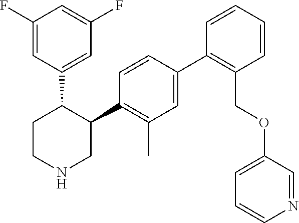
Structure 37 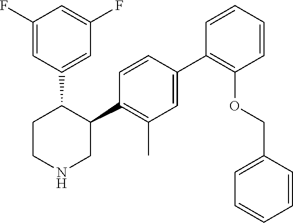
Structure 38 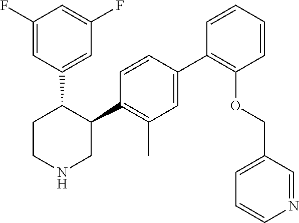
Structure 39 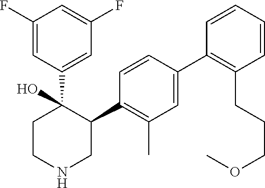
Structure 40 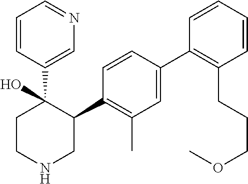
Structure 41 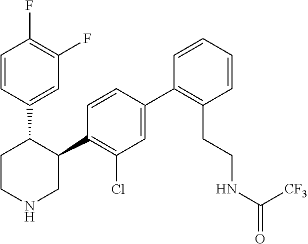
Structure 42 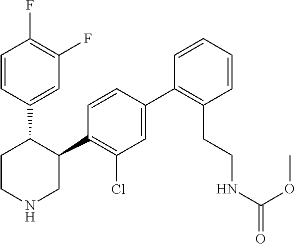
Structure 43 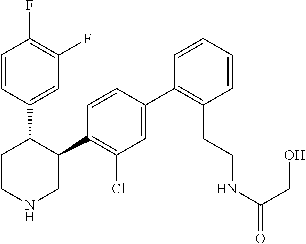
Structure 44 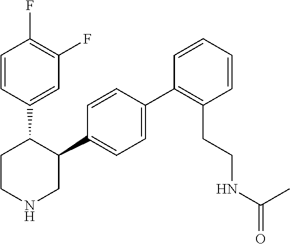
Structure 45 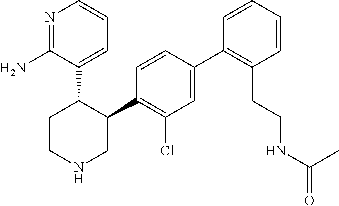
Structure 46 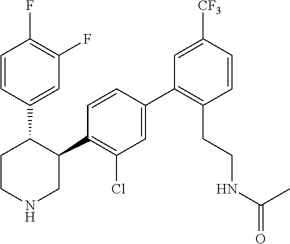
Structure 47 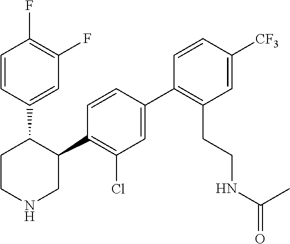
Structure 48 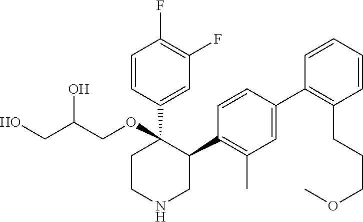
Structure 49 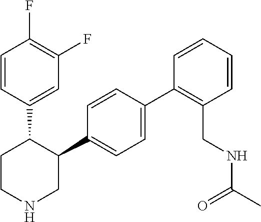
Structure 50 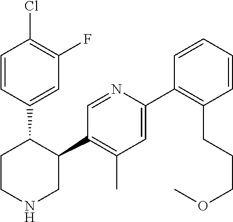
Structure 51 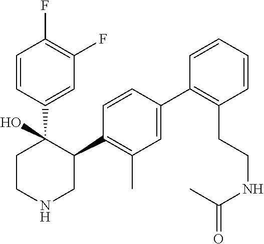
Structure 52 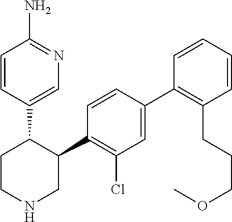
Structure 53 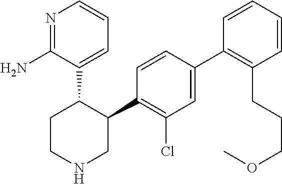
Structure 54 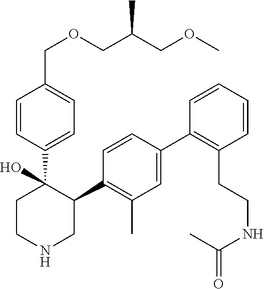
Structure 55 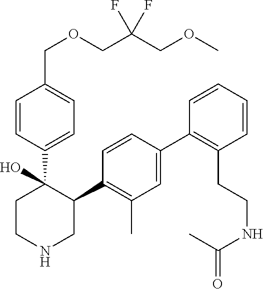
Structure 56 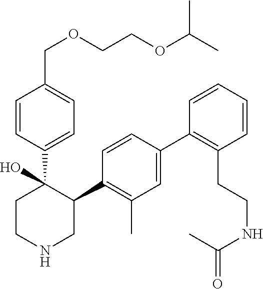
Structure 57 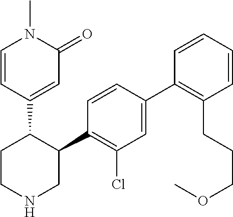
Structure 58 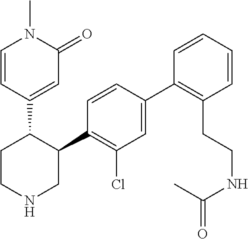
Structure 59 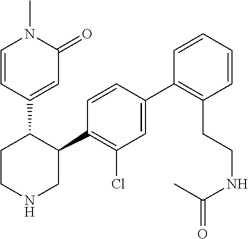
Structure 60 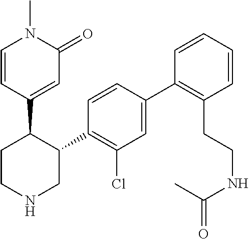
Structure 61 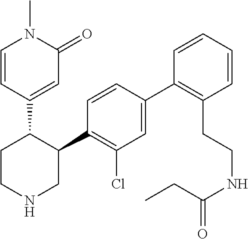
Structure 62 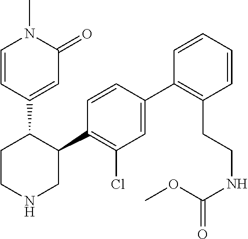
Structure 63 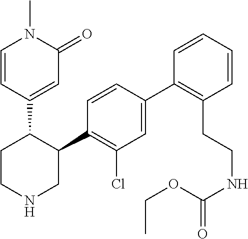
Structure 64 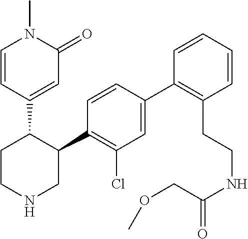
Structure 65 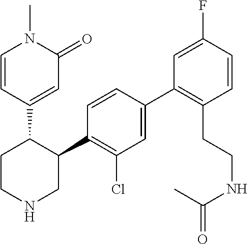
Structure 66 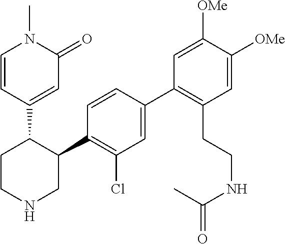
Structure 67 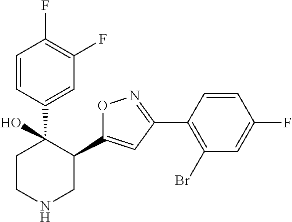
Structure 68 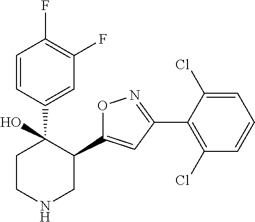
Structure 69 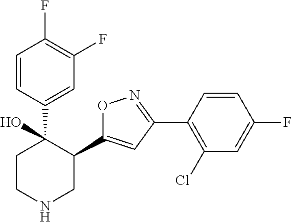
Structure 70 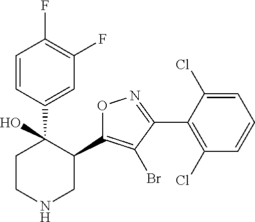
Structure 71 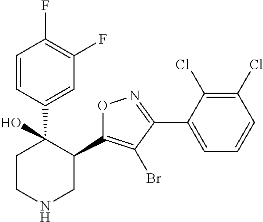
Structure 72 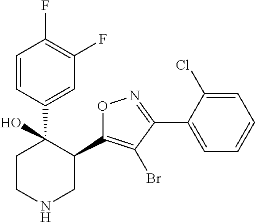
Structure 73 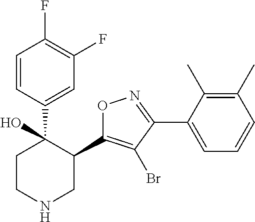
Structure 74 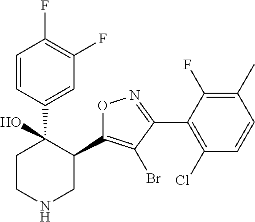
Structure 75 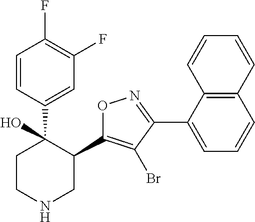
Structure 76 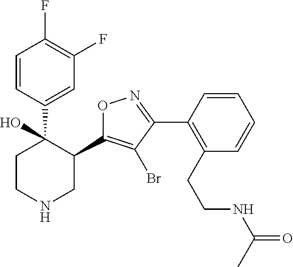
Structure 77 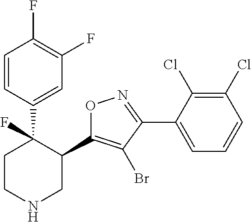
Structure 78 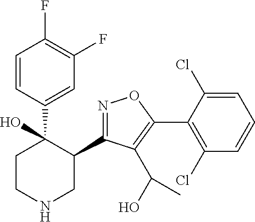
Structure 79 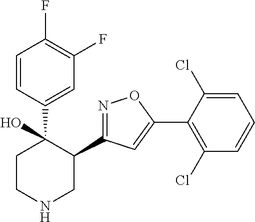
Structure 80 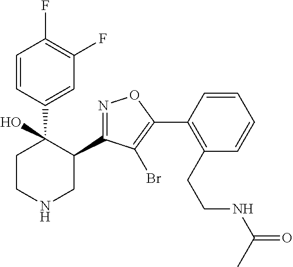
Structure 81 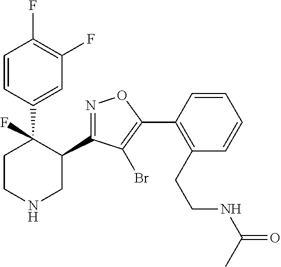
Structure 82 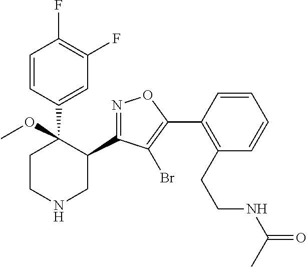
Structure 83 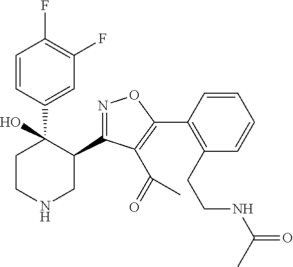
Structure 84 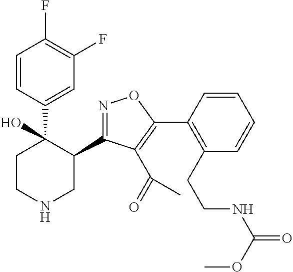
Structure 85 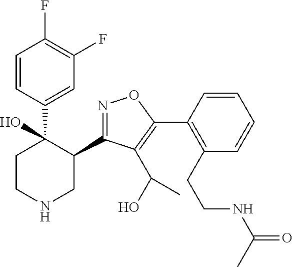
Structure 86 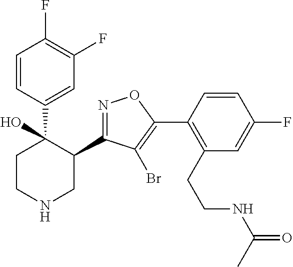
Structure 87 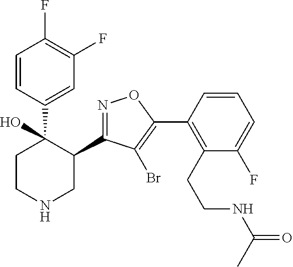
Structure 88 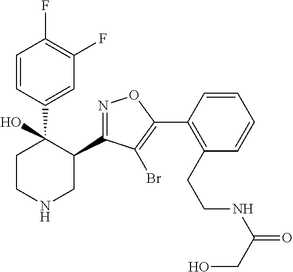
Structure 89 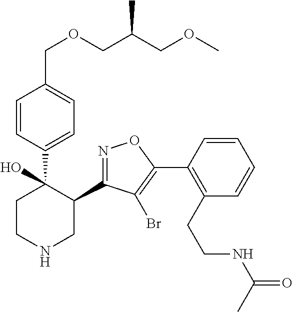
Structure 90 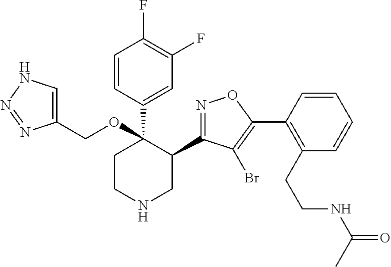
Structure 91 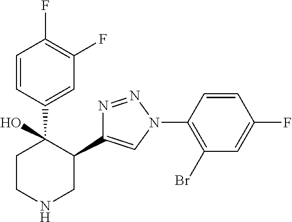
Structure 92 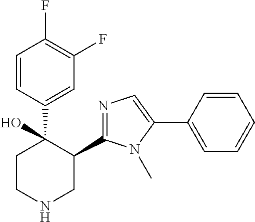
Structure 93 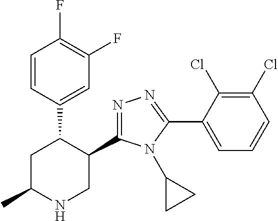
Structure 94 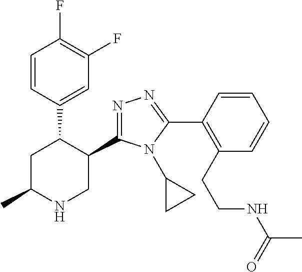
Structure 95 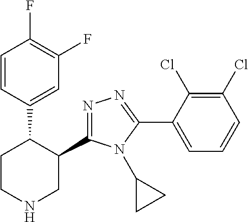
Structure 96 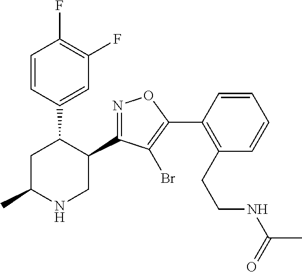
Structure 97 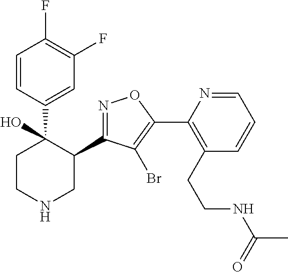
Structure 98 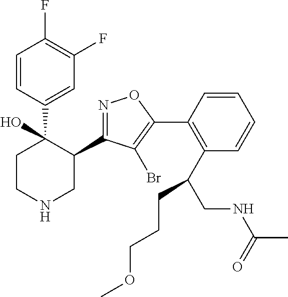
Structure 99 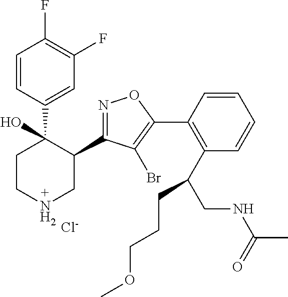
Structure 100 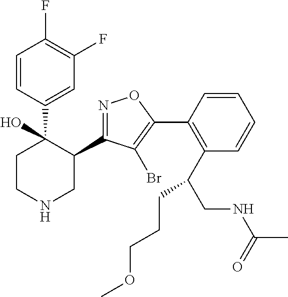
Structure 101 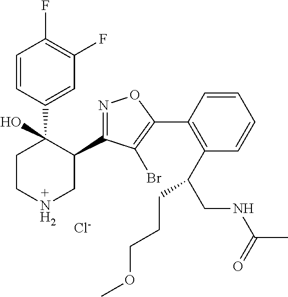
Structure 102 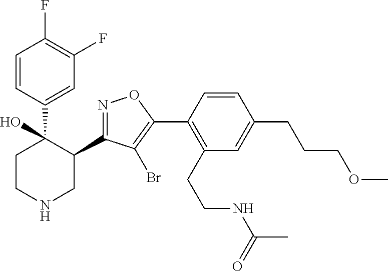
Structure 103 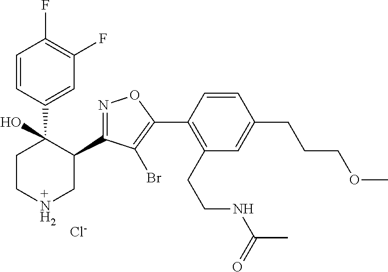
Structure 104 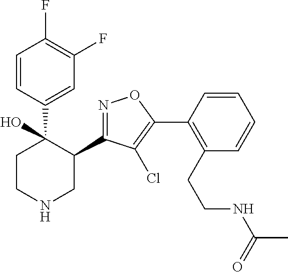
Structure 105 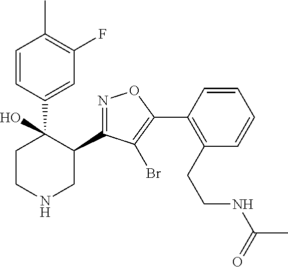
Structure 106 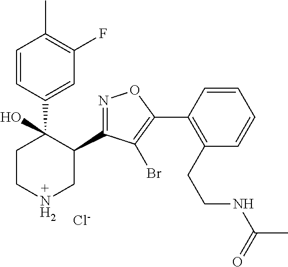
Structure 107 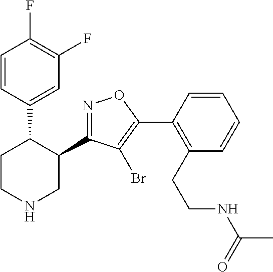
Structure 108 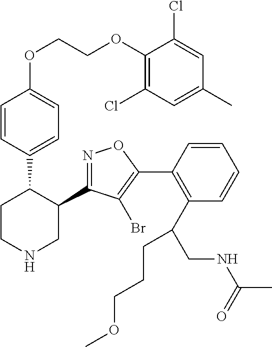
Structure 109 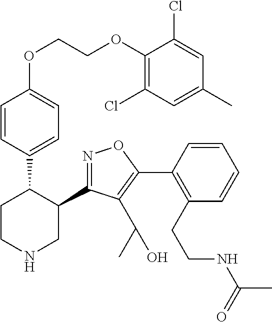
Structure 110 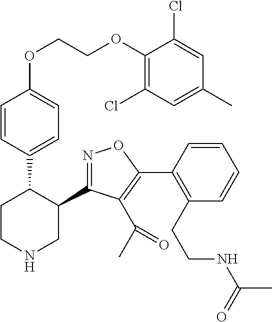
Structure 111 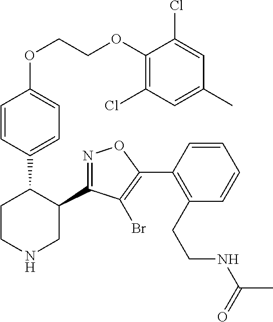
Structure 112 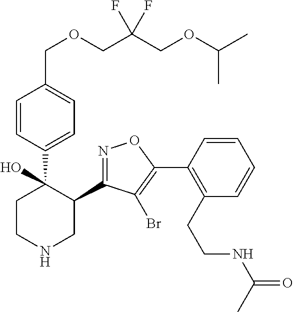
Structure 113 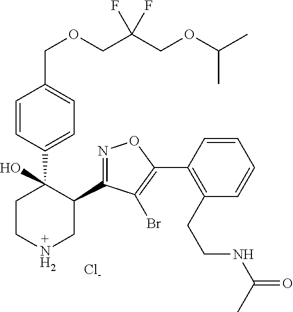
Structure 114 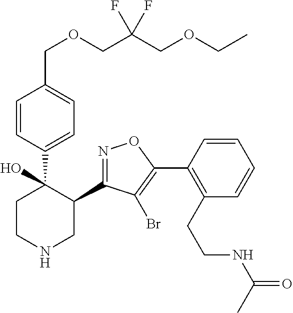
Structure 115 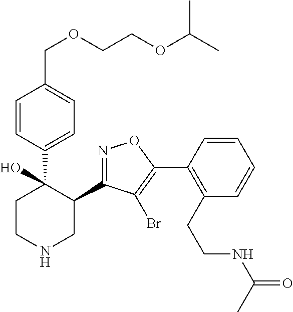
Structure 116 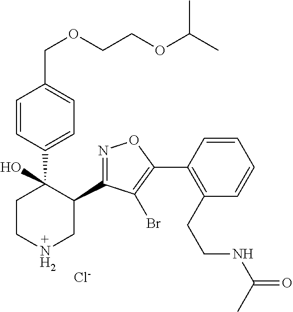
Structure 117 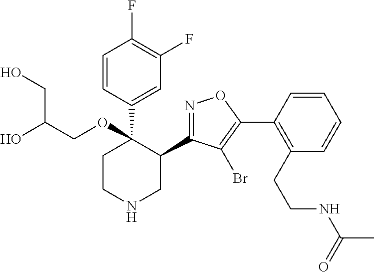
Structure 118 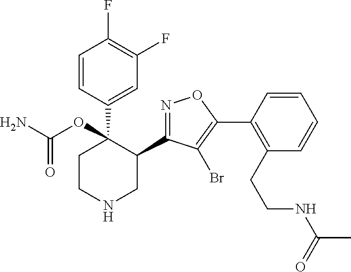
Structure 119 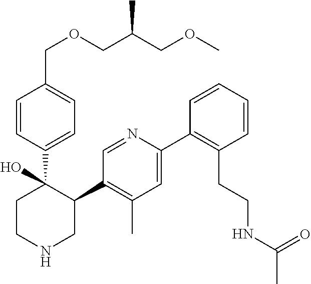
Structure 120 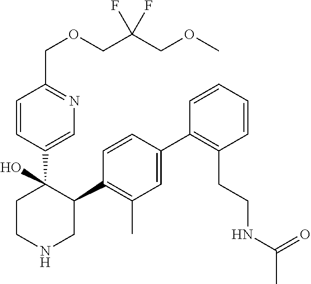
Structure 121 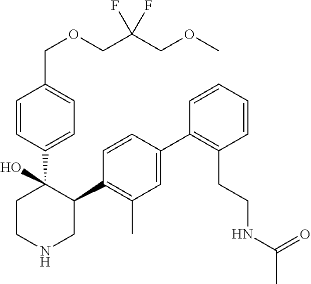
Structure 122 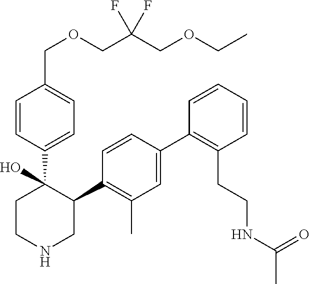
Structure 123 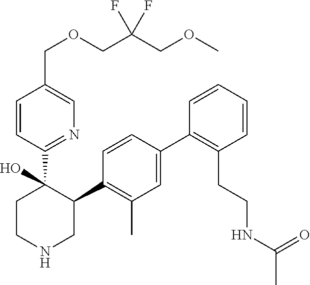
Structure 124 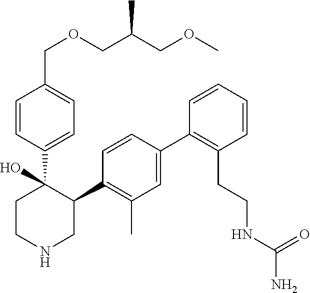
Structure 125 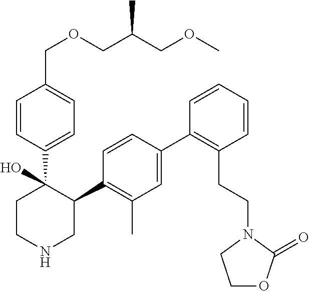
Structure 126 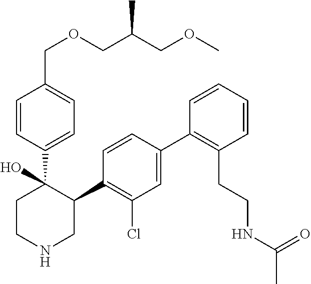
Structure 127 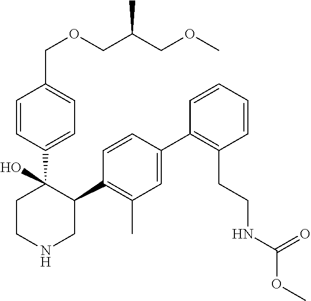
Structure 128 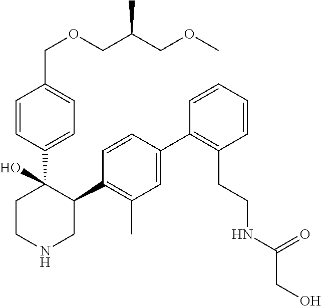
Structure 129 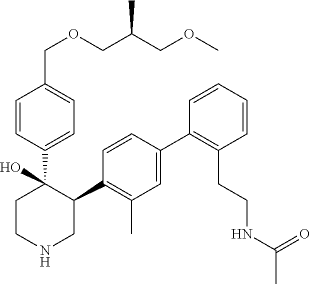
Structure 130 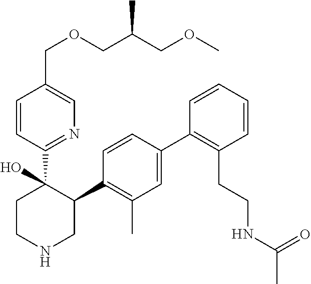
Structure 131 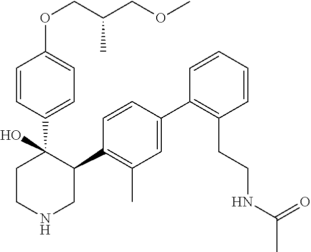
Structure 132 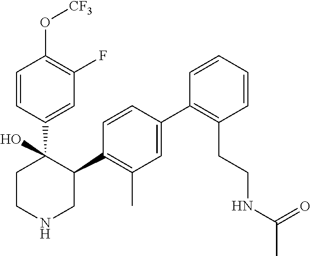
or a pharmaceutically acceptable salt thereof.

37. A compound from the following table:
Structure 54 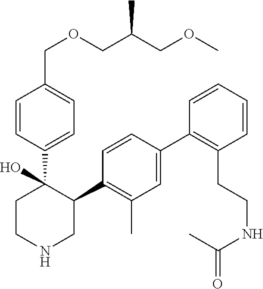
Structure 55 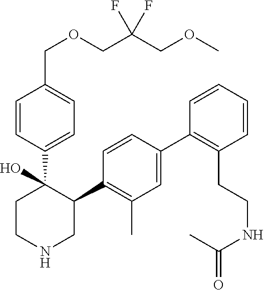
Structure 59 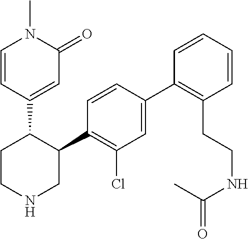
Structure 76 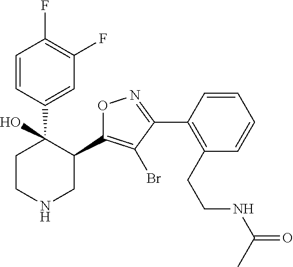
Structure 80 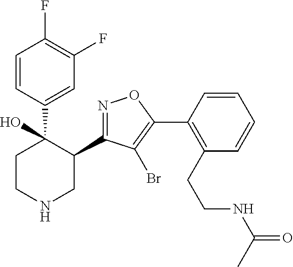
Structure 89 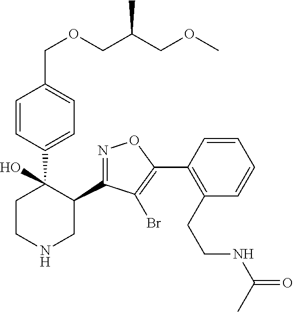
Structure 93 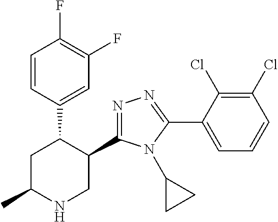
Structure 94 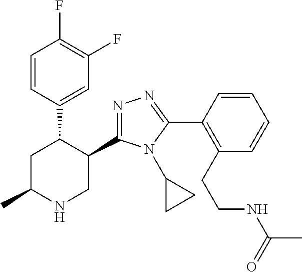
Structure 95 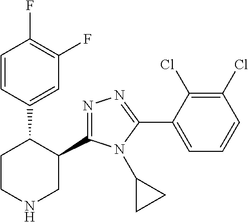
Structure 96 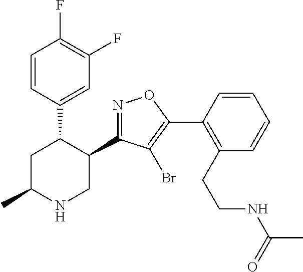
Structure 107 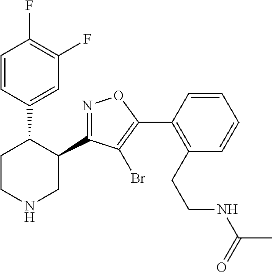
Structure 112 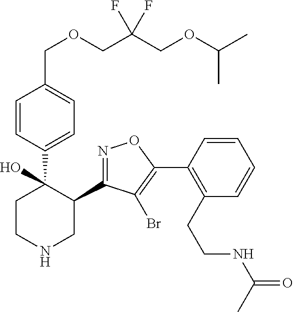
Structure 113 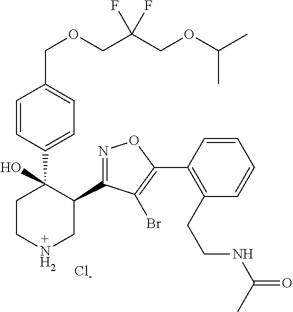
Structure 114 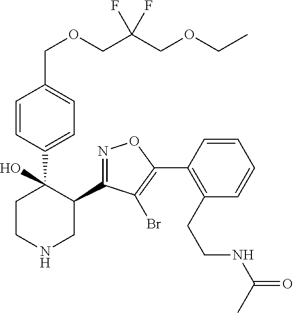
Structure 121 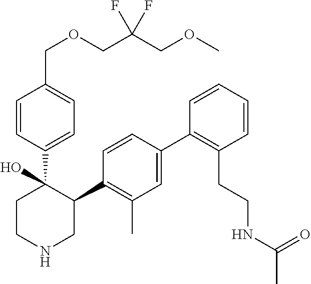
Structure 122 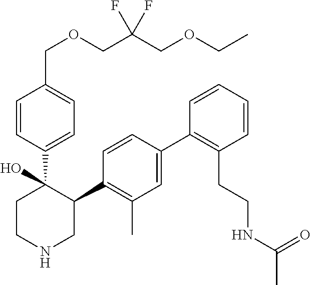
Structure 126 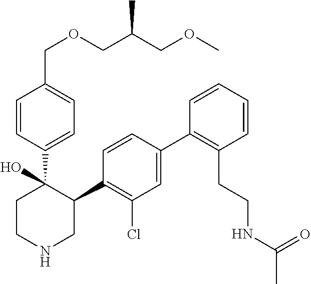
Structure 129 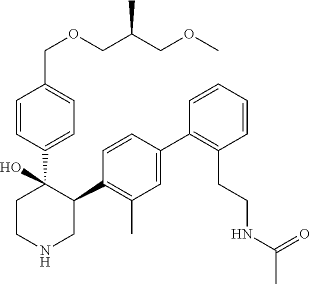
or a pharmaceutically acceptable salt thereof.

38. A pharmaceutical composition comprising an effective amount of a compound according to claim 21, or a pharmaceutically acceptable salt thereof, and a pharmaceutically acceptable carrier.

39. A method for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising the administration to a patient of a pharmaceutically active amount of a compound according to claim 21, or a pharmaceutically acceptable salt thereof.
